(19) United States
(12) Patent Application Publication
(10) Pub. No.: US 2026/0055413 A1
(43) Pub. Date: Feb. 26, 2026

(54) REVERSIR MOLECULES AND METHODS OF USE THEREOF

(71) Applicant: Alnylam Pharmaceuticals, Inc., Cambridge, MA (US)

(72) Inventors: Ivan Zlatev, Cambridge, MA (US); Adam Castoreno, Framingham, MA (US); Vasant R. Jadhav, Sharon, MA (US); Martin A. Maier, Belmont, MA (US); Anne Kasper, Roslindale, MA (US); Ho Chou TU, San Diego, CA (US); Donald Foster, Attleboro, MA (US)

(21) Appl. No.: 19/281,945

(22) Filed: Jul. 28, 2025

Related U.S. Application Data
(63) Continuation of application No. PCT/US2024/014761, filed on Feb. 7, 2024.
(60) Provisional application No. 63/444,375, filed on Feb. 9, 2023.

(51) Int. Cl. C12N 15/113

(52) U.S. Cl. CPC C12N 15/1136; C12N 2310/14; C12N 2310/315; C12N 2310/321; C12N 2310/3231; C12N 2310/351

(57) ABSTRACT

The present invention provides REVERSIR compounds which inhibit the RNAi interference activity of dsRNA agents comprising a thermally destabilizing nucleotide modification in the antisense strand, and methods of use thereof.

### RELATED APPLICATIONS

This application is a 35 § U.S.C. 111(a) continuation application which claims the benefit of priority to International Application No. PCT/US2024/014761, filed on Feb. 7, 2024, which, in turn, claims the benefit of priority to U.S. Provisional Patent Application No. 63/444,375, filed on Feb. 9, 2023. The entire contents of each of the foregoing applications are incorporated herein by reference.

### SEQUENCE LISTING

The instant application contains a Sequence Listing which has been submitted electronically in XML format and is hereby incorporated by reference in its entirety. Said XML copy, created on Jul. 27, 2025, is named 121301_13202_SL.xml and is 1,520,250 bytes in size.

### BACKGROUND

RNA interference (RNAi) is an evolutionarily conserved mechanisn in which endogenous (microRNA) or exogenous (siRNA, shRNA) short non-coding RNAs downregulate gene expression of mRNA transcripts in sequence-dependent manner. As a native pathway that leverages an efficient cellular catalytic inechanisn, RNAi can be used to achieve robust, durable, and specific silencing of gene transcripts of interest. In recent years, several RNAi-based drugs have been successfully validated in the clinical studies, with demonstrated benefit at low doses and dosing, as infrequent as up to 6 months, compared to alternative gene silencing strategies. Novel delivery solutions along with highly chemically modified siRNAs have improved potency, durability, and safety and, thus, greatly expanded the reach of RNAi therapeutics, culminating in four approved drugs and several others in clinical development. In liver, infrequent delivery of metabolically stabilized siRNA conjugated to N-galactosamine (GalNAc) results in potent gene silencing that persists for months in humans with favorable safety and tolerability profiles. With their extended duration of action, RNAi therapeutics can benefit from a technology that enables rapid reversal of silencing activity and provides tailored control over RNAi pharmacology, a desired attribute for personalized precision medicines.

However, in some circumstances, a subject may respond poorly to treatment with a dsRNA agent or receive too high a dose. In such instances, a compound which reverses the iRNA silencing activity of the dsRNA agent could be administered to at least partially reduce the RNAi activity of the dsRNA agent. In other instances, the long-lasting effect of dsRNA makes waiting for that effect to slowly diminish through natural clearance an unattractive option.

Accordingly, there is a need in the art for compositions and methods that provide tailored control of RNAi pharmacology and, therefore, the therapeutic activity and/or side effects of siRNA based therapeutics in vivo.

### SUMMARY OF THE INVENTION

The present invention provides oligonucleotides that inhibit RNAi activity (REVERSIRs) of a double stranded ribonucleic acid (dsRNA) agent comprising a thermally destabilizing nucleotide in the antisense strand. The present invention also provides methods of use of such oligonucleotides to inhibit RNAi activity of a double stranded ribonucleic acid (dsRNA) agent comprising a thermally destabilizing nucleotide in the antisense strand in a subject, such as a subject in need thereof, e.g., a subject who received too high a dose of the dsRNA agent or is experiencing a side-effect from administration of the dsRNA agent.

The present invention is based, at least, in part, on the discovery that the activity of REVERSIRs, oligonucleotides which reverse the iRNA silencing activity of dsRNA agents used to control and tailor RNAi pharmacology, is abolished when a thermally destabilizing nucleotide is included in the antisense strand of the dsRNA agents previously demonstrated to be inhibited by the REVERSIRs. The present invention is also based, at least in part, on the discovery of REVERSIRs that inhibit the RNAi interference activity of dsRNA agents comprising a thermally destabilizing nucleotide in the antisense strand of the dsRNA agents.

Accordingly, in one aspect, the present invention provides a single stranded oligonucleotide for inhibiting RNAi activity of a double stranded ribonucleic acid (dsRNA) agent comprising a thermally destabilizing nucleotide in the antisense strand. The single stranded oligonucleotide includes a nucleotide sequence substantially complementary to the antisense strand of the dsRNA agent, wherein the single stranded oligonucleotide is 16-30 nucleotides in length, wherein substantially all of the nucleotides of the single stranded oligonucleotide comprise a nucleotide modification, and wherein at least three of the nucleotide modifications are a high affinity nucleotide modification.

In one embodiment, substantially all of the nucleotides comprise a nucleotide modification selected from the group consisting of a 2′-O-alkyl modification, a 2-substituted alkoxy modification, a 2′-substituted alkyl modification, a 2-halo modification, a deoxynucleotide modification, a locked nucleic acid (LNA) modification, a D-Methyleneoxy (4′-CH2-O-2′) locked nucleic acid (LNA) modification, a 2-O-(2-Methoxyethyl)(MOE) modification, bridged nucleic acid (2′,4′-BNA), 2-O-Ethyl (cEt) and a 2-methyl modification.

In another embodiment, all of the nucleotides comprise a nucleotide modification selected from the group consisting o a 2 W-alkyl modification, a 2′-substituted alkoxy modification, a 2′-substituted alkyl modification, a 2′-halo modification, a deoxynucleotide modification, a locked nucleic acid (LNA) modification, a D-Methyleneoxy (4′-CH2-O-2′) locked nucleic acid (LNA) modification, a 2′O-(2-Methoxyethyl) (MOE) modification, bridged nucle c acid (2′,4-BNA), 2′-O-Ethyl (cEt), and a 2′-O-methyl modification.

In one embodiment, at least four or five, e.g., 4, 5, or 6, of the nucleotide modifications may be high affinity nucleotide modifications.

In one embodiment, at least two of the high affinity nucleotide modifications are at positions 2 and 6; positions 2 and 5; positions 2 and 7; positions 2 and 8; positions 2 and 9; positions 2 and 14; positions 2 and 15; and/or positions 2 and 16, counting from the 3′-end of the oligonucleotide. In one embodiment, the high affinity nucleotide modifications are at positions 2, 6, 8, and 14; 2, 4, 5, 6, and 7; 2, 4, 6, 8, and 13; 2, 4, 6, 8, and 14; 2, 4, 6, 8, and 15; 2, 4, 6, 8, and 16; 2, 8, 10, and 14; 2, 4, 6, 8, and 14; or 2, 8, 12, and 14, counting from the 3′-end of the oligonucleotide.

In one embodiment, at least one of the nucleotides comprising a high affinity nucleotide modification is base paired with the nucleotide comprising the thermally destabilizing nucleotide in the antisense strand of the dsRNA agent.

In one embodiment, the high affinity modification is a locked nucleic acid (LNA) modification.

In one embodiment, the high affinity modification is a constrained Ethyl nucleic acid (cEtNA) modification.

In one embodiment, the high affinity modification is a bridged nucleic acid (BNA) modification.

The single stranded oligonucleotide may further comprise at least five, e.g., 5, 6, 7, 8, 9, 10, 11, 12, 13, 14, or 15, phosphorothioate internucleotide modifications.

In one embodiment, the single stranded oligonucleotide is conjugated to at least one ligand.

In one embodiment, the ligand is an N-acetylgalactosamine (GalNAc) derivative.

In one embodiment, the ligand is one or more GalNAc derivatives attached through a monovalent, bivalent, or trivalent branched linker.

In one embodiment, the ligand is

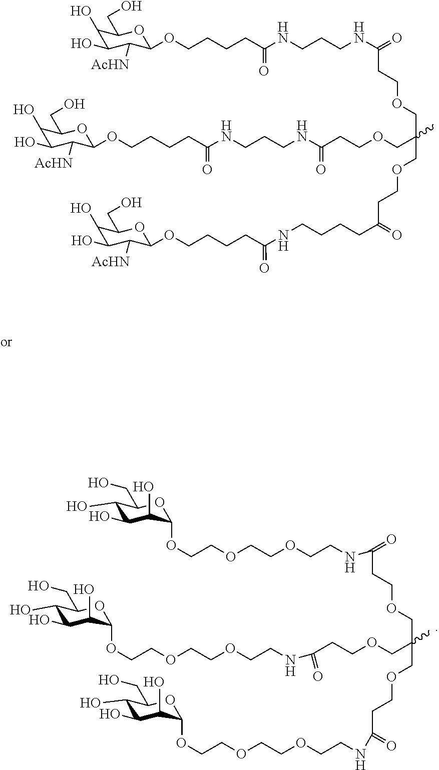

In one embodiment, the ligand is conjugated to a nucleoside comprising a deoxy sugar in the single stranded oligonucleotide.

In one embodiment, the deoxy sugar is a 2′-deoxy ribose.

In one embodiment, the ligand is conjugated to 3′-terminus of the single stranded oligonucleotide.

In another aspect, the present invention provides a single stranded oligonucleotide for inhibiting RNAi activity of a double stranded ribonucleic acid (dsRNA) agent comprising a thermally destabilizing nucleotide in the antisense strand,
wherein the single stranded oligonucleotide comprises a nucleotide sequence substantially complementary to the antisense strand of the dsRNA agent,
wherein the single stranded oligonucleotide is 18-24 nucleotides in length,
wherein the single stranded oligonucleotide comprises at least five phosphorothioate internucleotide modifications and is represented by formula (I):

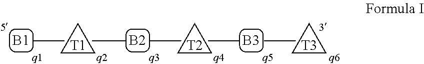

wherein:
B1, B2 and B3 each independently represent a nucleotide comprising a nucleotide modification independently selected from the group consisting of a 2′-deoxy, 2′-ribo, 2′-O-alkyl modification, a 2′-substituted alkoxy modification, a 2′-substituted alkoxy alkkyl modification, a 2′-substituted alkyl modification, and a 2′-halo modification;
T1, T2, and T3 each independently represent a nucleotide comprising a nucleotide modification selected from the group consisting of a deoxynucleotide modification, a D-Methyleneoxy (4′-CH2-O-2′) locked nucleic acid (LNA) modification, a 2′-O-(2-Methoxyethyl) (MOE) modification, a cEt modification, or a different BNA modification, a 2′-deoxy-2′-Fluoro, and a 2′-O-methyl modification;
q¹ q³ and q⁵ are each independently 3-12 nucleotides in length;
q², q⁴ and q⁶ are independently 1-6 nucleotide(s) in length; and
wherein the single stranded oligonucleotide is conjugated to at least one ligand.

In one embodiment, the single stranded oligonucleotide comprises 5-15 phosphorothioate internucleotide modifications; 5-14 phosphorothioate internucleotide modifications; 5-13 phosphorothioate internucleotide modifications; 5-12 phosphorothioate internucleotide modifications; 5-11 phosphorothioate internucleotide modifications; 5-10 phosphorothioate internucleotide modifications; 5-9 phosphorothioate internucleotide modifications; 5-8 phosphorothioate internucleotide modifications; 5-7 phosphorothioate internucleotide modifications; or 5-6 phosphorothioate internucleotide modifications.

In one embodiment, the single stranded oligonucleotide comprises 6-14 phosphorothioate internucleotide modifications.

In one embodiment, the single stranded oligonucleotide is 18-22 or 18-20 nucleotides in length.

In one embodiment, the single stranded oligonucleotide is at least about 90% complementary to the entire length of the antisense strand of the dsRNA agent.

In one embodiment, the single stranded oligonucleotide is 90% complementary to nucleotides 2-16 of the antisense stand of the dsRNA agent.

In one embodiment, the single stranded oligonucleotide is fully complementary to the antisense strand of the dsRNA agent.

In one embodiment, the nucleotide sequence of the antisense strand of the dsRNA agent comprises the nucleotide sequence 5′-UGUACUCUCAUUGUGGAUGACGA-3′ of SEQ ID NO: 9.

In one embodiment, the thermally destabilizing nucleotide modification is selected from the group consisting of an abasic modification; a mismatch with the opposing nucleotide in the duplex; a destabilizing sugar modification, a 2′-deoxy modification, an acyclic nucleotide, an unlocked nucleic acid (UNA), and a glycerol nucleic acid (GNA).

In one embodiment, the nucleotide sequence of the antisense strand of the dsRNA agent comprises the nucleotide sequence 5′-usGfsuac(Tgn)cucauugUfgGfaugacsgsa-3′ of SEQ ID NO: 11.

In one embodiment, the nucleotide sequence of the single stranded oligonucleotide is at least 90% identical to the entire nucleotide sequence of any one of the unmodified nucleotide sequences in Table 6.

In one embodiment, the ligand is an N-acetylgalactosamine (GalNAc) derivative.

In one embodiment, the ligand is one or more GalNAc derivatives attached through a monovalent, bivalent, or trivalent branched linker.

In one embodiment, the ligand is

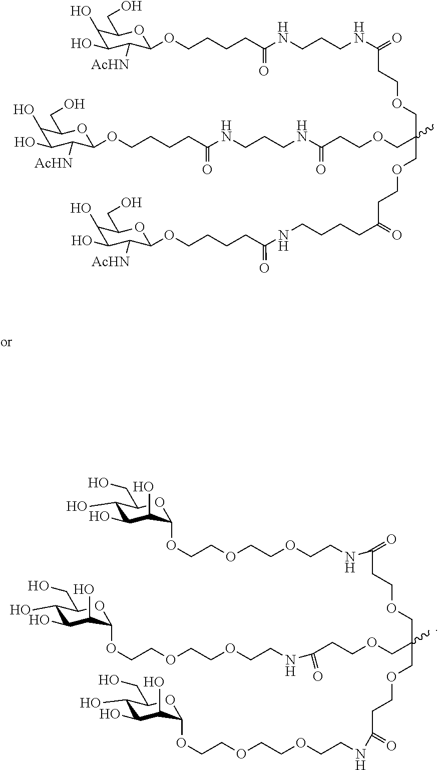

In one embodiment, the ligand is conjugated to a nucleoside comprising a deoxy sugar in the single stranded oligonucleotide.

In one embodiment, the deoxy sugar is a 2′-deoxy ribose.

In one embodiment, the ligand is conjugated to 3′-terminus of the single stranded oligonucleotide.

In one embodiment, the single stranded oligonucleotide comprises a modified nucleotide sequence differing by no more than 4 modified nucleotides from any one of the modified nucleotide sequences in Table 6.

The present invention also provides cells and pharmaceutical compositions, e.g., comprising a buffer (e.g., acetate, citrate, prolamin, carbonate, or phosphate, or any combination thereof) or unbuffered (e.g., saline or water) solution, comprising the single-stranded oligonucleotides of the invention.

In one aspect, the present invention provides a method of ameliorating in a subject a side effect of a dsRNA agent which inhibits the expression of a target gene and comprises a thermally destabilizing nucleotide modification in the antisense strand. The method includes administering to the subject an effective amount of the single stranded oligonucleotide or pharmaceutical composition of the invention, thereby ameliorating the side effect of the dsRNA agent in the subject.

In one aspect, the present invention provides a method of inhibiting the RNAi inhibitory activity of a dsRNA agent comprising a thermally destabilizing nucleotide modification in the antisense strand. The method includes contacting the dsRNA agent with the single stranded oligonucleotide or pharmaceutical composition of the invention, thereby inhibiting the RNAi inhibitory activity of a dsRNA agent comprising a thermally destabilizing nucleotide modification in the antisense strand.

In one embodiment, the dsRNA agent is in a cell.

In one embodiment, the cell is within a human subject.

In another aspect, the present invention provides a method of treating a subject in need thereof. The method includes administering to the subject a therapeutically effective amount of the single stranded oligonucleotide or pharmaceutical composition of the invention, thereby treating the subject.

In one embodiment, the subject in need thereof was previously administered a double stranded RNAi agent that inhibits the expression of a target gene and comprises a thermally destabilizing nucleotide modification in the antisense strand.

In one embodiment, the target gene is angiotensinogen (AGT).

In one embodiment, the subject in need thereof is suffering from hypotension.

In one embodiment, the subject in need thereof is suffering from hyperkalemia.

In one embodiment, the subject in need thereof is suffering from renal dysfunction.

In one embodiment, the method further comprises administering to the subject an additional therapy or therapeutic agent selected from the group consisting of increased dietary fluid/salt, fludrocortisone/midodrine treatment, intravenous fluids, vasopressor medications, down-titration or interruption of concomitant antihypertensive medications, a low potassium diet, thiazide/loop diuretic medications, oral potassium binders, calcium, glucose, insulin, and hemodialysis, or combinations thereof.

In one embodiment, the single stranded oligonucleotide or pharmaceutical composition is administered to the subject subcutaneously.

In one embodiment, the single stranded oligonucleotide or pharmaceutical composition is administered to the subject intravenously.

In one embodiment, the dose of the single stranded oligonucleotide or pharmaceutical composition is at a ratio of about 1:1, 2:1 or 3:1 to the dose of dsRNA agent previously administered to the subject.

In one embodiment, the dose of the single stranded oligonucleotide or pharmaceutical composition is at a ratio of about 1:1 to the dose of dsRNA agent previously administered to the subject.

In one embodiment, the dose of the single stranded oligonucleotide or pharmaceutical composition is at a ratio of about 1:2 to the dose of dsRNA agent previously administered to the subject.

In one embodiment, the dose of the single stranded oligonucleotide or pharmaceutical composition is at a ratio of about 1:3 to the dose of dsRNA agent previously administered to the subject.

In one embodiment, the dose of the single stranded oligonucleotide or pharmaceutical composition is at a ratio of about 1:4 to the dose of dsRNA agent previously administered to the subject.

In one embodiment, the dose of the single stranded oligonucleotide or pharmaceutical composition is at a ratio of about 1:5 to the dose of dsRNA agent previously administered to the subject.

In one embodiment, the dose of the single stranded oligonucleotide or pharmaceutical composition is at a ratio of about 1:10 to the dose of dsRNA agent previously administered to the subject.

In one embodiment, the dose of the single stranded oligonucleotide or pharmaceutical composition is at a ratio of about 2:1 to the dose of dsRNA agent previously administered to the subject.

In one embodiment, the dose of the single stranded oligonucleotide or pharmaceutical composition is at a ratio of about 3:1 to the dose of dsRNA agent previously administered to the subject.

In one embodiment, the dose of the single stranded oligonucleotide or pharmaceutical composition is at a ratio of about 5:1 to the dose of dsRNA agent previously administered to the subject.

In one embodiment, the dose of the single stranded oligonucleotide or pharmaceutical composition is at a ratio of about 10:1 to the dose of dsRNA agent previously administered to the subject.

In one embodiment, the dose of the single stranded oligonucleotide or pharmaceutical composition is split into three doses and administered to the subject at 24 hour intervals.

In one embodiment, the dose of the single stranded oligonucleotide or pharmaceutical composition is split into two doses and administered to the subject at 24 hour intervals.

In one embodiment, the dose of the single stranded oligonucleotide or pharmaceutical composition is split into three doses and administered to the subject at 12 hour intervals.

In one embodiment, the dose of the single stranded oligonucleotide or pharmaceutical composition is split into two doses and administered to the subject at 12 hour intervals.

In one embodiment, the dose of the single stranded oligonucleotide or pharmaceutical composition is split into three doses and administered to the subject at 8 hour intervals.

In one embodiment, the dose of the single stranded oligonucleotide or pharmaceutical composition is split into two doses and administered to the subject at 8 hour intervals.

### BRIEF DESCRIPTION OF THE DRAWINGS

FIG. 1A is a graph depicting the inhibition of silencing of a dsRNA agent targeting TTR and conjugated to a GalNAc ligand by a REVERSIR in mice.

FIG. 1B is a graph depicting the inhibition of silencing of a dsRNA agent targeting TTR and conjugated to a GalNAc ligand by a REVERSIR in mice.

FIG. 1C is a graph depicting the inability of a REVERSIR previously demonstrated to inhibit the silencing of dsRNA agents conjugated to a GalNAc ligand (the dsRNA agent in FIGS. 1A and 1B) to inhibit silencing of dsRNA agents targeting the same region of the mRNA and comprising the same nucleotide sequence and substantially the same nucleotide modifications with the exception of inclusion of a thermally destabilizing nucleotide modification in mice.

FIG. 2 is a graph depicting the effect of lengthening the REVERSIRs and administering a dose up to 100 times higher than that of the REVERSIRs targeting non-destabilized dsRNAs on the ability to inhibit the silencing of dsRNA agents comprising a thermally destabilizing nucleotide modification and conjugated to a GalNAc ligand in mice. The graph also depicts the successful redosing of the GalNAc dsRNA agent 30-days after the administration of the REVERSIR agent and at recovery. The successful redosing means resumption of RNAi pharmacology, with similar profile to the control (no REVERSIR) group in mice.

FIG. 3A is a graph depicting the effect of intravenous administration of 0.3 mg/kg of a REVERSIR formulated in a LNP on the RNAi silencing activity of a dsRNA agent comprising a thermally destabilizing nucleotide modification in the antisense strand and the effect of subcutaneous administration of 3 mg/kg in mice of a GalNAc conjugated REVERSIR on the RNAi silencing activity of a dsRNA agent comprising a thermally destabilizing nucleotide modification in the antisense strand in mice.

FIG. 3B is a graph depicting the effect of subcutaneous administration of 3 mg/kg of a GalNAc conjugated REVERSIR on the RNAi silencing activity of a dsRNA agent comprising a thermally destabilizing nucleotide modification in the antisense strand in mice.

FIG. 4A is a graph depicting the in vitro effect of the indicated REVERSIRs on the RNAi silencing activity of AD-85481 when the cells were transfected with the REVERSIRs.

FIG. 4B is a graph depicting the in vitro effect of the indicated REVERSIRs on the RNAi silencing activity of AD-85481 when the REVERSIRs were assessed by free uptake into the cells.

FIG. 5 is a Table showing the in vitro activity of the REVERSIRs A-515518, A-515556, A-515559, A-515586, and A-515589.

FIG. 6A is a graph depicting the effect of intravenous administration of 0.3 mg/kg of the indicated REVERSIRs formulated in a LNP on the RNAi silencing activity of a AD-85481 in mice.

FIG. 6B is a graph depicting the effect of subcutaneous administration of 3 mg/kg of the indicated GalNAc conjugated REVERSIRs on the RNAi silencing activity of AD-85481 in mice.

FIG. 7A schematically depicts the modified nucleotide sequences of A-762636, A-762655, A-762680, A-762689, A-762722, and A-762645.

FIG. 7B is a graph depicting human AGT mRNA levels in mice administered an AAV encoding human AGT at the indicated time points following subcutaneous administration of a single 3 mg/kg dose of AD-85481 and a single 3 mg/kg dose of the GalNAc conjugated indicated REVERSIRs.

FIG. 8A schematically depicts the modified nucleotide sequences of A-762689, A-762722, and A-762645.

FIG. 83 is a graph depicting human AGT mRNA levels in mice administered an AAV encoding human ACT at the indicated time points following subcutaneous administration of a single 3 tug/kg dose of AD-85481 and a single 1 mg/kg or 3 tug/kg dose of the indicated REVERSIRs.

FIG. 9 schematically depicts the study design for assessment of A-762722 and A-762645 in non-human primates and the modified nucleotide sequences of A-762722 and A-762645.

FIG. 10A is a graph depicting the effect of subcutaneous administration of 1 mg/kg or 3 mg/kg of the indicated GalNAc conjugated REVERSIRs on the RNAi silencing activity of AD-85481 in non-human primates.

FIG. 10B is a graph depicting effect of intravenous administration of 0.3 nm/kg of the indicated REVERSIR formulated in a LNP on the RNAi silencing activity of a AD-85481 in non-human primates.

FIG. 11A schematically depicts the modified nucleotide sequences of A-762645, A-809917, A80991.8, A-809919, A-809920, A-809921, A-809922, A-809923, A-809924, A809924, and A-809925.

FIG. 11B is a graph depicting human AGT mRNA levels in mice administered an AAV encoding human AGT at the indicated time points following subcutaneous administration of a single 3 mg/kg dose of AD-85481 and a single 3 mg/kg dose of the indicated REVERSIRs.

FIG. 12A schematically depicts the modified nucleotide sequences of A-762645, A809918, A-809925, A2423818, or A2423819.

FIG. 12B is a graph depicting human AGT mRNA levels in mice administered an AAV encoding human AGT at the indicated time points following subcutaneous administration of a single 3 mg/kg dose of AD-85481 and a single 3 mg/kg dose of the indicated REVERSIRs.

FIG. 12C is a graph depicting human AGT mRNA levels in mice administered an AAV encoding human AGT at the indicated time points following subcutaneous administration of a single 3 mg/kg dose of AD-85481 and a single 3 mg/kg dose of the indicated REVERSIRs.

FIG. 13A is a graph depicting the effect of subcutaneous administration of 3 mg/kg of the indicated REVERSIRs on the RNAi silencing activity of AD-85481 in non-human primates.

FIG. 13B is a graph depicting the reversal effect of subcutaneous administration of 3 mg/kg of the indicated REVERSIRs on the RNAi silencing activity of AD-85481 in non-human primates.

FIG. 13C is a Table depicting the effect of subcutaneous administration of 3 mg/kg of the indicated REVERSIRs on the RNAi silencing activity of AD-85481 in non-human primates.

FIG. 14A are graphs depicting the effect of the indicated REVERSIRs on IL-6, IL-8, Il1B, and TNFalpha levels in a diluted human whole blood transfection assay (24-hour incubation).

FIG. 14B is a graph depicting the effect of the indicated REVERSIRs on IL-8 and MCP-1 levels in a 6-hour human whole blood assay.

FIG. 14C are graphs depicting the effects of the indicated REVERSIRs on non-human primate platelet counts and whole blood cell counts.

FIG. 15A are graphs depicting % of AGT remaining and AGT silencing reversal using varying RVR/dsRNA ratio and GalNAc conjugated and LNP formulations in non-human primates.

FIG. 15B is a graph depicting REVERSIR A-762645 resulted in potent and durable pharmacology following AD-85481 (Zilebesiran) dosing in non-human primates.

FIG. 15C is a graph depicting % AGT remaining following subcutaneous or intravenous delivery of the indicated REVERSIR for varying dose levels.

FIG. 16A is a graph depicting % AGT remaining normalized to predose following subcutaneous administration of the indicated REVERSIR for varying dose levels in an hAGT-AAV mouse model pretreated with 10 mg/kg of AD-85481. For the hAGT-AAV mouse model, a human AGT gene was expressed in the mouse hepatocytes by transduction with liver-specific AAVS virus.

FIG. 16B is a graph depicting % AGT remaining following subcutaneously administered REVERSIR in an hAGT-AAV mouse model pretreated with 3 mg/kg of AD-85481. For the hAGT-AAV mouse model, a human AGT gene was expressed in the mouse hepatocytes by transduction with liver-specific AAVS virus.

FIG. 17 is a graph depicting % AGT remaining following subcutaneously administered REVERSIR A-762645 in an hAGT transgenic mouse model pretreated with 10 mg/kg of AD-85481. For the hAGT-AAV mouse model, a human AGT gene was randomly inserted in multiple areas in the mouse genome (generated during embryonic stein cell stage).

FIG. 18 is a graph depicting % AGT remaining following subcutaneously administered GalNAc conjugated REVERSIR A-762645 in a PXB mouse model pretreated with 10 mg/kg of AD-85481.

FIG. 19A is a graph depicting % AGT remaining following administration of varying ratio of REVERSIR to dsRNA in a rat.

FIG. 19B is a graph depicting % AGT remaining following administration of varying AD-85481 dsRNA load.

FIG. 19C is a graph depicting reversal of AGT knockdown with a REVERSIR in an LNP formulation and a REVERSIR with a GalNac ligand subcutaneously administered to a rat.

FIG. 19D is a graph depicting % ACT remaining following bolus intravenous administration and subcutaneous administration of the REVERSIR A-762645.

FIG. 20 schematically depicts the modified nucleotide sequences of REVERSIRs A-3903617, A-3903618, A-3903619, A-3903620, A-3903621, A-3903622, A-3903623, A-3903624, A-3903625, A-3903626, A-3903627, A-3903628, A-3903629, A-3903630, and A-3903631.

FIG. 21 is a graph depicting % ACT remaining following subcutaneous administration of the indicated GalNAc conjugated REVERSIR in an hAGT-AAV mouse model pretreated with 3 mg/kg or 10 mg/kg of AD-85481. For the hAGT-AAV mouse model, a human AGT gene was expressed in the mouse hepatocytes by transduction with liver-specific AAV8 virus.

FIG. 22 is a graph depicting % ACT remaining following subcutaneous administration of the indicated GalNAc conjugated REVERSIR in an hAGT-AAV mouse model pretreated with 3 mg/kg or 10 mg/kg of AD-85481. For the hAGT-AAV mouse model, a human AGT gene was expressed in the mouse hepatocytes by transduction with liver-specific AAV8 virus.

FIG. 23 is a graph depicting % ACT remaining following subcutaneous administration of the indicated GalNAc conjugated REVERSIR in an hAGT-AAV mouse model pretreated with 3 mg/kg or 10 mg/kg of AD-85481. For the hAGT-AAV mouse model, a human AGT gene was expressed in the mouse hepatocytes by transduction with liver-specific AAV8 virus.

### DETAILED DESCRIPTION OF THE INVENTION

The present invention provides single stranded oligonucleotides (REVERSIRs) that inhibit RNAi activity of a double stranded ribonucleic acid (dsRNA) agent comprising a thermally destabilizing nucleotide in the antisense strand. The present invention also provides methods of use of such oligonucleotides to inhibit RNAi activity of a double stranded ribonucleic acid (dsRNA) agent comprising a thermally destabilizing nucleotide in the antisense strand in a subject, such as a subject in need thereof, e.g., a subject that received too high a dose of the dsRNA agent or is experiencing a side-effect from administration of the dsRNA agent, e.g., off-target effect.

The present invention is based, at least, in part, on the discovery that the activity of REVERSIRs, oligonucleotides which reverse the iRNA silencing activity of dsRNA agents used to control and tailor RNAi pharmacology, is abolished when a thermally destabilizing nucleotide is included in the antisense strand of the dsRNA agents previously demonstrated to be inhibited by the REVERSIRs.

The present invention is also based, at least in part, on the identification of REVERSIRs that are able to inhibit RNAi activity of a class of dsRNA agents comprising a thermally destabilizing nucleotide in the antisense strand. This new class of REVERSIRs is characterized by a combination of structural properties, e.g., a combination of length, phosphorothioate (PS) content and specific placement of a high affinity nucleotide modification, such as a locked nucleic acid (LNA) modification, relative to the thermally destabilizing nucleotide in the antisense strand of the dsRNA agent.

The following detailed description discloses how to make and use oligonucleotides that inhibit RNAi activity of a double stranded ribonucleic acid (dsRNA) agent comprising a thermally destabilizing nucleotide in the antisense strand, as well as uses and methods for treating subjects in need thereof.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

The term “at least”, “no less than”, or “or more” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 19 nucleotides of a 21 nucleotide nucleic acid molecule” means that 19, 20, or 21 nucleotides have the indicated property. When at least is present before a series of numbers or a range, it is understood that “at least” can modify each of the numbers in the series or range.

As used herein, “no more than” or “or less” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

In the event of a conflict between an indicated target site and the nucleotide sequence for a sense or antisense strand, the indicated sequence takes precedence.

In the event of a conflict between a sequence and its indicated site on a transcript or other sequence, the nucleotide sequence recited in the specification takes precedence.

As used herein, the term “nucleoside” refers to a glycosylamine comprising a nucleobase and a sugar. Nucleosides includes, but are not limited to, naturally occurring nucleosides, abasic nucleosides, modified nucleosides, and nucleosides having mimetic bases and/or sugar groups.

As used herein, the term “nucleotide” refers to a glycosomine comprising a nucleobase and a sugar having a phosphate group covalently linked to the sugar. Nucleotides may be modified with any of a variety of substituents.

As used herein, the term “nucleobase” refers to the base portion of a nucleoside or nucleotide.

A nucleobase may comprise any atom or group of atoms capable of hydrogen bonding to a base of another nucleic acid.

As used herein, the term “heterocyclic base moiety” refers to a nucleobase comprising a heterocycle.

As used herein, the term “oligomeric compound” refers to a polymeric structure comprising two or more sub-structures and capable of hybridizing to a region of a nucleic acid molecule. In certain embodiments, oligomeric compounds are oligonucleosides. In certain embodiments, oligomeric compounds are oligonucleotides. In certain embodiments, oligomeric compounds are antisense compounds. In certain embodiments, oligomeric compounds are REVERSIR compounds. In certain embodiments, oligomeric compounds comprise conjugate groups.

As used herein “oligonucleoside” refers to an oligonucleotide in which the internucleoside linkages do not contain a phosphorus atom.

As used herein, the term “oligonucleotide” refers to an oligomeric compound comprising a plurality of linked nucleosides. In certain embodiment, one or more nucleotides of an oligonucleotide is modified. In certain embodiments, an oligonucleotide comprises ribonucleic acid (RNA) or deoxyribonucleic acid (DNA). In certain embodiments, oligonucleotides are composed of naturally- and/or non-naturally-occurring nucleobases, sugars and covalent internucleoside linkages, and may further include non-nucleic acid conjugates.

As used herein, the term “REVERSIR compound” or “REVERSIR” refers to a single-stranded oligomeric compound, such as a single stranded oligonucleotide, that is complementary to and capable of hybridizing (targeted to) with at least one strand of a dsRNA agent comprising a thermally destabilizing nucleotide in the antisense strand. Without limitations, the REVERSIR compound may not only block unintended target pharmacodynamic (PD) effects, but also block any potential off-target activity that could occur with a dsRNA agent, e.g., a conjugated or unconjugated dsRNA agent. REVERSIRs bind to and are internalized into a cell through the asialoglycoprotein receptor (ASPGR) and irreversibly bind to the antisense strand of a dsRNA agent in a functional RISC complex. The binding of the REVERSIR abrogates the mRNA target recognition and cleavage triggered by the hybridization of the dsRNA agent.

As used herein, the term “REVERSIR activity” refers to any decrease in intensity and/or duration of any dsRNA activity attributable to hybridization of a REVERSIR compound to one of the strands of the dsRNA.

The REVERSIR compounds disclosed herein are particularly effective in reducing the activity of dsRNAs containing a thermally destabilizing nucleotide in the antisense strand. For example, the REVERSIR compounds disclosed herein can reduce within 24 hours to 7 days the activity of a dsRNA by at least about 20% or at least about 30%, or at least about 40%, or at least about 50%, or at least about 60%, or at least about 70%, or at least about 80%, or at least about 90%, or at least about 95%, or at least about 97%, or at least about 99% or up to and including a 100% decrease (i.e., absent level as compared to a reference sample), or any decrease between 20-100% or 50-100% as compared to a reference level. The reference level can be dsRNA activity in absence of the REVERSIR compound.

In some embodiments, the REVERSIR compounds described herein can reduce the activity of the dsRNA by at least 5%, at least 10%, at least 15%, at least 20%, for example by 5%, 10%, 15%, 20%, 30%, 40%, 50%, 60%, 70%, 75%, 80%, 85%, 90%, 95% or more and up to and including complete reduction or inhibition of dsRNA activity, within less than seven (e.g., within six days, five days, four days, three days, two days or one day) of administering or use of the REVERSIR compound.

In some embodiments, the REVERSIR compounds can completely reduce the dsRNA activity within four days of administering or use of the REVERSIR compound. By complete reduction of dsRNA activity is meant a reduction of the dsRNA activity by at least 80% relative to a reference level.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of a target mRNA sequence, e.g., in a cell, e.g., a cell within a subject, such as a mammalian subject.

In certain embodiments, an “iRNA” for use in the compositions, uses, and methods of the invention is a double stranded RNA and is referred to herein as a “double stranded RNA agent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA. In some embodiments of the invention, a double stranded RNA (dsRNA) triggers the degradation of a target RNA, e.g., an mRNA, through a post-transcriptional gene-silencing mechanism referred to herein as RNA interference or RNAi.

The term “antisense strand” or “guide strand” refers to the strand of an iRNA, e.g., a dsRNA, which includes a region that is substantially complementary to a target sequence.

The term “sense strand” or “passenger strand” as used herein, refers to the strand of an iRNA that includes a region that is substantially complementary to a region of the antisense strand as that term is defined herein.

The term “thermally destabilizing modification(s)” includes modification(s) that would result with a dsRNA with a lower overall melting temperature (Tₘ) than the Tₘ of the dsRNA without having such modification(s). For example, the thermally destabilizing modification(s) can decrease the Tₘ of the dsRNA by 1-4° C., such as one, two, three or four degrees Celsius. And, the term “thermally destabilizing nucleotide” refers to a nucleotide containing one or more thermally destabilizing modifications.

Exemplary thermally destabilizing modifications include abasic modifications; mismatches with the opposing nucleotide in the duplex; and sugar modifications such as 2′-deoxy modifications or acyclic nucleotides e.g., unlocked nucleic acids (UNA) or glycol nucleic acids (GNA), or 2′-5′-linked ribonucleotides (“3′-RNA”).

As used herein the term “detecting dsRNA activity” or “measuring dsRNA activity” means that a test for detecting or measuring dsRNA activity is performed on a particular sample and compared to that of a control sample. Such detection and/or measuring can include values of zero. Thus, if a test for detection of dsRNA activity results in a finding of no dsRNA activity (dsRNA activity of zero), the step of “detecting dsRNA activity” has nevertheless been performed.

As used herein the term “control sample” refers to a sample that has not been contacted with an oligomeric compound.

As used herein, the term “motif” refers to the pattern of unmodified and modified nucleotides in an oligomeric compound.

As used herein “internucleoside linkage” refers to a covalent linkage between adjacent nucleosides.

As used herein “naturally occurring internucleoside linkage” refers to a 3′ to 5′ phosphodiester linkage.

As used herein, the term “chimeric oligomer” refers to an oligomeric compound, having at least one sugar, nucleobase or internucleoside linkage that is differentially modified as compared to at least one other sugar, nucleobase or internucleoside linkage within the same oligomeric compound. The remainder of the sugars, nucleobases and internucleoside linkages can be independently modified or unmodified, the same or different.

As used herein, the term “chimeric oligonucleotide” refers to an oligonucleotide, having at least one sugar, nucleobase or internucleoside linkage that is differentially modified as compared to at least one other sugar, nucleobase or internucleoside linkage within the same oligonucleotide. The remainder of the sugars, nucleobases and internucleoside linkages can be independently modified or unmodified, the same or different.

As used herein, the term “mixed-backbone oligomeric compound” refers to an oligomeric compound wherein at least one internucleoside linkage of the oligomeric compound is different from at least one other internucleoside linkage of the oligomeric compound.

As used herein, the term “target protein” refers to a protein, the modulation of which is desired. For example, the target protein is Angiotensinogen (AGT).

As used herein, the term “target gene” refers to a gene encoding a target protein. For example, the target gene is Angiotensinogen (AGT).

As used herein, the term “target nucleic acid” refers to any nucleic acid molecule the expression or activity of which is capable of being modulated by a conjugated or unconjugated dsRNA compound. Target nucleic acids include, but are not limited to, RNA (including, but not limited to pre-mRNA and mRNA or portions thereof) transcribed from DNA encoding a target protein, and also cDNA derived from such RNA, and miRNA. For example, the target nucleic acid can be a cellular gene (or mRNA transcribed from the gene) whose expression is associated with a particular disorder or disease state, or a nucleic acid molecule from an infectious agent.

As used herein, the term “target siRNA” and “target dsRNA” refers to a compound that is targeted by a REVERSIR compound.

As used herein, the term “targeting” or “targeted to” refers to the association of antisense strand of a dsRNA to a particular target nucleic acid molecule or a particular region of nucleotides within a target nucleic acid molecule.

As used herein, the term “nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, the term “non-complementary nucleobase” refers to a pair of nucleobases that do not form hydrogen bonds with one another or otherwise support hybridization.

As used herein, the term “complementary” refers to the capacity of an oligomeric compound to hybridize to another oligomeric compound or nucleic acid through nucleobase complementarity. In certain embodiments, an oligomeric compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are oligomeric compounds that may comprise up to about 20% nucleotides that are mismatched (i.e., are not nucleobase complementary to the corresponding nucleotides of the target). Preferably the oligomeric compounds, contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% complementary to a target nucleic acid.

The terms “complementary,” “fully complementary” and “substantially complementary” herein can be used with respect to the base matching between between two oligonucletoides or polynucleotides, such as the antisense strand of a double stranded RNA agent and a REVERSIR, as will be understood from the context of their use.

As used herein, “hybridization” means the pairing of complementary oligomeric compounds (e.g., an antisense strand of a dsRNA and its target nucleic acid or a REVERSIR to its target dsRNA). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

As used herein, the term “specifically hybridizes” refers to the ability of an oligomeric compound to hybridize to one nucleic acid site with greater affinity than it hybridizes to another nucleic acid site. In certain embodiments, the antisense strand of an dsRNA specifically hybridizes to more than one target site.

As used herein, the term “modulation” refers to a perturbation of function or activity when compared to the level of the function or activity prior to modulation. For example, modulation includes the change, either an increase (stimulation or induction) or a decrease (inhibition or reduction) in gene expression. As further example, modulation of expression can include perturbing splice site selection of pre-mRNA processing.

As used herein, the term “expression” refers to all the functions and steps by which a gene's coded information is converted into structures present and operating in a cell. Such structures include, but are not limited to the products of transcription and translation.

As used herein, “variant” refers to an alternative RNA transcript that can be produced from the same genomic region of DNA. Variants include, but are not limited to “pre-mRNA variants” which are transcripts produced from the same genomic DNA that differ from other transcripts produced from the same genomic DNA in either their start or stop position and contain both intronic and exonic sequence. Variants also include, but are not limited to, those with alternate splice junctions, or alternate initiation and termination codons.

As used herein, “high affinity nucleotide modification” refers to a nucleotide having at least one modified nucleobase, internucleoside linkage or sugar moiety, when compared to naturally occurring nucleotides, such that the modification increases the affinity of an antisense compound comprising the high affinity modified nucleotide to its target nucleic acid. High affinity modifications include, but are not limited to, nucleotides comprising 2′-modified sugars.

As used herein, the term “2′-modified” or “2′-substituted” means a sugar comprising substituent at the 2′ position other than H or OH. 2′-modified monomers, include, but are not limited to, BNA's and monomers (e.g., nucleosides and nucleotides) with 2′-substituents, such as allyl, amino, azido, thio, O-allyl, O—C₁-C₁₀ alkyl, —OCF3, O—(CH₂)₂O—CH₃, 2′-O(CH₂)₂SCH₃, O—(CH₂)₂—O—N(Rm)(Rn), or O—CH₂—C(═O)—N(Rm)(Rn), where each Rm and Rn is, independently, H or substituted or unsubstituted C₁-C₁₀ alkyl. In certain embodiments, oligomeric compounds comprise a 2′ modified monomer that does not have the formula 2′-O(CH₂)ₙH, wherein n is one to six. In certain embodiments, oligomeric compounds comprise a 2′ modified monomer that does not have the formula 2′-OCH₃. In certain embodiments, oligomeric compounds comprise a 2′ modified monomer that does not have the formula or, in the alternative, 2′-O(CH₂)₂OCH₃.

As used herein, the term “locked nucleic acid” or “LNA” or “locked nucleoside” or “locked nucleotide” refers to a nucleoside or nucleotide wherein the furanose portion of the nucleoside includes a bridge connecting two carbon atoms on the furanose ring, thereby forming a bicyclic ring system. Locked nucleic acids are also referred to as bicyclic nucleic acids (BNA).

As used herein, unless otherwise indicated, the term “methyleneoxy LNA” alone refers to (3-D-methyleneoxy LNA.

As used herein, the term “MOE” refers to a 2′-O-methoxyethyl substituent.

As used herein, the term “pharmaceutically acceptable salts” refers to salts of active compounds that retain the desired biological activity of the active compound and do not impart undesired toxicological effects thereto.

As used herein, the term “cap structure” or “terminal cap moiety” refers to chemical modifications, which have been incorporated at either terminus of an antisense compound.

The phrase “contacting a cell,” such as contacting a cell with a REVERSIR, as used herein, includes contacting a cell by any possible means. Contacting a cell includes contacting a cell in vitro or contacting a cell in vivo. The contacting may be done directly or indirectly. Thus, for example, the REVERSIR may be put into physical contact with the cell by the individual performing the method, or alternatively, the REVERSIR may be put into a situation that will permit or cause it to subsequently come into contact with the cell.

Contacting a cell in vitro may be done, for example, by incubating the cell with the REVERSIR. Contacting a cell in vivo may be done, for example, by injecting the REVERSIR into or near the tissue where the cell is located, or by injecting the REVERSIR into another area, e.g., the bloodstream or the subcutaneous space, such that the agent will subsequently reach the tissue where the cell to be contacted is located. Combinations of in vitro and in vivo methods of contacting are also possible. For example, a cell may also be contacted in vitro with a REVERSIR and subsequently transplanted into a subject.

In certain embodiments, contacting a cell with a REVERSIR includes “introducing” or “delivering the REVERSIR into the cell” by facilitating or effecting uptake or absorption into the cell. Absorption or uptake of a REVERSIR can occur through unaided diffusion or active cellular processes, or by auxiliary agents or devices. Introducing an REVERSIR into a cell may be in vitro or in vivo. For example, for in vivo introduction, REVERSIR can be injected into a tissue site or administered systemically. In vitro introduction into a cell includes methods known in the art such as electroporation and lipofection. Further approaches are described herein below or are known in the art.

The term “lipid nanoparticle” or “LNP” is a vesicle comprising a lipid layer encapsulating a pharmaceutically active molecule, such as a nucleic acid molecule, e.g., an iRNA or a plasmid from which an iRNA is transcribed. LNPs are described in, for example, U.S. Pat. Nos. 6,858,225, 6,815,432, 8,158,601, and 8,058,069, the entire contents of which are hereby incorporated herein by reference. In some embodiments, one or more of the oligonucleotides (REVERSIRs) that inhibit RNAi activity of a double stranded ribonucleic acid (dsRNA) agent described herein are encapsulated in an LNP.

“Therapeutically effective amount,” as used herein, is intended to include the amount of a REVERSIR that, when administered to a subject, is sufficient to effect treatment of the disease (e.g., by diminishing, ameliorating, or maintaining the existing disease or one or more symptoms of disease). The “therapeutically effective amount” may vary depending on the REVERSIR, how the REVERSIR is administered, the disease and its severity and the history, age, weight, family history, genetic makeup, the types of preceding or concomitant treatments, if any, and other individual characteristics of the subject to be treated.

A “therapeutically-effective amount” also includes an amount of a REVERSIR that produces some desired effect at a reasonable benefit/risk ratio applicable to any treatment. The REVERSIR employed in the methods of the present invention may be administered in a sufficient amount to produce a reasonable benefit/risk ratio applicable to such treatment.

As used herein, “administering” means providing an RNAi agent and/or REVERSIR to animal subject such as a human, and includes, but is not limited to administering by a medical professional and self-administering.

As used herein, the term “co-administering” means providing the RNAi agent and REVERSIR to a subject, such as a human subject. In certain embodiments, the RNAi agent and REVERSIR are administered together. In certain embodiments, the RNAi agent and REVERSIR are administered separately. In certain embodiments, the RNAi agent and REVERSIR are administered at the same time. In certain embodiments, the RNAi agent and REVERSIR are administered at different times. In certain embodiments, the RNAi agent and REVERSIR are administered through the same route of administration. In certain embodiments, the RNAi agent and REVERSIR are administered through different routes of administration. In certain embodiments, the RNAi agent and REVERSIR are contained in the same pharmaceutical formulation. In certain embodiments, the RNAi agent and REVERSIR are in separate formulations.

The phrase “pharmaceutically acceptable” is employed herein to refer to those compounds, materials (including salts), compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Such carriers are known in the art. Pharmaceutically acceptable carriers include carriers for administration by injection.

As used herein, the term “in vitro” refers to events that occur in an artificial environment, e.g., in a test tube or reaction vessel, in cell culture, etc., rather than within an organism (e.g. animal or a plant). As used herein, the term “ex vivo” refers to cells which are removed from a living organism and cultured outside the organism (e.g., in a test tube). As used herein, the term “in vivo” refers to events that occur within an organism (e.g. animal, plant, and/or microbe).

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that expresses the universal target sequence, either endogenously or heterologously. In an embodiment, the subject is a human. In some embodiments, the subject is a female human. In other embodiments, the subject is a male human. In one embodiment, the subject is an adult subject. In another embodiment, the subject is a pediatric subject.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

As used herein, the term “dosage unit” refers to a form in which an RNAi agent and/or REVERSIR is provided. In certain embodiments, a dosage unit is a vial comprising lyophilized RNAi agent and/or REVERSIR. In certain embodiments, a dosage unit is a vial comprising reconstituted RNAi agent and/or REVERSIR.

As used herein, the term “active pharmaceutical ingredient” refers to the substance in a pharmaceutical composition that provides a desired effect.

As used herein, the term “side effects” refers to physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. For example, increased aminotransferase levels in serum may indicate liver toxicity or liver function abnormality. For example, increased bilirubin may indicate liver toxicity or liver function abnormality.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs, or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In some embodiments, a “sample derived from a subject” refers to urine obtained from the subject. A “sample derived from a subject” can refer to blood or blood derived serum or plasma from the subject.

As used herein, the term “alkyl,” as used herein, refers to a saturated straight or branched hydrocarbon radical containing up to twenty four carbon atoms. Examples of alkyl groups include, but are not limited to, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C1-C12 alkyl) with from 1 to about 6 carbon atoms being more preferred. The term “lower alkyl” as used herein includes from 1 to about 6 carbon atoms. Alkyl groups as used herein may optionally include one or more further substituent groups.

As used herein, the term “alkenyl,” as used herein, refers to a straight or branched hydrocarbon chain radical containing up to twenty four carbon atoms and having at least one carbon-carbon double bond. Examples of alkenyl groups include, but are not limited to, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

As used herein, the term “alkynyl,” as used herein, refers to a straight or branched hydrocarbon radical containing up to twenty four carbon atoms and having at least one carbon-carbon triple bond. Examples of alkynyl groups include, but are not limited to, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substitutent groups.

As used herein, the term “aminoalkyl” as used herein, refers to an amino substituted alkyl radical. This term is meant to include C1-C12 alkyl groups having an amino substituent at any position and wherein the alkyl group attaches the aminoalkyl group to the parent molecule. The alkyl and/or amino portions of the aminoalkyl group can be further substituted with substituent groups.

As used herein, the term “aliphatic,” as used herein, refers to a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation polyalkoxys, such as polyalkylene glycols, polyamines, and polyimines. Aliphatic groups as used herein may optionally include further substitutent groups.

As used herein, the term “alicyclic” or “alicyclyl” refers to a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substitutent groups. As used herein, the term “alkoxy,” as used herein, refers to a radical formed between an alkyl group and an oxygen atom wherein the oxygen atom is used to attach the alkoxy group to a parent molecule. Examples of alkoxy groups include, but are not limited to, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substitutent groups. As used herein, the terms “halo” and “halogen,” as used herein, refer to an atom selected from fluorine, chlorine, bromine and iodine.

As used herein, the terms “aryl” and “aromatic,” as used herein, refer to a mono- or polycyclic carbocyclic ring system radicals having one or more aromatic rings. Examples of aryl groups include, but are not limited to, phenyl, naphthyl, tetrahydronaphthyl, indanyl, idenyl and the like. Preferred aryl ring systems have from about 5 to about 20 carbon atoms in one or more rings. Aryl groups as used herein may optionally include further substitutent groups.

As used herein, the terms “aralkyl” and “arylalkyl,” as used herein, refer to a radical formed between an alkyl group and an aryl group wherein the alkyl group is used to attach the aralkyl group to a parent molecule. Examples include, but are not limited to, benzyl, phenethyl and the like. Aralkyl groups as used herein may optionally include further substitutent groups attached to the alkyl, the aryl or both groups that form the radical group.

As used herein, the term “heterocyclic radical” as used herein, refers to a radical mono-, or poly-cyclic ring system that includes at least one heteroatom and is unsaturated, partially saturated or fully saturated, thereby including heteroaryl groups. Heterocyclic is also meant to include fused ring systems wherein one or more of the fused rings contain at least one heteroatom and the other rings can contain one or more heteroatoms or optionally contain no heteroatoms. A heterocyclic group typically includes at least one atom selected from sulfur, nitrogen or oxygen. Examples of heterocyclic groups include, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like. Heterocyclic groups as used herein may optionally include further substitutent groups. As used herein, the terms “heteroaryl,” and “heteroaromatic,” as used herein, refer to a radical comprising a mono- or poly-cyclic aromatic ring, ring system or fused ring system wherein at least one of the rings is aromatic and includes one or more heteroatom. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. Heteroaryl radicals can be attached to a parent molecule directly or through a linking moiety such as an aliphatic group or hetero atom. Heteroaryl groups as used herein may optionally include further substitutent groups.

As used herein, the term “heteroarylalkyl,” as used herein, refers to a heteroaryl group as previously defined having an alky radical that can attach the heteroarylalkyl group to a parent molecule. Examples include, but are not limited to, pyridinylmethyl, pyrimidinylethyl, napthyridinylpropyl and the like. Heteroarylalkyl groups as used herein may optionally include further substitutent groups on one or both of the heteroaryl or alkyl portions.

As used herein, the term “mono or poly cyclic structure” as used in the present invention includes all ring systems that are single or polycyclic having rings that are fused or linked and is meant to be inclusive of single and mixed ring systems individually selected from aliphatic, alicyclic, aryl, heteroaryl, aralkyl, arylalkyl, heterocyclic, heteroaryl, heteroaromatic, heteroarylalkyl. Such mono and poly cyclic structures can contain rings that are uniform or have varying degrees of saturation including fully saturated, partially saturated or fully unsaturated. Each ring can comprise ring atoms selected from C, N, O and S to give rise to heterocyclic rings as well as rings comprising only C ring atoms which can be present in a mixed motif such as for example benzimidazole wherein one ring has only carbon ring atoms and the fused ring has two nitrogen atoms. The mono or poly cyclic structures can be further substituted with substituent groups such as for example phthalimide which has two=0 groups attached to one of the rings. In another aspect, mono or poly cyclic structures can be attached to a parent molecule directly through a ring atom, through a substituent group or a bifunctional linking moiety.

As used herein, the term “acyl,” as used herein, refers to a radical formed by removal of a hydroxyl group from an organic acid and has the general formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substitutent groups.

As used herein, the term “hydrocarbyl” includes groups comprising C, O and H. Included are straight, branched and cyclic groups having any degree of saturation. Such hydrocarbyl groups can include one or more heteroatoms selected from N, O and S and can be further mono or poly substituted with one or more substituent groups.

As used herein, the terms “substituent” and “substituent group,” as used herein, include groups that are typically added to other groups or parent compounds to enhance desired properties or give desired effects. Substituent groups can be protected or unprotected and can be added to one available site or to many available sites in a parent compound. Substituent groups may also be further substituted with other substituent groups and may be attached directly or via a linking group such as an alkyl or hydrocarbyl group to a parent compound. Such groups include without limitation, halogen, hydroxyl, alkyl, alkenyl, alkynyl, acyl (—C(O)Raa), carboxyl (—C(O)O-Raa), aliphatic groups, alicyclic groups, alkoxy, substituted oxo (—O—Raa), aryl, aralkyl, heterocyclic, heteroaryl, heteroarylalkyl, amino (—NRbbRcc), imino (═NRbb), amido (—C(O)N-RbbRcc or —N(Rbb)C(O)Raa), azido (—N3), nitro (—NO2), cyano (—CN), carbamido (—OC(O)NRbbRcc or —N(Rbb)C(O)ORaa), ureido (—N(Rbb)C(O)NRbbRcc), thioureido (—N(Rbb)C(S)NRbbRcc), guanidinyl (—N(Rbb)C(═NRbb)NRbbRcc), amidinyl (—C(═NRbb)-NRbbRcc or —N(Rbb)C(NRbb)Raa), thiol (—SRbb), sulfinyl (—S(O)Rbb), sulfonyl (—S(O)2Rbb), sulfonamidyl (—S(O)2NRbbRcc or —N(Rbb)S(O)2Rbb) and conjugate groups. Wherein each Raa, Rbb and Rcc is, independently, H, an optionally linked chemical functional group or a further substituent group with a preferred list including without limitation H, alkyl, alkenyl, alkynyl, aliphatic, alkoxy, acyl, aryl, aralkyl, heteroaryl, alicyclic, heterocyclic and heteroarylalkyl.

### II. REVERSIR Compounds of the Invention

The present invention provides REVERSIR compounds which inhibit the RNAi inhibitory activity of a dsRNA agent comprising a thermally destabilizing nucleotide modification in the antisense strand.

Generally, the REVERSIR compounds of the invention are single stranded oligonucleotides (oligomers) 16-30 nucleotides in length, e.g., 16-24, 18-22, or 18-20 nucleotides in length. The single stranded oligonucleotides comprise a nucleotide sequence substantially complementary to the antisense strand of a dsRNA agent comprising a thermally destabilizing nucleotide modification. The nucleotide sequence of the oligonucleotides may be at least about 90%, e.g., 90%, 91%, 92%, 93%, 94%, 95%, 96%, 97%, 98%, 99%, or 100% complementary to the entire nucleotide sequence of the antisense strand of the dsRNA agent.

In certain embodiments, the REVERSIR compounds are chemically modified oligomeric compounds, compared to naturally occurring oligomers, such as DNA or RNA.

Thus, in certain embodiment, the REVERSIR compounds of the invention comprise a least one modified nucleotide, i.e., at least one modified monomer.

In other embodiments, substantially all of the nucleotides of the oligonucleotide are modified nucleotides, e.g., not more than 5, 4, 3, 2, or 1 of the nucleotides are unmodified nucleotides, e.g., substantially all of the nucleotides comprise a nucleotide modification selected from the group consisting of a 2′-O-alkyl modification, a 2′-substituted alkoxy modification, a 2′-substituted alkyl modification, a 2′-halo modification, a deoxynucleotide modification, a D-Methyleneoxy (4′-CH2-O-2′) locked nucleic acid (LNA) modification, a 2′-O-(2-Methoxyethyl) (MOE) modification, bri dged nucleic acid (2′4′-BN A), 2′-O-Ethyl (cEt), and a 2′-O-methyl modification.

In still other embodiment, all of the nucleotides of the oligonucleotide are modified nucleotides, e.g., all of the nucleotides comprise a nucleotide modification selected from the group consisting of a 2′-O-alkyl modification, a 2′-substituted alkoxy modification, a 2′-substituted alkyl modification, a 2′-halo modification, a deoxynucleotide modification, a D-Methyleneoxy (4′-CH2-O-2′) locked nucleic acid (LNA) modification, bridged nuclec acid (2′,4′ABNA), 2′-O-Ethyl (cEt), and a 2′-O-methyl modification.

In certain such embodiments, the REVERSIR compounds of the invention comprise one or more high affinity modifications. In one embodiment, REVERSIR compounds of the invention comprise four high affinity modifications. In one embodiment, REVERSIR compounds of the invention comprise five high affinity modifications.

In certain embodiments, such high affinity modification is selected from modifications (e.g., nucleosides and nucleotides) comprising 2′-modified sugars, including, but not limited to: BNA's and modifications (e.g., nucleosides and nucleotides) with 2′-substituents such as allyl, amino, azido, thio, O-allyl, O—C₁-C₁₀ alkyl, —OCF₃, O—(CH₂)₂—O—CH3, 2′-O(CH₂)₂SCH₃, O—(CH₂)₂—O—N(Rm)(Rn), or O—CH₂—C(═O)—N(Rm)(Rn), where each Rm and Rn is, independently, H or substituted or unsubstituted C₁-C₁₀ alkyl.

In certain embodiments, the REVERSIR compounds of the invention comprise one or more β-D-Methyleneoxy (4′-CH₂—O-2′) LNA modifications.

In certain embodiments, the REVERSIR compounds of the invention comprise one or more α-D-Methyleneoxy (4′-CH₂—O-2′) LNA modifications.

In certain embodiments, the REVERSIR compounds of the invention comprise one or more (S)-cEt modifications.

In certain embodiments, the REVERSIR compounds of the invention comprise one or more high affinity modifications provided that the compound does not comprise a nucleotide comprising a 2′-O(CH₂)ₙH, wherein n is one to six.

In certain embodiments, the REVERSIR compounds of the invention comprise one or more high affinity modifications provided that the compound does not comprise a nucleotide comprising a 2′-OCH₃ or a 2′-O(CH₂)₂OCH₃.

In certain embodiments, the REVERSIR compounds of the invention comprise one or more (e.g., 1, 2, 3, 4, 5, 6, 7, 8, 9, 10, 11, 12, 13, 14, 15 or more) high affinity modifications provided that the compound does not comprise a α-L-Methyleneoxy (4′-CH₂—O-2′) LNA.

In certain embodiments, the REVERSIR compounds of the invention comprise one or more high affinity modifications provided that the compound does not comprise a β-D-Methyleneoxy (4′-CH₂—O-2′) LNA.

In certain embodiments, the REVERSIR compounds of the invention comprise one or more high affinity modifications provided that the compound does not comprise a α-L-Methyleneoxy (4′-CH₂—O-2′) LNA or β-D-Methyleneoxy (4′-CH₂—O-2′) LNA.

In some embodiments, at least one of the nucleotides comprising a high affinity nucleotide modification is base paired with the nucleotide comprising the thermally destabilizing nucleotide in the antisense strand of the dsRNA agent.

In some embodiments, the REVERSIR compounds of the invention comprise at least two high affinity nucleotide modifications, e.g., LNAs. In some embodiments, the at least two high affinity nucleotide mofifications, e.g., LNAs, are at positions 2 and 6; positions 2 and 5; positions 2 and 7; positions 2 and 8; positions 2 and 9; positions 2 and 14; positions 2 and 15; and/or positions 2 and 16, counting from the 3′-end of the oligonucleotide.

In one embodiment, REVERSIR compounds of the invention comprise four high affinity modifications, e.g., four LNAs. In one embodiment, REVERSIR compounds of the invention comprise five high affinity modifications, e.g., five LNAs.

In certain embodiments, the high affinity nucleotide modifications, e.g., LNAs, are at positions 2, 6, 8, and 14; 2, 4, 5, 6, and 7; 2, 4, 6, 8, and 13; 2, 4, 6, 8, and 14; 2, 4, 6, 8, and 15; 2, 4, 6, 8, and 16; 2, 8, 10, and 14; 2, 4, 6, 8, and 14; or 2, 8, 12, and 14, counting from the 3′-end of the oligonucleotide.

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside or internucleotide backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide oligonucleotides having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

A REVERSIR compound as described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the oligonucleotide duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

In some embodiements, the REVERSIR compounds of the invention comprise at least one (e.g., 1, 2, 3, 4, 5, 6, 7, 8, 9, or 10) G-clamp nucleobase selected from the following:

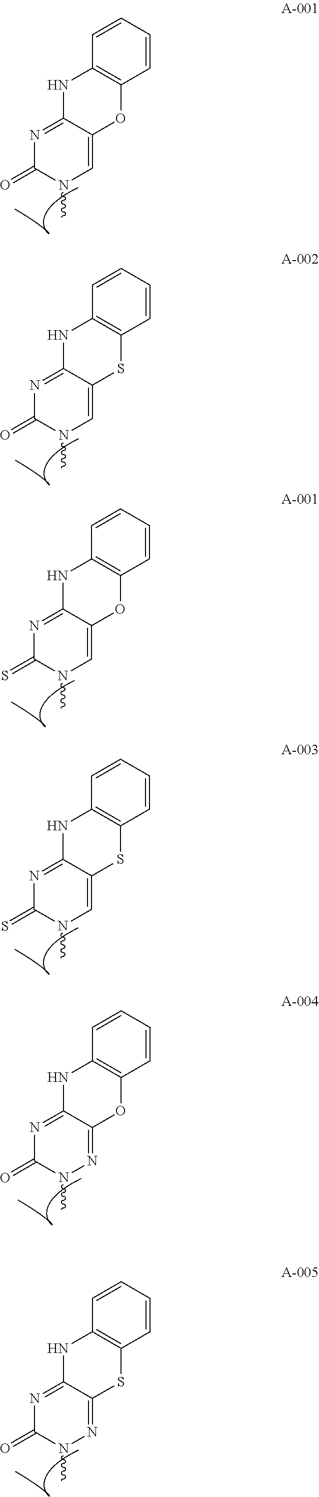

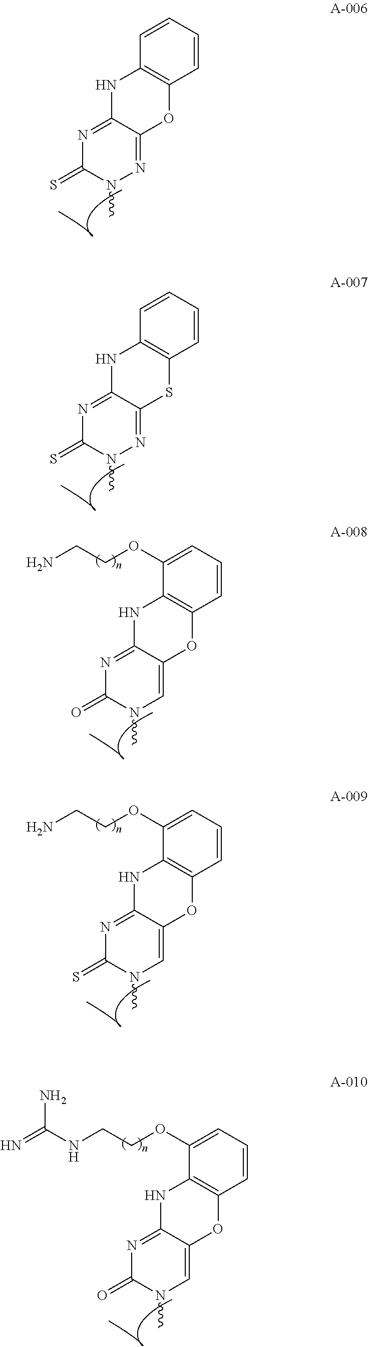

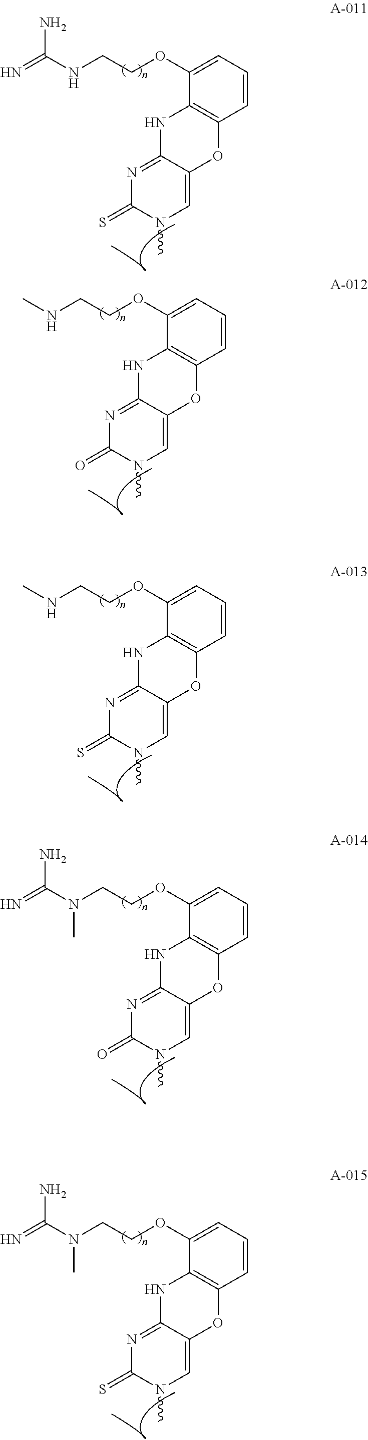

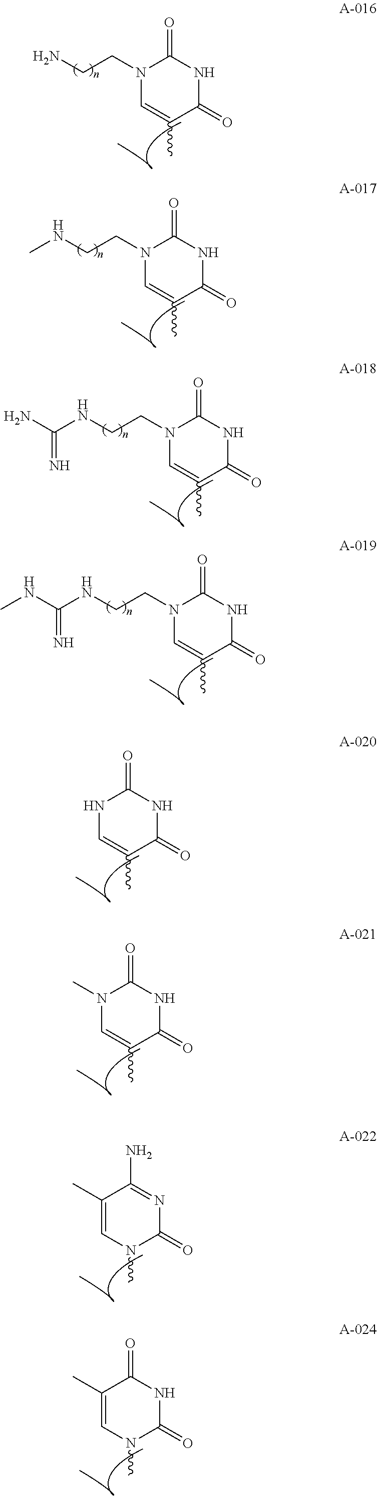

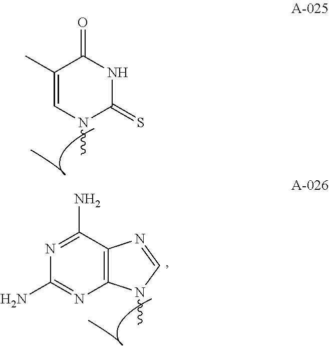

where n is 0, 1, 2, 3, 4, 5 or 6.

The REVERSIR compounds provided herein can comprise one or more monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, compounds comprise one or more monomers that are LNA.

In some embodiments of a locked nucleic acid, the 2′ position of furnaosyl is connected to the 4′ position by a linker selected independently from —[C(R1)(R2)]ₙ-, —[C(R1)(R2)]ₙ—O—, —[C(R1)(R2)]ₙ—N(R1)-, —[C(R1)(R2)]ₙ—N(R1)-O—, —[C(R1R2)]ₙ-O—N(R1)-, —C(R1)=C(R2)-O—, —C(R1)=N—, —C(R1)=N—O—, —C(═NR1)-, —C(═NR1)-O—, —C(═O)—, —C(═O)O—, —C(═S)—, —C(═S)O—, —C(═S)S—, —O—, —Si(R1)2-, —S(═O)ₓ— and —N(R1)-;
wherein:
x is 0, 1, or 2;
n is 1, 2, 3, or 4;
each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and
each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In one embodiment, each of the linkers of the LNA compounds is, independently, —[C(R1)(R2)]n-, —[C(R1)(R2)]n-O—, —C(R1R2)-N(R1)-O— or —C(R1R2)-O—N(R1)-. In another embodiment, each of said linkers is, independently, 4′-CH₂-2′, 4′-(CH₂)₂-2′, 4′—(CH₂)₃-2′, 4′—CH₂—O-2′, 4′—(CH₂)₂—O-2′, 4′—CH₂—O—N(R1)-2′ and 4′-CH₂—N(R1)-O-2′- wherein each RI is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel comformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)2-O-2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application PublicationNo. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, a REVERSIR compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

The REVERSIR compounds of the invention can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. REVERSIR compounds can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in a configuration.

Sugar modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

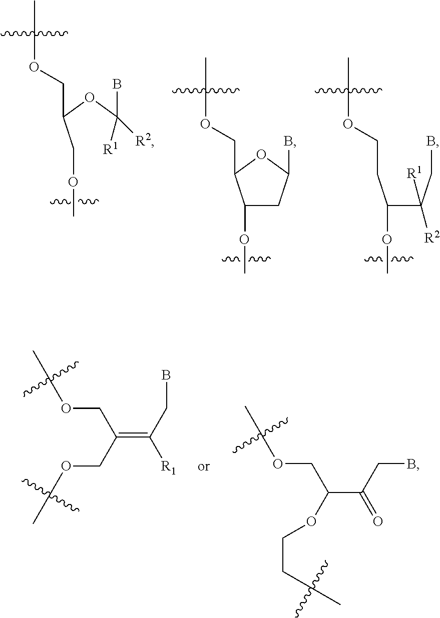

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O-[2-(methylamino)-2-oxoethyl](2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

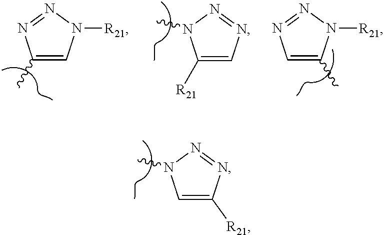

NR₂₁R₃₁, CONR₂₁R₃₁, CON(H)NR₂₁R₃₁, ONR₂₁R₃₁, CON(H)N═CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R₅₁, SO₂R₁₁, SOR₁₁, SR₁₁, and substituted or unsubstituted heterocyclic: R₂₁ and R₃₁ for each occurtence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heter Earyi, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the oligonucleotide.

In certain embodiments, LNA's include bicyclic nucleotide having the formula:

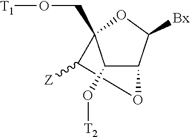

wherein:
Bx is a heterocyclic base moiety;
T1 is H or a hydroxyl protecting group;
T2 is H, a hydroxyl protecting group or a reactive phosphorus group;
Z is C1-C6 alkyl, C2-C6 alkenyl, C2-C6 alkynyl, substituted C1-C6 alkyl, substituted C2-C6 alkenyl, substituted C₂-C₆ alkynyl, acyl, substituted acyl, or substituted amide.

In one embodiment, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C1-C6 alkyl, and X is O, S or NJ1.

In certain such embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H, C1-C6 alkyl, or substituted C1-C6 alkyl and X is O or NJ1.

In certain embodiments, the Z group is C1-C6 alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, the Z group is C1-C6 alkyl substituted with one or more Xx, wherein each Xx is independently halo (e.g., fluoro), hydroxyl, alkoxy (e.g., CH3O—), substituted alkoxy or azido.

In certain embodiments, the Z group is —CH2Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, the Z group is —CH2Xx, wherein Xx is halo (e.g., fluoro), hydroxyl, alkoxy (e.g., CH3O—) or azido.

In certain such embodiments, the Z group is in the (R)-configuration:

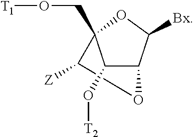

In certain such embodiments, the Z group is in the (S)-configuration:

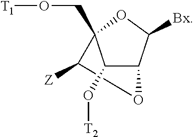

In certain embodiments, each T1 and T2 is a hydroxyl protecting group. A preferred list of hydroxyl protecting groups includes benzyl, benzoyl, 2,6-dichlorobenzyl, t-butyldimethylsilyl, t-butyldiphenylsilyl, mesylate, tosylate, dimethoxytrityl (DMT), 9-phenylxanthine-9-yl (Pixyl) and 9-(p-methoxyphenyl)xanthine-9-yl (MOX). In certain embodiments, T1 is a hydroxyl protecting group selected from acetyl, benzyl, t-butyldimethylsilyl, t-butyldiphenylsilyl and dimethoxytrityl wherein a more preferred hydroxyl protecting group is T1 is 4,4′-dimethoxytrityl.

In certain embodiments, T2 is a reactive phosphorus group wherein preferred reactive phosphorus groups include diisopropylcyanoethoxy phosphoramidite and H-phosphonate. In certain embodiments T1 is 4,4′-dimethoxytrityl and T2 is diisopropylcyanoethoxy phosphoramidite.

In certain embodiments, REVERSIR compounds have at least one monomer of the formula:

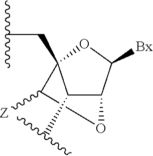

or of the formula:

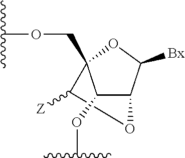

or of the formula:

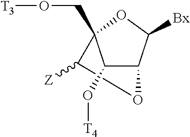

wherein
Bx is a heterocyclic base moiety;
T3 is H, a hydroxyl protecting group, a linked conjugate group or an internucleoside linking group attached to a nucleoside, a nucleotide, an oligonucleoside, an oligonucleotide, a monomeric subunit or an oligomeric compound;
T4 is H, a hydroxyl protecting group, a linked conjugate group or an internucleoside linking group attached to a nucleoside, a nucleotide, an oligonucleoside, an oligonucleotide, a monomeric subunit or an oligomeric compound;
wherein at least one of T3 and T4 is an internucleoside linking group attached to a nucleoside, a nucleotide, an oligonucleoside, an oligonucleotide, a monomeric subunit or an oligomeric compound; and
Z is C1-C6 alkyl, C2-C6 alkenyl, C2-C6 alkynyl, substituted C1-C6 alkyl, substituted C2-C6 alkenyl, substituted C2-C6 alkynyl, acyl, substituted acyl, or substituted amide.

In one embodiment, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C1-C6 alkyl, and X is O, S or NJ1.

In one embodiment, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H or C1-C6 alkyl, and X is O or NJ1.

In certain such embodiments, at least one Z is C1-C6 alkyl or substituted C1-C6 alkyl. In certain embodiments, each Z is, independently, C1-C6 alkyl or substituted C1-C6 alkyl. In certain embodiments, at least one Z is C1-C6 alkyl. In certain embodiments, each Z is, independently, C1-C6 alkyl. In certain embodiments, at least one Z is methyl. In certain embodiments, each Z is methyl. In certain embodiments, at least one Z is ethyl. In certain embodiments, each Z is ethyl. In certain embodiments, at least one Z is substituted C1-C6 alkyl. In certain embodiments, each Z is, independently, substituted C1-C6 alkyl. In certain embodiments, at least one Z is substituted methyl. In certain embodiments, each Z is substituted methyl. In certain embodiments, at least one Z is substituted ethyl. In certain embodiments, each Z is substituted ethyl.

In certain embodiments, at least one substituent group is C1-C6 alkoxy (e.g., at least one Z is C1-C6 alkyl substituted with one or more C1-C6 alkoxy). In another embodiment, each substituent group is, independently, C1-C6 alkoxy (e.g., each Z is, independently, C1-C6 alkyl substituted with one or more C1-C6 alkoxy).

In certain embodiments, at least one C1-C6 alkoxy substituent group is CH3O— (e.g., at least one Z is CH₃OCH₂—). In another embodiment, each C1-C6 alkoxy substituent group is CH₃O— (e.g., each Z is CH₃OCH₂—).

In certain embodiments, at least one substituent group is halogen (e.g., at least one Z is C1-C6 alkyl substituted with one or more halogen). In certain embodiments, each substituent group is, independently, halogen (e.g., each Z is, independently, C1-C6 alkyl substituted with one or more halogen). In certain embodiments, at least one halogen substituent group is fluoro (e.g., at least one Z is CH₂FCH₂—, CHF₂CH₂— or CF₃CH₂—). In certain embodiments, each halo substituent group is fluoro (e.g., each Z is, independently, CH₂FCH₂—, CHF₂CH₂— or CF₃CH₂—).

In certain embodiments, at least one substituent group is hydroxyl (e.g., at least one Z is C1-C6 alkyl substituted with one or more hydroxyl). In certain embodiments, each substituent group is, independently, hydroxyl (e.g., each Z is, independently, C1-C6 alkyl substituted with one or more hydroxyl). In certain embodiments, at least one Z is HOCH₂—. In another embodiment, each Z is HOCH₂—.

In certain embodiments, at least one Z is CH₃—, CH₃CH₂—, CH₂OCH₃—, CH₂F— or HOCH₂—. In certain embodiments, each Z is, independently, CH₃—, CH₃CH₂—, CH₂OCH₃—, CH₂F— or HOCH₂—.

In certain embodiments, at least one Z group is C1-C6 alkyl substituted with one or more Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, at least one Z group is C1-C6 alkyl substituted with one or more Xx, wherein each Xx is, independently, halo (e.g., fluoro), hydroxyl, alkoxy (e.g., CH3O—) or azido.

In certain embodiments, each Z group is, independently, C1-C6 alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, each Z group is, independently, C1-C6 alkyl substituted with one or more Xx, wherein each Xx is independently halo (e.g., fluoro), hydroxyl, alkoxy (e.g., CH3O—) or azido.

In certain embodiments, at least one Z group is —CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1 In certain embodiments, at least one Z group is —CH₂Xx, wherein Xx is halo (e.g., fluoro), hydroxyl, alkoxy (e.g., CH₃O—) or azido.

In certain embodiments, each Z group is, independently, —CH₂Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C1-C6 alkyl, and X is O, S or NJ1. In another embodiment, each Z group is, independently, —CH₂Xx, wherein each Xx is, independently, halo (e.g., fluoro), hydroxyl, alkoxy (e.g., CH₃O—) or azido.

In certain embodiments, at least one Z is CH₃—. In another embodiment, each Z is, CH₃.

In certain embodiments, the Z group of at least one monomer is in the (R)-configuration represented by the formula:

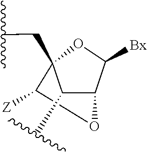

or the formula:

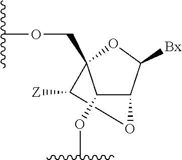

or the formula:

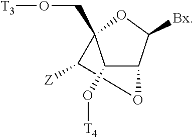

In certain embodiments, the Z group of each monomer of the formula is in the (R)-configuration.

In certain embodiments, the Z group of at least one monomer is in the (S)-configuration represented by the formula:

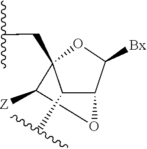

or the formula:

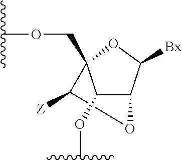

or the formula:

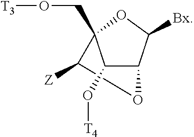

In certain embodiments, the Z group of each monomer of the formula is in the (S)-configuration.

In certain embodiments, T3 is H or a hydroxyl protecting group. In certain embodiments, T4 is H or a hydroxyl protecting group. In a further embodiment T3 is an internucleoside linking group attached to a nucleoside, a nucleotide or a monomeric subunit. In certain embodiments, T4 is an internucleoside linking group attached to a nucleoside, a nucleotide or a monomeric subunit. In certain embodiments, T3 is an internucleoside linking group attached to an oligonucleoside or an oligonucleotide. In certain embodiments, T4 is an internucleoside linking group attached to an oligonucleoside or an oligonucleotide. In certain embodiments, T3 is an internucleoside linking group attached to an oligomeric compound. In certain embodiments, T4 is an internucleoside linking group attached to an oligomeric compound. In certain embodiments, at least one of T3 and T4 comprises an internucleotide linking group selected from phosphodiester or phosphorothioate.

In certain embodiments, REVERSIR compounds have at least one region of at least two contiguous monomers of the formula:

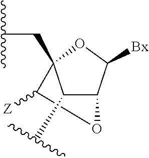

or of the formula:

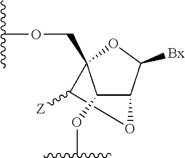

or of the formula:

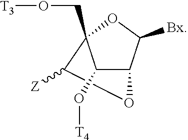

In certain such embodiments, LNAs include, but are not limited to, (A) α-L-Methyleneoxy (4′-CH2-O-2′) LNA, (B) β-D-Methyleneoxy (4′-CH2-O-2′) LNA, (C) Ethyleneoxy (4′-(CH2)2-O-2′) LNA, (D) Aminooxy (4′-CH2-O—N(R)-2′) LNA and (E) Oxyamino (4′-CH2-N(R)—O-2′) LNA, as depicted below:

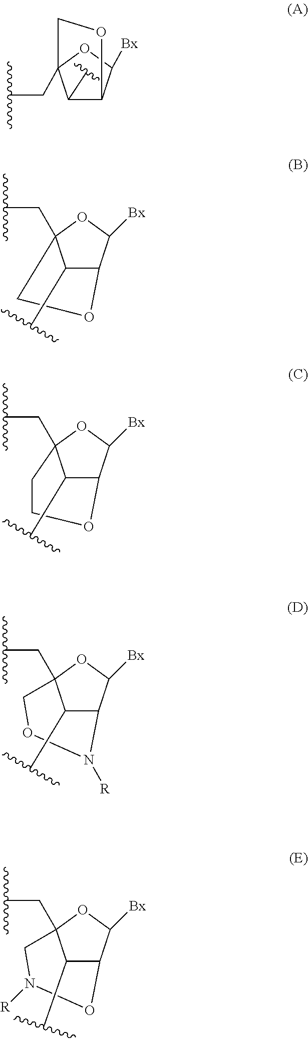

In certain embodiments, the REVERSIR compounds of the invention comprises at least two regions of at least two contiguous monomers of the above formula. In certain embodiments, the compound comprises a gapped oligomeric compound. In certain embodiments, the REVERSIR compounds of the invention comprises at least one region of from about 8 to about 14 contiguous β-D-2′-deoxyribofuranosyl nucleosides. In certain embodiments, the compound comprises at least one region of from about 9 to about 12 contiguous β-D-2′-deoxyribofuranosyl nucleosides.

In certain embodiments, the REVERSIR compound comprises at least one (e.g., 1, 2, 3, 4, 5, 6, 7, 8, 9, 10, 11, 12, 13, 14, 15 or more)S-cEt monomer of the formula:

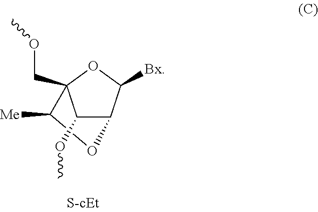

wherein Bx IS heterocyclic base moiety.

In some embodiments, the REVERSIR compounds of the invention comprises at least one (e.g., 1, 2, 3, 4, 5, 6, 7, 8, 9, 10, 11, 12, 13, 14, 15 or more) nucleotide selected from the following:

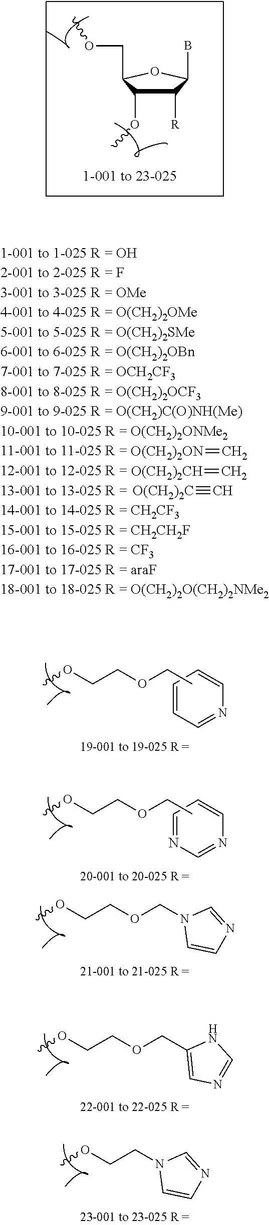

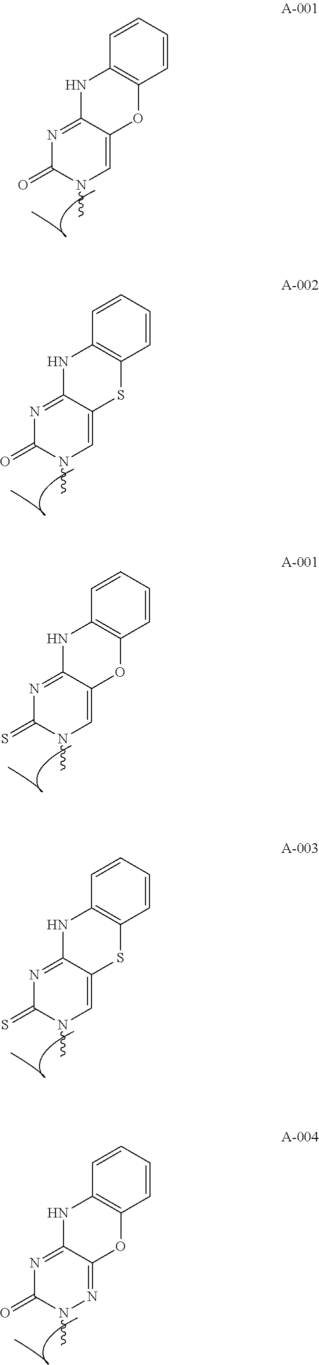

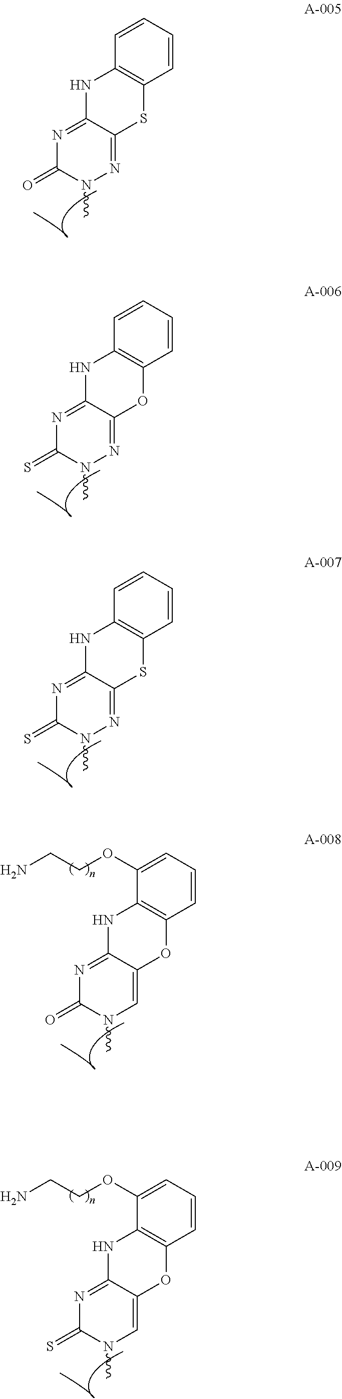

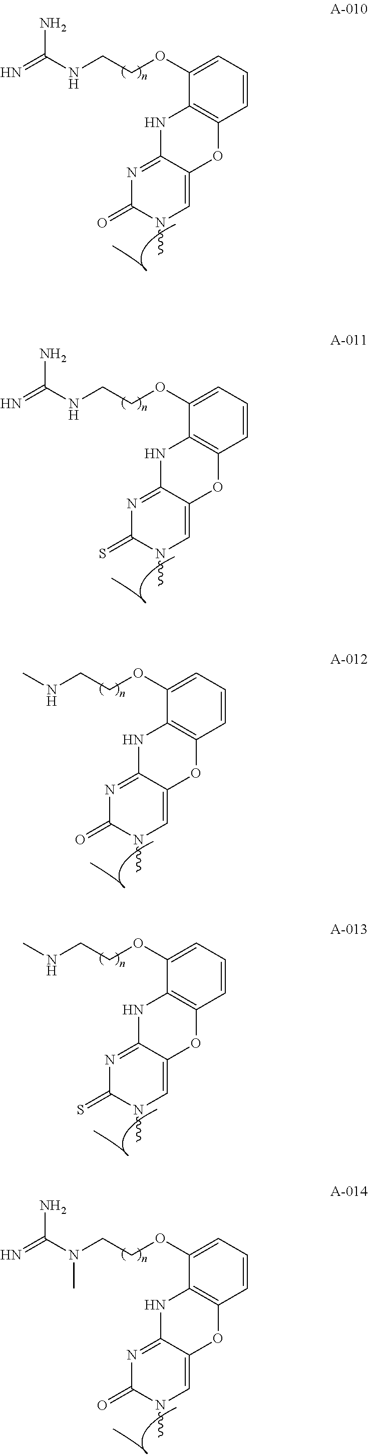

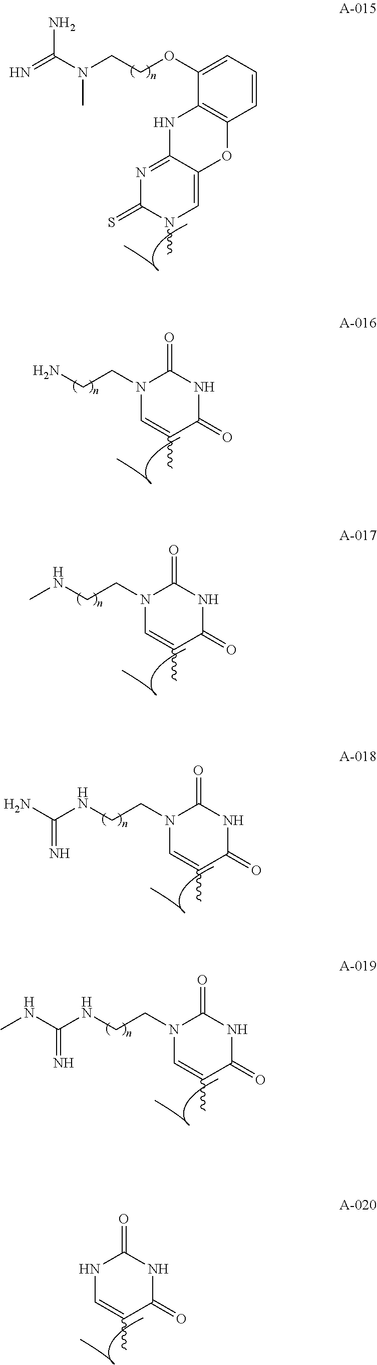

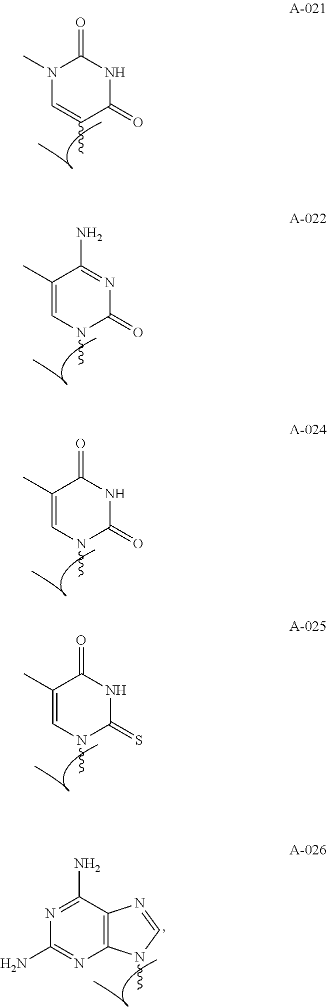

where B is A-001 to A-026 and n is 0-6 (e.g., 0, 1, 2, 3, 4, 5 or 6).

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside, nucleotide and nucleobase mimetics are well known to those skilled in the art.

In certain embodiments, the REVERSIR compounds of the invention comprise at least one monomer that is LNA and at least one G-clamp nucleobase. For example, the REVERSIR compound can comprise 1, 2, 3, 4, 5, 6, 7, 8, 9, 10, 11, 12, 13, 14, 15 or more monomers that are LNA 1, 2, 3, 4, 5, 6, 7, 8, 9, 10, 11, 12, 13, 14, 15 or more G-clamp nucleobases.

In some embodiments, the REVERSIR compounds of the invention comprise at least one (e.g., 1, 2, 3, 4, 5, 6, 7, 8, 9, 10, 11, 12, 13, 14, 15 or more) peptide nucleic acid monomer. In certain embodiments, the REVERSIR compound comprises at least one monomer that is LNA and at least one monomer that is PNA. For example, the REVERSIR compound can comprise 1, 2, 3, 4, 5, 6, 7, 8, 9, 10, 11, 12, 13, 14, 15 or more monomers that are LNA 1, 2, 3, 4, 5, 6, 7, 8, 9, 10, 11, 12, 13, 14, 15 or more monomers that are PNA.

In certain embodiments, the REVERSIR compounds of the invention comprise at least one PNA monomer and at least one G-clamp nucleobase. For example, the REVERSIR compound can comprise 1, 2, 3, 4, 5, 6, 7, 8, 9, 10, 11, 12, 13, 14, 15 or more PNA monomers and 1, 2, 3, 4, 5, 6, 7, 8, 9, 10, 11, 12, 13, 14, 15 or more G-clamp nucleobases.

In certain embodiments, the REVERSIR compounds of the invention comprise at least one LNA monomer, at least one PNA monomer and at least one G-clamp nucleobase. For example, the REVERSIR compound can comprise 1, 2, 3, 4, 5, 6, 7, 8, 9, 10, 11, 12, 13, 14, 15 or more LNA monomers; 1, 2, 3, 4, 5, 6, 7, 8, 9, 10, 11, 12, 13, 14, 15 or more PNA monomers and 1, 2, 3, 4, 5, 6, 7, 8, 9, 10, 11, 12, 13, 14, 15 or more G-clamp nucleobases.

Described herein are linking groups that link monomers (including, but not limited to, modified and unmodified nucleosides and nucleotides) together, thereby forming an oligomeric compound, i.e., a REVERSIR compound comprising an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P=O), phosphotriesters, methylphosphonates, phosphoramidate, and phosphorothioates (P=S). Representative non-phosphorus containing linking groups include, but are not limited to, methylenemethylimino (—CH2-N(CH3)-O—CH2-), thiodiester (—O—C(O)—S—), thionocarbamate (—O—C(O)(NH)—S—); siloxane (—O—Si(H)2-O—); and N,N′-dimethylhydrazine (—CH2-N(CH3)-N(CH3)-). Oligomeric compounds having non-phosphorus linking groups are referred to as oligonucleosides. Modified linkages, compared to natural phosphodiester linkages, can be used to alter, typically increase, nuclease resistance of the oligomeric compound. In certain embodiments, linkages having a chiral atom can be prepared a racemic mixtures, as separate enantomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc.,), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.” In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH2 component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

In some embodiments, the REVERSIR compounds of the invention comprise at least one (e.g., 1, 2, 3, 4, 5, 6, 7, 8, 9, 10, 11, 12, 13, 14, 15 or more and upto including all) modified or nonphosphodiester linkages. In one embodiment, the REVERSIR compounds of the invention comprise at least one (e.g., 1, 2, 3, 4, 5, 6, 7, 8, 9, 10, 11, 12, 13, 14, 15 or more and up to including all) phosphorothioate linkages.

In some embodiments, the REVERSIR compounds of the invention comprise at least five phosphorothioate internucleotide modifications. In other embodiments, the REVERSIR compounds of the invention comprise 5-15 phosphorothioate internucleotide modifications; 5-14 phosphorothioate internucleotide modifications; 5-13 phosphorothioate internucleotide modifications; 5-12 phosphorothioate internucleotide modifications; 5-11 phosphorothioate internucleotide modifications; 5-10 phosphorothioate internucleotide modifications; 5-9 phosphorothioate internucleotide modifications; 5-8 phosphorothioate internucleotide modifications; 5-7 phosphorothioate internucleotide modifications; or 5-6 phosphorothioate internucleotide modifications. In still other embodiments, the REVERSIR compounds of the invention comprise 6-14 phosphorothioate internucleotide modifications.

In some embodiments, all internucleotide linkages in the reverser compounds are phosphorothioate (PS) internucleotide linkages. In certain embodiments, the REVERSIR compounds comprise at least one phosphorothioate (PS) internucleotide linkage, but not all internucleotide linkages in said REVERSIR compound are a phosphorothioate linkage. In other words, in some embodiments, less than 100% (e.g., 95%, 90%, 85%, 80%, 75%, 70%, 65%, 60%, 55%, 50%, 45%, 40% or fewer) of the internucleotide linkages are phosphorothioate linkages.

In some embodiments, the REVERSIR compounds comprise at least one phosphorothioate internucleotide linkage and at least one internucleoside or internucleotide linkage that is not a phosphorothioate. For example, the REVERSIR compounds comprise at least one phosphorothioate internucleotide linkage and at least one phosphodiester internucleotide linkage. In some embodiments, the non-phosphorothioate internucleotide linkage is between the terminus and the penultimate nucleotides.

In some embodiments, the internucleotide linkage between the nucleobase at the 3′-terminus of the REVERSIR compound and the rest of the REVERSIR compound is a phosphodiester linkage. In some embodiments, all internucleotide linkages in the REVERSIR compounds are phosphorothioate except for the internucleotide linkage between the nucleotide at the 3′-terminus of the REVERSIR compound and the rest of the REVERSIR compound.

REVERSIR compounds can also be constructed wherein the phosphate linker and the sugar are replaced by nuclease resistant nucleoside, nucleotide or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. A preferred surrogate is a PNA surrogate.

The REVERSIR compounds described herein may contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the antisense compounds provided herein are all such possible isomers, as well as their racemic and optically pure forms.

Ends of the REVERSIR compounds of the invention may be modified. Such modifications can be at one end or both ends. For example, the 3′ and/or 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligomeric compound, this array can substitute for a hairpin loop in a hairpin-type compound.

Terminal modifications useful for modulating activity include modification of the 5′ end of compound with phosphate or phosphate analogs. In certain embodiments, the 5′end of compound is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the compound comprises the modification

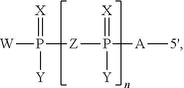

wherein W, X and Y are each independently selected from the group consisting of O, OR (R is hydrogen, alkyl, aryl), S, Se, BR₃ (R is hydrogen, alkyl, aryl), BH₃—, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently 0, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modificaitons include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}-5′, ((HO)2(X)P—O[—(CH₂)ₐ—P(X)(OH)—O]_{b}-5′, ((HO)2(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

In certain embodiments, the REVERSIR compounds of the invention are chimeric oligomeric compounds, i.e., chimeric oligonucleotides. In certain such embodiments, the chimeric oligonucleotides comprise differently modified nucleotides. In certain embodiments, chimeric oligonucleotides are mixed-backbone antisense oligonucleotides.

In general, a chimeric oligomeric compound will have modified nucleosides that can be in isolated positions or grouped together in regions that will define a particular motif. Any combination of modifications and/or mimetic groups can comprise a chimeric oligomeric compound as described herein.

In certain embodiments, chimeric oligomeric compounds typically comprise at least one region modified so as to confer increased resistance to nuclease degradation, increased cellular uptake, and/or increased binding affinity for the target nucleic acid. In certain embodiments, an additional region of the oligomeric compound may serve as a substrate for enzymes capable of cleaving RNA:DNA or RNA:RNA hybrids.

In certain embodiments, chimeric oligomeric compounds are gapmers. In certain such embodiments, a mixed-backbone oligomeric compound has one type of internucleotide linkages in one or both wings and a different type of internucleoside linkages in the gap. In certain such embodiments, the mixed-backbone oligonucleotide has phosphodiester linkages in the wings and phosphorothioate linkages in the gap. In certain embodiments in which the internucleotide linkages in a wing is different from the internucleotide linkages in the gap, the internucleotide linkage bridging that wing and the gap is the same as the internucleotide linkage in the wing. In certain embodiments in which the internucleotide linkages in a wing is different from the internucleotide linkages in the gap, the internucleotide linkage bridging that wing and the gap is the same as the internucleotide linkage in the gap.

In certain embodiments, the present invention provides REVERSIR compounds of any of a variety of ranges of lengths. In certain embodiments, the invention provides compounds consisting of 16-30, e.g., 16, 17, 18, 19, 20, 21, 22, 23, 24, 25, 26, 27, 28, 29, or 30 nucleotides. For example, in certain embodiments, the invention provides compounds comprising: 16-17, 16-18, 16-19, 16-25, 16-21, 16-22, 16-23, 16-24, 16-25, 16-26, 16-27, 16-28, 16-29, 16-30, 17-18, 17-19, 17-20, 17-21, 17-22, 17-23, 17-24, 17-25, 17-26, 17-27, 17-28, 17-29, 17-30, 18-19, 18-20, 18-21, 18-22, 18-23, 18-24, 18-25, 18-26, 18-27, 18-28, 18-29, 18-30, 19-20, 19-21, 19-22, 19-23, 19-24, 19-25, 19-26, 19-29, 19-28, 19-29, 19-30, 20-21, 20-22, 20-23, 20-24, 20-25, 20-26, 20-27, 20-28, 20-29, 20-30, 21-22, 21-23, 21-24, 21-25, 21-26, 21-27, 21-28, 21-29, 21-30, 22-23, 22-24, 22-25, 22-26, 22-27, 22-28, 22-29, 22-30, 23-24, 23-25, 23-26, 23-27, 23-28, 23-29, 23-30, 24-25, 24-26, 24-27, 24-28, 24-29, 24-30, 25-26, 25-27, 25-28, 25-29, 25-30, 26-27, 26-28, 26-29, 26-30, 27-28, 27-29, 27-30, 28-29, 28-30, or 29-30 linked nucleotides.

As noted-above, REVERSIR compounds can be of any length. For example, in some embodiments, the REVERSIR compound is a modified oligonucleotide consisting of 16-30 nucleotides. For example, the REVERSIR compound can consist of 6, 7, 8, 9, 10, 11, 12, 13, 14, 15, 16, 17, 18, 19, 20, 21, 22, 23, 24, 25, 26, 27, 28, 29 or 30 linked nucleobases. In some embodiments, the REVERSIR compound consists of 16-24, 16-22 or 18-20 linked nucleobases.

As discussed herein, REVERSIR compounds are oligonucleotides, e.g., modified oligonucleotides, that are substantially complementary to the antisense strand of a dsRNA agent comprising a thermally destabilizing nucleotide modification in the antisense strand. Now without wishing to be bound by a theory, REVERSIR compounds that are substantially complementary to the seed region of the antisense strand of the dsRNA (i.e., at positions 2-8, or 2-9 of the 5′-end of the antisense strand) are particularly effective in reducing dsRNA activity. Thus, in many embodiments, the REVERSIR compound is substantially complementary to nucleotides 2-8, 2-9, 2-10, 2-11, 2-12, 2-13, 2-14, 2-15 or 2-16 of the antisense strand of the dsRNA agents described herein. By substantially complementary in this context is meant a complementarity of at least 90%, preferably at least 95%, and more preferably complete complementarity.

### A. Modified REVERSIR Compounds Comprising Motifs of the Invention

The present invention also includes oligomeric compounds 18-24 nucleotides in length which comprise a nucleotide sequence substantially complementary to the antisense strand of the dsRNA agent. Such oligomeric compounds generally include at least five phosphorothioate internucleotide modifications and is represented by formula (I):

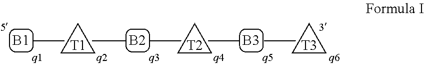

wherein:
B1, B2 and B3 each independently represent a nucleotide comprising a nucleotide modification independently selected from the group consisting of a 2′-deoxy, 2′-ribo, 2′-O-alkyl modification, a 2′-substituted alkoxy modification, 2-substituted alkoxy alkyl modification, a 2′-substituted alkyl modification, and a 2′-halo modification;
T1, T2, and T3 each independently represent a nucleotide comprising a nucleotide modification selected from the group consisting of a deoxynucleotide modification, a. D-Methyleneoxy (4′-CH2-O-2′) locked nucleic acid (LNA) modification, a 2′-O-(2-˜Methoxyethyl) (MOE) modification, a cEt modification, or a different BNA modification, a 2′-deoxy-2′-Fluoro, and a 2′-O-methyl modification;
q¹, q³ and q⁵ are each independently 3-12 nucleotides in length;
q², q⁴ and q⁶ are independently 1-6 nucleotides) in length.

In other embodiments, the present invention provides oligomeric compounds which are chimeric oligomeric compounds, i.e. chimeric REVERSIR compounds. “Chimeric” oligomeric compounds or “chimeras,” in the context of this invention, are oligomeric compounds which contain two or more chemically distinct regions, each made up of at least one monomer unit, i.e., a modified or unmodified nucleotide in the case of an oligonucleotide. Chimeric oligomeric compounds can be described as having a particular motif. In some embodiments, the motifs include, but are not limited to, an alternating motif, a gapped motif, a hemimer motif, a uniformly fully modified motif and a positionally modified motif. As used herein, the phrase “chemically distinct region” refers to an oligomeric region which is different from other regions by having a modification that is not present elsewhere in the oligomeric compound or by not having a modification that is present elsewhere in the oligomeric compound. An oligomeric compound can comprise two or more chemically distinct regions. As used herein, a region that comprises no modifications is also considered chemically distinct.

A chemically distinct region can be repeated within an oligomeric compound. Thus, a pattern of chemically distinct regions in an oligomeric compound can be realized such that a first chemically distinct region is followed by one or more second chemically distinct regions. This sequence of chemically distinct regions can be repeated one or more times. Preferably, the sequence is repeated more than one time. Both strands of a double-stranded oligomeric compound can comprise these sequences. Each chemically distinct region can actually comprise as little as a single monomers, e.g., nucleotides. In some embodiments, each chemically distinct region comprises 1, 2, 3, 4, 5, 6, 7, 8, 9, 10, 11, 12, 13, 14, 15, 16, 17 or 18 monomers, e.g., nucleotides.

In some embodiments, alternating nucleotides comprise the same modification, e.g. all the odd number nucleotides in a strand have the same modification and/or all the even number nucleotides in a strand have the similar modification to the first strand. In some embodiments, all the odd number nucleotides in an oligomeric compound have the same modification and all the even numbered nucleotides have a modification that is not present in the odd number nucleotides and vice versa.

In some embodiments, the oligonucleotide comprises two chemically distinct regions, wherein each region is 1, 2, 3, 4, 5, 6, 7, 8, 9 or 10 nucleotides in length.

In other embodiments, the oligomeric compound comprises three chemically distinct region. The middle region is about 5-15, (e.g., 5, 6, 7, 8, 9, 10, 11, 12, 13, 14 or 15) nucleotide in length and each flanking or wing region is independently 1-10 (e.g., 1, 2, 3, 4, 5, 6, 7, 8, 9 or 10) nucleotides in length. All three regions can have different modifications or the wing regions can be similarly modified to each other. In some embodiments, the wing regions are of equal length, e.g. 1, 2, 3, 4, 5, 6, 7, 8, 9 or 10 nucleotides long.

As used herein the term “alternating motif” refers to an oligomeric compound comprising a contiguous sequence of linked monomer subunits wherein the monomer subunits have two different types of sugar groups that alternate for essentially the entire sequence of the oligomeric compound. Oligomeric compounds having an alternating motif can be described by the formula: 5′-A(-L-B-L-A)n(-L-B)nn-3′ where A and B are monomelic subunits that have different sugar groups, each L is an internucleoside linking group, n is from about 4 to about 12 and nn is 0 or 1. This permits alternating oligomeric compounds from about 9 to about 26 monomer subunits in length. This length range is not meant to be limiting as longer and shorter oligomeric compounds are also amenable to the present invention. In one embodiment, one of A and B is a 2′-modified nucleoside as provided herein.

As used herein, “type of modification” in reference to a nucleoside or a nucleoside of a “type” refers to the modification of a nucleoside and includes modified and unmodified nucleosides. Accordingly, unless otherwise indicated, a “nucleoside having a modification of a first type” may be an unmodified nucleoside.

As used herein, “type region” refers to a portion of an oligomeric compound wherein the nucleosides and internucleoside linkages within the region all comprise the same type of modifications; and the nucleosides and/or the internucleoside linkages of any neighboring portions include at least one different type of modification. As used herein the term “uniformly fully modified motif” refers to an oligonucleotide comprising a contiguous sequence of linked monomer subunits that each have the same type of sugar group. In one embodiment, the uniformly fully modified motif includes a contiguous sequence of nucleosides of the invention. In one embodiment, one or both of the 3′ and 5′-ends of the contiguous sequence of the nucleosides provided herein, comprise terminal groups such as one or more unmodified nucleosides.

As used herein the term “hemimer motif” refers to an oligomeric compound having a short contiguous sequence of monomer subunits having one type of sugar group located at the 5′ or the 3′ end wherein the remainder of the monomer subunits have a different type of sugar group. In general, a hemimer is an oligomeric compound of uniform sugar groups further comprising a short region (1, 2, 3, 4 or about 5 monomelic subunits) having uniform but different sugar groups and located on either the 3′ or the 5′ end of the oligomeric compound. In one embodiment, the hemimer motif comprises a contiguous sequence of from about 10 to about 28 monomer subunits of one type with from 1 to 5 or from 2 to about 5 monomer subunits of a second type located at one of the termini. In one embodiment, a hemimer is a contiguous sequence of from about 8 to about 20 β-D-2′-deoxyribonucleosides having from 1-12 contiguous nucleosides of the invention located at one of the termini. In one embodiment, a hemimer is a contiguous sequence of from about 8 to about 20 β-D-2′-deoxyribonucleosides having from 1-5 contiguous nucleosides of the invention located at one of the termini. In one embodiment, a hemimer is a contiguous sequence of from about 12 to about 18 β-D-2′-deoxyribo-nucleosides having from 1-3 contiguous nucleosides of the invention located at one of the termini. In one embodiment, a hemimer is a contiguous sequence of from about 10 to about 14 β-D-2′-deoxyribonucleosides having from 1-3 contiguous nucleosides of the invention located at one of the termini.

As used herein the term “blockmer motif” refers to an oligonucleotide comprising an otherwise contiguous sequence of monomer subunits wherein the sugar groups of each monomer subunit is the same except for an interrupting internal block of contiguous monomer subunits having a different type of sugar group. A blockmer overlaps somewhat with a gapmer in the definition but typically only the monomer subunits in the block have non-naturally occurring sugar groups in a blockmer and only the monomer subunits in the external regions have non-naturally occurring sugar groups in a gapmer with the remainder of monomer subunits in the blockmer or gapmer being β-D-2′-deoxyribonucleosides or β-D-ribonucleosides. In one embodiment, blockmer oligonucleotides are provided herein wherein all of the monomer subunits comprise non-naturally occurring sugar groups.

As used herein the term “positionally modified motif” is meant to include an otherwise contiguous sequence of monomer subunits having one type of sugar group that is interrupted with two or more regions of from 1 to about 5 contiguous monomer subunits having another type of sugar group. Each of the two or more regions of from 1 to about 5 contiguous monomer subunits are independently uniformly modified with respect to the type of sugar group. In one embodiment, each of the two or more regions have the same type of sugar group. In one embodiment, each of the two or more regions have a different type of sugar group. In one embodiment, positionally modified oligonucleotides are provided comprising a sequence of from 8 to 20 β-D-2′-deoxyribonucleosides that further includes two or three regions of from 2 to about 5 contiguous nucleosides of the invention. Positionally modified oligonucleotides are distinguished from gapped motifs, hemimer motifs, blockmer motifs and alternating motifs because the pattern of regional substitution defined by any positional motif does not fit into the definition provided herein for one of these other motifs. The term positionally modified oligomeric compound includes many different specific substitution patterns.

As used herein the term “gapmer” or “gapped oligomeric compound” refers to an oligomeric compound having two external regions or wings and an internal region or gap. The three regions form a contiguous sequence of monomer subunits with the sugar groups of the external regions being different than the sugar groups of the internal region and wherein the sugar group of each monomer subunit within a particular region is the same. When the sugar groups of the external regions are the same the gapmer is a symmetric gapmer and when the sugar group used in the 5′-external region is different from the sugar group used in the 3′-external region, the gapmer is an asymmetric gapmer. In one embodiment, the external regions are small (each independently 1, 2, 3, 4 or about 5 monomer subunits) and the monomer subunits comprise non-naturally occurring sugar groups with the internal region comprising β-D-2′-deoxyribonucleosides. In one embodiment, the external regions each, independently, comprise from 1 to about 5 monomer subunits having non-naturally occurring sugar groups and the internal region comprises from 6 to 18 unmodified nucleosides. The internal region or the gap generally comprises β-D-2′-deoxyribo-nucleosides but can comprise non-naturally occurring sugar groups.

In one embodiment, the gapped oligomeric compounds comprise an internal region of β-D-2′-deoxyribonucleosides with one of the external regions comprising nucleosides of the invention. In one embodiment, the gapped oligonucleotide comprise an internal region of β-D-2′-deoxyribonucleosides with both of the external regions comprising nucleosides of the invention. In one embodiment, the gapped oligonucleotide comprise an internal region of β-D-2′-deoxyribonucleosides with both of the external regions comprising nucleosides of the invention. In one embodiment, gapped oligonucleotides are provided herein wherein all of the monomer subunits comprise non-naturally occurring sugar groups. In one embodiment, gapped oliogonucleotides are provided comprising one or two nucleosides of the invention at the 5′-end, two or three nucleosides of the invention at the 3′-end and an internal region of from 10 to 16 β-D-2′-deoxyribonucleosides. In one embodiment, gapped oligonucleotides are provided comprising one nucleoside of the invention at the 5′-end, two nucleosides of the invention at the 3′-end and an internal region of from 10 to 16 β-D-2′-deoxyribonucleosides. In one embodiment, gapped oligonucleotides are provided comprising two nucleosides of the invention at the 5′-end, two nucleosides of the invention at the 3′-end and an internal region of from 10 to 14 β-D-2′-deoxyribonucleosides. In one embodiment, gapped oligonucleotides are provided that are from about 10 to about 21 monomer subunits in length. In one embodiment, gapped oligonucleotides are provided that are from about 12 to about 16 monomer subunits in length. In one embodiment, gapped oligonucleotides are provided that are from about 12 to about 14 monomer subunits in length.

In certain embodiments, the 5′-terminal monomer of an oligomeric compound of the invention comprises a phosphorous moiety at the 5′-end. In certain embodiments the 5′-terminal monomer comprises a 2′-modification. In certain such embodiments, the 2′-modification of the 5′-terminal monomer is a cationic modification. In certain embodiments, the 5′-terminal monomer comprises a 5′-modification. In certain embodiments, the 5′-terminal monomer comprises a 2′-modification and a 5′-modification. In certain embodiments, the 5′-terminal monomer is a 5′-stabilizing nucleoside. In certain embodiments, the modifications of the 5′-terminal monomer stabilize the 5′-phosphate. In certain embodiments, oligomeric compounds comprising modifications of the 5′-terminal monomer are resistant to exonucleases. In certain embodiments, oligomeric compounds comprising modifications of the 5′-terminal monomer have improved REVERSIR properties. In certain such embodiments, oligomeric compound comprising modifications of the 5′-terminal monomer have improved association with a strand of the dsRNA.

In certain embodiments, the 5′terminal monomer is attached to rest of the oligomeric compound a modified linkage. In certain such embodiments, the 5′terminal monomer is attached to rest of the oligomeric compound by a phosphorothioate linkage.

In certain embodiments, oligomeric compounds of the present invention comprise one or more regions of alternating modifications. In certain embodiments, oligomeric compounds comprise one or more regions of alternating nucleoside modifications. In certain embodiments, oligomeric compounds comprise one or more regions of alternating linkage modifications. In certan embodiments, oligomeric compounds comprise one or more regions of alternating nucleoside and linkage modifications.

In certain embodiments, oligomeric compounds of the present invention comprise one or more regions of alternating 2′-F modified nucleosides and 2′-O-Me modified nucleosides. In certain such embodiments, such regions of alternating 2′F modified and 2′O-Me modified nucleosides also comprise alternating linkages. In certain such embodiments, the linkages at the 3′ end of the 2′-F modified nucleosides are phosphorothioate linkages. In certain such embodiments, the linkages at the 3′end of the 2′O-Me nucleosides are phosphodiester linkages.

In certain embodiments, such alternating regions are:

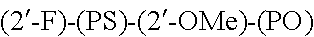

In certain embodiments, oligomeric compounds comprise 2, 3, 4, 5, 6, 7, 8, 9, 10, or 11 such alternating regions. Such regions may be contiguous or may be interrupted by differently modified nucleosides or linkages.

In certain embodiments, one or more alternating regions in an alternating motif include more than a single nucleoside of a type. For example, oligomeric compounds of the present invention may include one or more regions of any of the following nucleoside motifs:

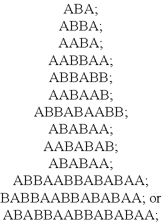

wherein A is a nucleoside of a first type and B is a nucleoside of a second type. In certain embodiments, A and B are each selected from 2′-F, 2′-OMe, LNA, DNA and MOE.

In certain embodiments, A is DNA. In certain embodiments B is DNA. In some embodiments, A is 4′-CH₂O-2′-LNA. In certain embodiments, B is 4′-CH₂O-2′-LNA. In certain embodiments, A is DNA and B is 4′-CH₂O-2′-LNA. In certain embodiments A is 4′-CH₂O-2′-LNA and B is DNA.

In certain embodiments, A is 2′-OMe. In certain embodiments B is 2′-OMe. In certain embodiments, A is 2′-OMe and B is 4′-CH₂O-2′-LNA. In certain embodiments A is 4′-CH₂O-2′-LNA and B is 2′-OMe. In certain embodiments, A is 2′-OMe and B is DNA. In certain embodiments A is DNA and B is 2′-OMe.

In certain embodiments, A is (S)-cEt. In some embodiments, B is (S)-cEt. In certain embodiments, A is 2′-OMe and B is (S)-cEt. In certain embodiments A is (S)-cEt and B is 2′-OMe. In certain embodiments, A is DNA and B is (S)-cEt. In certain embodiments A is (S)-cEt and B is DNA.

In certain embodiments, A is 2′-F. In certain embodiments B is 2′-F. In certain embodiments, A is 2′-F and B is 4′-CH₂O-2′-LNA. In certain embodiments A is 4′-CH₂O-2′-LNA and B is 2′-F. In certain embodiments, A is 2′-F and B is (S)-cEt. In certain embodiments A is (S)-cEt and B is 2′-F. In certain embodiments, A is 2′-F and B is DNA. In certain embodiments A is DNA and B is 2′-F. In certain embodiments, A is 2′-OMe and B is 2′-F. In certain embodiments, A is DNA and B is 2′-OMe. In certain embodiments, A is 2′-OMe and B is DNA.

In certain embodiments, oligomeric compounds having such an alternating motif also comprise a 5′ terminal nucleoside comprising a phosphate stabilizing modification. In certain embodiments, oligomeric compounds having such an alternating motif also comprise a 5′ terminal nucleoside comprising a 2′-cationic modification. In certain embodiments, oligomeric compounds having such an alternating motif also comprise a 5′ terminal modification.

In certain embodiments, oligomeric compounds of the present invention comprise a region having a 2-2-3 motif. Such regions comprises the following motif:
5′-(E)_{w}-(A)₂-(B)ₓ-(A)₂-(C)_{y}-(A)₃-(D)_{z}
wherein: A is a first type of modified nucleoside;
B, C, D, and E are nucleosides that are differently modified than A, however, B, C, D, and E may have the same or different modifications as one another;
w and z are from 0 to 15;
x and y are from 1 to 15.

In certain embodiments, A is a 2′-OMe modified nucleoside. In certain embodiments, B, C, D, and E are all 2′-F modified nucleosides. In certain embodiments, A is a 2′-OMe modified nucleoside and B, C, D, and E are all 2′-F modified nucleosides.

In certain embodiments, the linkages of a 2-2-3 motif are all modifed linkages. In certain embodiments, the linkages are all phosphorothioate linkages. In certain embodiemtns, the linkages at the 3′-end of each modification of the first type are phosphodiester.

In certain embodiments, Z is 0. In such embodiments, the region of three nucleosides of the first type are at the 3′-end of the oligonucleotide. In certain embodiments, such region is at the 3′-end of the oligomeric compound, with no additional groups attached to the 3′ end of the region of three nucleosides of the first type. In certain embodiments, an oligomeric compound comprising an oligonucleotide where Z is 0, may comprise a terminal group attached to the 3′-terminal nucleoside.

Such terminal groups may include additional nucleosides. Such additional nucleosides are typically non-hybridizing nucleosides.

In certain embodiments, Z is 1-3. In certain embodiments, Z is 2. In certain embodiments, the nucleosides of Z are 2′-MOE nucleosides. In certain embodiments, Z represents non-hybridizing nucleosides. To avoid confussion, it is noted that such non-hybridizing nucleosides might also be described as a 3′-terminal group with Z=0.

It is to be understood, that certain of the above described motifs and modifications can be combined. Since a motif may comprise only a few nucleosides, a particular oligomeric compound can comprise two or more motifs. By way of non-limiting example, in certain embodiments, oligomeric compounds can have two or more nucleotide motifs selected from LNAs, phosphorthioate linkages, 2′-OMe, conjugated ligand(s).

Oligomeric compounds having any of the various nucleoside motifs described herein, can have also have any linkage motif. For example, in the oligomeric compounds first 1, 2, 3, 4 or 5 at the 5′-end be modified intrersugar linkages and first 4, 5, 6, 7 or 8 intersugar linkages at the 3′-end can be modified intersugar linkages. The central region of such modified oligomeric compound can have intersugar linkages based on the any of the other motifs described herein, for example, uniform, alternating, hemimer, gapmer, and the like. In some embodiments, the oligomeric compound comprise a phosphorothioate linkage between the first and second monomer at the 5′-terminus, alternating phosphorothioate/phosphodiester linkages in the central region and 6, 7, or 8 phosphorothioate linkages at the 3′-terminus.

It is to be noted that the lengths of the regions defined by a nucleoside motif and that of a linkage motif need not be the same.

In some embodiments, single-stranded oligomeric compounds include at least one of the following motifs:
(a) 5′-phosphorothioate or 5′-phosphorodithioate;
(b) a cationic modification of nucleotides 1 and 2 on the 5′ terminal, wherein the cationic modification is at C5 position of pyrimidines and C2, C6, C8, exocyclic N2 or exocyclic N6 of purines;
(c) at least one G-clamp nucleotide in the first two terminal nucleotides at the 5′ end and the other nucleotide having a cationic modification, wherein the cationic modification is at C5 position of pyrimidines or C2, C6, C8, exocyclic N2 or exocyclic N6 position of purines;
(d) at least one 2′-F modified nucleotide comprising a nucleobase base modification;
(e) at least one gem-2′-O-methyl/2′-F modified nucleotide comprising a nucleobase modification, preferably the methyl substituent is in the up configuration, e.g. in the arabinose configuration;
(f) a 5′-PuPu-3′ dinucleotide at the 3′ terminal wherein both nucleotides comprise a modified MOE at 2′-position as described in U.S. Patent Application Publication No. 20130130378, content of which is incorporated herein by reference in its entirety.
(g) a 5′-PuPu-3′ dinucleotide at the 5′ terminal wherein both nucleotides comprise a modified MOE at 2′-position as described in U.S. Patent Application Publication No. 20130130378;
(h) nucleotide at the 5′ terminal having a modified MOE at 2′-position as described in U.S. Patent Application Publication No. 20130130378;
(i) nucleotide at the 5′ terminal having a 3′-F modification;
(j) 5′ terminal nucleotide comprising a 4′-substituent;
(k) 5′ terminal nucleotide comprising a 04′ modification;
(l) 3′ terminal nucleotide comprising a 4′-substituent; and
(m) combinations thereof.

The above examples are provided solely to illustrate how the described motifs may be used in combination and are not intended to limit the invention to the particular combinations or the particular modifications used in illustrating the combinations. Further, specific examples herein are intended to encompass more generic embodiments. All of the examples throughout this specification contemplate such generic interpretation.

It is also noted that the lengths of oligomeric compounds can be easily manipulated by lengthening or shortening one or more of the described regions, without disrupting the motif.

In some embodiments, oligomeric compound comprises two or more chemically distinct regions and has a structure as described in International Application No. PCT/US09/038433, filed Mar. 26, 2009, contents of which are herein incorporated in their entirety.

### III. Ligands of the Invention

In certain embodiments, oligomeric compounds are modified by covalent attachment of one or more conjugate groups. In general, conjugate groups modify one or more properties of the attached oligomeric compound including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactose (GalNAc), multivalent GalNAc, e.g. GalNAc2 and GalNAc3; D-mannose, multivalent mannose, multivalent lactose, N-acetyl-galactosamine, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, scuh as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties. As used herein, when a ligand is linked to more than oligomeric strand, point of attachment for an oligomeric compound can be an atom of the ligand self or an atom on a carrier molecule to which the ligand itself is attached.

Ligands can be coupled to the oligomeric compounds at various places, for example, 3′-end, 5′-end, and/or at an internal position. When two or more ligands are present, the ligand can be on opposite ends of an oligomeric compound. In preferred embodiments, the ligand is attached to the oligomeric compound via an intervening tether/linker. The ligand or tethered ligand can be present on a monomer when said monomer is incorporated into the growing strand. In some embodiments, the ligand can be incorporated via coupling to a “precursor” monomer after said “precursor” monomer has been incorporated into the growing strand. For example, a monomer having, e.g., an amino-terminated tether (i.e., having no associated ligand), e.g., monomer-linker-NH2 can be incorporated into a growing oligomeric compound strand. In a subsequent operation, i.e., after incorporation of the precursor monomer into the strand, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, the ligand can be conjugated to nucleobases, sugar moieties, or internucleosidic linkages of oligomeric compound. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

In some embodiments, the REVERSIR compound is conjugated with a ligand.

While useful in delivery of the REVERSIR compound to a desired location of action, the ligand conjugated with the REVERSIR compound can negatively affect the ability of the REVERSIR compound to reduce dsRNA activity. Therefore, in some embodiments, the linkage between the ligand and the REVERSIR compound can be designed to undergo cleavage after the REVERSIR compound reaches a desired location of action. This can be accomplished in a number of ways. For example, the linker connecting the REVERSIR compound to the ligand can be a cleavable linker.

In some embodiments, the nucleoside conjugated with the ligand comprises a deoxy sugar, for example, a 2′-deoxy sugar.

In some embodiments of the various aspects disclosed herein, the ligand is attached to the nucleoside at the 3′-terminus of the REVERSIR compound. In some embodiments, the ligand conjugated nucleotide is attached to the rest of the REVERSIR compound via a cleavable internucleotide linage. In some embodiment, the cleavable internucleotide linkage is a phosphodiester internucleotide linkage.

In some embodiments, the ligand conjugated nucleotide comprises a deoxy sugar and is linked to rest of the REVERSIR compound via a cleavable internucleotide linkage. In some further embodiments, of this the cleavable internucleotide linkage is a phosphodiester linkage.

In some embodiments, the ligand conjugated nucleotide comprises a deoxy sugar and is linked to rest of the REVERSIR compound via an internucleotide linkage that is not a phosphodiester linkage.

In some embodiments, the ligand is conjugated to the nucleotide at the 3′-terminus of the REVERSIR compound.

In some embodiments, the ligand is conjugated at the 5′-terminus of the REVERSIR compound. In some embodiments, a first ligand is conjugated at the 5′-terminus of the REVERSIR compound and a second ligand conjugated to the first ligand.

There are numerous methods for preparing conjugates of oligomeric compounds. Generally, an oligomeric compound is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative U.S. patents that teach the preparation of conjugates of oligomeric compounds, e.g., oligonucleotides, include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082, 830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; contents of which are herein incorporated in their entireties by reference.

In certain embodiments, the oligomeric compound described herein, including but not limited to REVERSIR compounds, comprises a ligand having a structure shown below:

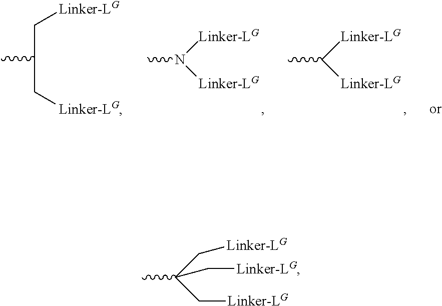

wherein:
L^{G} is independently for each occurrence a ligand, e.g., carbohydrate, e.g. monosaccharide, disaccharide, trisaccharide, tetrasaccharide, polysaccharide; and
Z′, Z″, Z′″ and Z″″ are each independently for each occurrence O or S.

In certain embodiments, the oligomeric compound described herein, including but not limited to REVERSIR compounds, comprises a ligand of Formula (II), (III), (IV) or (V):

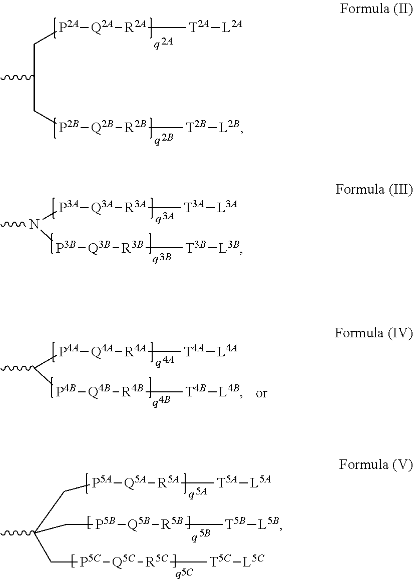

wherein:
q^{2A}, q^{2B}, q^{3A}, q^{3B}, q4^{A}, q^{5A}, q^{5B} and q^{5C} represent independently for each occurrence 0-20 and wherein the repeating unit can be the same or different;
Q and Q′ are independently for each occurrence is absent, —(P⁷-Q⁷-R⁷)ₚ-T⁷- or -T⁷-Q⁷-T^{7′}-B-T^{8′}-Q⁸-T⁸;
P^{2A}, P^{2B}, P^{3A}, P^{3B}, P^{4A}, P^{4B}, P^{5A}, P^{5B}, P^{5C}, P⁷, T^{2A}, T^{2B}, T^{3A}, T^{3B}, T^{4A}, T^{4B}, T^{4A}, T^{5B}, T^{5C}, T⁷, T^{7′}, T⁸ and T^{8′} are each independently for each occurrence absent, CO, NH, O, S, OC(O), NHC(O), CH₂, CH₂NH or CH₂O;
B is —CH₂—N(B^{L})—CH₂—;
B^{L} is -T^{B}-Q^{B}-T^{B′}-R^{x};
Q^{2A}, Q^{2B}, Q^{3A}, Q^{3B}, Q^{4A}, Q^{4B}, Q^{5A}, Q^{5B}, Q^{5C}, Q⁷, Q⁸ and Q^{B} are independently for each occurrence absent, alkylene, substituted alkylene and wherein one or more methylenes can be interrupted or terminated by one or more of 0, S, S(O), SO₂, N(R^{N}), C(R′)═C(R′), C≡C or C(O);
T^{B} and T^{B′} are each independently for each occurrence absent, CO, NH, O, S, OC(O), OC(O)O, NHC(O), NHC(O)NH, NHC(O)O, CH₂, CH₂NH or CH₂O;
R^{x} is a lipophile (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid;
R¹, R², R^{2A}, R^{2B}, R^{3A}, R^{3B}, R^{4A}, R^{4B}, R^{5A}, R^{5B}, R^{5C}, R⁷ are each independently for each occurrence absent, NH, O, S, CH₂, C(O)O, C(O)NH, NHCH(R^{a})C(O), —C(O)—CH(R^{a})—NH—, CO,

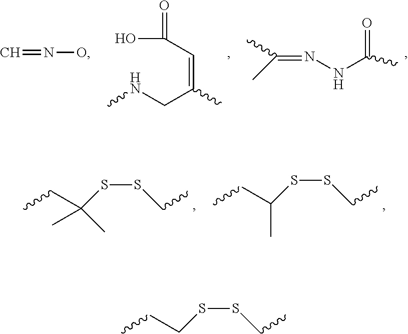

or heterocyclyl;
L¹, L^{2A}, L^{2B}, L^{3A}, L^{3B}, L^{4A}, L^{4B} L^{5A} L^{5B} and L^{5C} are each independently for each occurrence a carbohydrate, e.g., monosaccharide, disaccharide, trisaccharide, tetrasaccharide, oligosaccharide and polysaccharide;
R′ and R″ are each independently H, C¹-C₆ alkyl, OH, SH, or N(R^{N})₂;
R^{N} is independently for each occurrence H, methyl, ethyl, propyl, isopropyl, butyl or benzyl;
R^{a} is H or amino acid side chain;
Z′, Z″, Z′″ and Z″″ are each independently for each occurrence O or S;
p represent independently for each occurrence 0-20.

In certain embodiments, the oligomeric compound described herein, including but not limited to REVERSIR compounds, comprises a ligand of structure:

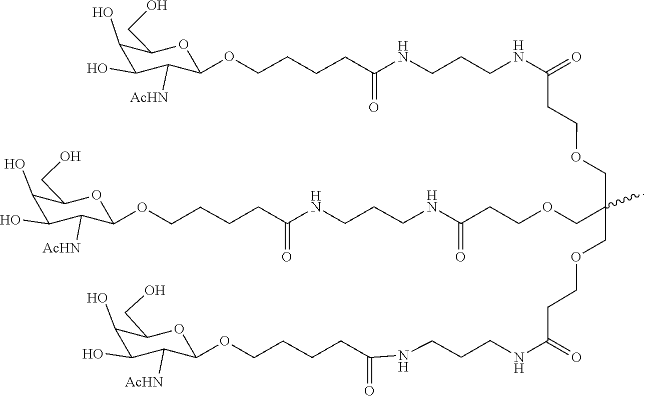 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, the oligomeric compound described herein, including but not limited to REVERSIR compounds, comprises a ligand of structure:

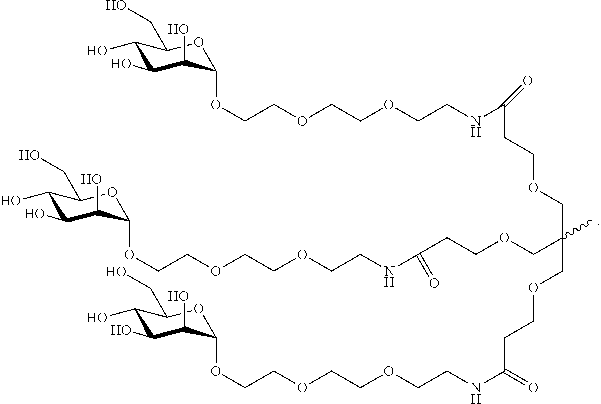

In certain embodiments, the oligomeric compound described herein, including but not limited to REVERSIR compounds, comprises a ligand of structure:

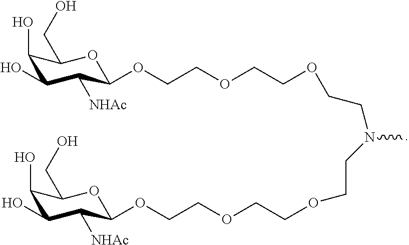

In certain embodiments, the oligomeric compound described herein, including but not limited to REVERSIR compounds, comprises a ligand of structure:

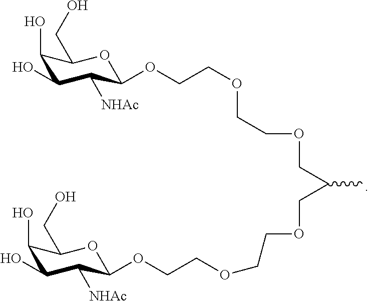

In certain embodiments, the oligomeric compound described herein, including but not limited to REVERSIR compounds, comprises a ligand of structure:

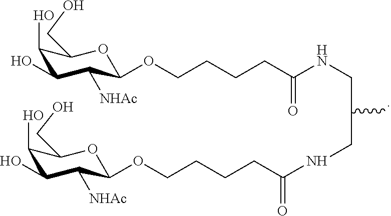

In certain embodiments, the oligomeric compound described herein, including but not limited to REVERSIR compounds, comprises a ligand of structure:

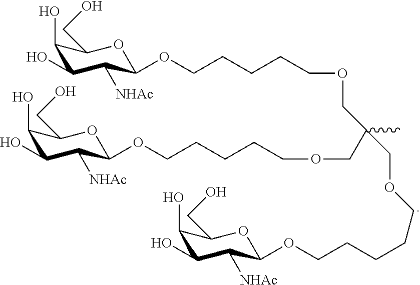

In certain embodiments, the oligomeric compound described herein, including but not limited to REVERSIR compounds, comprises a ligand of structure:

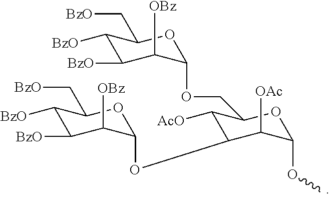

In certain embodiments, the oligomeric compound described herein, including but not limited to REVERSIR compounds, comprises a ligand of structure:

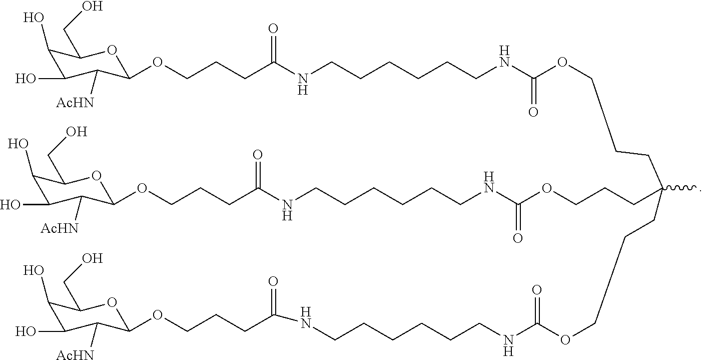 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In certain embodiments, the oligomeric compound described herein, including but not limited to REVERSIR compounds, comprises a ligand of structure:

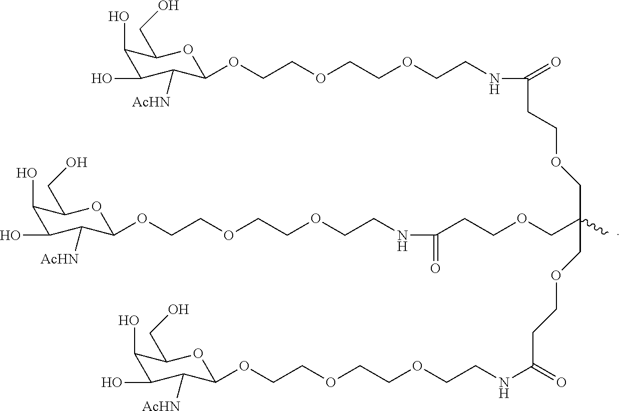 [SMILES: CC(=O)NC1C(OCCOCCOCCNC(=O)CCOCC(C)(COCCC(=O)NCCOCCOCCOC2OC(CO)C(O)C(O)C2NC(C)=O)COCCC(=O)NCCOCCOCCOC2OC(CO)C(O)C(O)C2NC(C)=O)OC(CO)C(O)C1O]

In certain embodiments, the oligomeric compound described herein, including but not limited to REVERSIR compounds, comprises a ligand of structure:

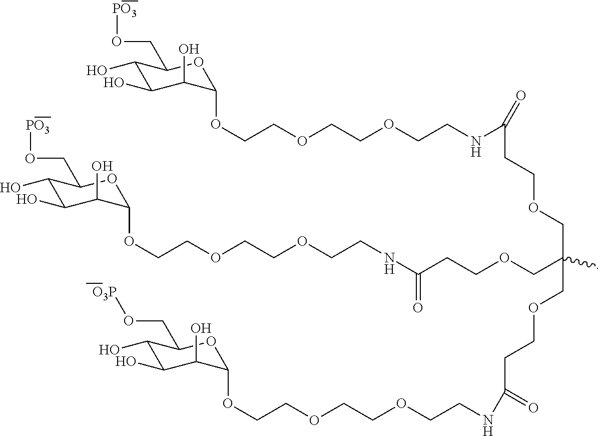

In certain embodiments, the oligomeric compound described herein, including but not limited to REVERSIR compounds, comprises a ligand of structure:

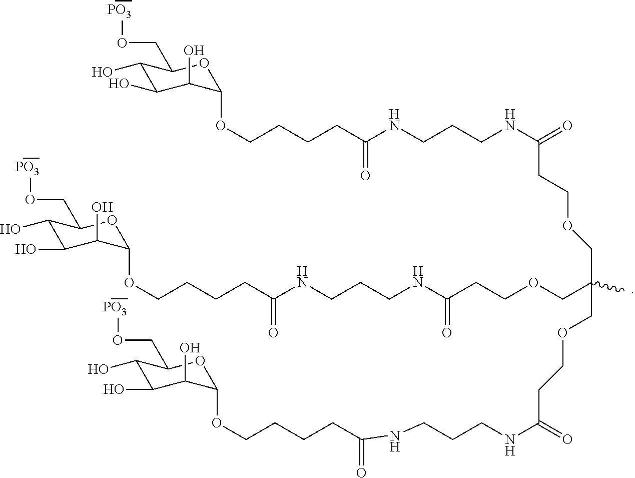

In certain embodiments, the oligomeric compound described herein, including but not limited to REVERSIR compounds, comprises a monomer of structure:

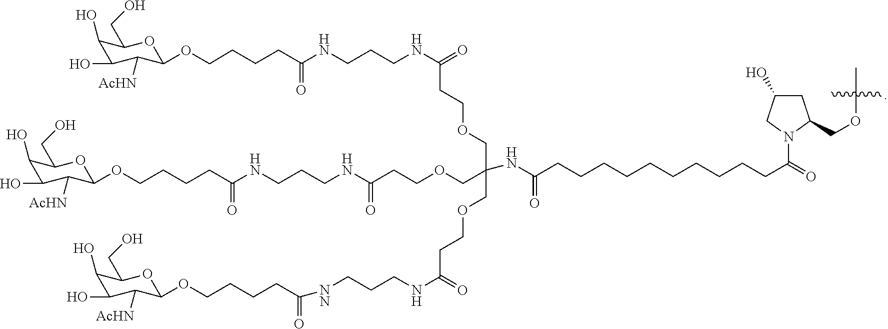

In certain embodiments, the oligomeric compound described herein, including but not limited to REVERSIR compounds, comprises a ligand of structure:

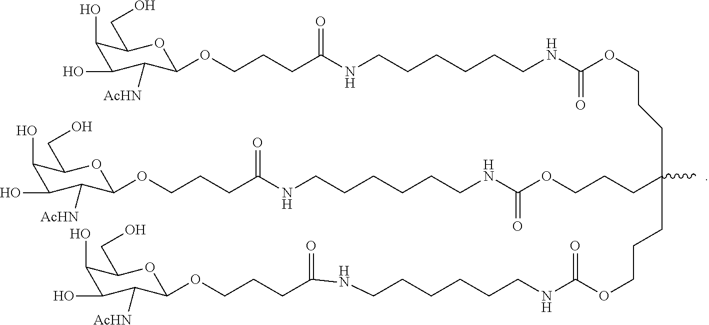 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In certain embodiments, the oligomeric compound described herein, including but not limited to REVERSIR compounds, comprises a monomer of structure:

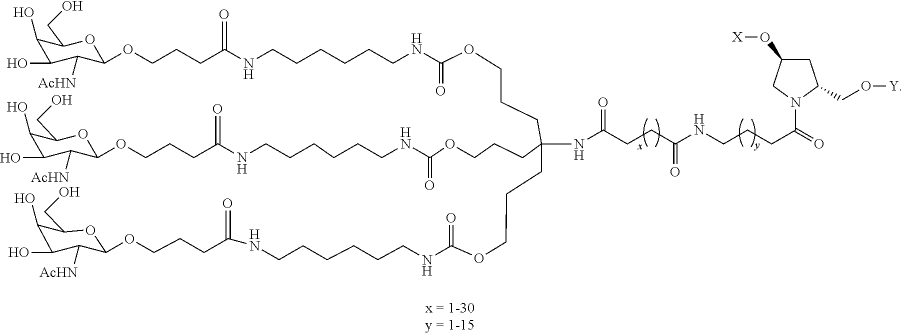

In certain embodiments, the oligomeric compound described herein, including but not limited to REVERSIR compounds, comprises a monomer of structure:

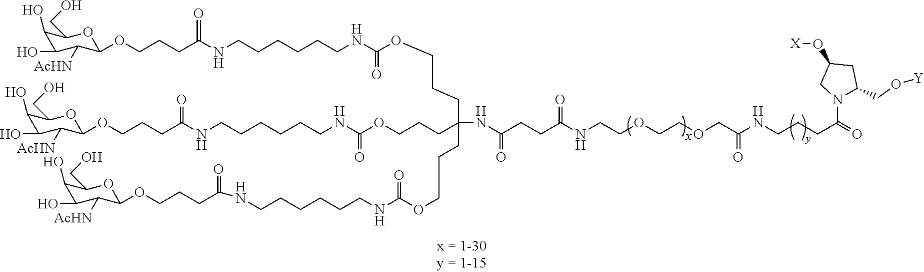

In certain embodiments, the oligomeric compound described herein, including but not limited to REVERSIR compounds, comprises a monomer of structure:

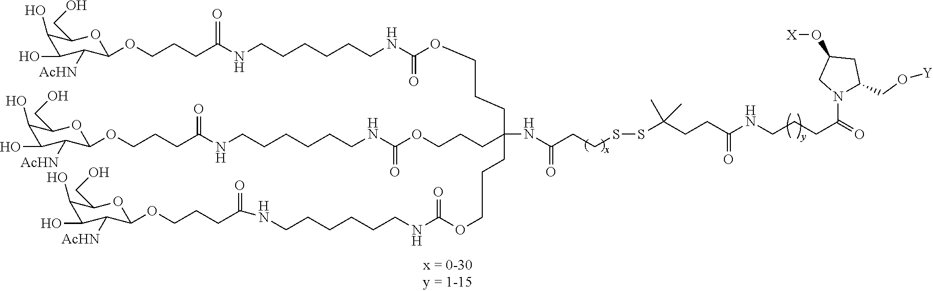

In certain embodiments, the oligomeric compound described herein, including but not limited to REVERSIR compounds comprises a monomer of structure:

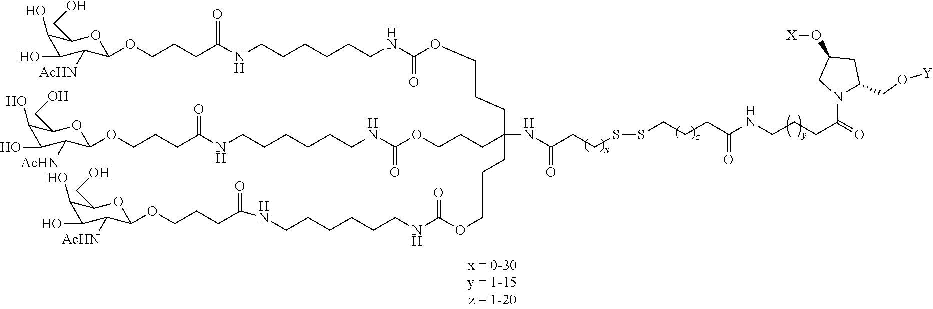

In certain embodiments, the oligomeric compound described herein, including but not limited to REVERSIR compounds, comprises a monomer of structure:

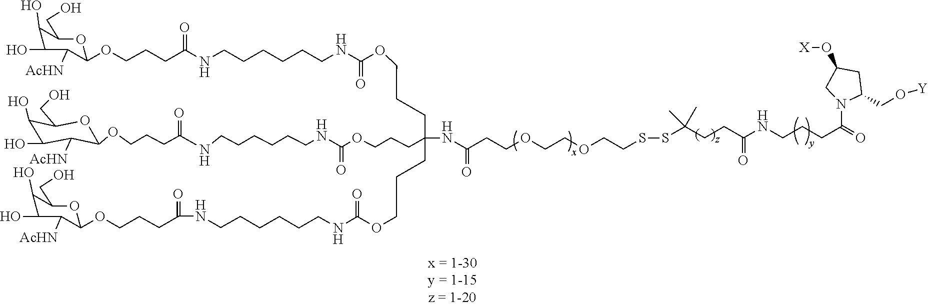

In certain embodiments, the oligomeric compound described herein, including but not limited to REVERSIR compounds, comprises a monomer of structure:

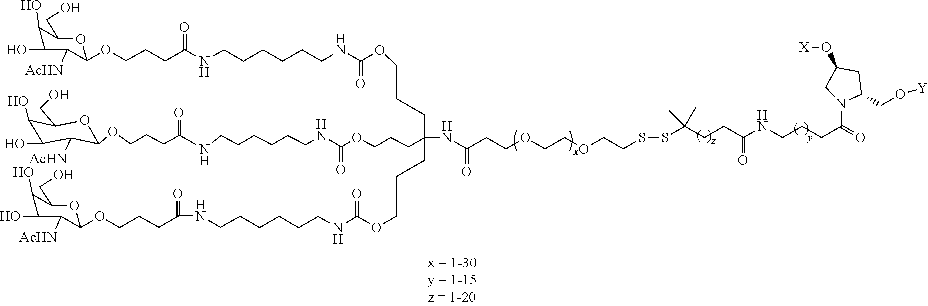

In certain embodiments, the oligomeric compound described herein, including but not limited to REVERSIR compounds, comprises a ligand of structure:

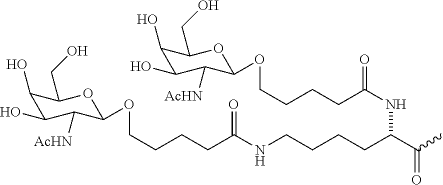

In certain embodiments, the oligomeric compound described herein, including but not limited to REVERSIR compounds, comprises a ligand of structure:

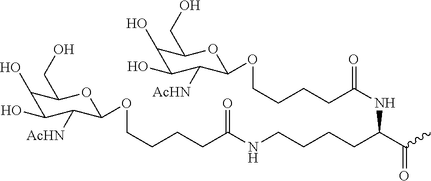

In certain embodiments, the oligomeric compound described herein, including but not limited to REVERSIR compounds, comprises a ligand of structure:

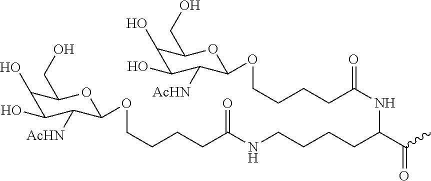

In certain embodiments, the oligomeric compound described herein, including but not limited to REVERSIR compounds, comprises a ligand of structure:

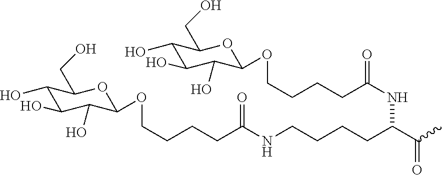

In certain embodiments, the oligomeric compound described herein, including but not limited to REVERSIR compounds, comprises a ligand of structure:

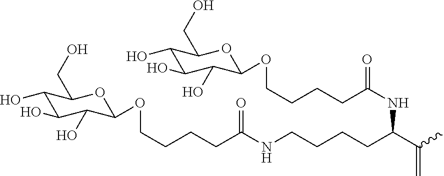

In certain embodiments, the oligomeric compound described herein, including but not limited to REVERSIR compounds, comprises a ligand of structure:

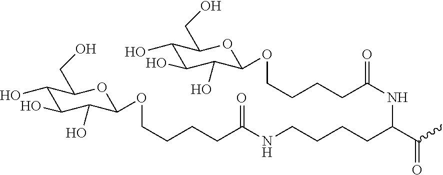

In certain embodiments, the oligomeric compound described herein, including but not limited to REVERSIR compounds, comprises a ligand of structure:

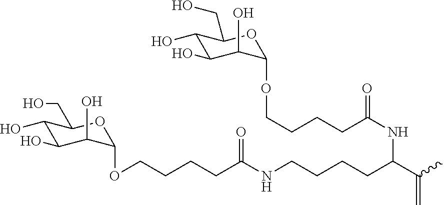

In certain embodiments, the oligomeric compound described herein, including but not limited to REVERSIR compounds, comprises a ligand of structure:

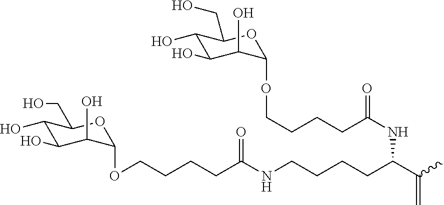

In certain embodiments, the oligomeric compound described herein, including but not limited to REVERSIR compounds, comprises a ligand of structure:

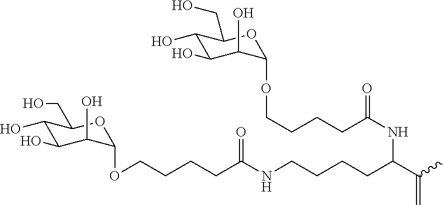

In certain embodiments, the oligomeric compound described herein, including but not limited to REVERSIR compounds, comprises a monomer of structure:

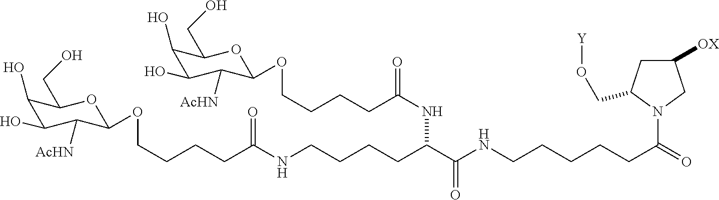

In certain embodiments, the oligomeric compound described herein, including but not limited to REVERSIR compounds, comprises a monomer of structure:

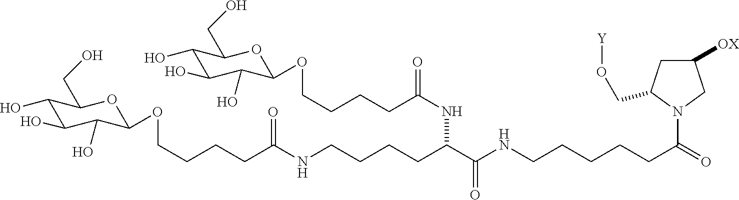

In certain embodiments, the oligomeric compound described herein, including but not limited to REVERSIR compounds, comprises a monomer of structure:

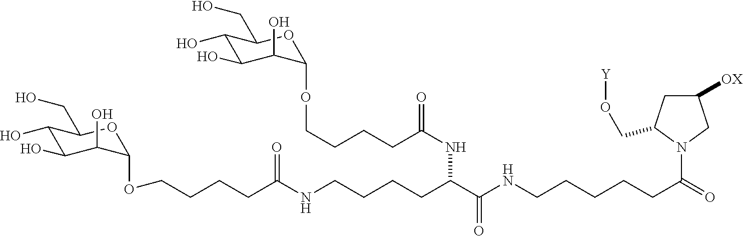

In certain embodiments, the oligomeric compound described herein, including but not limited to REVERSIR compounds, comprises a monomer of structure:

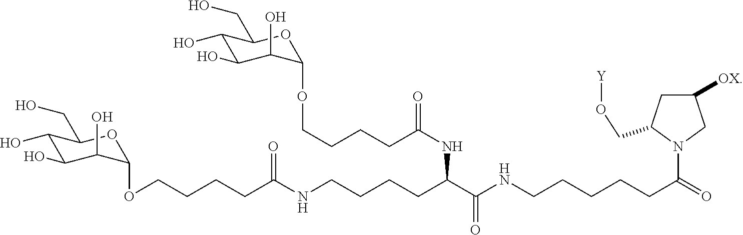

In certain embodiments, the oligomeric compound described herein, including but not limited to REVERSIR compounds, comprises a monomer of structure:

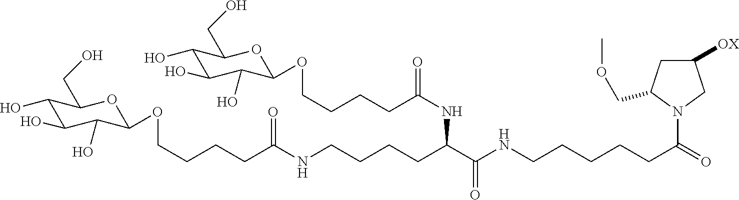

In certain embodiments, the oligomeric compound described herein, including but not limited to REVERSIR compounds, comprises a monomer of structure:

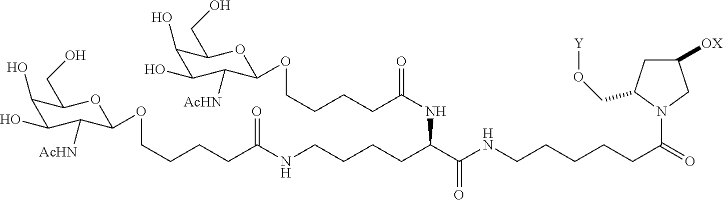

In some embodiments both L^{2A} and L^{2B} are different.

In some preferred embodiments both L^{3A} and L^{3B} are the same.

In some embodiments both L^{3A} and L^{3B} are different.

In some preferred embodiments both L^{4A} and L^{4B} are the same.

In some embodiments both L^{4A} and L^{4B} are different.

In some preferred embodiments all of L^{5A}, L^{5B} and L^{5C} are the same.

In some embodiments two of L^{5A}, L^{5B} and L^{5C} are the same.

In some embodiments L^{5A} and L^{5B} are the same.

In some embodiments L^{5A} and L^{5C} are the same.

In some embodiments L^{5B} and L^{5C} are the same.

In certain embodiments, the oligomeric compound described herein, including but not limited to REVERSIR compounds, comprises a monomer of structure:

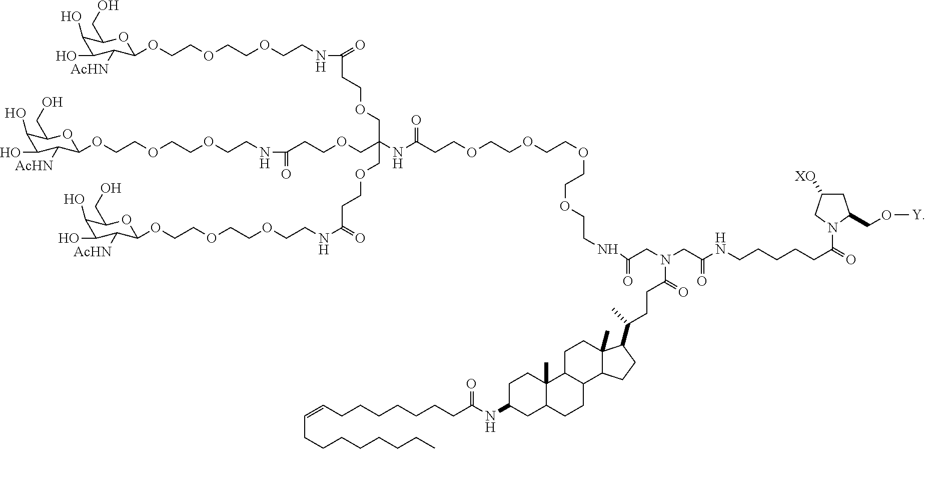

In certain embodiments, the oligomeric compound described herein, including but not limited to REVERSIR compounds, comprises a monomer of structure:

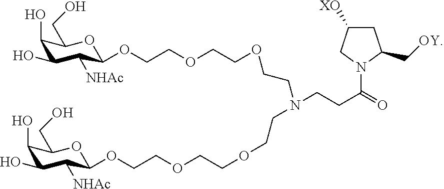

In certain embodiments, the oligomeric compound described herein, including but not limited to REVERSIR compounds, comprises a monomer of structure:

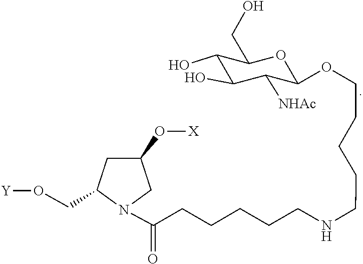

In certain embodiments, the oligomeric compound described herein, including but not limited to REVERSIR compounds, comprises a monomer of structure:

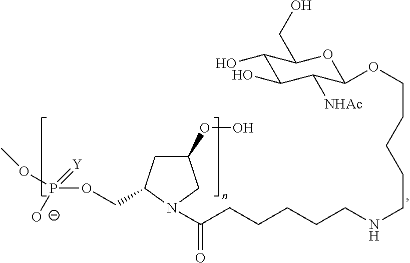

wherein Y is O or S and n is 3-6.

In certain embodiments, the oligomeric compound described herein, including but not limited to REVERSIR compounds, comprises a monomer of structure:

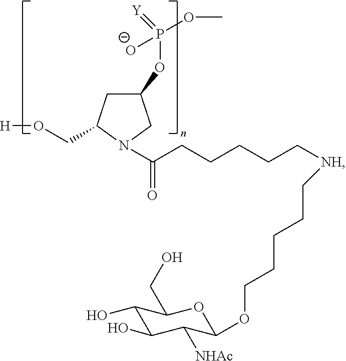

wherein Y is O or S and n is 3-6.

In certain embodiments, the oligomeric compound described herein, including but not limited to REVERSIR compounds, comprises a monomer of structure:

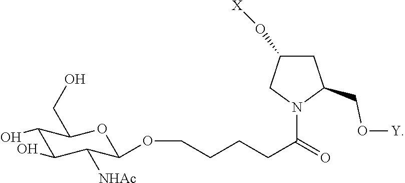

In certain embodiments, the oligomeric compound described herein, including but not limited to REVERSIR compounds, comprises a monomer of structure:

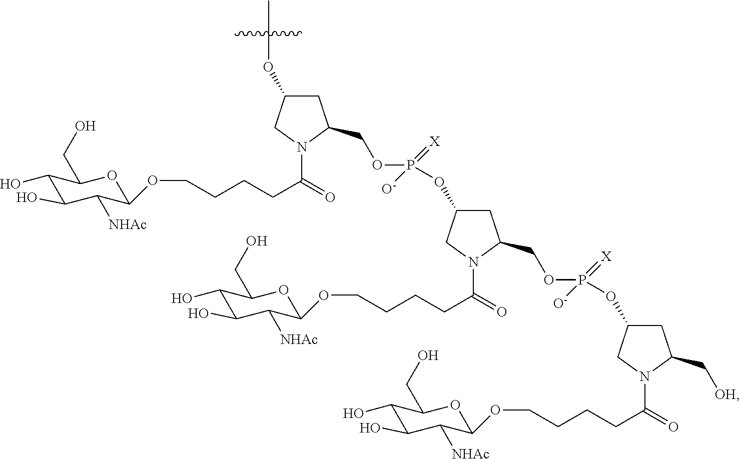

wherein X is O or S.

In certain embodiments, the oligomeric compound described herein, including but not limited to REVERSIR compounds, comprises a monomer selected from the group consisting of:

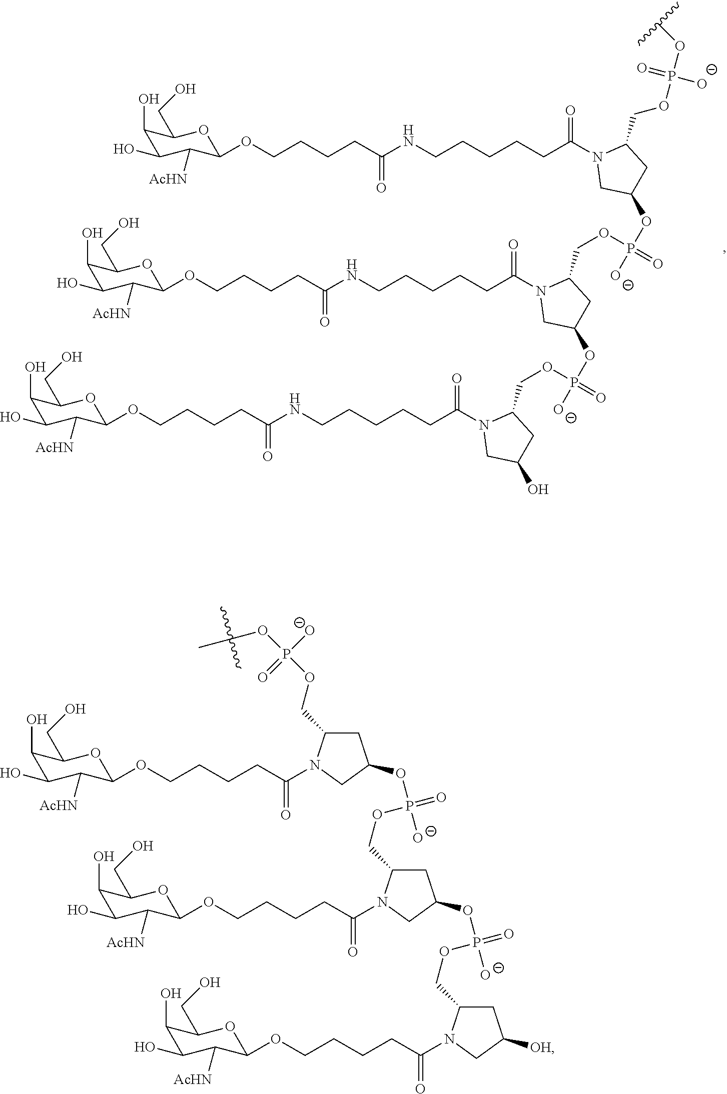 [SMILES: *OP(=O)([O-])OC[C@@H]1C[C@@H](OP(=O)([O-])OC[C@@H]2C[C@@H](OP(=O)([O-])OC[C@@H]3C[C@@H](O)CN3C(=O)CCCCCNC(=O)CCCCOC3OC(CO)C(O)C(O)C3NC(C)=O)CN2C(=O)CCCCCNC(=O)CCCCOC2OC(CO)C(O)C(O)C2NC(C)=O)CN1C(=O)CCCCCNC(=O)CCCCOC1OC(CO)C(O)C(O)C1NC(C)=O ; *OP(=O)([O-])OC[C@@H]1C[C@@H](OP(=O)([O-])OC[C@@H]2C[C@@H](OP(=O)([O-])OC[C@@H]3C[C@@H](O)CN3C(=O)CCCCOC3OC(CO)C(O)C(O)C3NC(C)=O)CN2C(=O)CCCCOC2OC(CO)C(O)C(O)C2NC(C)=O)CN1C(=O)CCCCOC1OC(CO)C(O)C(O)C1NC(C)=O]

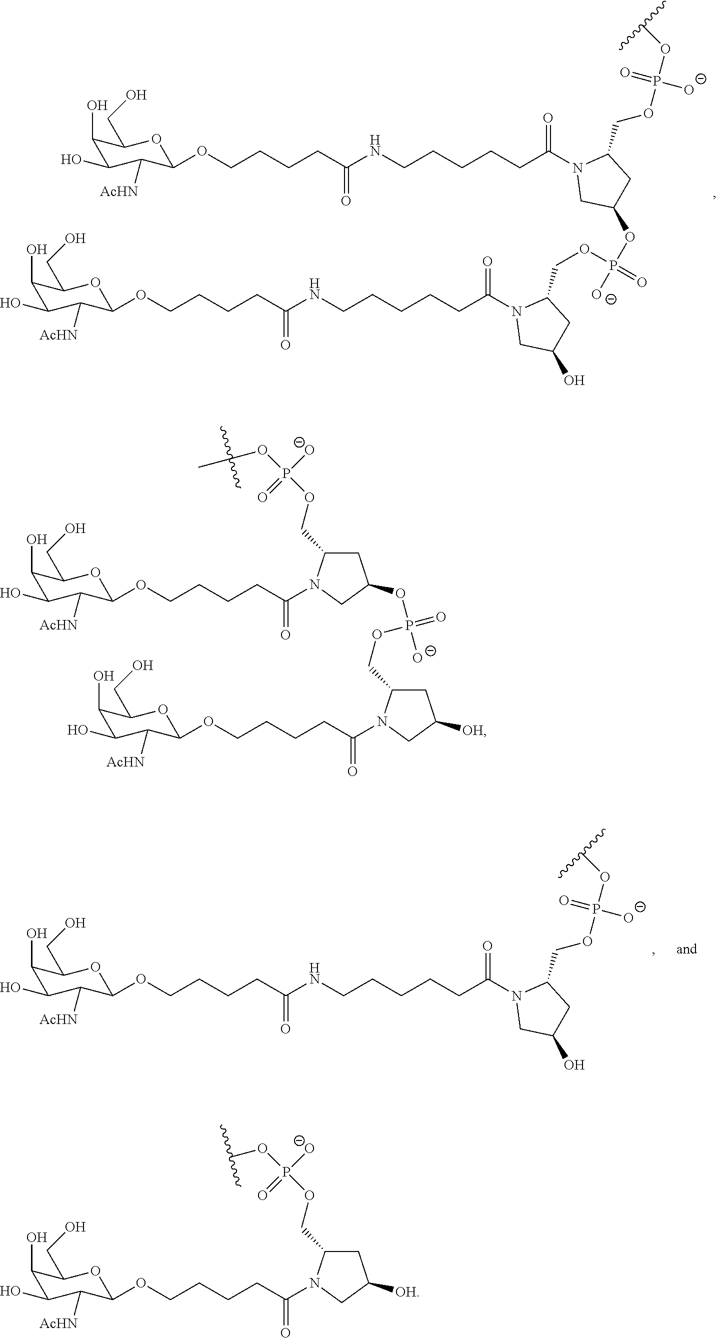 [SMILES: *OP(=O)([O-])OC[C@@H]1C[C@@H](OP(=O)([O-])OC[C@@H]2C[C@@H](O)CN2C(=O)CCCCCNC(=O)CCCCOC2OC(CO)C(O)C(O)C2NC(C)=O)CN1C(=O)CCCCCNC(=O)CCCCOC1OC(CO)C(O)C(O)C1NC(C)=O ; *OP(=O)([O-])OC[C@@H]1C[C@@H](OP(=O)([O-])OC[C@@H]2C[C@@H](O)CN2C(=O)CCCCOC2OC(CO)C(O)C(O)C2NC(C)=O)CN1C(=O)CCCCOC1OC(CO)C(O)C(O)C1NC(C)=O ; *OP(=O)([O-])OC[C@@H]1C[C@@H](O)CN1C(=O)CCCCCNC(=O)CCCCOC1OC(CO)C(O)C(O)C1NC(C)=O ; *OP(=O)([O-])OC[C@@H]1C[C@@H](O)CN1C(=O)CCCCOC1OC(CO)C(O)C(O)C1NC(C)=O]

In certain embodiments, the oligomeric compound described herein, including but not limited to REVERSIR compounds, comprises a monomer of structure:

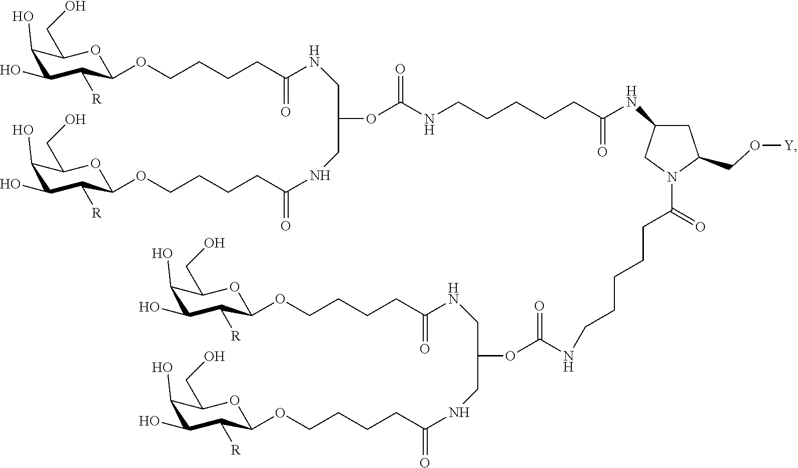

wherein R is OH or NHCOOH.

In certain embodiments, the oligomeric compound described herein, including but not limited to REVERSIR compounds, comprises a monomer of structure:

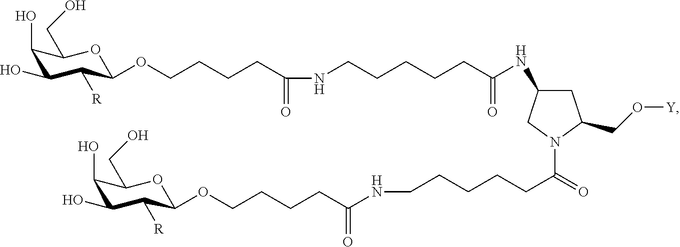

wherein R is OH or NHCOOH.

In certain embodiments, the oligomeric compound described herein, including but not limited to REVERSIR compounds, comprises a monomer of structure:

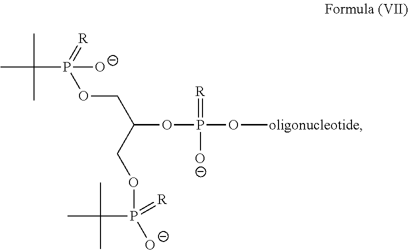

wherein R is O or S.

In certain embodiments, the oligomeric compound described herein, including but not limited to REVERSIR compounds, comprises a monomer of structure:

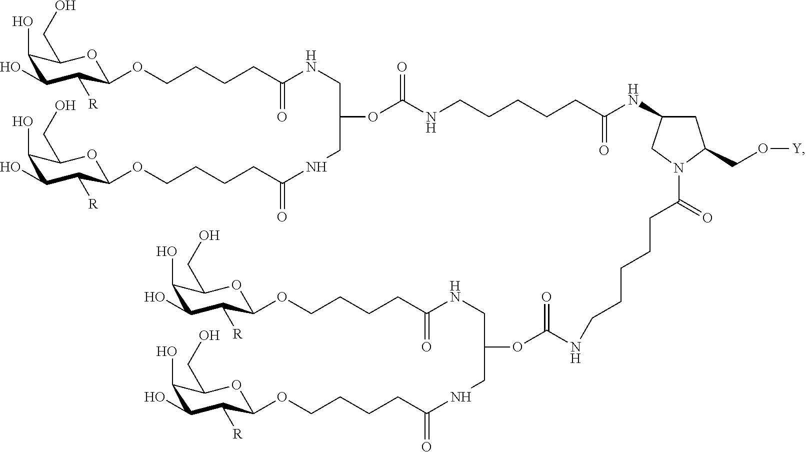

wherein R is OH or NHCOOH.

In certain embodiments, the oligomeric compound described herein, including but not limited to REVERSIR compounds, comprises a monomer of structure:

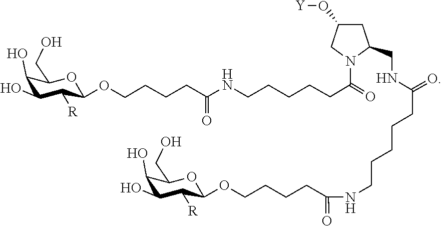

In certain embodiments, the oligomeric compound described herein, including but not limited to REVERSIR compounds, comprises a monomer of structure:

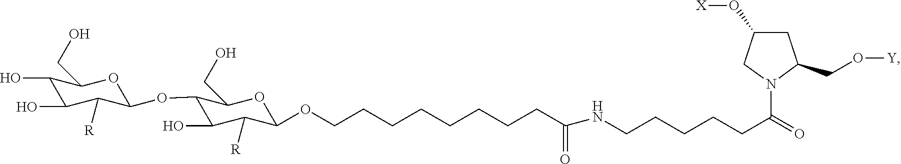

where in R is OH or NHCOOH.

In certain embodiments, the oligomeric compound described herein, including but not limited to REVERSIR compounds, comprises a monomer of structure:

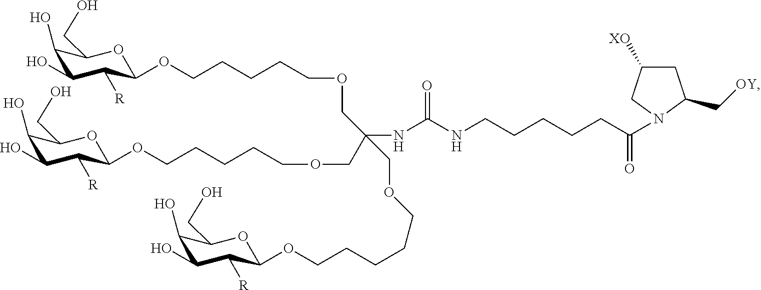

wherein R is OH or NHCOOH.

In certain embodiments, the oligomeric compound described herein, including but not limited to REVERSIR compounds, comprises a monomer of structure:

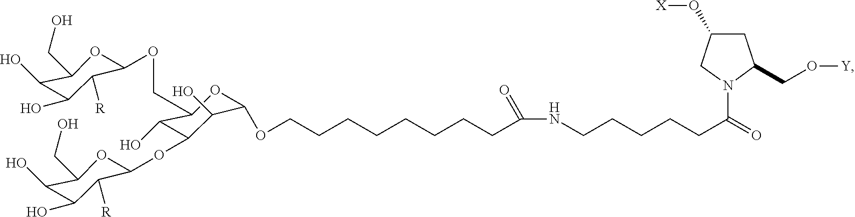

wherein R is OH or NHCOOH.

In certain embodiments, the oligomeric compound described herein, including but not limited to REVERSIR compounds, comprises a monomer of structure:

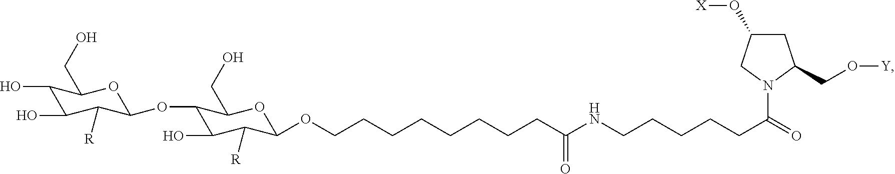

wherein R is OH or NHCOOH.

In certain embodiments, the oligomeric compound described herein, including but not limited to REVERSIR compounds, comprises a monomer of structure:

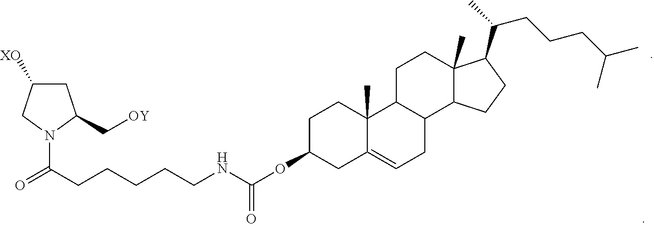

In the above described monomers, X and Y are each independently for each occurrence H, a protecting group, a phosphate group, a phosphodiester group, an activated phosphate group, an activated phosphite group, a phosphoramidite, a solid support, —P(Z′)(Z″)O-nucleoside, —P(Z′)(Z″)O-oligonucleotide, a lipid, a PEG, a steroid, a polymer, a nucleotide, a nucleoside, or an oligonucleotide; and Z′ and Z″ are each independently for each occurrence O or S.

In certain embodiments, the REVERSIR compound is conjugated with a ligand of structure:

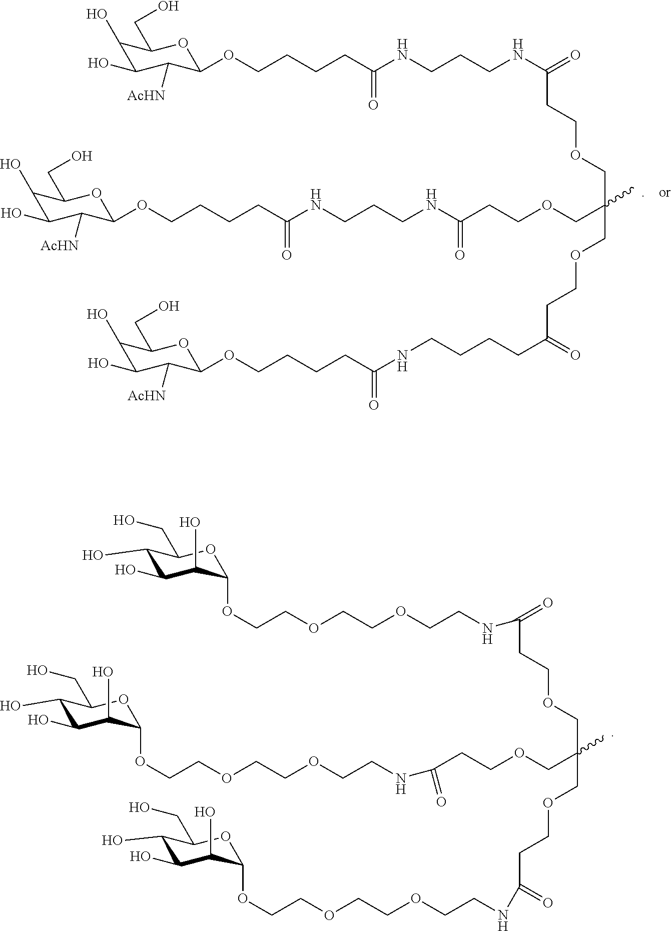

In certain embodiments, the conjugated dsRNA comprises a ligand of structure:

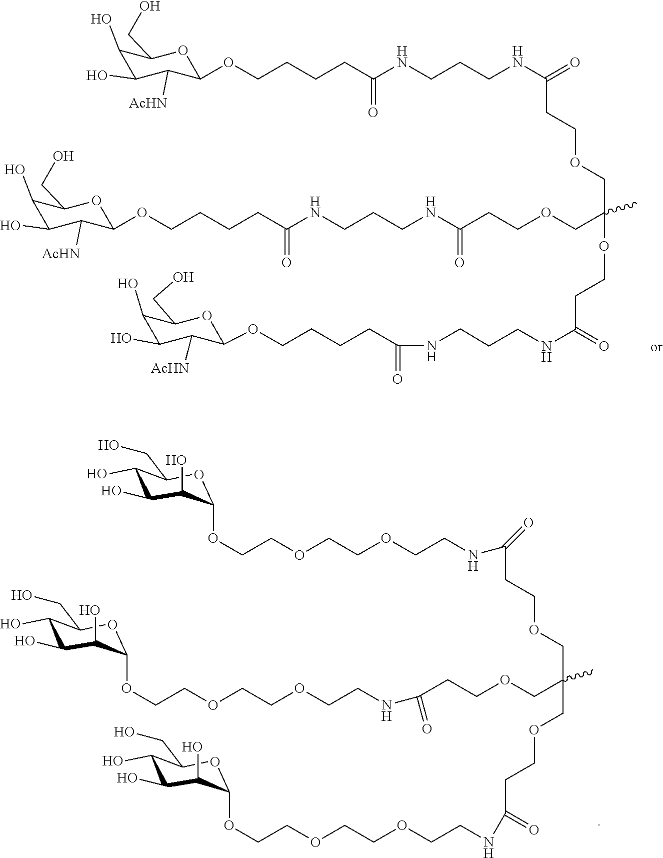

In certain embodiments, the REVERSIR compound comprises a monomer of structure:

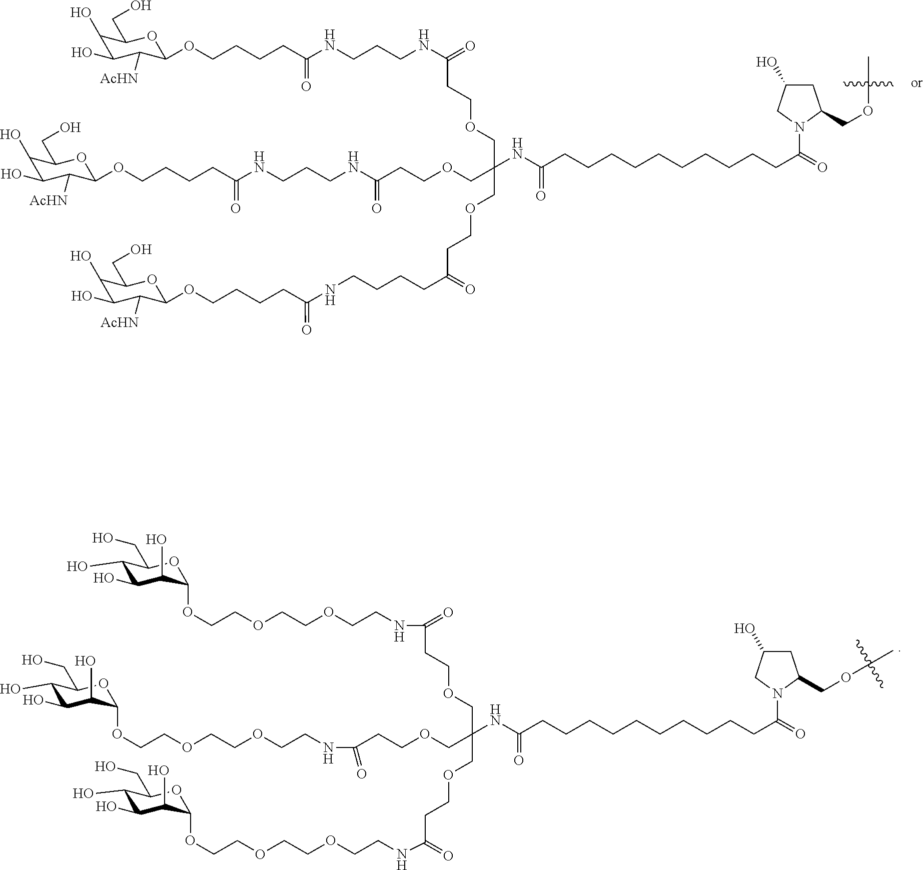

Synthesis of above described ligands and monomers is described, for example, in U.S. Pat. No. 8,106,022, content of which is incorporated herein by reference in its entirety.

In certain embodiments, the oligomeric compound described herein, including but not limited to REVERSIR compounds, comprises a ligand of structure:

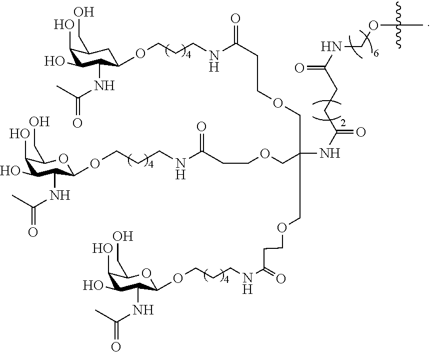

In certain embodiments, the oligomeric compound described herein, including but not limited to REVERSIR compounds, comprises a ligand from those described in U.S. Pat. No. 9,181,549 to Prakash et al., the content of which is incorporated herein by reference in its entirety.

Linking groups or bifunctional linking moieties such as those known in the art are amenable to the compounds provided herein. Linking groups are useful for attachment of chemical functional groups, conjugate groups, reporter groups and other groups to selective sites in a parent compound such as for example an oligomeric compound. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like. Some nonlimiting examples of bifunctional linking moieties include 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other linking groups include, but are not limited to, substituted C1-C10 alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, the ligand is conjugated with the oligomeric compound via a linker.

As used herein, the term “linker” means an organic moiety that connects two parts of a compound. Linkers typically comprise a direct bond or an atom such as oxygen or sulfur, a unit such as NR¹, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R′)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In one embodiment, the linker is —[(P-Q″-R)_{q}—X—(P′-Q′″—R′)_{q}]_{q″}-T-, wherein: P, R, T, P′, R′ and T are each independently for each occurrence absent, CO, NH O, S, OC(O), NHC(O), CH₂, CH₂NH, CH₂O; NHCH(R^{a})C(O), —C(O)—CH(R^{a})—NH—, CH═N—O,

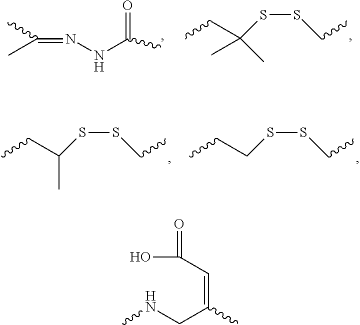

or heterocyclyl; Q″ and Q′″ are each independently for each occurrence absent, —(CH₂)ₙ—, —C(R¹)(R²)(CH₂)ₙ—, —(CH₂)ₙC(R¹)(R²)—, —(CH₂CH₂O)ₘCH₂CH₂—, or —(CH₂CH₂O)ₘCH₂CH₂NH—;
X is absent or a cleavable linking group;
R^{a} is H or an amino acid side chain;
R¹ and R² are each independently for each occurrence H, CH₃, OH, SH or N(R^{N})₂;
R^{N} is independently for each occurrence H, methyl, ethyl, propyl, isopropyl, butyl or benzyl;
q, q′ and q″ are each independently for each occurrence 0-20 and wherein the repeating unit can be the same or different;
n is independently for each occurrence 1-20; and
m is independently for each occurrence 0-50.

In some embodiments, the linker comprises at least one cleavable linking group.

In some embodiments, the linker is a branched linker. The branchpoint of the branched linker may be at least trivalent, but can be a tetravalent, pentavalent or hexavalent atom, or a group presenting such multiple valencies. In some embodiments, the branchpoint is, —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branchpoint is glycerol or derivative thereof.

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In some embodiments, cleavable linking group is cleaved at least 1.25, 1.5, 1.75, 2, 3, 4, 5, 10, 25, 50, or 100 times faster in the cell (or under in vitro conditions selected to mimic intracellular conditions) as compared to blood or serum (or under in vitro conditions selected to mimic extracellular conditions). In some embodiments, the cleavable linking group is cleaved by less than 90%, 80%, 70%, 60%, 50%, 40%, 30%, 20%, 10%, 5%, or 1% in the blood (or in vitro conditions selected to mimic extracellular conditions) as compared to in the cell (or under in vitro conditions selected to mimic intracellular conditions).

Exemplary cleavable linking groups include, but are not limited to, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid celavable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR^{A}C(O)NHCHR^{B}C(O)—, where R^{A} and R^{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease found in cells.

In some embodiments, an acid cleavable linking group is cleaveable in an acidic environment with a pH od about 6.5 or lower (e.g., about 6.-, 5.5, 5.0, or lower), or by agents such as enzymes that can act as a general acid.

In some embodiments, the linker is an oligonucleotide linker including, but not limited to, (N)ₙ; wherein N is independently a modified or unmodified nucleotide and n is 1-23. In some embodiments, n is 1-10, e.g., 1, 2, 3, 4, 5, 6, 7, 8, 9, or 10. In some embodiments, the oligonucleotide linker is selected from the group consisting of GNRA, (G)₄, (U)₄, and (dT)₄, wherein N is a modified or unmodified nucleotide and R is a modified or unmodified purine nucleotide. Some of the nucleotides in the linker can be involved in base-pair interactions with other nucleotides in the linker. It will be appreciated by one of skill in the art that any oligonucleotide chemical modifications or variations describe herein can be used in the oligonucleotide linker. In certain embodiments, the linker is dA.

### IV. Synthesis, Purification and Analysis of the REVERSIRs of the Invention

Oligomerization of modified and unmodified nucleosides and nucleotides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligomeric compounds provided herein can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The invention is not limited by the method of antisense compound synthesis.

Methods of purification and analysis of oligomeric compounds are known to those skilled in the art. Analysis methods include capillary electrophoresis (CE) and electrospray-mass spectroscopy. Such synthesis and analysis methods can be performed in multi-well plates. The method of the invention is not limited by the method of oligomer purification.

The oligomeric compounds of the invention can be prepared using solution-phase or solid-phase organic synthesis, or enzymatically by methods known in the art. Organic synthesis offers the advantage that the oligomeric strands comprising non-natural or modified nucleotides can be easily prepared. Any other means for such synthesis known in the art can additionally or alternatively be employed. It is also known to use similar techniques to prepare other oligomeric compounds, such as those comprising phosphorothioates, phosphorodithioates and alkylated derivatives of intersugar linkages. The double-stranded oligomeric compounds of the invention can be prepared using a two-step procedure. First, the individual strands of the double-stranded molecule are prepared separately. Then, the component strands are annealed.

Regardless of the method of synthesis, the oligomeric compounds can be prepared in a solution (e.g., an aqueous and/or organic solution) that is appropriate for formulation. For example, the oligonmeric preparation can be precipitated and redissolved in pure double-distilled water, and lyophilized. The dried oligomeric compound can then be resuspended in a solution appropriate for the intended formulation process.

Teachings regarding the synthesis of particular modified oligomeric compounds can be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having beta-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups can be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. Nos. 5,223,168, and 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

### V. Pharmaceutical Compositions of the Invention

Oligomeric compounds can be admixed with pharmaceutically acceptable active and/or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Oligomeric compounds, including REVERSIR compounds, can be utilized in pharmaceutical compositions by combining such oligomeric compounds with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising a REVERSIR compound and a pharmaceutically acceptable diluent. In certain embodiments, the pharmaceutically acceptable diluent is PBS.

Pharmaceutical compositions comprising oligomeric compounds encompass any pharmaceutically acceptable salts, esters, or salts of such esters. In certain embodiments, pharmaceutical compositions comprising oligomeric compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an oligomeric compound which are cleaved by endogenous nucleases within the body, to form the active oligomeric compound.

The pharmaceutical compositions of the present invention may be administered in a number of ways depending upon whether local or systemic treatment is desired and upon the area to be treated. Administration may be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

The oligomeric compounds can be delivered in a manner to target a particular tissue, such as the liver (e.g., the hepatocytes of the liver).

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Suitable topical formulations include those in which the iRNAs featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). iRNAs featured in the invention may be encapsulated within liposomes or may form complexes thereto, in particular to cationic liposomes. Alternatively, iRNAs may be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g., as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P.Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C_{1215G}, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Liposome compositions can be prepared by a variety of methods that are known in the art. See e.g., U.S. Pat. Nos. 4,235,871; 4,737,323; 4,897,355 and 5,171,678; published International Applications WO 96/14057 and WO 96/37194; Felgner, P. L. et al., Proc. Natd. Acad. Sci., USA (1987) 8:7413-7417, Bangham, et al. M. Mol. Biol. (1965) 23:238, Olson, et al. Biochim. Biophys. Acta (1979) 557:9, Szoka, et al. Proc. Natd. Acad. Sci. (1978) 75: 4194, Mayhew, et al. Biochim. Biophys. Acta (1984) 775:169, Kim, et al. Biochim. Biophys. Acta (1983) 728:339, and Fukunaga, et al. Endocrinol. (1984) 115:757.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

### A. Lipid Particles

In some embodiments, the REVERSIR can be fully encapsulated in a lipid formulation, e.g., a LNP, or other nucleic acid-lipid particle. The REVERSIR encapsulated in the lipid formulation can be unconjugated or conjugated with a ligand (i.e., a conjugated REVERSIR).

As used herein, the term “LNP” refers to a stable nucleic acid-lipid particle. LNPs contain a cationic lipid, a non-cationic lipid, and a lipid that prevents aggregation of the particle (e.g., a PEG-lipid conjugate). LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In some embodiments, the lipid to drug ratio (mass/mass ratio) (e.g., lipid to REVERSIR ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

The cationic lipid can be, for example, N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol, or a mixture thereof. The cationic lipid can comprise from about 20 mol % to about 50 mol % or about 40 mol % of the total lipid present in the particle.

In some embodiments, the compound 2,2-Dilinoleyl-4-dimethylaminoethyl-[1,3]-dioxolane can be used to prepare lipid-REVERSIR nanoparticles. Synthesis of 2,2-Dilinoleyl-4-dimethylaminoethyl-[1,3]-dioxolane is described in International application no. PCT/US2009/061897, published as WO/2010/048536, which is herein incorporated by reference.

In some embodiments, the lipid-REVERSIR particle includes 40% 2, 2-Dilinoleyl-4-dimethylaminoethyl-[1,3]-dioxolane: 10% DSPC: 40% Cholesterol: 10% PEG-C-DOMG (mole percent) with a particle size of 63.0±20 nm and a 0.027 REVERSIR/Lipid Ratio.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be from about 5 mol % to about 90 mol %, about 10 mol %, or about 58 mol % ifcholesterol is included, ofthe total lipid present in the particle.

The conjugated lipid that inhibits aggregation ofparticles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (C The conjugated lipid that prevents aggregation of particles can be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

Additional exemplary lipid-REVERSIR formulations are described in the Table below.

### Exemplary Lipid REVERSIR Formulations

| | | cationic lipid/non-cationic |
|---|---|---|
| | | lipid/cholesterol/PEG-lipid conjugate |
| Formulation | Ionizable/Cationic Lipid | Lipid:REVERSIR ratio |
| LNP_DLinDMA | 1,2-Dilinolenyloxy-N,N- | DLinDMA/DPPC/Cholesterol/PEG- |
| | dimethylaminopropane (DLinDMA) | CDMA |
| | | (57.1/7.1/34.4/1.4) |
| | | lipid:REVERSIR~7:1 |
| 2-XTC | 2,2-Dilinoleyl-4-dimethylaminoethyl- | XTC/DPPC/Cholesterol/PEG-cDMA |
| | [1,3]-dioxolane (XTC) | 57.1/7.1/34.4/1.4 |
| | | lipid:REVERSIR~7:1 |
| LNP05 | 2,2-Dilinoleyl-4-dimethylaminoethyl- | XTC/DSPC/Cholesterol/PEG-DMG |
| | [1,3]-dioxolane (XTC) | 57.5/7.5/31.5/3.5 |
| | | lipid:REVERSIR~6:1 |
| LNP06 | 2,2-Dilinoleyl-4-dimethylaminoethyl- | XTC/DSPC/Cholesterol/PEG-DMG |
| | [1,3]-dioxolane (XTC) | 57.5/7.5/31.5/3.5 |
| | | lipid:REVERSIR~11:1 |
| LNP07 | 2,2-Dilinoleyl-4-dimethylaminoethyl- | XTC/DSPC/Cholesterol/PEG-DMG |
| | [1,3]-dioxolane (XTC) | 60/7.5/31/1.5, |
| | | lipid:REVERSIR~6:1 |
| LNP08 | 2,2-Dilinoleyl-4-dimethylaminoethyl- | XTC/DSPC/Cholesterol/PEG-DMG |
| | [1,3]-dioxolane (XTC) | 60/7.5/31/1.5, |
| | | lipid:REVERSIR~11:1 |
| LNP09 | 2,2-Dilinoleyl-4-dimethylaminoethyl- | XTC/DSPC/Cholesterol/PEG-DMG |
| | [1,3]-dioxolane (XTC) | 50/10/38.5/1.5 |
| | | Lipid:REVERSIR 10:1 |
| LNP10 | (3aR,5s,6aS)-N,N-dimethyl-2,2- | ALN100/DSPC/Cholesterol/PEG- |
| | di((9Z,12Z)-octadeca-9,12- | DMG |
| | dienyl)tetrahydro-3aH- | 50/10/38.5/1.5 |
| | cyclopenta[d][1,3]dioxol-5-amine | Lipid:REVERSIR 10:1 |
| | (ALN100) | |
| LNP11 | (6Z,9Z,28Z,31Z)-heptatriaconta- | MC-3/DSPC/Cholesterol/PEG-DMG |
| | 6,9,28,31-tetraen-19-yl 4- | 50/10/38.5/1.5 |
| | (dimethylamino)butanoate (MC3) | Lipid:REVERSIR 10:1 |
| LNP12 | 1,1′-(2-(4-(2-((2-(bis(2- | Tech G1/DSPC/Cholesterol/PEG- |
| | hydroxydodecyl)amino)ethyl)(2- | DMG |
| | hydroxydodecyl)amino)ethyl)piperazin- | 50/10/38.5/1.5 |
| | 1-yl)ethylazanediyl)didodecan-2-ol | Lipid:REVERSIR 10:1 |
| | (C12-200> | |
| LNP13 | XTC | XTC/DSPC/Chol/PEG-DMG |
| | | 50/10/38.5/1.5 |
| | | Lipid:REVERSIR: 33:1 |
| LNP14 | MC3 | MC3/DSPC/Chol/PEG-DMG |
| | | 40/15/40/5 |
| | | Lipid:REVERSIR: 11:1 |
| LNP15 | MC3 | MC3/DSPC/Chol/PEG-DSG/GalNAc- |
| | | PEG-DSG |
| | | 50/10/35/4.5/0.5 |
| | | Lipid:REVERSIR: 11:1 |
| LNP16 | MC3 | MC3/DSPC/Chol/PEG-DMG |
| | | 50/10/38.5/1.5 |
| | | Lipid:REVERSIR: 7:1 |
| LNP17 | MC3 | MC3/DSPC/Chol/PEG-DSG |
| | | 50/10/38.5/1.5 |
| | | Lipid:REVERSIR: 10:1 |
| LNP18 | MC3 | MC3/DSPC/Chol/PEG-DMG |
| | | 50/10/38.5/1.5 |
| | | Lipid:REVERSIR: 12:1 |
| LNP19 | MC3 | MC3/DSPC/Chol/PEG-DMG |
| | | 50/10/35/5 |
| | | Lipid:REVERSIR: 8:1 |
| LNP20 | MC3 | MC3/DSPC/Chol/PEG-DPG |
| | | 50/10/38.5/1.5 |
| | | Lipid:REVERSIR: 10:1 |
| LNP21 | C12-200 | C12-200/DSPC/Chol/PEG-DSG |
| | | 50/10/38.5/1.5 |
| | | Lipid:REVERSIR: 7:1 |
| LNP22 | XTC | XTC/DSPC/Chol/PEG-DSG |
| | | 50/10/38.5/1.5 |
| | | Lipid:REVERSIR: 10:1 |
| LNPX | (13Z,16Z)-N,N-dimethyl-3- | (13Z,16Z)-N,N-dimethyl-3- |
| | nonyldocosa-13,16-dien-1-amine | nonyldocosa-13,16-dien-1- |
| | | amine/DSPC/Chol/PEG-DSG |
| | | 50/10/38.5/1.5 |
| | | Lipid:REVERSIR: 10:1 |
| LNPY | Biodegradable lipid | Biodegradable lipid/DSPC/Chol/PEG- |
| | | DSG |
| | | 50/10/38.5/1.5 |
| | | Lipid:REVERSIR: 10:1 |

*The REVERSIR can be an unconjugated or conjugated with a ligand (i.e., conjugated REVERSIR).

Abbreviations in the Table above include the following: DSPC: distearoylphosphatidylcholine; DPPC: dipalmitoylphosphatidylcholine; PEG-DMG: PEG-didimyristoyl glycerol (C14-PEG, or PEG-C14) (PEG with avg mol wt of 2000); PEG-DSG: PEG-distyryl glycerol (C18-PEG, or PEG-C18) (PEG with avg mol wt of 2000); PEG-cDMA: PEG-carbamoyl-1,2-dimyristyloxypropylamine (PEG with avg mol wt of 2000).

DLinDMA (1,2-Dilinolenyloxy-N,N-dimethylaminopropane) comprising formulations are described in International Publication No. WO2009/127060, filed Apr. 15, 2009, which is hereby incorporated by reference.

XTC comprising formulations are described, e.g., in U.S. Provisional Ser. No. 61/148,366, filed Jan. 29, 2009; U.S. Provisional Ser. No. 61/156,851, filed Mar. 2, 2009; U.S. Provisional Serial No. filed Jun. 10, 2009; U.S. Provisional Ser. No. 61/228,373, filed Jul. 24, 2009; U.S. Provisional Ser. No. 61/239,686, filed Sep. 3, 2009, and International Application No. PCT/US2010/022614, filed Jan. 29, 2010, which are hereby incorporated by reference.

MC3 comprising formulations are described, e.g., in U.S. Publication No. 2010/0324120, filed Jun. 10, 2010, the entire contents of which are hereby incorporated by reference.

Biodegradable lipid comprising formulations are described, e.g., PCT Publications No. WO2011/153493, filed Jun. 3, 2011 and WO/2013/086354, filed Dec. 7, 2012, the entire contents of which are hereby incorporated by reference.

(13Z,16Z)—N,N-dimethyl-3-nonyldocosa-13,16-dien-1-amine comprising formulations are described, e.g., in PCT Publications No. WO/2012/040184, filed Sep. 20, 2011, the entire contents of which are hereby incorporated by reference.

The oligomeric compounds of the invention can be prepared and formulated as micelles. As used herein, “micelles” are a particular type of molecular assembly in which amphipathic molecules are arranged in a spherical structure such that all hydrophobic portions on the molecules are directed inward, leaving the hydrophilic portions in contact with the surrounding aqueous phase. The converse arrangement exists if the environment is hydrophobic.

In some embodiments, the formulations comprises micelles formed from an oligonucleotide of the invention and at least one amphiphilic carrier, in which the micelles have an average diameter of less than about 100 nm, preferably. More preferred embodiments provide micelles having an average diameter less than about 50 nm, and even more preferred embodiments provide micelles having an average diameter less than about 30 nm, or even less than about 20 nm.

Micelle formulations can be prepared by mixing an aqueous solution of the oligonucleotide composition, an alkali metal C₈ to C₂₂ alkyl sulphate, and an amphiphilic carrier. The amphiphilic carrier can be added at the same time or after addition of the alkali metal alkyl sulphate. Micelles will form with substantially any kind of mixing of the ingredients but vigorous mixing in order to provide smaller size micelles.

The oligomeric compounds of the present invention can be prepared and formulated as emulsions. As used herein, “emulsion” is a heterogenous system of one liquid dispersed in another in the form of droplets.

Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials is also included in emulsion formulations and contributes to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In some embodiments, the compositions are formulated as microemulsions. As used herein, “microemulsion” refers to a system of water, oil and amphiphile which is a single optically isotropic and thermodynamically stable liquid solution. Microemuslions also include thermodynamically stable, isotropically clear dispersions of two immiscible liquids that are stabilized by interfacial films of surface-active molecules.

A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or dsRNAs. Microemulsions have also been effective in the transdermal delivery of active components in both cosmetic and pharmaceutical applications. It is expected that the microemulsion compositions and formulations of the present invention will facilitate the increased systemic absorption of dsRNAs and nucleic acids from the gastrointestinal tract, as well as improve the local cellular uptake of dsRNAs and nucleic acids.

Microemulsions of the present invention may also contain additional components and additives such as sorbitan monostearate (Grill 3), Labrasol, and penetration enhancers to improve the properties of the formulation and to enhance the absorption of the dsRNAs and nucleic acids of the present invention. Penetration enhancers used in the microemulsions of the present invention may be classified as belonging to one of five broad categories-surfactants, fatty acids, bile salts, chelating agents, and non-chelating non-surfactants (Lee et al., Critical Reviews in Therapeutic Drug Carrier Systems, 1991, p. 92). Each of these classes has been discussed above.

The application of emulsion formulations via dermatological, oral and parenteral routes and methods for their manufacture have been reviewed in the literature, for example see Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., volume 1, p. 245; and Block, in Pharmaceutical Dosage Forms, Lieberman, Rieger and Banker (Eds.), 1988, Marcel Dekker, Inc., New York, N.Y., volume 1, p. 335, contents of which are herein incorporated by reference in their entirety.

The oligomeric compounds of the present invention can be prepared and formulated as lipid particles, e.g., formulated lipid particles (FLiPs) comprising (a) an oligonucleotide of the invention, where said oligonucleotide has been conjugated to a lipophile and (b) at least one lipid component, for example an emulsion, liposome, isolated lipoprotein, reconstituted lipoprotein or phospholipid, to which the conjugated oligonucleotide has been aggregated, admixed or associated. The stoichiometry of oligonucleotide to the lipid component can be 1:1. Alternatively the stoichiometry can be 1:many, many:1 or many:many, where many is two or more.

The FLiP can comprise triacylglycerols, phospholipids, glycerol and one or several lipid-binding proteins aggregated, admixed or associated via a lipophilic linker molecule with an oligonucleotide. Surprisingly, it has been found that due to said one or several lipid-binding proteins in combination with the above mentioned lipids, the FLiPs show affinity to liver, gut, kidney, steroidogenic organs, heart, lung and/or muscle tissue. These FLiPs can therefore serve as carrier for oligonucleotides to these tissues. For example, lipid-conjugated oligonucleotides, e.g., cholesterol-conjugated oligonucleotides, bind to HDL and LDL lipoprotein particles which mediate cellular uptake upon binding to their respective receptors thus directing oligonucleotide delivery into liver, gut, kidney and steroidogenic organs, see Wolfrum et al. Nature Biotech. (2007), 25:1145-1157.

The FLiP can be a lipid particle comprising 15-25% triacylglycerol, about 0.5-2% phospholipids and 1-3% glycerol, and one or several lipid-binding proteins. FLiPs can be a lipid particle having about 15-25% triacylglycerol, about 1-2% phospholipids, about 2-3% glycerol, and one or several lipid-binding proteins. In some embodiments, the lipid particle comprises about 20% triacylglycerol, about 1.2% phospholipids and about 2.25% glycerol, and one or several lipid-binding proteins.

Another suitable lipid component for FLiPs is lipoproteins, for example isolated lipoproteins or more preferably reconstituted lipoprotieins. Exemplary lipoproteins include chylomicrons, VLDL (Very Low Density Lipoproteins), IDL (Intermediate Density Lipoproteins), LDL (Low Density Lipoproteins) and HDL (High Density Lipoproteins). Methods of producing reconstituted lipoproteins are known in the art, for example see A. Jones, Experimental Lung Res. 6, 255-270 (1984), U.S. Pat. Nos. 4,643,988 and 5,128,318, PCT publication WO87/02062, Canadian Pat. No. 2,138,925. Other methods of producing reconstituted lipoproteins, especially for apolipoproteins A-I, A-II, A-IV, apoC and apoE have been described in A. Jonas, Methods in Enzymology 128, 553-582 (1986) and G. Franceschini et al. J. Biol. Chem., 260(30), 16321-25 (1985).

One preferred lipid component for FLiP is Intralipid. Intralipid® is a brand name for the first safe fat emulsion for human use. Intralipid® 20% (a 20% intravenous fat emulsion) is made up of 20% soybean oil, 1.2% egg yolk phospholipids, 2.25% glycerin, and water for injection. It is further within the present invention that other suitable oils, such as saflower oil, can serve to produce the lipid component of the FLiP.

FLiP can range in size from about 20-50 nm or about 30-50 nm, e.g., about 35 nm or about 40 nm. In some embodiments, the FLiP has a particle size of at least about 100 nm. FLiPs can alternatively be between about 100-150 nm, e.g., about 110 nm, about 120 nm, about 130 nm, or about 140 nm, whether characterized as liposome- or emulsion-based. Multiple FLiPs can also be aggregated and delivered together, therefore the size can be larger than 100 nm.

The process for making the lipid particles comprises the steps of: (a) mixing a lipid components with one or several lipophile (e.g. cholesterol) conjugated oligonucleotides that can be chemically modified; and (b) fractionating this mixture. In some embodiments, the process comprises the additional step of selecting the fraction with particle size of 30-50 nm, preferably of about 40 nm in size.

Some exemplary lipid particle formulations amenable to the invention are described in U.S. patent application Ser. No. 12/412,206, filed Mar. 26, 2009, content of which is herein incorporated by reference in its entirety.

In some embodiments, the oligomeric compounds can be formulated in yeast cell wall particles (“YCWP”). A yeast cell wall particle comprises an extracted yeast cell wall exterior and a core, the core comprising a payload (e.g., oligonucleotides). Exterior of the particle comprises yeast glucans (e.g. beta glucans, beta-1,3-glucans, beta-1,6-glucans), yeast mannans, or combinations thereof. Yeast cell wall particles are typically spherical particles about 1-4 μm in diameter.

Preparation of yeast cell wall particles is known in the art, and is described, for example in U.S. Pat. Nos. 4,992,540; 5,082,936; 5,028,703; 5,032,401; 5,322,841; 5,401,727; 5,504,079; 5,607,677; 5,741,495; 5,830,463; 5,968,811; 6,444,448; and 6,476,003, U.S. Pat. App. Pub. Nos. 2003/0216346 and 2004/0014715, and Int. App. Pub. No. WO 2002/12348, contents of which are herein incorporated by reference in their entirety. Applications of yeast cell like particles for drug delivery are described, for example in U.S. Pat. Nos. 5,032,401; 5,607,677; 5,741,495; and 5,830,463, and U.S. Pat. Pub Nos. 2005/0281781 and 2008/0044438, contents of which are herein incorporated by reference in their entirety. U.S. Pat. App. Pub. No. 2009/0226528, contents of which are herein incorporated by reference, describes formulation of nucleic acids with yeast cell wall particles for delivery of oligonucleotide to cells.

Exemplary formulations for oligomeric compounds are described in U.S. Pat. Nos. 4,897,355; 4,394,448; 4,235,871; 4,231,877; 4,224,179; 4,753,788; 4,673,567; 4,247,411; 4,814,270; 5,567,434; 5,552,157; 5,565,213; 5,738,868; 5,795,587; 5,922,859; 6,077,663; 7,906,484; and 8,642,076; PCT Publication No. WO2009/132131 and U.S. Pat. Pub. Nos. 2006/0240093, 2007/0135372, 2011/0117125, 2009/0291131, 2012/0316220, 2009/0163705 and 2013/0129785, contents of all of which is herein incorporated by reference in its entirety. Behr (1994) Bioconjugate Chem. 5:382-389, and Lewis et al. (1996) PNAS 93:3176-3181), also describe formulations for oligonucleotides that are amenable to the invention, contents of which are herein incorporated by reference in their entirety.

### VI. dsRNAs Targeted by REVERSIRs

The present invention provides REVERSIRs™ that inhibit the activity of dsRNA agents comprising a thermally destabilizing nucleotide modification in the antisense strand.

In particular embodiments, the thermally destabilizing nucleotide modification is selected from the group consisting of an abasic modification; a mismatch with the opposing nucleotide in the duplex; a destabilizing sugar modification, a 2′-deoxy modification, an acyclic nucleotide, an unlocked nucleic acid (UNA), and a glycerol nucleic acid (GNA).

In some embodiments, the thermally destabilizing nucleotide modification is an abasic modification. In some embodiments, the thermally destabilizing nucleotide modification is a mismatch with the opposing nucleotide in the duplex. In some embodiments, the thermally destabilizing nucleotide modification is a destabilizing sugar modification. In some embodiments, the thermally destabilizing nucleotide modification is a 2′-deoxy modification. In some embodiments, the thermally destabilizing nucleotide modification is an acyclic nucleotide. In some embodiments, the thermally destabilizing nucleotide modification is an unlocked nucleic acid (UNA). In some embodiments, the thermally destabilizing nucleotide modification is a glycerol nucleic acid (GNA).

In some embodiments, the dsRNA agent comprises at least one, at least two, at least three, at least five, or at least ten thermally destabilizing nucleotide modifications described herein.

As discussed above, the term “dsRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, or dsRNA agents herein.

As used herein, the terms “dsRNA activity,” “siRNA activity,” and “RNAi activity” refer to gene silencing or RNAi interference by adsRNA agent.

As used herein, “gene silencing” by a RNA interference molecule refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, gene expression modulation happens when the expression of the gene, or level of RNA molecule or equivalent RNA molecules encoding one or more proteins or protein subunits is at least 5%, 10%, 20%, 30%, 40%, 50%, 60%, 70%, 80%, 90%, 100%, 2-fold, 3-fold, 4-fold, 5-fold or more different from that observed in the absence of the dsRNA, e.g., RNAi agent. The % and/or fold difference can be calculated relative to the control or the non-control, for example,
[expression with dsRNA−expression without dsRNA]
% difference=- - -
expression without dsRNA

As used herein, the term “inhibit”, “down-regulate”, or “reduce” in relation to gene expression, means that the expression of the gene, or level of RNA molecules or equivalent RNA molecules encoding one or more proteins or protein subunits, or activity of one or more proteins or protein subunits, is reduced below that observed in the absence of modulator. The gene expression is down-regulated when expression of the gene, or level of RNA molecules or equivalent RNA molecules encoding one or more proteins or protein subunits, or activity of one or more proteins or protein subunits, is reduced at least 10% lower relative to a corresponding non-modulated control, and preferably at least 10%, 20%, 30%, 40%, 50%, 60%, 70%, 80%, 90%, 95%, 98%, 99% or most preferably, 100% (i.e., no gene expression).

As used herein, the term “increase” or “up-regulate” in relation to gene expression, means that the expression of the gene, or level of RNA molecules or equivalent RNA molecules encoding one or more proteins or protein subunits, or activity of one or more proteins or protein subunits, is increased above that observed in the absence of modulator. The gene expression is up-regulated when expression of the gene, or level of RNA molecules or equivalent RNA molecules encoding one or more proteins or protein subunits, or activity of one or more proteins or protein subunits, is increased at least 10% relative to a corresponding non-modulated control, and preferably at least 10%, 20%, 30%, 40%, 50%, 60%, 70%, 80%, 90%, 95%, 98%, 100%, 1.1-fold, 1.25-fold, 1.5-fold, 1.75-fold, 2-fold, 3-fold, 4-fold, 5-fold, 10-fold, 50-fold, 100-fold or more.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The dsRNA agent targeted by one or more REVERSIRs of the invention includes two RNA strands that are complementary and hybridize to form a duplex structure under conditions in which the dsRNA will be used. One strand of a dsRNA (the antisense strand) includes a region of complementarity that is substantially complementary, and generally fully complementary, to a universal target sequence. The target sequence can be derived from the sequence of an mRNA formed during the expression of a universal target sequence. The other strand (the sense strand) includes a region that is complementary to the antisense strand, such that the two strands hybridize and form a duplex structure when combined under suitable conditions.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24,20-23, 20-22, 20-21, 21-25, 21-24, 21-23, 21-22, 22-25, 22-24, 22-23, 23-25, 23-24 or 24-25 base pairs in length, for example, 19-21 base pairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the dsRNA is about 19 to about 23 nucleotides in length, or about 25 to about 30 nucleotides in length. In general, the dsRNA is long enough to serve as a substrate for the Dicer enzyme. For example, it is well-known in the art that dsRNAs longer than about 21-23 nucleotides in length may serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 19 to about 30 base pairs, e.g., about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target expression of a universal target sequence is not generated in the target cell by cleavage of a larger dsRNA.

A dsRNA as described herein can further include one or more single-stranded nucleotide overhangs, e.g., 1-4, 2-4, 1-3, 2-3, 1, 2, 3, or 4 nucleotides, dsRNAs having at least one nucleotide overhang can have superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of an antisense or sense strand of a dsRNA.

A dsRNA can be synthesized by standard methods known in the art. Double stranded RNAi compounds of the invention may be prepared using a two-step procedure. First, the individual strands of the double stranded RNA molecule are prepared separately. Then, the component strands are annealed. The individual strands of the dsRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

In an aspect, a dsRNA of the invention includes at least two nucleotide sequences, a sense sequence and an anti-sense sequence. In this aspect, one of the two sequences is complementary to the other of the two sequences, with one of the sequences being substantially complementary to a sequence of an mRNA generated in the expression of a universal target sequence.

In certain embodiments, the substantially complementary sequences of the dsRNA are contained on separate oligonucleotides. In other embodiments, the substantially complementary sequences of the dsRNA are contained on a single oligonucleotide.

In some embodiments, the dsRNA agent inhibits expression of angiotensinogen (AGT), wherein the dsRNA agent comprises a sense strand and an antisense strand forming a double stranded region.

In some embodiments, the nucleotide sequence of the antisense strand of the dsRNA agent comprises a nucleotide sequence comprising at least 19, at least 20, at least 21, or at least 22 contiguous nucleotides of the nucleotide sequence UGUACUCUCAUUGUGGAUGACGA of SEQ ID NO: 9. In some embodiments, the nucleotide sequence of the sense strand of the dsRNA agent comprises a nucleotide sequence comprising at least 19, at least 20, at least 21, or at least 22 contiguous nucleotides of the nucleotide sequence GUCAUCCACAAUGAGAGUACA of SEQ ID NO: 10.

In some embodiments, the antisense strand of the dsRNA agent comprises the nucleotide sequence UGUACUCUCAUUGUGGAUGACGA of SEQ ID NO: 9.

In some embodiments, the sense strand of the dsRNA agent comprises the nucleotide sequence GUCAUCCACAAUGAGAGUACA of SEQ ID NO: 10.

In some embodiments, the antisense strand of the dsRNA agent comprises the nucleotide sequence UGUACUCUCAUUGUGGAUGACGA of SEQ ID NO: 9, and the sense strand of the dsRNA agent comprises the nucleotide sequence GUCAUCCACAAUGAGAGUACA of SEQ ID NO: 10.

In some embodiments, the antisense strand of the dsRNA agent consists of the nucleotide sequence UGUACUCUCAUUGUGGAUGACGA of SEQ ID NO: 9, and the sense strand of the dsRNA agent consists of the nucleotide sequence GUCAUCCACAAUGAGAGUACA of SEQ ID NO: 10.

In some embodiments, the dsRNA agent is AD-85481, also known as Zilebesiran®.

AD-85481 comprises a sense strand and an antisense strand forming a double stranded region, wherein the sense strand comprises the nucleotide sequence 5′-gsuscaucCfaCfAfAfugagaguaca-3′ and the antisense strand comprises the nucleotide sequence 5′-usGfsuac(Tgn)cucauugUfgGfaugacsgsa-3′, wherein a, g, c, and u are 2′-O-methyl (2′-OMe) A, G, C, and U, respectively; Af, Gf, Cf and Uf are 2′-fluoro A, G, C and U, respectively; s is a phosphorothioate linkage; and (Tgn) is a thymidine-glycol nucleic acid (GNA)S-Isomer; and wherein the 3′-end of the sense strand is conjugated to a ligand as shown in the following schematic

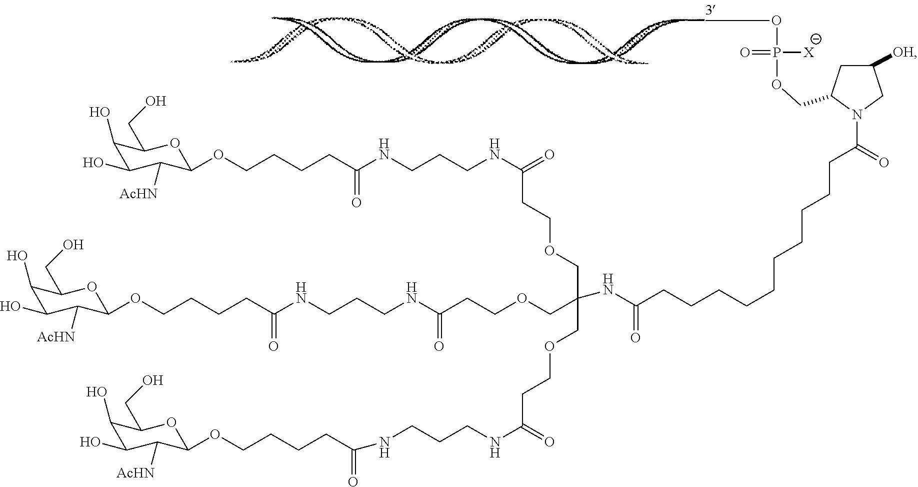

wherein X is O.

Further, a pharmaceutically acceptable salt form of the dsRNA agent includes any salt that is pharmaceutically acceptable, e.g., a sodium salt of the dsRNA agent. In some embodiments, the pharmaceutically acceptable salt of the dsRNA has the following structure:

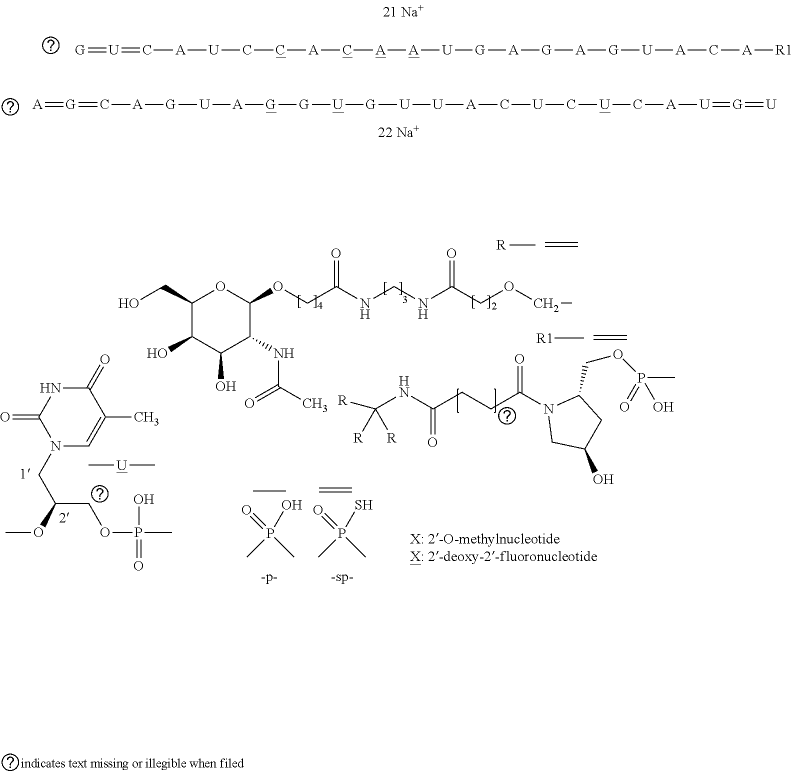

Additional dsRNA agents targeted by one or more REVERSIRs of the invention are described in International PCT Publication No.s WO 2015/179724 and WO 2019/222166, the entire contents of each of which are incorporated herein by reference.

The skilled person is well aware that dsRNAs having a duplex structure of about 20 to 23 base pairs, e.g., 21, base pairs have been hailed as particularly effective in inducing RNA interference (Elbashir et al., EMBO 2001, 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, dsRNAs described herein can include at least one strand of a length of minimally 21 nucleotides. It can be reasonably expected that shorter duplexes minus only a few nucleotides on one or both ends can be similarly effective as compared to the dsRNAs described above. Hence, dsRNAs having a sequence of at least 19, 20, or more contiguous nucleotides, and differing in their ability to inhibit the expression of a universal target sequence by not more than about 5, 10, 15, 20, 25, or 30% inhibition from a dsRNA comprising the full sequence, are contemplated to be within the scope of the present invention.

In addition, the RNAs identify a site(s) in a universal target sequence transcript that is susceptible to RISC-mediated cleavage. As such, the present invention further features iRNAs that target within one of these sites. As used herein, an iRNA is said to target within a particular site of an RNA transcript if the iRNA promotes cleavage of the transcript anywhere within that particular site. Such an iRNA will generally include at least about 19 contiguous nucleotides coupled to additional nucleotide sequences taken from the region contiguous to the selected sequence in a universal target sequence.

In certain embodiments, the universal iRNA of the invention e.g., a dsRNA, is un-modified, and does not comprise, e.g., chemical modifications or conjugations known in the art and described herein. In other embodiments, the universal iRNA of the invention, e.g., a dsRNA, is chemically modified to enhance stability or other beneficial characteristics. In certain embodiments of the invention, substantially all of the nucleotides of a universal iRNA of the invention are modified, i.e., not more than 5, 4, 3, 2, or 1 unmodified nucleotides are present in a strand of the iRNA. In other embodiments of the invention, all of the nucleotides of a universal iRNA are modified.

The nucleic acids featured in the invention can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of iRNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified iRNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments of the invention, the dsRNA agents of the invention are in a free acid form. In other embodiments of the invention, the dsRNA agents of the invention are in a salt form. In one embodiment, the dsRNA agents of the invention are in a sodium salt form. In certain embodiments, when the dsRNA agents of the invention are in the sodium salt form, sodium ions are present in the agent as counterions for substantially all of the phosphodiester and/or phosphorothiotate groups present in the agent. Agents in which substantially all of the phosphodiester and/or phosphorothioate linkages have a sodium counterion include not more than 5, 4, 3, 2, or 1 phosphodiester and/or phosphorothioate linkages without a sodium counterion. In some embodiments, when the dsRNA agents of the invention are in the sodium salt form, sodium ions are present in the agent as counterions for all of the phosphodiester and/or phosphorothiotate groups present in the agent.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. No. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Suitable RNA mimetics are contemplated for use in iRNAs provided herein, in which both the sugar and the internucleoside linkage, i.e., the backbone, of the nucleotide units are replaced with novel groups. The base units are maintained for hybridization with an appropriate nucleic acid target compound. One such oligomeric compound in which an RNA mimetic that has been shown to have excellent hybridization properties is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative US patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the iRNAs of the invention are described in, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506. The native phosphodiester backbone can be represented as O—P(O)(OH)—OCH2-.

Modified RNAs can also contain one or more substituted sugar moieties. The iRNAs, e.g., dsRNAs, featured herein can include one of the following at the 2′-position: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₙCH₃, O(CH₂)·ₙOCH₃, O(CH₂)ₙNH₂, O(CH₂)ₙCH₃, O(CH₂)nONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₃)2. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar. Representative US patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

A universal iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases such as deoxythimidine (dT), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In some embodiments, an RNAi agent of the disclosure can also be modified to include one or more bicyclic sugar moieties. A “bicyclic sugar” is a furanosyl ring modified by a ring formed by the bridging of two carbons, whether adjacent or non-adjacent. A “bicyclic nucleoside” (“BNA”) is a nucleoside having a sugar moiety comprising a ring formed by bridging two carbons, whether adjacent or non-adjacent, of the sugar ring, thereby forming a bicyclic ring system. In certain embodiments, the bridge connects the 4′-carbon and the 2′-carbon of the sugar ring, optionally, via the 2′-acyclic oxygen atom. Thus, in some embodiments an agent of the invention may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to dsRNAs has been shown to increase dsRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193). Examples of bicyclic nucleosides for use in the polynucleotides of the invention include without limitation nucleosides comprising a bridge between the 4′ and the 2′ ribosyl ring atoms. In certain embodiments, the antisense polynucleotide agents of the invention include one or more bicyclic nucleosides comprising a 4′ to 2′ bridge.

A locked nucleoside can be represented by the structure (omitting stereochemistry),

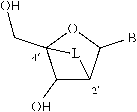

wherein B is a nucleobase or modified nucleobase and L is the linking group that joins the 2′-carbon to the 4′-carbon of the ribose ring. Examples of such 4′ to 2′ bridged bicyclic nucleosides, include but are not limited to 4′-(CH₂)—O-2′ (LNA); 4′-(CH₂)—S-2′; 4′-(CH₂)2-O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)-0-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)-2′ (see, e.g., U.S. Patent Publication No. 2004/0171570); 4′-CH₂—N(R)—O-2′, wherein R is H, C1-C12 alkyl, or a nitrogen protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. patents and U.S. Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

An iRNA of the invention can also be modified to include one or more constrained ethyl nucleotides. As used herein, a “constrained ethyl nucleotide” or “cEt” is a locked nucleic acid comprising a bicyclic sugar moiety comprising a 4′-CH(CH₃)—O-2′ bridge (i.e., L in the preceding structure). In one embodiment, a constrained ethyl nucleotide is in the S conformation referred to herein as “S-cEt.”

An iRNA of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, U.S. Patent Publication No. 2013/0190383; and PCT publication WO 2013/036868, the entire contents of each of which are hereby incorporated herein by reference.

In some embodiments, the dsRNA of the invention comprises one or more modifications that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference.

In certain embodiments, the compositions and methods of the disclosure include a vinyl phosphonate (VP) modification of an RNAi agent as described herein. In exemplary embodiments, a 5′ vinyl phosphonate modified nucleotide of the disclosure has the structure:

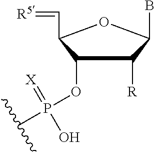

wherein X is O or S;
R is hydrogen, hydroxy, fluoro, or C₁₋₂₀alkoxy (e.g., methoxy or n-hexadecyloxy);
R^{5′} is ═C(H)—P(O)(OH)₂ and the double bond between the C5′ carbon and R^{5′} is in the E or Z orientation (e.g., E orientation); and
B is a nucleobase or a modified nucleobase, optionally where B is adenine, guanine, cytosine, thymine, or uracil.

A vinyl phosphonate of the instant disclosure may be attached to either the antisense or the sense strand of a dsRNA of the disclosure. In certain embodiments, a vinyl phosphonate of the instant disclosure is attached to the antisense strand of a dsRNA, optionally at the 5′ end of the antisense strand of the dsRNA.

Vinyl phosphonate modifications are also contemplated for the compositions and methods of the instant disclosure. An exemplary vinyl phosphonate structure includes the preceding structure, where R5′ is ═C(H)—OP(O)(OH)2 and the double bond between the C5′ carbon and R5′ is in the E or Z orientation (e.g., E orientation).

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3′-phosphate, inverted 2′-deoxy-modified ribonucleotide, such as inverted dT(idT), inverted dA (idA), and inverted abasic 2′-deoxyribonucleotide (iAb) and others. Disclosure of this modification can be found in WO 2011/005861.

In one example, the 3′ or 5′ terminal end of a oligonucleotide is linked to an inverted 2′-deoxy-modified ribonucleotide, such as inverted dT(idT), inverted dA (idA), or a inverted abasic 2′-deoxyribonucleotide (iAb). In one particular example, the inverted 2′-deoxy-modified ribonucleotide is linked to the 3′end of an oligonucleotide, such as the 3′-end of a sense strand described herein, where the linking is via a 3′-3′ phosphodiester linkage or a 3′-3′-phosphorothioate linkage.

In another example, the 3′-end of a sense strand is linked via a 3′-3′-phosphorothioate linkage to an inverted abasic ribonucleotide (iAb). In another example, the 3′-end of a sense strand is linked via a 3′-3′-phosphorothioate linkage to an inverted dA (idA).

In one particular example, the inverted 2′-deoxy-modified ribonucleotide is linked to the 3′end of an oligonucleotide, such as the 3′-end of a sense strand described herein, where the linking is via a 3′-3′ phosphodiester linkage or a 3′-3′-phosphorothioate linkage.

In another example, the 3′-terminal nucleotides of a sense strand is an inverted dA (idA) and is linked to the preceding nucleotide via a 3′-3′-linkage (e.g., 3′-3′-phosphorothioate linkage).

Other modifications of the nucleotides of an iRNA of the invention include a 5′ phosphate or 5′ phosphate mimic, e.g., a 5′-terminal phosphate or phosphate mimic on the antisense strand of an iRNA. Suitable phosphate mimics are disclosed in, for example U.S. Patent Publication No. 2012/0157511, the entire contents of which are incorporated herein by reference.

### VII. Delivery Methods of the Invention

The delivery of a nucleic acid molecule, i.e., a dsRNA and/or a REVERSIR compound as described herein to a cell e.g., a cell within a subject, such as a human subject (e.g., a subject in need thereof) can be achieved in a number of different ways. For example, delivery may be performed by contacting a cell with a dsRNA agent, or a REVERSIR compound as described herein either in vitro or in vivo. In vivo delivery may also be performed directly by administering a composition comprising a dsRNA and/or a REVERSIR compound as described herein to a subject. Alternatively, in vivo delivery may be performed indirectly by administering one or more vectors that encode and direct the expression of the dsRNA agents and/or REVERSIR compound. These embodiments are discussed further below.

For delivery of nucleic acid molecules, e.g., REVERSIR and dsRNA agents, in general, any method of delivering a nucleic acid molecule (in vitro or in vivo) can be adapted for use with the present invention (see e.g., Akhtar S. and Julian R L. (1992) Trends Cell. Biol. 2(5):139-144 and WO94/02595, which are incorporated herein by reference in their entireties). For in vivo delivery, factors to consider include, for example, biological stability of the delivered molecule, prevention of non-specific effects, and accumulation of the delivered molecule in the target tissue. RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602). Modification of the RNA or the pharmaceutical carrier can also permit targeting of the iRNA to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J., et al (2004) Nature 432:173-178).

In an alternative embodiment, delivery can include use of drug delivery systems such as a nanoparticle, a dendrimer, a polymer, liposomes, or a cationic delivery system. Positively charged cationic delivery systems which facilitate binding of an nucleic acid molecule (negatively charged) and also enhance interactions at the negatively charged cell membrane to permit efficient uptake by the cell. Cationic lipids, dendrimers, or polymers can either be bound to a nucleic acid, or induced to form a vesicle or micelle (see e.g., Kim S H, et al (2008) Journal of Controlled Release 129(2):107-116) that encases an iRNA. The formation of vesicles or micelles further prevents degradation of the nucleic acid when administered systemically. Methods for making and administering cationic-nucleic acid complexes are well within the abilities of one skilled in the art (see e.g., Sorensen, D R, et al (2003) J. Mol. Biol 327:761-766; Verma, U N, et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N, et al (2003), supra), “solid nucleic acid lipid particles” (Zimmermann, T S, et al (2006) Nature 441:111-114), cardiolipin (Chien, P Y, et al (2005) Cancer Gene Ther. 12:321-328; Pal, A, et al (2005) Int J. Oncol. 26:1087-1091), polyethyleneimine (Bonnet M E, et al (2008) Pharm. Res. Aug 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A, et al (2007) Biochem. Soc. Trans. 35:61-67; Yoo, H., et al (1999) Pharm. Res. 16:1799-1804). In some embodiments, a nucleic acid forms a complex with cyclodextrin for systemic administration. Methods for administration and pharmaceutical compositions of nucleic acid and cyclodextrins can be found in U.S. Pat. No. 7,427,605, which is herein incorporated by reference in its entirety.

### VIII. Methods of the Invention

The present invention also provides methods of using the REVERSIR compounds of the invention.

For example, in certain instances it is desirable to inhibit the RNAi interference activity of a dsRNA agent thereby restoring expression of a target mRNA/protein. In particular, certain dsRNA agents have been used therapeutically and are long-acting. In certain instances, such long acting dsRNA agents are desirable, for their convenience. In such instances, though, it can also be desirable to have a means to reverse the activity of a dsRNA agent. For example, a patient may respond poorly to treatment or receive too high a dose. In such an instance, a REVERSIR compound can be administered as, e.g., an antedote, to at least partially reduce the RNAi activity of the dsRNA agent. In certain embodiments, the long-lasting effect of dsRNA agent makes waiting for that effect to slowly diminish through natural clearance an unattractive option.

By way of example, and without limiting the present invention, certain dsRNAs are useful for inhibiting blood clotting factors (e.g., Factor II (prothrombin), Factor VII, Factor IX, etc.). Such dsRNAs have therapeutic potential as anticoagulants. Long half-lives make such dsRNAs particularly attractive, however, if a patient receives too high a dose, has surgery (where anti-coagulation is undesirable) or otherwise desires a decrease in the anti-coagulant effect, a REVERSIR compound to the anti-coagulant dsRNA can be administered. Such REVERSIR compound will restore coagulation function more quickly than simply waiting for natural clearance of the dsRNA. This example is provided for illustrative purposes. Many dsRNAs have been designed to a vast number of targets, including without limitation, a vast number of messenger RNA (mRNA) targets and pre-mRNA targets, as well as a vast number of non-coding RNA targets. REVERSIR compounds provided herein are suitable for any dsRNA, regardless of the target or mechanism of the dsRNA compound.

In certain embodiments, the invention provides REVERSIR compounds to an dsRNA targeted to an mRNA. In certain such embodiments, the target mRNA encodes a protein involved in metabolism. In certain such embodiments, the target mRNA encodes a protein involved in cardiac function. In certain embodiments, the target mRNA encodes a protein involved in blood-clotting. Exemplary dsRNA compounds targeting any of a variety of target proteins are known in the art. Further, methods for preparing dsRNA against a target gene are well known in the art and readily available to one of skill in the art.

Without limitations, target genes for dsRNAs include, but are not limited to genes promoting unwanted cell proliferation, growth factor gene, growth factor receptor gene, genes expressing kinases, an adaptor protein gene, a gene encoding a G protein super family molecule, a gene encoding a transcription factor, a gene which mediates angiogenesis, a viral gene, a gene required for viral replication, a cellular gene which mediates viral function, a gene of a bacterial pathogen, a gene of an amoebic pathogen, a gene of a parasitic pathogen, a gene of a fungal pathogen, a gene which mediates an unwanted immune response, a gene which mediates the processing of pain, a gene which mediates a neurological disease, an allene gene found in cells characterized by loss of heterozygosity, or one allege gene of a polymorphic gene.

Specific exemplary target genes for the dsRNAs include, but are not limited to, AT3, AGT, ALAS1, TMPR, HAO1, AGT, C5, CCR-5, PDGF beta gene; Erb-B gene, Src gene; CRK gene; GRB2 gene; RAS gene; MEKK gene; JNK gene; RAF gene; Erkl/2 gene; PCNA(p21) gene; MYB gene; c-MYC gene; JUN gene; FOS gene; BCL-2 gene; Cyclin D gene; VEGF gene; EGFR gene; Cyclin A gene; Cyclin E gene; WNT-1 gene; beta-catenin gene; c-MET gene; PKC gene; NFKB gene; STAT3 gene; survivin gene; Her2/Neu gene; topoisomerase I gene; topoisomerase II alpha gene; p73 gene; p21(WAF1/CIP1) gene, p27(KIP1) gene; PPM1D gene; caveolin I gene; MIB I gene; MTAI gene; M68 gene; tumor suppressor genes; p53 gene; DN-p63 gene; pRb tumor suppressor gene; APC1 tumor suppressor gene; BRCA1 tumor suppressor gene; PTEN tumor suppressor gene; MLL fusion genes, e.g., MLL-AF9, BCR/ABL fusion gene; TEL/AML1 fusion gene; EWS/FLIl fusion gene; TLS/FUS1 fusion gene; PAX3/FKHR fusion gene; AML1/ETO fusion gene; alpha v-integrin gene; Flt-1 receptor gene; tubulin gene; Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, I-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNL1A4 gene, SCA7 gene, SCA8 gene, allele gene found in loss of heterozygosity (LOH) cells, one allele gene of a polymorphic gene and combinations thereof.

In one aspect, the present invention provides a method of inhibiting the RNAi inhibitory activity of a dsRNA agent comprising a thermally destabilizing nucleotide modification in the antisense strand. The method includes contacting the dsRNA agent with the single stranded oligonucleotide of the invention or the pharmaceutical composition of the invention, thereby inhibiting the RNAi inhibitory activity of a dsRNA agent comprising a thermally destabilizing nucleotide modification in the antisense strand.

Contacting of a cell may be done in vitro or in vivo. Contacting a cell in vivo includes contacting a cell or group of cells within a subject, e.g., a human subject. Combinations of in vitro and in vivo methods of contacting a cell are also possible. Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc₃ ligand, or any other ligand that directs the RNAi agent and/or REVERSIR compound to a site of interest.

Inhibition of the RNAi interference activity of a dsRNA agent may be manifested by a reduction of the amount of mRNA expressed by a first cell or group of cells as compared to a second cell or group of cells substantially identical to the first cell or group of cells.

In other embodiments, inhibition of the RNAi interference activity of a dsRNA agent may be assessed in terms of a reduction of a parameter that is functionally linked to the target mRNA expression, e.g., protein level in blood or serum from a subject. Expression may be determined in any cell by any assay known in the art.

Inhibition of a protein may be manifested by a reduction in the level of the protein that is expressed by a cell or group of cells or in a subject sample (e.g., the level of protein in a blood sample derived from a subject). As explained above, for the assessment of mRNA suppression, the inhibition of protein expression levels in a treated cell or group of cells may similarly be expressed as a percentage of the level of protein in a control cell or group of cells, or the change in the level of protein in a subject sample, e.g., blood or serum derived therefrom.

The level of mRNA that is expressed by a cell or group of cells may be determined using any method known in the art for assessing mRNA expression. In one embodiment, the level of expression in a sample is determined by detecting a transcribed polynucleotide, or portion thereof, e.g., mRNA. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene™ (PreAnalytix™, Switzerland). Typical assay formats utilizing ribonucleic acid hybridization include nuclear run-on assays, RT-PCR, RNase protection assays, northern blotting, in situ hybridization, and microarray analysis.

In some embodiments, the level of expression is determined using a nucleic acid probe. The term “probe”, as used herein, refers to any molecule that is capable of selectively binding to a specific transgene. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of mRNA levels involves contacting the isolated mRNA with a nucleic acid molecule (probe) that can hybridize to transgene mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix® gene chip array. A skilled artisan can readily adapt known mRNA detection methods for use in determining the level of transgene mRNA.

An alternative method for determining the level of expression in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression is determined by quantitative fluorogenic RT-PCR (i.e., the TaqMan™ System).

The expression levels of mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of transgene expression level may also comprise using nucleic acid probes in solution.

In some embodiments, the level of mRNA expression is assessed using branched DNA (bDNA) assays or real time PCR (qPCR). The use of these methods is described and exemplified in the Examples presented herein.

The level of protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like.

In some embodiments, the efficacy of the methods of the invention are assessed by a decrease in mRNA or protein level (e.g., in a liver biopsy).

In another aspect, the present invention provides a method of treating a subject in need thereof. The method includes administering to the subject a therapeutically effective amount of the single stranded oligonucleotide of the invention or the pharmaceutical composition of the invention, thereby treating the subject.

In some embodiment, the subject in need thereof was previously administered a double stranded RNAi agent that inhibits the expression of a target gene and comprises a thermally destabilizing nucleotide modification in the antisense strand.

The subject in need thereof may have been administered too high a dose of the dsRNA agent and/or is experiencing off-target effects. The administration of the REVERSIR to the subject, thus, would act as an antidote to reverse the inhibition to the target gene by the dsRNA agent.

In one embodiment, the target gene is angiotensinogen (AGT). The subject in need thereof, may have been administered too high a dose of the dsRNA agent and is suffering from hypotension, hyperkalemia, and/or renal dysfunction. In certain embodiments, the methods may further comprise administering to the subject an additional therapy or therapeutic agent selected from the group consisting of increased dietary fluid/salt, fludrocortisone/midodrine treatment, intravenous fluids, vasopressor medications, down-titration or interruption of concomitant antihypertensive medications, a low potassium diet, thiazide/loop diuretic medications, oral potassium binders, calcium, glucose, insulin, and hemodialysis, or combinations thereof.

In another aspect, the present invention provides a method of ameliorating in a subject a side effect of a dsRNA agent which inhibits the expression of a target gene and comprises a thermally destabilizing nucleotide modification in the antisense strand, such as an off-target effect and/or allergic reaction and/or immunostimulatory effect of administration of the dsRNA agent. The method includes, administering to the subject an effective amount of the single stranded oligonucleotide of the invention or the pharmaceutical composition of the invention, thereby ameliorating the side effect of the dsRNA agent in the subject.

The in vivo methods of the invention may include administering to a subject a composition by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection. In certain embodiments, the compositions are administered by intramuscular injection.

An compositions of the invention may be administered as a “free iRNA” or a “free REVERSIR compound.” A free iRNA or free REVERSIR compound is administered in the absence of a pharmaceutical composition. The naked iRNA or naked REVERSIR compound may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution can be adjusted such that it is suitable for administering to a subject.

Alternatively, a composition of the invention may be administered as a pharmaceutical composition, such as a liposomal formulation.

### IX. Kits of the Invention

In certain aspects, the instant disclosure provides kits that include a suitable container containing a single stranded oligonucleotide for inhibiting RNAi activity of a double stranded ribonucleic acid (dsRNA) agent comprising a thermally destabilizing nucleotide in the antisense strand or a pharmaceutical formulation of a single stranded oligonucleotide for inhibiting RNAi activity of a double stranded ribonucleic acid (dsRNA) agent comprising a thermally destabilizing nucleotide in the antisense strand.

Such kits include one or more single stranded oligonucleotides(s) and instructions for use, e.g., instructions for administering a prophylactically or therapeutically effective amount of a single stranded oligonucleotide for inhibiting RNAi activity of a double stranded ribonucleic acid (dsRNA) agent comprising a thermally destabilizing nucleotide in the antisense strand or a pharmaceutical formulation of a single stranded oligonucleotide for inhibiting RNAi activity of a double stranded ribonucleic acid (dsRNA) agent comprising a thermally destabilizing nucleotide in the antisense strand. The single stranded oligonucleotide or pharmaceutical composition may be in a vial or a pre-filled syringe. The kits may optionally further comprise a double stranded RNAi agent that inhibits the expression of a target gene and comprises a thermally destabilizing nucleotide modification in the antisense strand, a pharmaceutical composition comprising a double stranded RNAi agent that inhibits the expression of a target gene and comprises a thermally destabilizing nucleotide modification in the antisense strand, and/or means for administering the single stranded oligonucleotide, pharmaceutical compostions, and/or double stranded RNAi agent (e.g., an injection device, such as a pre-filled syringe), or means for measuring the inhibition of the RNAi activity of the double stranded ribonucleic acid (dsRNA) agent comprising a thermally destabilizing nucleotide in the antisense strand (e.g., means for measuring the inhibition of target gene mRNA, target gene protein, and/or target gene activity). Such means for measuring the inhibition of RNAi activity may comprise a means for obtaining a sample from a subject, such as, e.g., a plasma sample. The kits of the invention may optionally further comprise means for determining the therapeutically effective or prophylactically effective amount.

In certain embodiments the individual components of the pharmaceutical formulation may be provided in one container, e.g., a vial or a pre-filled syringe. Alternatively, it may be desirable to provide the components of the pharmaceutical formulation separately in two or more containers, e.g., one container for a single stranded oligonucleotide and/or dsRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

This invention is further illustrated by the following examples which should not be construed as limiting. The entire contents of all references, patents and published patent applications cited throughout this application, as well as the informal Sequence Listing and Figures, are hereby incorporated herein by reference.

### EXAMPLES

### Materials and Methods

The following materials and methods were used in the Examples below.

Where the source of a reagent is not specifically given herein, such reagent can be obtained from any supplier of reagents for molecular biology at a quality/purity standard for application in molecular biology.

It is to be understood that, throughout the application, an oligonucleotide name without a decimal is equivalent to an oligonucleotide name with a decimal which merely references the batch number of the oligonucleotide. For example, A-959917 is equivalent to A-959917.1.

### Oligonucleotide Synthesis

Oligonucleotides were designed, synthesized, and prepared using methods known in the art.

Briefly, oligonucleotides were synthesized on a 1 μmol scale using a Mermade 192 synthesizer (BioAutomation) with phosphoramidite chemistry on solid supports. The solid support was controlled pore glass (500-1000 Å) loaded with a custom GalNAc ligand (3′-GalNAc conjugates), universal solid support (AM Chemicals), or the first nucleotide of interest. Ancillary synthesis reagents and standard 2-cyanoethyl phosphoramidite monomers (2′-deoxy-2′-fluoro, 2′-O-methyl, 2′-O-methoxyethyl, RNA, DNA, LNA) were obtained from Thermo-Fisher (Milwaukee, WI), Hongene (China), or Chemgenes (Wilmington, MA, USA). Additional phosphoramidite monomers were procured from commercial suppliers, prepared in-house, or procured using custom synthesis from various CMOs. Phosphoramidites were prepared at a concentration of 100 mM in either acetonitrile or 9:1 acetonitrile:DMF and were coupled using 5-Ethylthio-1H-tetrazole (ETT, 0.25 M in acetonitrile) with a reaction time of 400 s. Phosphorothioate linkages were generated using a 100 mM solution of 3-((Dimethylamino-methylidene) amino)-3H-1,2,4-dithiazole-3-thione (DDTT, obtained from Chemgenes (Wilmington, MA, USA)) in anhydrous acetonitrile/pyridine (9:1 v/v). Oxidation time was 5 minutes. All sequences were synthesized with final removal of the DMT group (“DMT-Off”).

Upon completion of the solid phase synthesis, solid-supported oligoribonucleotides were treated with 300 μL of Methylamine (40% aqueous) at room temperature in 96 well plates for approximately 2 hours to afford cleavage from the solid support and subsequent removal of all additional base-labile protecting groups. For sequences containing any natural ribonucleotide linkages (2′-OH) protected with a tert-butyl dimethyl silyl (TBDMS) group, a second deprotection step was performed using TEA.3HF (triethylamine trihydrofluoride). To each oligonucleotide solution in aqueous methylamine was added 200 μL of dimethyl sulfoxide (DMSO) and 300 μL TEA.3HF and the solution was incubated for approximately 30 mins at 60° C. After incubation, the plate was allowed to come to room temperature and crude oligonucleotides were precipitated by the addition of 1 mL of 9:1 acetontrile:ethanol or 1:1 ethanol:isopropanol. The plates were then centrifuged at 4° C. for 45 mins and the supernatant carefully decanted with the aid of a multichannel pipette. The oligonucleotide pellet was resuspended in 20 mM NaOAc and subsequently desalted using a HiTrap size exclusion column (5 mL, GE Healthcare) on an Agilent LC system equipped with an autosampler, UV detector, conductivity meter, and fraction collector. Desalted samples were collected in 96 well plates and then analyzed by LC-MS and UV spectrometry to confirm identity and quantify the amount of material, respectively.

Larger scale oligonucleotides were synthesized on a MerMade-12 DNA/RNA synthesizer at scales of 50-200 μmol. Sterling solvents/reagents from Glen Research, 500-Å controlled pore glass (CPG) solid supports from Prime Synthesis, 2′-deoxy 3′-phosphoramidites from Thermo, and 2′-OMe and 2′-F nucleoside and LNA 3′-phosphoramidites from Hongene were all used as received. GalNAc CPG support was prepared and used as previously described (Nair et al, JACS 2014). Low-water content acetonitrile was purchased from EMD Chemicals. A solution of 0.6 M 5-(S-ethylthio)-1H-tetrazole in acetonitrile was used as the activator. The phosphoramidite solutions were 0.15 M in anhydrous acetonitrile with 15% DMF as a co-solvent for 2′-OMe uridine and cytidine. The oxidizing reagent was 0.02 M 12 in THF/pyridine/water. N,N-Dimethyl-N′-(3-thioxo-3H-1,2,4-dithiazol-5-yl)methanimidamide (DDTT), 0.09 M in pyridine, was used as the sulfurizing reagent. The detritylation reagent was 3% dichloroacetic acid (DCA) in dichloromethane (DCM). After completion of the solid-phase synthesis, the CPG solid support was washed with 5% (v/v) piperidine in anhydrous acetonitrile three times with 5-min holds after each flow. The support was then washed with anhydrous acetonitrile and dried with argon. The oligonucleotides were then incubated with 28-30% (w/v) NH4OH, at 35° C. for 20 h. The solvent was collected by filtration, and the support was rinsed with water prior to analysis. Oligonucleotide solutions of approximately 1 OD260 units/mL were used for analysis of the crudes, and 30-50 μL of solution were injected. LC/ESI-MS was performed on an Agilent 6130 single quadrupole LC/MS system using an XBridge C8 column (2.1×50 mm, 2.5 m) at 60° C. Buffer A consisted of 200 mM 1,1,1,3,3,3-hexafluoro-2-propanol and 16.3 mM triethylamine in water, and buffer B was 100% methanol. A gradient from 0% to 40% of buffer B over 10 min followed by washing and recalibration at a flow rate of 0.70 mL/min. The column temperature was 75° C. (Supplementary Data). All oligonucleotides were purified and desalted, using previously reported methods (Nair et al., JACS, 2014).

### In Vitro Screening in Primary Cynomolgus Hepatocytes (PCH).

dsRNA Transfection Followed by REVERSIR Free Uptake.

PCHs were transfected first with dsRNA by adding 4.9 μL of Opti-MEM plus 0.1 μL of Lipofectamine RNAiMAX (Invitrogen) to 5 μL of 10 nM dsRNA per well in a 384-well Biocoat Collagen I-coated plate (Corning). Following a 15 min room temperature incubation, 40 μL of William's E Medium (Life Tech) containing 5×10³ cells was added to the dsRNA-Lipofectamine mixture (1 nM dsRNA final concentration). Cells were subsequently incubated for 4 hours, followed by a single PBS wash, media change, and addition of 5 μL REVERSIR molecules directly to 45 μL William's media (at final doses ranging from 100 nM-50 μM). After a 48 h incubation, cells were lysed and processed for RNA isolation, cDNA synthesis, and quantitative PCR analysis.

Co-Transfection of dsRNA and REVERSIR.

PCHs were co-transfected with dsRNA and REVERSIR by combining 4.8 μl of Opti-MEM, 0.2 μL of Lipofectamine 2000 (Invitrogen), 5 μL of dsRNA, and 5 μL REVERSIR per well in a 384-well Biocoat Collagen I-coated plate. After a 15 min incubation at room temperature, 35 μL of William's E Medium (Life Tech) containing 5×10³ cells was added to the dsRNA-REVERSIR-Lipofectamine mixture (1 nM dsRNA and 100-0.4 pM REVERSIR final concentrations). After a 24 h incubation, cells were lysed and processed for RNA isolation, cDNA synthesis, and quantitative PCR analysis.

### Transfections

Transfection was carried out by adding 14.8 I of Opti-MEM plus 0.2 μl of Lipofectamine RNAiMax per well (Invitrogen, Carlsbad CA. cat #13778-150) to 5 μl of each oligonucleotide to an individual well in a 96-well plate. The mixture was then incubated at room temperature for 15 minutes. Eighty μl of complete growth media without antibiotic containing ˜2×10⁴ cells were then added to the dsRNA mixture. Cells were incubated for 24 hours prior to RNA purification.

Total RNA Isolation Using DYNABEADS mRNA Isolation Kit (Invitrogen™, Part #: 610-12)

Cells were lysed in 75 μl of Lysis/Binding Buffer containing 3 μL of beads per well and mixed for 10 minutes on an electrostatic shaker. The washing steps were automated on a Biotek EL406, using a magnetic plate support. Beads were washed (in 90 μL) once in Buffer A, once in Buffer B, and twice in Buffer E, with aspiration steps in between. Following a final aspiration, complete 10 μL RT mixture was added to each well, as described below.

cDNA Synthesis Using ABI High Capacity cDNA Reverse Transcription Kit (Applied Biosystems, Foster City, CA, Cat #4368813)

A master mix of 1p l lOX Buffer, 0.4 μl 25× dNTPs, 1 μl Random primers, 0.5 μl Reverse Transcriptase, 0.5 μl RNase inhibitor and 6.6 μl of H₂O per reaction were added per well. Plates were sealed, agitated for 10 minutes on an electrostatic shaker, and then incubated at 37 degrees C. for 2 hours. Following this, the plates were agitated at 80 degrees C. for 8 minutes.

### Real Time PCR

Two microlitre (μl) of cDNA were added to a master mix containing 0.5 μl of human GAPDH TaqMan Probe (4326317E), 0.5 μl human PNPLA3, 2 μl nuclease-free water and 5 μl Lightcycler 480 probe master mix (Roche Cat #04887301001) per well in a 384 well plates (Roche cat #04887301001). Real time PCR was done in a LightCycler480 Real Time PCR system (Roche).

### Mouse AAV Study

Mice were transduced with AAV serotype 8 expressing human AGT (hAGT). 14 days after transduction on Day 0 of study, mice were administered phosphate-buffered saline (PBS) or AD-85481 at 3 mg/kg or 10 mg/kg via subcutaneous injection. On Day 7 mice were administered phosphate-buffered saline (PBS) or A-762645.12 at 3 mg/kg, 10 mg/kg, or 30 mg/kg via subcutaneous injection. Serum samples were collected on Day 0, 7, 9, 11, 14 and 21. Serum AGT protein was measured by ELISA.

### Evaluation of REVERSIR Compounds in Non-Human Primates (NHPs)

The objective of the study was to determine the pharmacodynamics of reversal agents A-762645 and A-762722, following dosing of AD-85481, to monkeys.

The study was conducted according to the appropriate protocols and amendments.

Male cynomolgus monkeys were, at dosing onset, 2.5 to 2.7 years old and weighed between 2.1 and 2.6 kg.

AD-85481, dosing formulations were prepared in 0.9% Sodium Chloride (Normal Saline) in order to reach intended concentrations. Formulations were administered to the appropriate animals by subcutaneous injection on Day 1. A-762645, A-762722, AF011-762645 and AF-011-762722 dosing formulations were prepared in 0.9% Sodium Chloride (Normal Saline) in order to reach intended concentrations. Formulations were administered to the appropriate animals by subcutaneous or intravenous injection on Day 22.

Dosing solutions containing AD-85481, A-762645, and A-762722 were within ±15% from the target concentrations and confirmed that the dosing solutions contained the Test Articles in the desired concentration range. The osmolality values for all dosing solutions were in the normal range for injection.

Blood samples were collected from Day −1 up to Day 99 post dose for Angiotensinogen (AGT) concentrations in serum. All animals survived until release to the Testing Facility Colony. 5 There were no Test Article-related variations in clinical signs, body weight or food evaluation. Single administration of AD-85481 on Day 1 at 3 mg/kg by subcutaneous injection followed by a subcutaneous dose ofA-762645 or A-762722 at 1 or 3 mg or an intravenous dose ofAFO1-762645 or AF-011-762722 at 0.3 mg/kg on Day 22 was well tolerated in monkeys. AD-85481 decreased circulating AGT levels and reversal agents (A 762645, A-762722, AFO11-762645 and AF-011-762722) restored AGT levels in monkeys.

TABLE 1
Abbreviations of nucleotide monomers used in nucleic acid sequence representation. It will
be understood that these monomers, when present in an oligonucleotide, are mutually linked by 5′-3′-
phosphodiester bonds; and it is understood that when the nucleotide contains a 2′-fluoro modification,
then the fluoro replaces the hydroxy at that position in the parent nucleotide (i.e., it is a 2′-deoxy-2′-
fluoronucleotide). It is to be further understood that the nucleotide abbreviations in the table omit the
3′-phosphate (i.e., they are 3′-OH) when placed at the 3′-terminal position of an oligonucleotide.

| Abbre- | |
|---|---|
| viation | Nucleotide(s) |
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L10 | N-(cholesterylcarboxamidocaproyl)-4-hydroxyprolinol (Hyp-C6-Chol) |
| L96 | N-[tris(GalNAc-alkyl)-amidodecanoyl)]-4-hydroxyprolinol |
| | (Hyp-(GalNAc-alkyl)3) |
| | (2S,4R)-1-[29-[[2-(acetylamino)-2-deoxy-β-D-galactopyranosyl]oxy]-14,14- |
| | bis[[3-[[3-[[5-[[2-(acetylamino)-2-deoxy-β-D-galactopyranosyl]oxy]-1- |
| | oxopentyl]amino]propyl]amino]-3-oxopropoxy]methyl]-1,12,19,25-tetraoxo-16- |
| | oxa-13,20,24-triazanonacos-1-yl]-4-hydroxy-2-hydroxymethylpyrrolidine |
| | 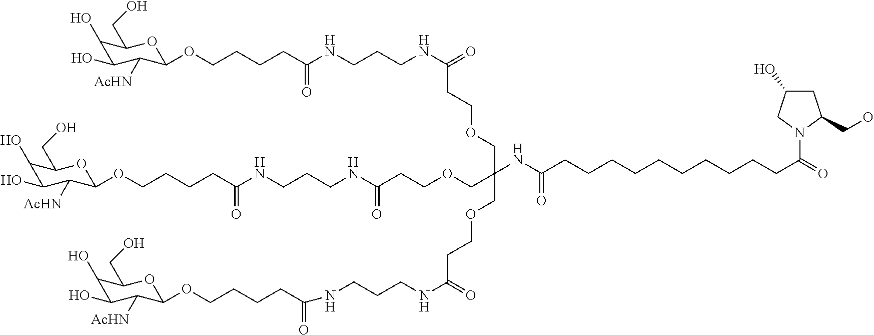 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| uL96 | 2′-O-methyluridine-3′-phosphate ((2S,4R)-1-[29-[[2-(acetylamino)-2-deoxy-β- |
| | D-galactopyranosyl]oxy]-14,14-bis [[3-[[3-[[5-[[2-(acetylamino)-2-deoxy-β-D- |
| | galactopyranosyl]oxy]-1-oxopentyl]amino]propyl]amino]-3- |
| | oxopropoxy]methyl]-1,12,19,25-tetraoxo-16-oxa-13,20,24-triazanonacos-1-yl]- |
| | 4-hydroxy-2-pyrrolidinyl)methyl ester |
| | 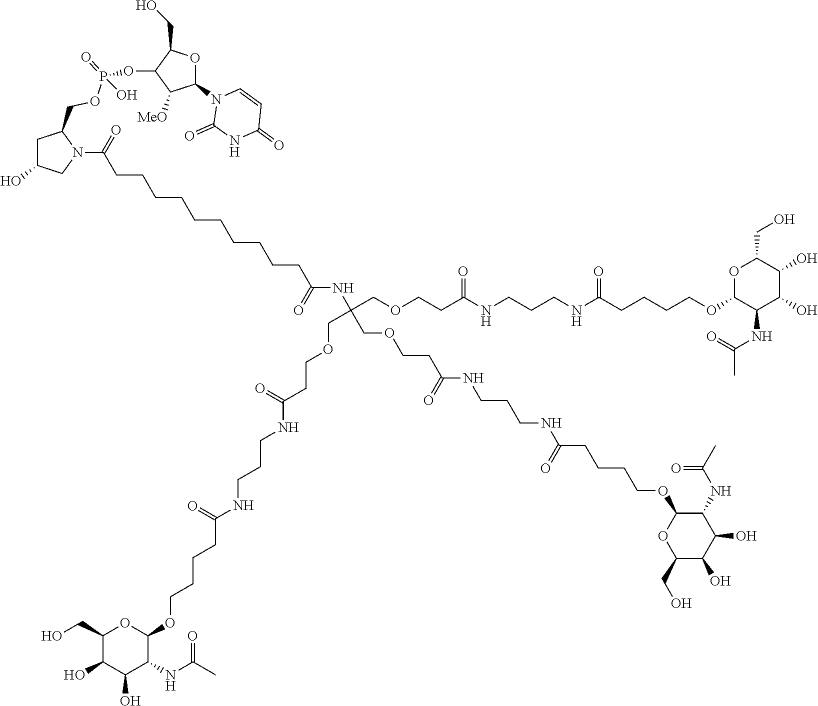 [SMILES: CO[C@@H]1C(O[P@](=O)(O)OC[C@@H]2C[C@@H](O)CN2C(=O)CCCCCCCCCCC(=O)NC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)COCCC(=O)NCCCNC(=O)CCCCO[C@@H]2O[C@H](CO)[C@H](O)[C@H](O)[C@H]2NC(C)=O)[C@@H](CO)O[C@H]1n1ccc(=O)[nH]c1=O] |
| Y34 | 2-hydroxymethyl-tetrahydrofurane-4-methoxy-3-phosphate (abasic 2′-OMe |
| | furanose) |
| | 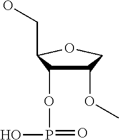 |
| Y44 | inverted abasic DNA (2-hydroxymethyl-tetrahydrofurane-5-phosphate) |
| | 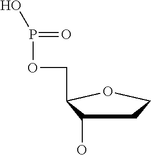 |
| L10 | N-(cholesterylcarboxamidocaproyl)-4-hydroxyprolinol (Hyp-C6-Chol) |
| | 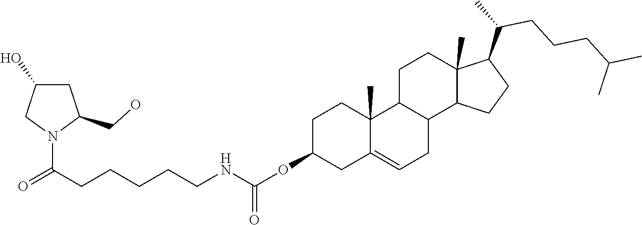 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C] |
| (Agn) | Adenosine-glycol nucleic acid (GNA) S-Isomer |
| (Cgn) | Cytidine-glycol nucleic acid (GNA) S-Isomer |
| (Ggn) | Guanosine-glycol nucleic acid (GNA) S-Isomer |
| (Tgn) | Thymidine-glycol nucleic acid (GNA) S-Isomer |
| P | Phosphate |
| VP | Vinyl-phosphonate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythimidine-3′-phosphate |
| dTs | 2′-deoxythimidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′-phosphate |
| (Chd) | 2′-O-hexadecyl-cytidine-3′-phosphate |
| (Ahd) | 2′-O-hexadecyl-adenosine-3′-phosphate |
| (Ghd) | 2′-O-hexadecyl-guanosine-3′-phosphate |
| (Uhd) | 2′-O-hexadecyl-uridine-3′-phosphate |
| Q191s | N-[tris(GalNAc-alkyl)-amidododecanoyl]-(S)-pyrrolidin-3-ol-phosphorothioate |
| | (p-C12-(GalNAc-alkyl)3) |
| | 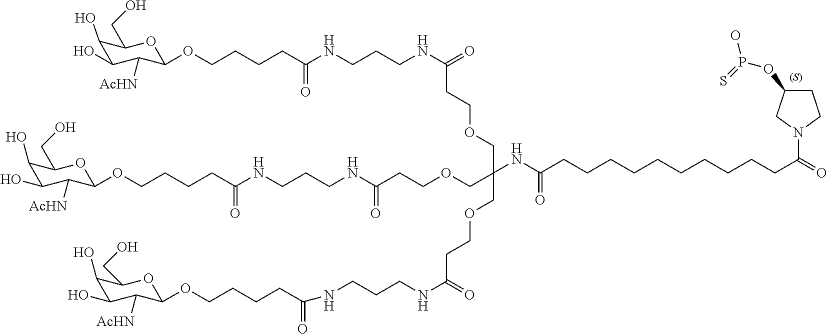 |
| (Aln) | 2′-O,4′-C-methylene adenosine-3′-phosphate |
| (Alns) | 2′-O,4′-C-methylene adenosine-3′-phosphorothioate |
| (m5- | 2′-O,4′-C-methylene 5-methylcytidine-3′-phosphate |
| Cln) | |
| (m5- | 2′-O,4′-C-methylene 5-methylcytidine-3′-phosphorothioate |
| Clns) | |
| (Gln) | 2′-O,4′-C-methylene guanosine-3′-phosphate |
| (Glns) | 2′-O,4′-C-methylene guanosine-3′-phosphorothioate |
| (Tln) | 2′-O,4′-C-methylene thymidine-3′-phosphate |
| (Tlns) | 2′-O,4′-C-methylene thymidine-3′-phosphorothioate |

### Example 1. Inclusion of a Thermally Destabilizing Nucleotide Modification in a dsRNA Agent Abolishes the Inhibitory Activity of REVERSIR™ Oligonucleotides

In recent years, safety and efficacy data from a number of investigationai clinical studies have demonstrated the therapeutic use of RNA interference (RNAi). The short interfering RNA (dsRNA) agents that utilize the endogenous RNAi pathway potently and specificality silence mRNAs, thereby preventing the formation of disease causing or disease pathway implicated proteins. Targeted delivery of RNAi therapeutics to liver hepatocytes is achieved by conjugating chemically modified dsRNAs to a trivalent N-acetylgalactosamine (GalNAc) ligand, which facilitates asialoglycoprotein receptor (ASGPR)-mediated tissue specific uptake. The development of dsRNA agents conjugated to a GalNAc ligand with enhanced stabilization chemistry has led to substantial improvements in efficacy and duration. In preclinical animal models, as well as in humans, dsRNA agents have demonstrated impressive duration of action following a single subcutaneous administration, with potent silencing sustained for several months. With their extended duration of action, RNAi therapeutics can benefit from a technology that enables rapid reversal of silencing activity and provides tailored control over RNAi pharmacology, a desired attribute for personalized precision medicines.

However, in some circumstances, a subject may respond poorly to treatment with a dsRNA agent or receive too high a dose. In such instances, a compound which reverses the iRNA silencing activity of the dsRNA agent could be administered to at least partially reduce the RNAi activity of the dsRNA agent. In other instances, the long-lasting effect of dsRNA makes waiting for that effect to slowly diminish through natural clearance an unattractive option.

U.S. Patent Publication No. US 2017/0369872 and PCT Publication Nos. WO 2016/100716 and WO 2019/036612 (the entire contents of each of which are incorporated herein by reference), describe the principles for the design of oligonucleotides which reverse the iRNA silencing activity of dsRNA agents regardless of the target mRNA. These oligonucleotides, termed REVERSIRs, were demonstrated to reverse the iRNA silencing activity of dsRNA agents conjugated to a GalNAc ligand in vitro and in vivo to thereby enable the control and tailoring of RNAi pharmacology.

GalNAc-REVERSIRs bind to and are internalized into a cell through the asialoglycoprotein receptor (ASPGR) and irreversibly bind to the antisense strand of a dsRNA agent in a functional RISC complex. The binding of the REVERSIR abrogates the mRNA target recognition and cleavage of the dsRNA agent.

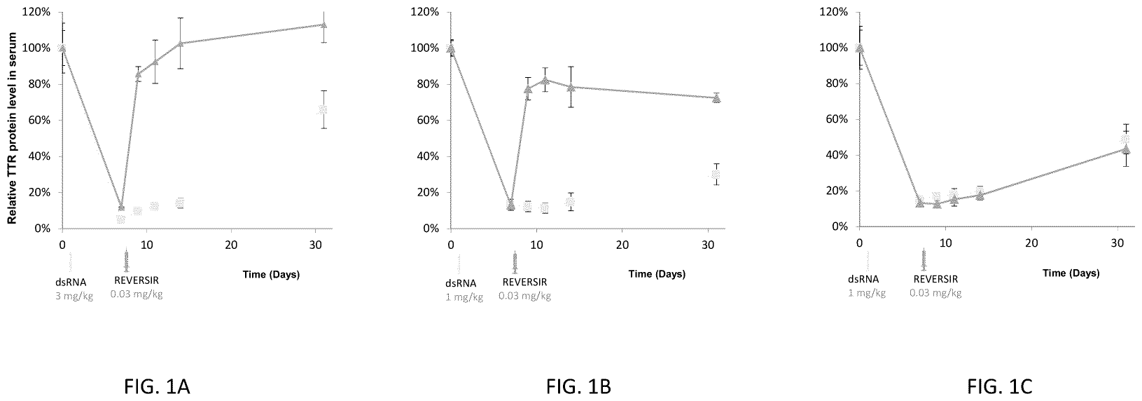

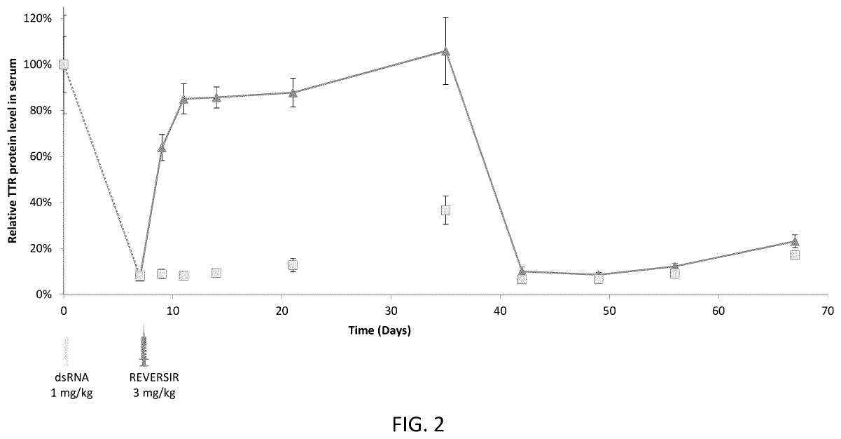

However, when these REVERSIRs, which were demonstrated to inhibit the silencing of dsRNA agents conjugated to a GalNAc ligand, were assessed for activity with dsRNA agents targeting the same region of the mRNA and comprising substantially the same nucleotide modifications with the exception of inclusion of a thermally destabilizing nucleotide modification [e.g., an abasic nucleotide, a 2′-deoxy nucleotide, an acyclic nucleotide (e.g., unlocked nucleic acid (UNA), a glycol nucleic acid (GNA) or an (S)-glycol nucleic acid (S-GNA)), a 2′-5′ linked nucleotide (3′-RNA), a threose nucleotide (TNA), a 2′ gem Me/F nucleotide or a mismatch with an opposing nucleotide in the other strand, e.g., opposite to the seed region of the antisense strand (i.e., at positions 2-8 or 2-9 counting from the 5′-end of the antisense strand) in the antisense strand], the activity of the REVERSIRs was abolished (FIGS. 1A-1C). Only when the REVERSIRs were lengthened, e.g., to about 18 nucleotides in length, and administered at a dose up to 100 times higher than that of the REVERSIRs targeting non-destabilized dsRNAs, were the REVERSIRs able to inhibit the silencing of dsRNA agents comprising a thermally destabilizing nucleotide modification and conjugated to a GalNAc ligand (FIG. 2). Nonetheless, even at the most optimal, the inhibition of silencing activity of the REVERSIRs was about 30-100 fold less potent when a thermally destabilizing nucleotide modification was present on the dsRNA agent as compared to the inhibition of silencing activity of the most optimal REVERSIRs when a thermally destabilizing nucleotide modification was not present on the dsRNA agent.

In view of the requirement for the higher dose and the REVERSIRs to have longer lengths and numerous phosphorothioate linkages and LNAs in order to inhibit the RNAi silencing activity of dsRNA agents comprising a thermally destabilizing nucleotide modification in the antisense strand, the potential immunostimulatory effects observed with antisense oligonucleotides (ASOs)), e.g., stimulation of cytokines and chemokines (e.g., IL-6, 11-8, MCP-1, elevated C-reactive protein (CRP) levels, elevated IgM levels, and infiltration of immune cells into organs, of the REVERSIRs were evaluated.

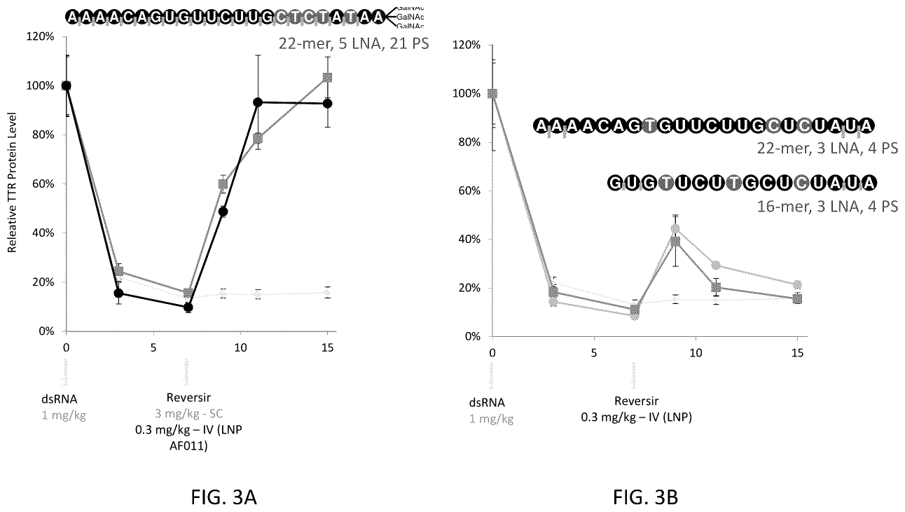

In order to decrease the dose of REVERSIRs, the oligonucleotides were formulated into lipid nanoparticles, LNPs, for intravenous administration at a lower dose or re-designed to include fewer LNAs and/or phosphorothiotae linkages and formulaetd into LNPs. As demonstrated in FIG. 3A, intravenous administration of 0.3 mg/kg of a REVERSIR formulated in a LNP has substantially the same inhibitory effect on the RNAi silencing activity of a dsRNA agent comprising a thermally destabilizing nucleotide modification in the antisense strand as does subcutaneous administration of 3 mg/kg of the same REVERSIR comprising a GalNAc ligand. As demonstrated in FIG. 3B, although the REVERSIRs were formulated in LNPs, decreasing the LNAs and/or phosphorothioate linkages did not provide sufficient metabolic stability to the REVERSIRs to efficiently inhibit the RNAi silencing activity of a dsRNA agent comprising a thermally destabilizing nucleotide modification in the antisense strand.

### Example 2. Identification of Oligonucleotides that Inhibit the RNAi Activity of a dsRNA Agent Comprising a Thermally Destabilizing Nucleotide Modification in the Antisense Strand

As described above, the inclusion of a thermally destabilizing nucleotide modification in the antisense strand of a dsRNA agent was demonstrated to abolish the inhibitory activity of REVERSIR oligonucleotides.

A dsRNA agent that potently and durably inhibits the expression of angiotensinogen (AGT), AD-85481 (Zilebesiran; also referred to as AGT-01) is currently being investigated in a Phase II/III clinical trial for the treatment of hypertension. AD-85481 comprises a thermally destabilizing nucleotide modification in the antisense strand. The unmodified and modified sense and antisense strand nucleotide sequences of duplex AD-85481 are shown in the Table below.

### Unmodified and Modified Nucleotide Sequence of Duplex AD-85481

| | | SEQ | | |
|---|---|---|---|---|
| | | ID | | SEQ ID |
| Strand | Unmodified (5′ to 3′) | NO. | Modified (5′ to 3′) | NO. |
| Antis | UGUACUCUCAUUGUGGAUG | 9 | usGfsuac(Tgn)cucauugUfgGfaugacs | 11 |
| | ACGA | | gsa | |
| Sense | GUCAUCCACAAUGAGAGUA | 10 | gsuscaucCfaCfAfAfugagaguaca | 12 |
| | CA | | | |

The chemical modifications are defined as follows: a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Ggn) is guanosine-glycol nucleic acid (GNA), and s is phosphorothioate linkage. The 3′end of the sense strand is conjugated to a ligand as shown in the following schematic

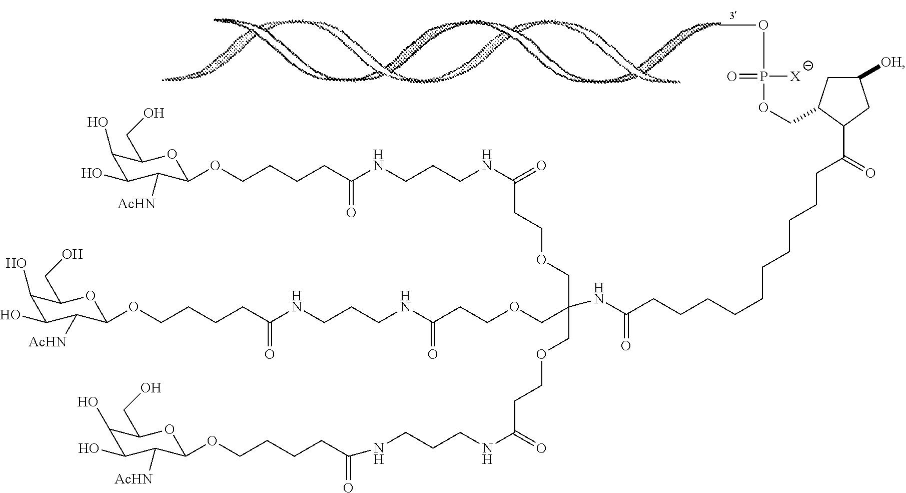

wherein X is O.

Given the potency and long duration of action of AD-85481, it is possible that some subjects being treated with AD-85481 may potentially experience recurrent, symptomatic, and persistent hypotension even with the use of conventional interventions to treat the hypotension, e.g., increased dietary fluid/salt, fludrocortisone/midodrine treatment, intravenous fluids, vasopressor medications, and/or down-titration or interruption of concomitant antihypertensive medications. It is also possible that some subjects being treated with AD-85481 may potentially experience hyperkalemia even with the use of conventional interventions to treat the hyperkalemia, e.g., a low potassium diet, thiazide/loop diuretic medications, oral potassium binders, calcium, glucose, insulin, and/or hemodialysis. Furthermore, it is also possible that some subjects may potentially experience renal dysfuction.

In view of these potential safety effects of AD-85481 administration, a treatment to reverse the RNAi inhibition of AGT by AD-85481, such as a REVERSIR of AD-85481, would be beneficial.

Accordingly, and using AD-85481 as a model system of dsRNA agents comprising a thermally destabilizing nucleotide modification in the antisense strand, REVERSIRs of AD-85481 were designed and assessed for in vitro activity by free uptake at a final concentration of 100 nM or 1000 nM and transfection at a final concentration of 1 nM or 10 nM in primary cynomolgus hepatocytes using standard methods (briefly described below). Tables 2-5 provide the modified nucleotide sequences of the REVERSIRs and the results of the in vitro analyses.

As can be seen in Tables 2-5, numerous 22-mer REVERSIRs comprising 2 LNA modifications inhibited the RNAi silencing activity of AD-85481 in vitro, although there was a loss in potency of the REVERSIRs when assessed by free uptake. With respect to the 18-mer REVERSIRs comprising 2 LNA modifications, they are overall less potent inhibitors as compared to the 22-mer REVERSIRs (Table 3) and the 16-mer REVERSIRs were less potent inhibitors as compared to either the 22-mer or 18-mer REVERSIRs (Table 4).

In addition, the data demonstrate that an LNA at position 2 (counting from the 3′-end of the REVERSIR) in combination with a second LNA at position 6 or 14-16 (counting from the 3′-end of the REVERSIR) in a 22-mer REVERSIR potently inhibited the activity of AD-85481 (Table 2). Similar results were observed with the 18-mer REVERSIRs, although an LNA at position 2 (counting from the 3′-end of the REVERSIR) in combination with a second LNA at postion 8 or 9 (counting from the 3′-end of the REVERSIR) were the most potent 18-mer REVERSIRs (Table 3). With respect to the 16-mer REVERSIRs, similar to the 18-mer REVERSIRs, LNA modifications at position 2 (counting from the 3′-end of the REVERSIR) in combination with a second LNA at position 8, 9, 13, or 14 (counting from the 3′-end of the REVERSIR) were the most potent (Table 4). With respect to the REVERSIRs comprising 5 LNA modificatons, as can be seen in Table 5, there is a clear length dependence on the inhibitory activity of the REVERSIRs.

The analyses demonstrate that inclusion of LNA modifications at positions 2 and 6 (counting from the 3′-end of the oligonucleotide) have good inhibitory activity of the RNAi inhibitory activity of AD-85481 as compared to positions 2 and 5 or 2 and 7 (counting from the 3′-end of the oligonucleotide). In some cases, e.g., the 18-mer REVERSIRs, positions 2 and 8 and 2 and 9 show good inhibitory activity. Other positions, 2 and 14, 2 and 15, or 2 and 16 also have good inhibitory activity.

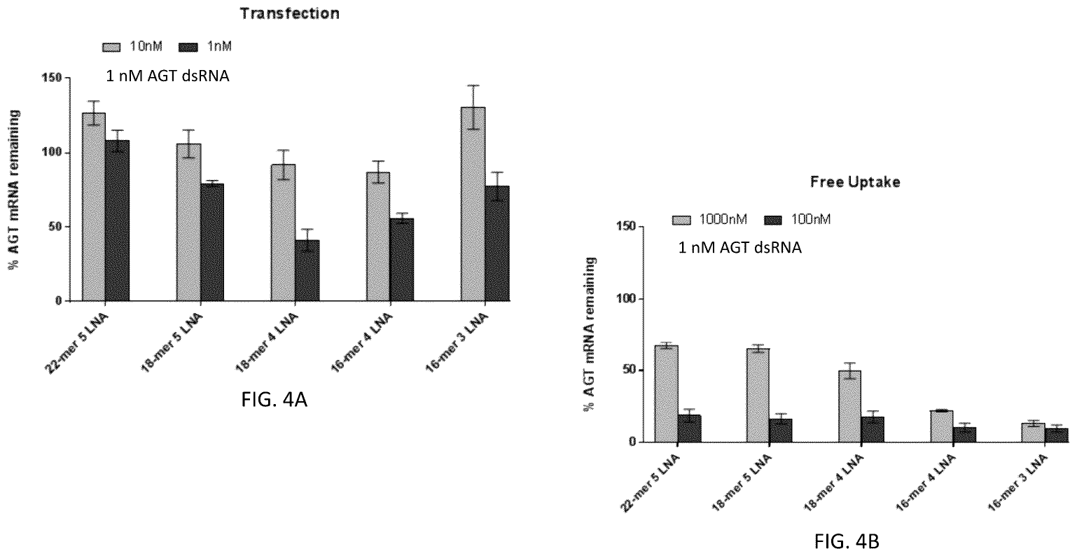

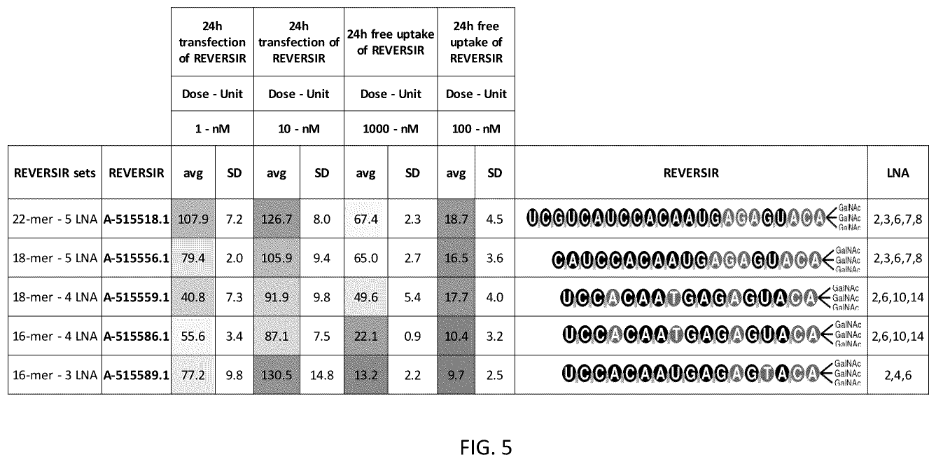

In addition, the inhibitory activity of the REVERSIRs was maintained for the 22-mer, 18-mer, and 16-mer oligonucleotides, although the 18-mer and 16-mer REVERSIRs were not as potent as the 22-mer REVERSIRs. In addition, 18-mer, and 16-mer REVERSIRs comprising 3 or 4 LNA modifications have comparable activity to 18-mer and 16-mer REVERSIRs comprising 5 LNA modifications (FIGS. 4A and 4B). Furthermore, it was demonstrated that 12-mer and 9-mer REVERSIRs comprising four or three LNA modifications had no inhibitory activity.

The top 5 inhibitory REVERSIRs from the in vitro analyses, A-515518, A-515556, A-515559, A-515586, and A-515589, were selected and assessed for in vivo activity (FIG. 5). Briefly, at pre-dose Day −21 wild-type mice (C57BL/6) were transduced with 2×10¹¹ viral particles of an adeno-associated virus 8 (AAV8) vector encoding human AGT via intravenous injection. At day 0, mice were subcutaneously administered a single 3 mg/kg dose of AD-85481. At Day 7, groups of three mice were subcutaneously administered a single 3 mg/kg dose of A-515518, A-515556, A-515559, A-515586, or A-515589, duplexes with GalNAc ligands, or were intravenously administered LNP formulated REVERSIRs A-515518, A-515556, A-515559, A-515586, or A-515589, or PBS control.

At Days 0, 7, 9, 11, and 14 serum samples were collected. At Day 14 animals were sacrificed, liver samples were collected and snap-frozen in liquid nitrogen. Serum and liver mRNA was extracted and analyzed by the RT-QPCR method.

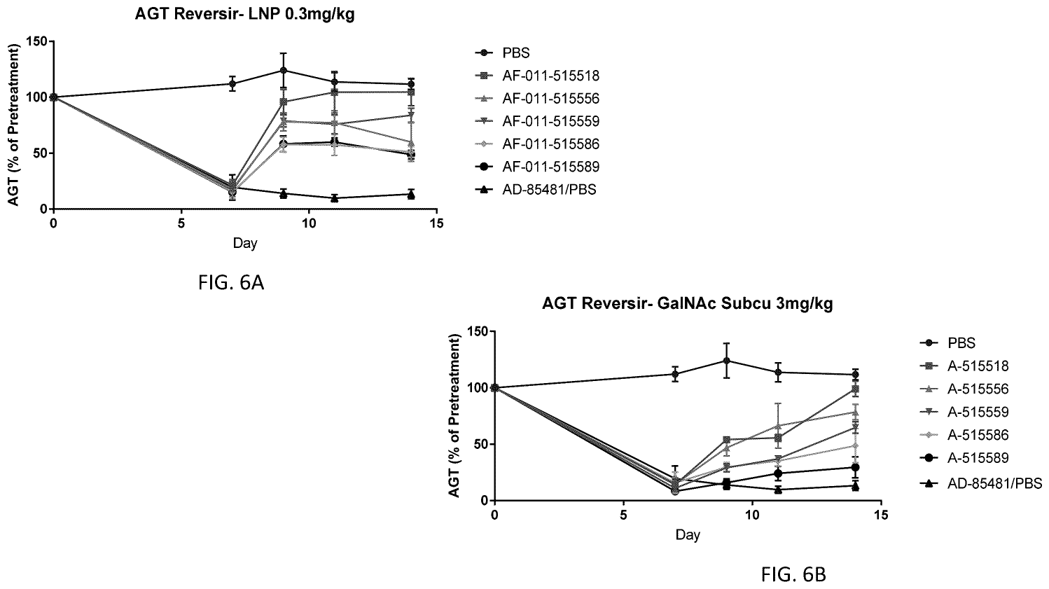

Human AGT mRNA levels were compared to a housekeeping gene, GAPDH and, as depicted in FIGS. 6A and 6B, both GalNAc-conjugated REVERSIRs and LNP formulated REVERSIRs potently inhibited the RNAi inhibitory activity of AD-85481, with the 22-mer REVERSIR (A-515518) having the most potent activity. Furthermore, it was observed that LNP formulated REVERSIRs and GalNAc-conjugated REVERSIRs had a rapid onset of action, however, the LNP formulated REVERSIRs had less durability than the GalNAc-conjugated REVERSIRs.

TABLE 2
22-mer 2 LNA AGT REVERSIR

| | | 24 h | | 24 h | | | | | | | | |
|---|---|---|---|---|---|---|---|---|---|---|---|---|
| | | transfection | | transfection | | 24 h free | | 24 h free | | | | |
| | | of | | of | | uptake of | | uptake of | | | | |
| | | REVERSIR | | REVERSIR | | REVERSIR | | REVERSIR | | | | |
| | | Dose - Unit | | Dose - Unit | | Dose - Unit | | Dose - Unit | | | LNA | SEQ |
| REVERSIR | | 1 - nM | | 10 - nM | | 1000 - nM | | 100 - nM | | | posi- | ID |
| sets | REVERSIR | avg | SD | avg | SD | avg | SD | avg | SD | REVERSIR (5′-3′) | tion | NO |
| 22-mer RVR | A-515494.1 | 72.9 | 9.1 | 109.7 | 5.6 | 17.4 | 3.0 | 9.0 | 2.6 | uscsgsuscsasuscscsascsasasusgsasgsasgsusas(m5Cln)dAL96 | 2 | 1 |
| set single | A-515495.1 | 61.5 | 7.6 | 119.8 | 10.6 | 23.6 | 5.4 | 10.0 | 2.2 | uscsgsuscsasuscscsascsasasusgsasgsasgsus(Aln)(m5Cln)dAL96 | 3 | 2 |
| LNA walk | A-515496.1 | 55.8 | 4.3 | 100.4 | 8.4 | 27.2 | 8.0 | 10.7 | 1.6 | uscsgsuscsasuscscsascsasasusgsasgsasgs(Tln)as(m5Cln)dAL96 | 4 | 3 |
| | A-515497.1 | 42.2 | 7.1 | 105.6 | 10.0 | 24.9 | 5.0 | 11.7 | 1.8 | uscsgsuscsasuscscsascsasasusgsasgsas(Gln)usas(m5Cln)dAL96 | 5 | 4 |
| | A-515498.1 | 102.4 | 16.4 | 90.1 | 5.9 | 31.0 | 1.8 | 13.4 | 4.1 | uscsgsuscsasuscscsascsasasusgsasgs(Aln)gsusas(m5Cln)dAL96 | 6 | 5 |
| | A-515499.1 | 21.2 | 3.6 | 74.6 | 11.5 | 20.3 | 4.7 | 11.9 | 2.4 | uscsgsuscsasuscscsascsasasusgsas(Gln)asgsusas(m5Cln)dAL96 | 7 | 6 |
| | A-515500.1 | 16.2 | 0.9 | 79.2 | 17.0 | 25.3 | 2.3 | 10.8 | 3.3 | uscsgsuscsasuscscsascsasasusgs(Aln)gsasgsusas(m5Cln)dAL96 | 8 | 7 |
| | A-515501.1 | 15.3 | 3.1 | 51.2 | 5.4 | 17.2 | 2.8 | 12.7 | 2.5 | uscsgsuscsasuscscsascsasasus(Gln)asgsasgsusas(m5Cln)dAL96 | 9 | 8 |
| | A-515502.1 | 14.3 | 1.8 | 63.7 | 26.3 | 15.6 | 1.9 | 11.8 | 6.2 | uscsgsuscsasuscscsascsasas(Tln)gsasgsasgsusas(m5Cln)dAL96 | 10 | 13 |
| | A-515503.1 | 12.2 | 2.1 | 50.8 | 10.8 | 14.2 | 3.1 | 11.7 | 3.3 | uscsgsuscsasuscscsascsas(Aln)usgsasgsasgsusas(m5Cln)dAL96 | 11 | 14 |
| | A-515504.1 | 13.8 | 2.7 | 66.4 | 4.6 | 20.0 | 4.8 | 11.8 | 1.9 | uscsgsuscsasuscscsascs(Aln)asusgsasgsasgsusas(m5Cln)dAL96 | 12 | 15 |
| | A-515505.1 | 15.4 | 3.3 | 63.8 | 12.7 | 23.3 | 7.6 | 9.7 | 1.7 | uscsgsuscsasuscscsas(m5Cln)asasusgsasgsasgsusas(m5Cln)dAL96 | 13 | 16 |
| | A-515506.1 | 86.1 | 9.6 | 115.3 | 8.9 | 32.9 | 3.0 | 10.2 | 2.5 | uscsgsuscsasuscscs(Aln)csasasusgsasgsasgsusas(m5Cln)dAL96 | 14 | 17 |
| | A-515507.1 | 74.8 | 9.1 | 119.9 | 3.7 | 32.3 | 2.9 | 13.1 | 1.4 | uscsgsuscsasuscs(m5Cln)ascsasasusgsasgsasgsusas(m5Cln)dAL96 | 15 | 18 |
| | A-515508.1 | 45.0 | 5.2 | 112.9 | 8.6 | 32.7 | 5.3 | 12.0 | 2.9 | uscsgsuscsasus(m5Cln)csascsasasusgsasgsasgsusas(m5Cln)dAL96 | 16 | 19 |
| | A-515509.1 | 24.2 | 2.6 | 86.0 | 10.2 | 25.2 | 4.7 | 10.3 | 1.3 | uscsgsuscsas(Tln)cscsascsasasusgsasgsasgsusas(m5Cln)dAL96 | 17 | 20 |
| | A-515510.1 | 21.6 | 3.8 | 70.5 | 8.0 | 23.8 | 4.0 | 10.7 | 3.3 | uscsgsuscs(Aln)uscscsascsasasusgsasgsasgsusas(m5Cln)dAL96 | 18 | 21 |
| | A-515511.1 | 18.4 | 4.4 | 73.1 | 6.2 | 33.0 | 7.9 | 11.2 | 1.1 | uscsgsus(m5Cln)asuscscsascsasasusgsasgsasgsusas(m5Cln)dAL96 | 19 | 22 |
| | A-515512.1 | 11.5 | 1.0 | 59.6 | 9.5 | 21.8 | 2.2 | 11.1 | 2.5 | uscsgs(Tln)csasuscscsascsasasusgsasgsasgsusas(m5Cln)dAL96 | 20 | 23 |
| | A-515513.1 | 10.2 | 0.9 | 53.7 | 4.6 | 22.1 | 3.0 | 13.5 | 3.4 | uscs(Gln)uscsasuscscsascsasasusgsasgsasgsusas(m5Cln)dAL96 | 21 | 24 |
| | A-515514.1 | 15.0 | 2.0 | 55.6 | 3.8 | 23.1 | 2.3 | 12.0 | 1.9 | us(m5Cln)gsuscsasuscscsascsasasusgsasgsasgsusas(m5Cln)dAL96 | 22 | 25 |
| | A-515515.1 | 9.8 | 2.6 | 30.5 | 0.7 | 11.5 | 3.0 | 11.2 | 2.8 | (Tln)csgsuscsasuscscsascsasasusgsasgsasgsusas(m5Cln)dAL96 | 23 | 26 |
| AGT dsRNA | AD-85481 | 5.4 | 1.1 | 6.1 | 0.3 | 6.2 | 0.9 | 5.8 | 0.8 | | | |
| alone | | | | | | | | | | | | |

TABLE 3
18-mer 2 LNA AGT REVERSIR

| | | 24 h | | 24 h | | | | | | | | |
|---|---|---|---|---|---|---|---|---|---|---|---|---|
| | | transfection | | transfection | | 24 h free | | 24 h free | | | | |
| | | of | | of | | uptake of | | uptake of | | | | |
| | | REVERSIR | | REVERSIR | | REVERSIR | | REVERSIR | | | | |
| | | Dose - Unit | | Dose - Unit | | Dose - Unit | | Dose - Unit | | | | |
| REVERSIR | | 1 - nM | | 10 - nM | | 1000 - nM | | 100 - nM | | | LNA | SEQ ID |
| set | REVERSIR | avg | SD | avg | SD | avg | SD | avg | SD | REVERSIR (5′-3′) | position | NO: |
| 18-mer | A-515536.1 | 7.1 | 1.4 | 17.5 | 2.8 | 10.6 | 2.6 | 8.3 | 1.6 | csasuscscsascsasasusgsasgsasgsusas(m5Cln)dAL96 | 2 | 27 |
| RVR set | A-515537.1 | 7.4 | 0.9 | 29.4 | 5.4 | 15.5 | 3.0 | 10.8 | 3.6 | csasuscscsascsasasusgsasgsasgsus(Aln)(m5Cln)dAL96 | 3 | 28 |
| single LNA | A-515538.1 | 9.0 | 1.1 | 29.2 | 4.2 | 13.7 | 1.6 | 9.4 | 2.9 | csasuscscsascsasasusgsasgsasgs(Tln)as(m5Cln)dAL96 | 4 | 29 |
| walk | A-515539.1 | 11.2 | 1.6 | 37.4 | 6.0 | 20.4 | 1.7 | 13.3 | 3.5 | csasuscscsascsasasusgsasgsas(Gln)usas(m5Cln)dAL96 | 5 | 30 |
| | A-515540.1 | 17.9 | 4.0 | 64.8 | 14.7 | 25.3 | 4.2 | 13.5 | 3.0 | csasuscscsascsasasusgsasgs(Aln)gsusas(m5Cln)dAL96 | 6 | 31 |
| | A-515541.1 | 14.0 | 1.8 | 52.9 | 4.7 | 12.8 | 3.9 | 15.3 | 3.5 | csasuscscsascsasasusgsas(Gln)asgsusas(m5Cln)dAL96 | 7 | 32 |
| | A-515542.1 | 7.4 | 2.7 | 109.3 | 9.0 | 33.5 | 7.1 | 18.5 | 4.7 | csasuscscsascsasasusgs(Aln)gsasgsusas(m5Cln)dAL96 | 8 | 33 |
| | A-515543.1 | 45.6 | 18.1 | 89.5 | 6.2 | 21.7 | 3.2 | 11.5 | 1.7 | csasuscscsascsasasus(Gln)asgsasgsusas(m5Cln)dAL96 | 9 | 34 |
| | A-515544.1 | 25.6 | 2.8 | 64.2 | 8.5 | 19.3 | 3.5 | 11.8 | 2.9 | csasuscscsascsasas(Tln)gsasgsasgsusas(m5Cln)dAL96 | 10 | 35 |
| | A-515545.1 | 7.2 | 3.6 | 45.9 | 7.3 | 21.5 | 4.0 | 10.9 | 1.9 | csasuscscsascsas(Aln)usgsasgsasgsusas(m5Cln)dAL96 | 11 | 36 |
| | A-515546.1 | 17.3 | 4.6 | 47.2 | 7.0 | 22.5 | 4.9 | 12.6 | 2.3 | csasuscscsascs(Aln)asusgsasgsasgsusas(m5Cln)dAL96 | 12 | 37 |
| | A-515547.1 | 15.0 | 3.6 | 52.0 | 6.3 | 26.0 | 1.6 | 13.4 | 2.7 | csasuscscsas(m5Cln)asasusgsasgsasgsusas(m5Cln)dAL96 | 13 | 38 |
| | A-515548.1 | 13.3 | 1.1 | 46.9 | 6.8 | 21.5 | 3.2 | 15.5 | 5.7 | csasuscscs(Aln)csasasusgsasgsasgsusas(m5Cln)dAL96 | 14 | 39 |
| | A-515549.1 | 14.7 | 1.2 | 54.6 | 9.5 | 22.7 | 0.8 | 14.2 | 2.6 | csasuscs(m5Cln)ascsasasusgsasgsasgsusas(m5Cln)dAL96 | 15 | 40 |
| | A-515550.1 | 11.8 | 1.6 | 45.0 | 6.4 | 19.4 | 3.1 | 13.2 | 4.8 | csasus(m5Cln)csascsasasusgsasgsasgsusas(m5Cln)dAL96 | 16 | 41 |
| | A-515551.1 | 11.9 | 0.5 | 39.4 | 5.7 | 22.8 | 5.0 | 13.2 | 1.6 | csas(Tln)cscsascsasasusgsasgsasgsusas(m5Cln)dAL96 | 17 | 42 |
| | A-515552.1 | 15.0 | 2.4 | 46.8 | 8.8 | 19.7 | 9.2 | 16.1 | 4.7 | cs(Aln)uscscsascsasasusgsasgsasgsusas(m5Cln)dAL96 | 18 | 43 |
| | A-515553.1 | 15.3 | 1.3 | 40.3 | 8.6 | 13.2 | 2.9 | 12.1 | 2.3 | (m5Cln)asuscscsascsasasusgsasgsasgsusas(m5Cln)dAL96 | 19 | 44 |

TABLE 4
16-mer 2 LNA AGT REVERSIR

| | | 24 h | | 24 h | | | | | | | | |
|---|---|---|---|---|---|---|---|---|---|---|---|---|
| | | transfection | | transfection | | 24 h free | | 24 h free | | | | |
| | | of | | of | | uptake of | | uptake of | | | | |
| | | REVERSIR | | REVERSIR | | REVERSI™ | | REVERSIR | | | | |
| | | Dose - Unit | | Dose - Unit | | Dose - Unit | | Dose - Unit | | | | |
| REVERSIR | | 1 - nM | | 10 - nM | | 1000 - nM | | 100 - nM | | | LNA | SEQ ID |
| set | REVERSIR | avg | SD | avg | SD | avg | SD | avg | SD | REVERSIR (5′-3′) | position | NO: |
| 16-mer | A-515565.1 | 6.5 | 1.0 | 12.9 | 2.1 | 4.1 | 1.2 | 10.3 | 2.3 | uscscsascsasasusgsasgsasgsusas(m5Cln)dAL96 | 2 | 45 |
| RVR set- | A-515566.1 | 6.7 | 0.9 | 13.0 | 2.5 | 7.3 | 0.4 | 6.4 | 1.3 | uscscsascsasasusgsasgsasgsus(Aln)(m5Cln)dAL96 | 3 | 46 |
| single LNA | A-515567.1 | 5.8 | 0.8 | 12.8 | 1.9 | 5.7 | 1.3 | 7.6 | 2.8 | uscscsascsasasusgsasgsasgs(Tln)as(m5Cln)dAL96 | 4 | 47 |
| walk | A-515568.1 | 7.2 | 1.3 | 16.5 | 1.1 | 7.1 | 1.4 | 8.3 | 2.3 | uscscsascsasasusgsasgsas(Gln)usas(m5Cln)dAL96 | 5 | 48 |
| | A-515569.1 | 11.1 | 1.7 | 48.2 | 5.1 | 13.9 | 1.5 | 11.6 | 2.4 | uscscsascsasasusgsasgs(Aln)gsusas(m5Cln)dAL96 | 6 | 49 |
| | A-515570.1 | 7.2 | 0.9 | 20.9 | 5.8 | 7.6 | 2.1 | 12.2 | 0.9 | uscscsascsasasusgsas(Gln)asgsusas(m5Cln)dAL96 | 7 | 50 |
| | A-515571.1 | 8.7 | 0.4 | 30.4 | 1.5 | 8.1 | 1.4 | 9.5 | 1.6 | uscscsascsasasusgs(Aln)gsasgsusas(m5Cln)dAL96 | 8 | 51 |
| | A-515572.1 | 32.2 | 2.2 | 55.6 | 7.1 | 4.4 | 0.8 | 4.8 | 0.4 | uscscsascsasasus(Gln)asgsasgsusas(m5Cln)dAL96 | 9 | 52 |
| | A-515573.1 | 23.4 | 4.7 | 32.3 | 3.2 | 4.5 | 0.6 | 6.7 | 2.8 | uscscsascsasas(Tln)gsasgsasgsusas(m5Cln)dAL96 | 10 | 53 |
| | A-515574.1 | 16.3 | 2.0 | 22.7 | 2.7 | 6.3 | 0.6 | 6.7 | 4.2 | uscscsascsas(Aln)usgsasgsasgsusas(m5Cln)dAL96 | 11 | 54 |
| | A-515575.1 | 14.4 | 2.3 | 25.5 | 2.0 | 8.2 | 0.7 | 7.4 | 1.9 | uscscsascs(Aln)asusgsasgsasgsusas(m5Cln)dAL96 | 12 | 55 |
| | A-515576.1 | 14.5 | 3.7 | 31.0 | 3.2 | 10.8 | 1.4 | 8.4 | 3.3 | uscscsas(m5Cln)asasusgsasgsasgsusas(m5Cln)dAL96 | 13 | 56 |
| | A-515577.1 | 10.0 | 2.5 | 21.9 | 1.9 | 10.7 | 1.0 | 6.1 | 1.2 | uscscs(Aln)csasasusgsasgsasgsusas(m5Cln)dAL96 | 14 | 57 |
| | A-515578.1 | 11.3 | 0.6 | 29.3 | 2.8 | 13.5 | 2.0 | 6.3 | 2.9 | uscs(m5Cln)ascsasasusgsasgsasgsusas(m5Cln)dAL96 | 15 | 58 |
| | A-515579.1 | 7.9 | 0.8 | 22.9 | 0.9 | 10.3 | 1.1 | 9.5 | 3.8 | us(m5Cln)csascsasasusgsasgsasgsusas(m5Cln)dAL96 | 16 | 59 |
| | A-515580.1 | 7.0 | 0.5 | 16.9 | 4.3 | 6.6 | 0.4 | 9.8 | 3.3 | (Tln)cscsascsasasusgsasgsasgsusas(m5Cln)dAL96 | 17 | 60 |

TABLE 5
Five LNA AGT REVERSIR

| | | 24 h | | 24 h | | | |
|---|---|---|---|---|---|---|---|
| | | transfection | | transfection | | | |
| | | of | | of | | | |
| | | REVERSIR | | REVERSIR | | | |
| | | Dose - Unit | | Dose - Unit | | | |
| REVERSIR | | 1 - nM | | 10 - nM | | | |
| sets | REVERSIR | avg | SD | avg | SD | REVERSIR | SEQ ID NO: |
| 22-mer RVR | A-515516.1 | 3.3 | 8.1 | 113.0 | 7.3 | uscsgsuscsasuscscsascsasasusgs(Aln)(Gln)(Aln)gs(Tln)as(m5Cln)dAL96 | 61 |
| set - 5 LNA | A-515517.1 | 20.5 | 4.4 | 101.9 | 18.2 | uscsgsuscsasuscscsascsasasusgs(Aln)(Gln)as(Gln)(Tln)as(m5Cln)dAL96 | 62 |
| | A-515518.1 | 107.9 | 7.2 | 126.7 | 8.0 | uscsgsuscsasuscscsascsasasusgs(Aln)(Gln)(Aln)gsus(Aln)(m5Cln)dAL96 | 63 |
| | A-515519.1 | 88.6 | 6.9 | 119.6 | 5.7 | uscsgsuscsasuscscsascsasasusgs(Aln)(Gln)as(Gln)us(Aln)(m5Cln)dAL96 | 64 |
| | A-515520.1 | 72.7 | 12.1 | 109.7 | 7.2 | uscsgsuscsasuscscsascsasasusgsas(Gln)(Aln)(Gln)us(Aln)(m5Cln)dAL96 | 65 |
| | A-515521.1 | 40.6 | 4.5 | 101.2 | 11.8 | uscsgsuscsasuscs(m5Cln)(Aln)csasasusgsasgsas(Gln)(Tln)as(m5Cln)dAL96 | 66 |
| | A-515522.1 | 48.9 | 9.7 | 96.8 | 7.4 | uscsgsuscsasuscs(m5Cln)(Aln)csasasusgsasgs(Aln)gs(Tln)as(m5Cln)dAL96 | 67 |
| | A-515523.1 | 30.2 | 7.3 | 91.0 | 8.1 | uscsgsuscsasuscs(m5Cln)(Aln)csasasusgsas(Gln)as(Gln)usas(m5Cln)dAL96 | 68 |
| | A-515524.1 | 19.5 | 5.7 | 77.9 | 7.4 | uscsgsuscsasus(m5Cln)cs(Aln)csasasusgsasgsas(Gln)(Tln)as(m5Cln)dAL96 | 69 |
| | A-515525.1 | 25.2 | 6.8 | 87.2 | 16.0 | uscsgsuscsasus(m5Cln)cs(Aln)csasasusgsasgs(Aln)gs(Tln)as(m5Cln)dAL96 | 70 |
| | A-515526.1 | 21.9 | 1.5 | 86.5 | 7.1 | uscsgsuscsasus(m5Cln)cs(Aln)csasasusgsas(Gln)as(Gln)usas(m5Cln)dAL96 | 71 |
| | A-515527.1 | 16.8 | 4.9 | 73.3 | 15.2 | uscsgsuscsasuscs(m5Cln)as(m5Cln)asasusgsasgsas(Gln)(Tln)as(m5Cln)dAL96 | 72 |
| | A-515528.1 | 28.3 | 8.5 | 91.8 | 11.9 | uscsgsuscsasuscs(m5Cln)as(m5Cln)asasusgsasgs(Aln)gs(Tln)as(m5Cln)dAL96 | 73 |
| | A-515529.1 | 27.6 | 4.5 | 117.0 | 20.8 | uscsgsuscsasuscs(m5Cln)as(m5Cln)asasusgsas(Gln)as(Gln)usas(m5Cln)dAL96 | 74 |
| 18-mer RVR | A-515554.1 | 88.3 | 4.0 | 108.2 | 7.0 | csasuscscsascsasasusgs(Aln)(Gln)(Aln)gs(Tln)as(m5Cln)dAL96 | 75 |
| set-5 LNA | A-515555.1 | 69.1 | 5.5 | 102.7 | 12.0 | csasuscscsascsasasusgs(Aln)(Gln)as(Gln)(Tln)as(m5Cln)dAL96 | 76 |
| | A-515556.1 | 79.4 | 2.0 | 105.9 | 9.4 | csasuscscsascsasasusgs(Aln)(Gln)(Aln)gsus(Aln)(m5Cln)dAL96 | 77 |
| | A-515557.1 | 50.8 | 6.3 | 97.2 | 6.9 | csasuscscsascsasasusgs(Aln)(Gln)as(Gln)us(Aln)(m5Cln)dAL96 | 78 |
| | A-515558.1 | 41.8 | 4.2 | 84.7 | 7.1 | csasuscscsascsasasusgsas(Gln)(Aln)(Gln)us(Aln)(m5Cln)dAL96 | 79 |
| 16-mer RVR | A-515581.1 | 24.3 | 2.5 | 63.4 | 8.1 | uscscsascsasasusgs(Aln)(Gln)(Aln)gs(Tln)as(m5Cln)dAL96 | 80 |
| set-5 LNA | A-515582.1 | 11.4 | 2.6 | 31.2 | 4.1 | uscscsascsasasusgs(Aln)(Gln)as(Gln)(Tln)as(m5Cln)dAL96 | 81 |
| | A-515583.1 | 18.1 | 3.4 | 74.4 | 3.7 | uscscsascsasasusgs(Aln)(Gln)(Aln)gsus(Aln)(m5Cln)dAL96 | 82 |
| | A-515584.1 | 63.6 | 3.2 | 94.3 | 10.6 | uscscsascsasasusgs(Aln)(Gln)as(Gln)us(Aln)(m5Cln)dAL96 | 83 |
| | A-515585.1 | 55.1 | 3.7 | 88.4 | 1.6 | uscscsascsasasusgsas(Gln)(Aln)(Gln)us(Aln)(m5Cln)dAL96 | 84 |
| 12-mer RVR | A-515604.1 | 22.2 | 3.9 | 46.8 | 1.8 | csasasusgs(Aln)(Gln)(Aln)gs(Tln)as(m5Cln)dAL96 | 85 |
| set-5 LNA | A-515605.1 | 11.0 | 2.0 | 12.9 | 1.2 | csasasusgs(Aln)(Gln)as(Gln)(Tln)as(m5Cln)dAL96 | 86 |
| | A-515606.1 | 11.1 | 1.7 | 23.1 | 1.9 | csasasusgs(Aln)(Gln)(Aln)gsus(Aln)(m5Cln)dAL96 | 87 |
| | A-515607.1 | 8.6 | 2.3 | 13.0 | 1.5 | csasasusgs(Aln)(Gln)as(Gln)us(Aln)(m5Cln)dAL96 | 88 |
| | A-515608.1 | 7.1 | 1.1 | 16.5 | 1.0 | csasasusgsas(Gln)(Aln)(Gln)us(Aln)(m5Cln)dAL96 | 89 |
| 9-mer RVR | A-515627.1 | 30.0 | 5.3 | 57.8 | 4.8 | usgs(Aln)(Gln)(Aln)gs(Tln)as(m5Cln)dAL96 | 90 |
| set- | A-515628.1 | 11.3 | 4.7 | 15.5 | 0.6 | usgs(Aln)(Gln)as(Gln)(Tln)as(m5Cln)dAL96 | 91 |
| 5 LNA | A-515629.1 | 12.7 | 2.0 | 38.0 | 3.6 | usgs(Aln)(Gln)(Aln)gsus(Aln)(m5Cln)dAL96 | 92 |
| | A-515630.1 | 8.0 | 2.1 | 20.2 | 2.7 | usgs(Aln)(Gln)as(Gln)us(Aln)(m5Cln)dAL96 | 93 |
| | A-515631.1 | 7.9 | 1.5 | 15.8 | 1.3 | usgsas(Gln)(Aln)(Gln)us(Aln)(m5Cln)dAL96 | 94 |

### Example 3. Additional REVERSIRs that Inhibit the RNAi Activity of a dsRNA Agent Comprising a Thermally Destabilizing Nucleotide Modification in the Antisense Strand

As described in Example 2, REVERSIRs that inhibit the RNAi interference activity of a dsRNA agent comprising a thermally destabilizing nucleotide modification in the antisense strand, i.e., AD-85481, were designed, synthesized, and assessed for activity in vitro and in vivo. Notably, however, although the REVERSIRs exhibited good activity by transfection, they exhibited poor activity by free uptake.

Accordingly, an additional ninety-seven 15-mer, 16-mer and 18-mer REVERSIRs comprising 4 or 5 LNA modifications were designed, synthesized, and assessed for in vitro activity to inhibit the RNAi interference activity of AD-85481 by free uptake at a final concentration of 100 nM or 1000 nM and transfection at a final concentration of 1 nM or 10 nM in primary cynomolgus hepatocytes using standard methods.

The results, demonstrated that significantly more of the REVERSIRs had activity by transfection and free uptake as compared to the REVERSIRs designed, synthesized and assayed in Example 2.

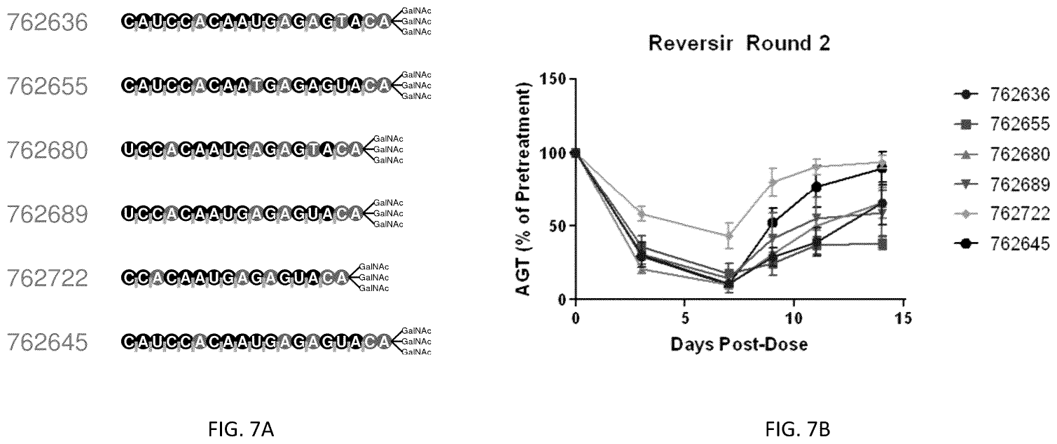

The top performing REVERSIRs identified from the in vitro analyses, A-762636, A-762655, A-762680, A-762689, A-762722, and A-762645, were assessed for activity in vivo as described in Example 3. FIG. 7A and Table 6 provide the modified nucleotide sequences of these REVERSIRs.

Briefly, at pre-dose Day −21 wild-type mice (C57BL/6) were transduced with 2×10¹⁰ viral particles of an adeno-associated virus 8 (AAV8) vector encoding human AGT via intravenous injection. At day 0, mice were subcutaneously administered a single 3 mg/kg dose of AD-85481. At Day 7 groups of three mice were subcutaneously administered a single 3 mg/kg dose of A-762636, A-762655, A-762680, A-762689, A-762722, and A-762645 (GalNAc-conjugated duplexes), or PBS control.

At Days 0, 7, 9, 11, and 14, serum samples were collected. Animals were sacrificed at Day 14 and liver samples were collected and snap-frozen in liquid nitrogen. Serum and liver mRNA was extracted and analyzed by the RT-QPCR method.

Human AGT mRNA levels were compared to a housekeeping gene, GAPDH and as depicted in FIG. 7B the REVERSIRs potently inhibited the RNAi inhibitory activity of AD-85481.

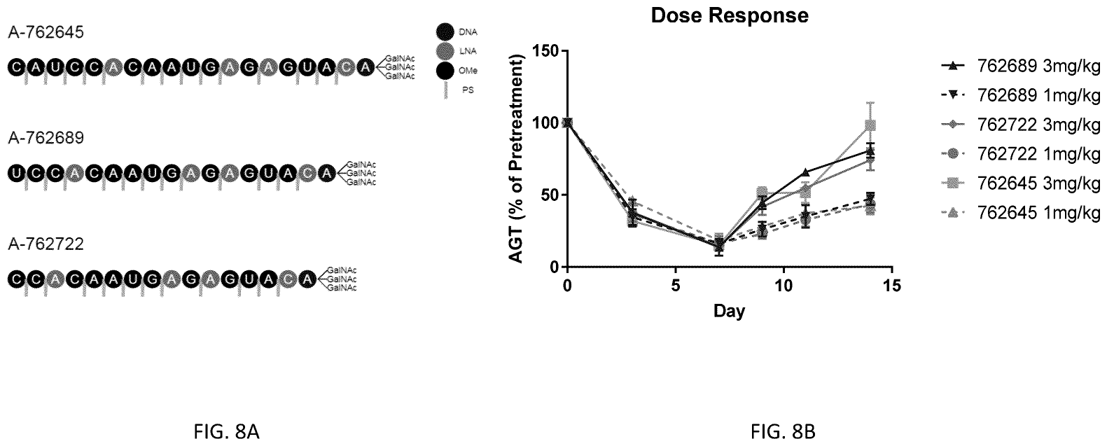

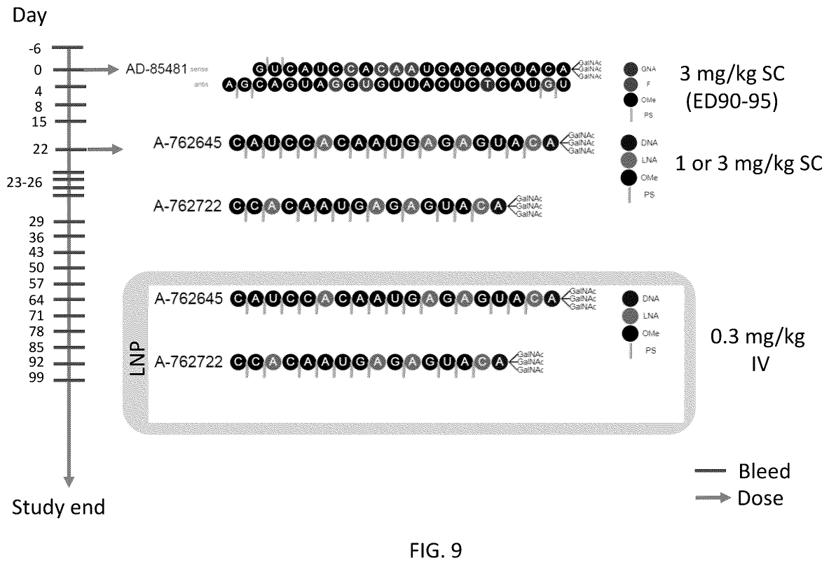

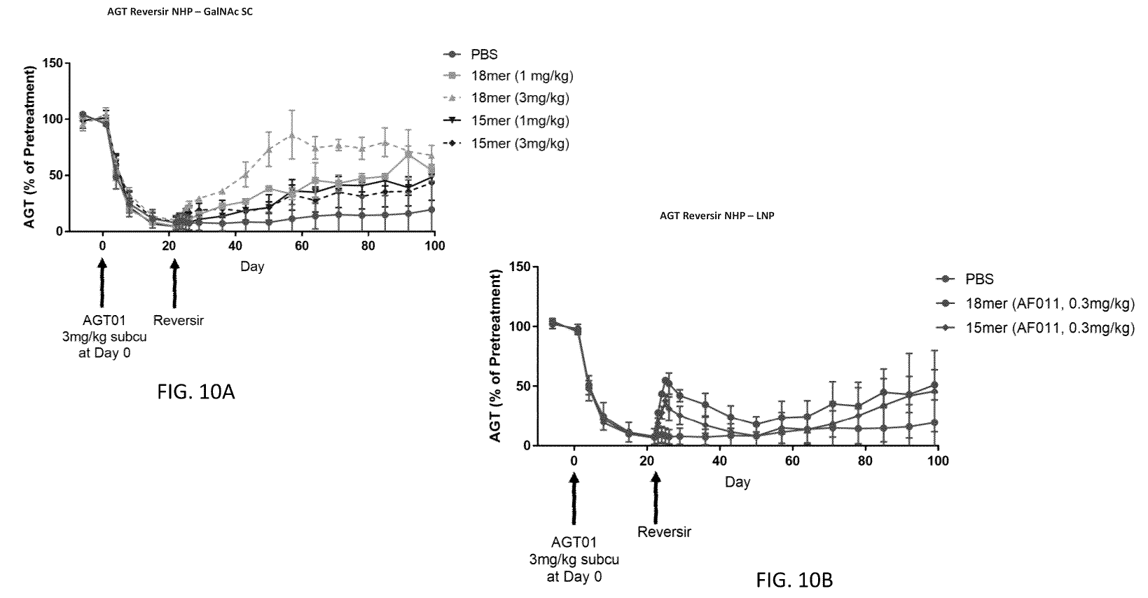

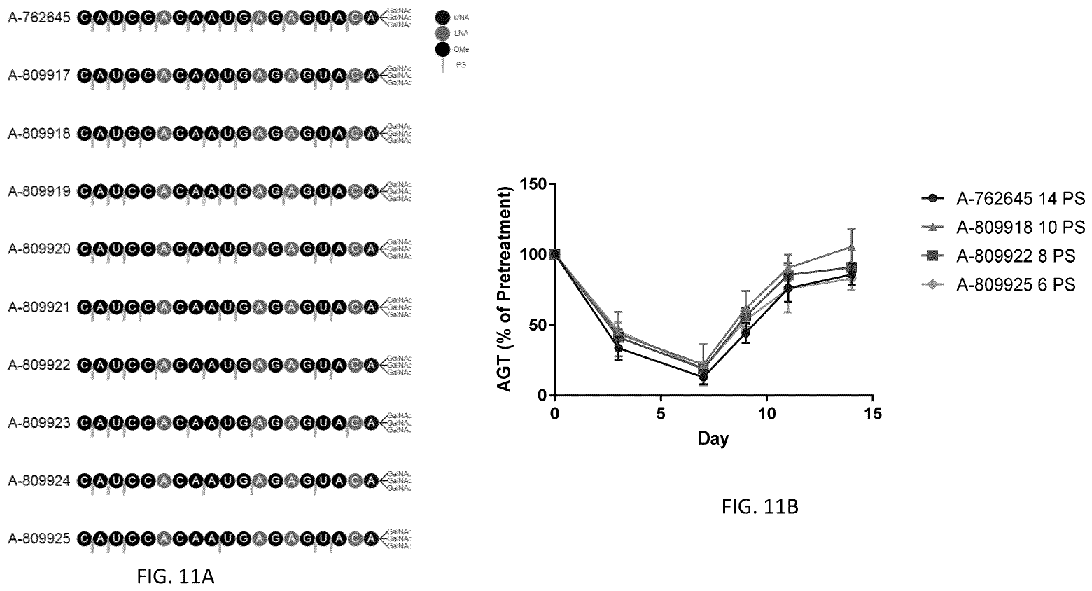

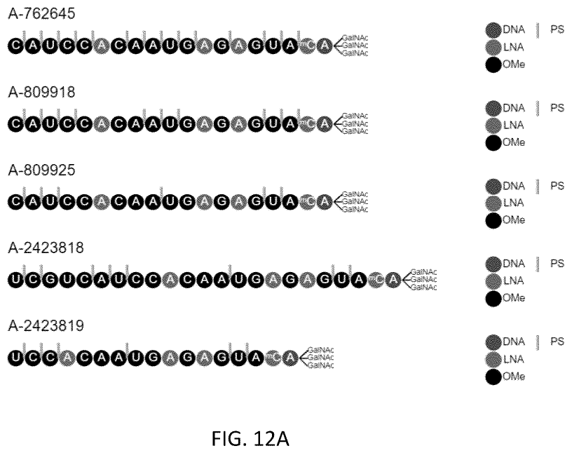

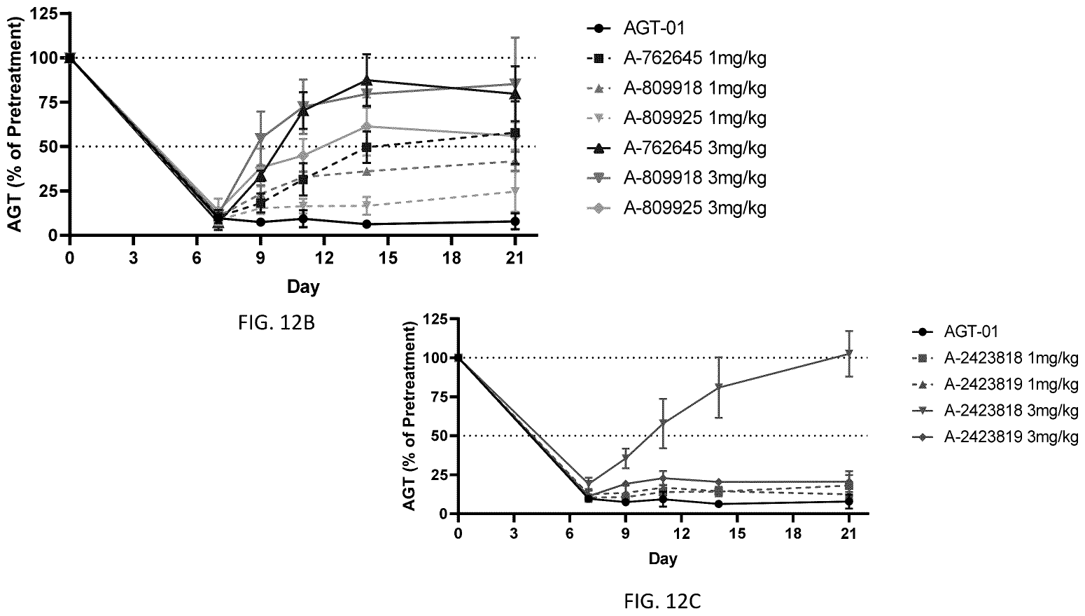

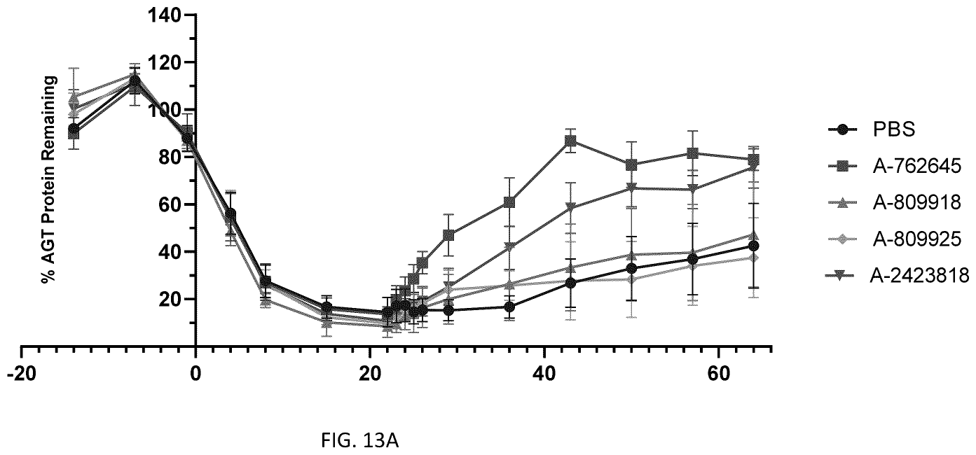

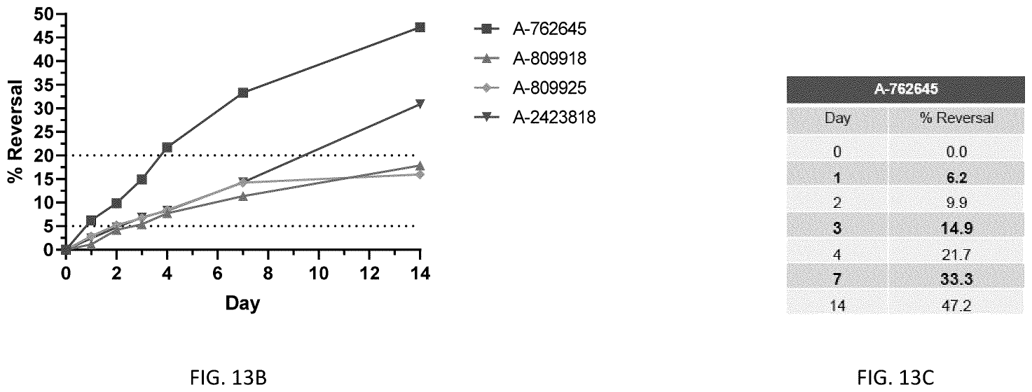

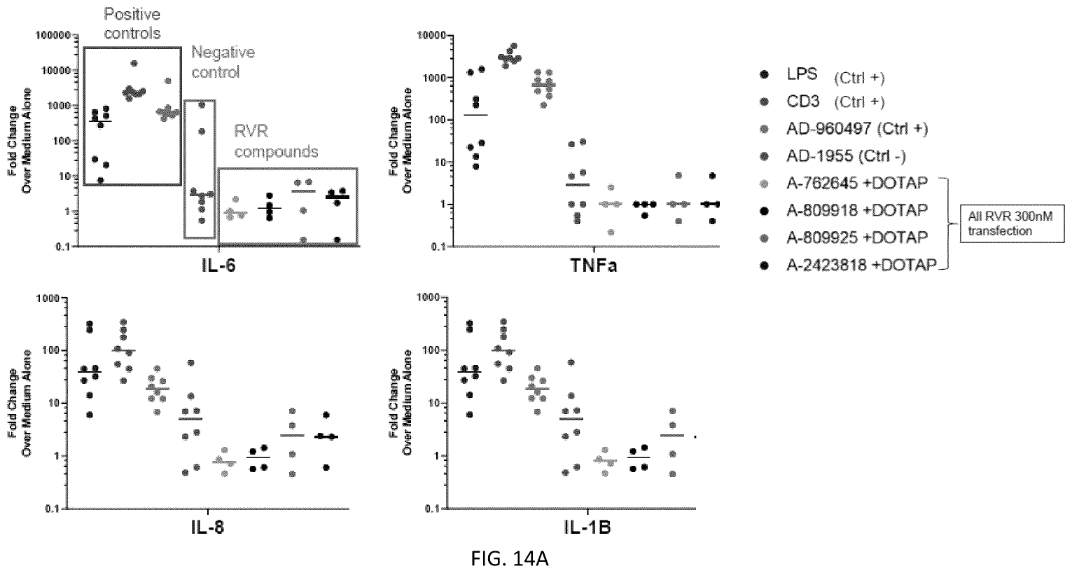

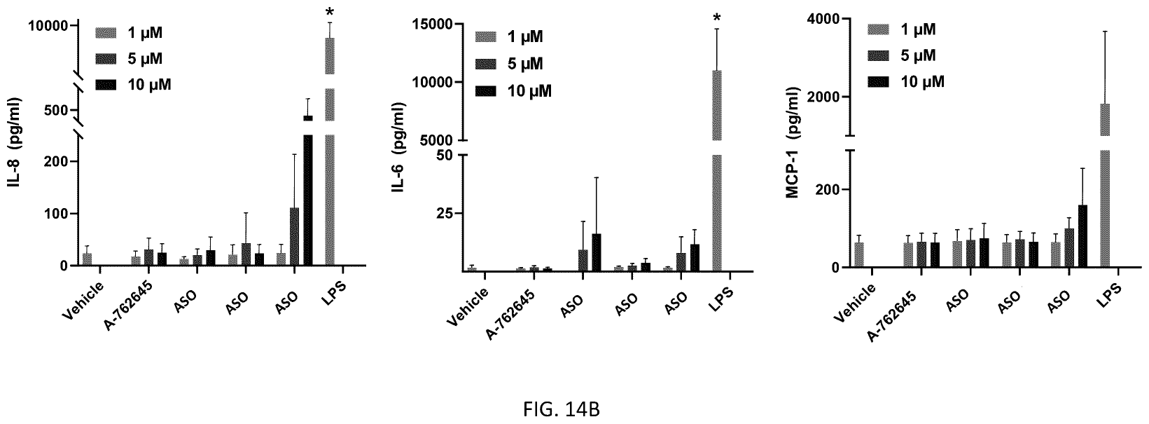

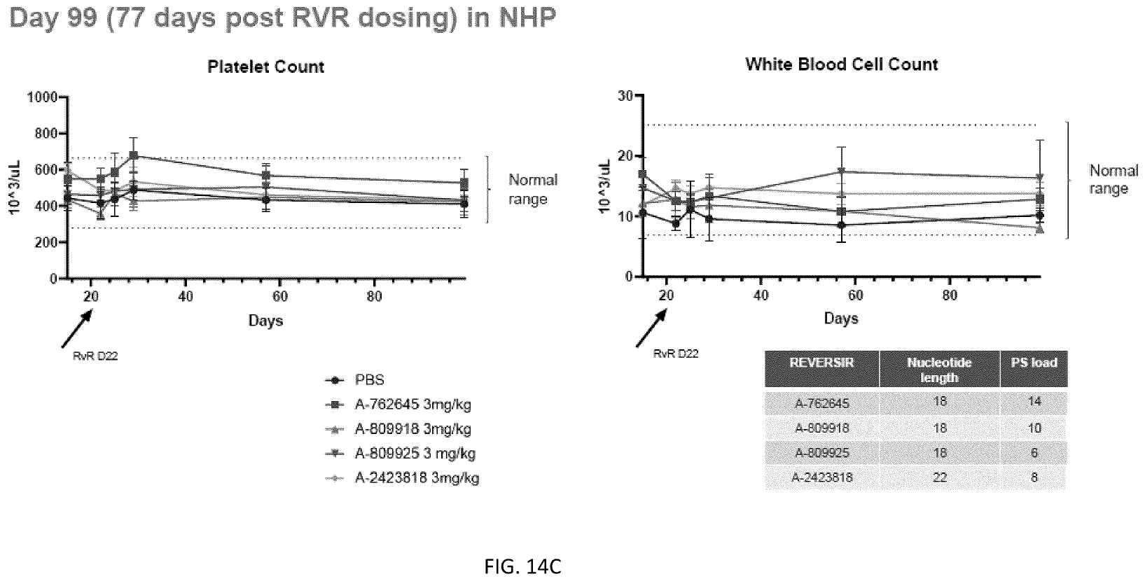

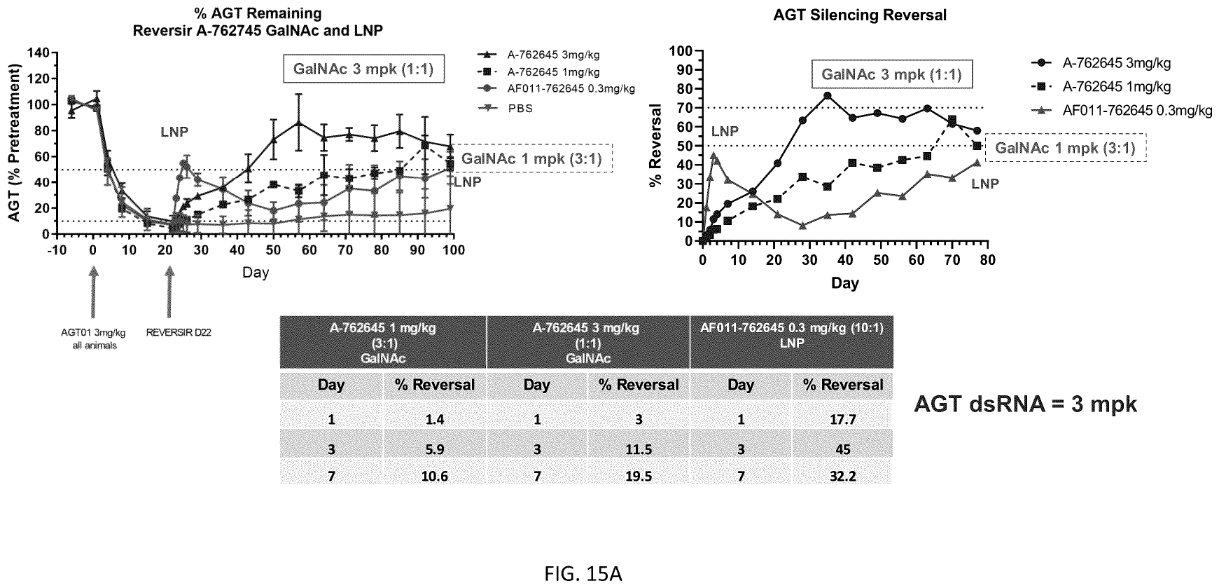

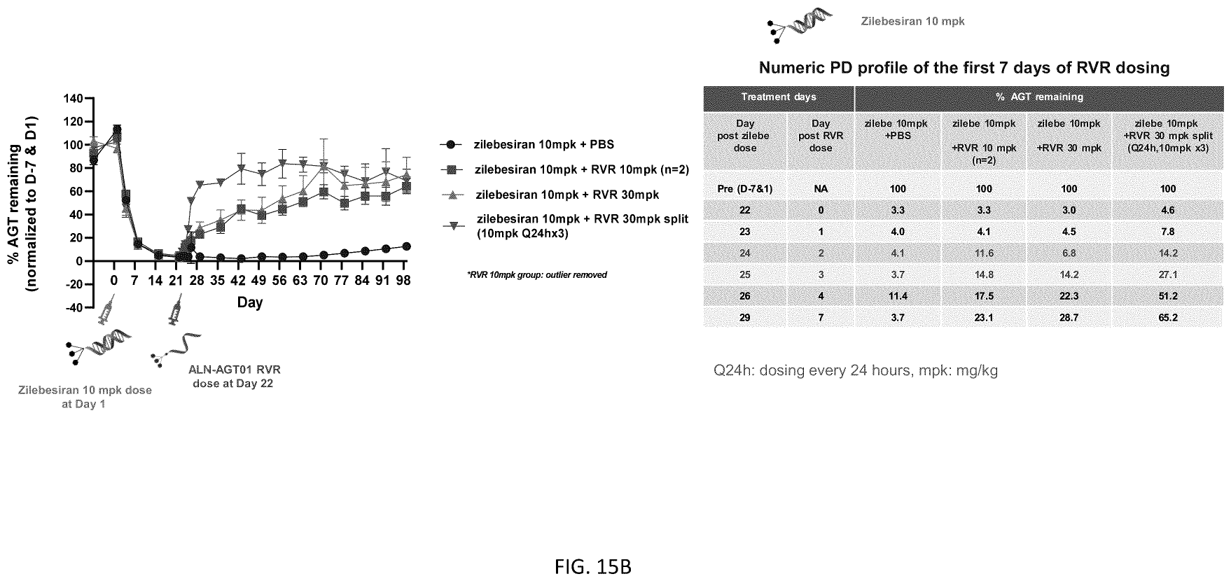

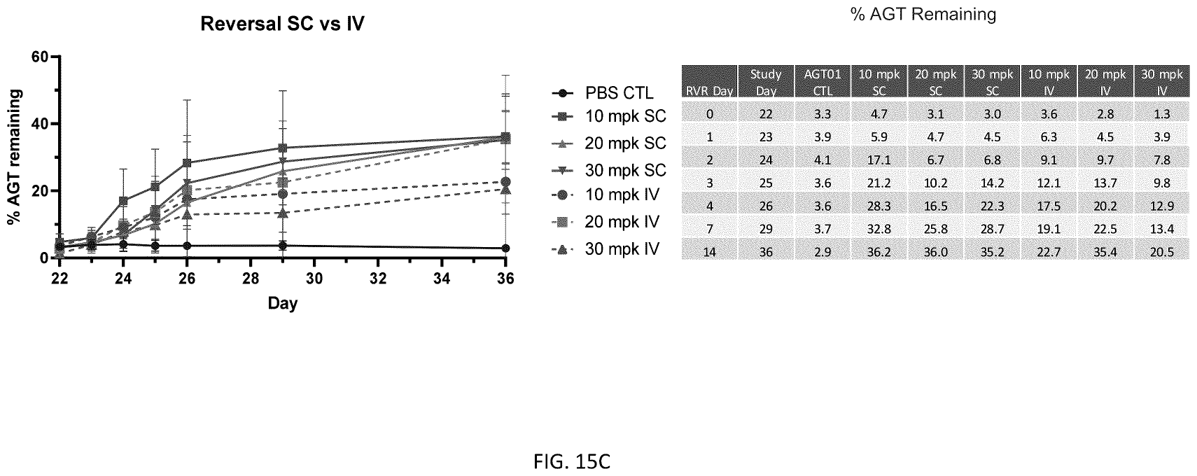

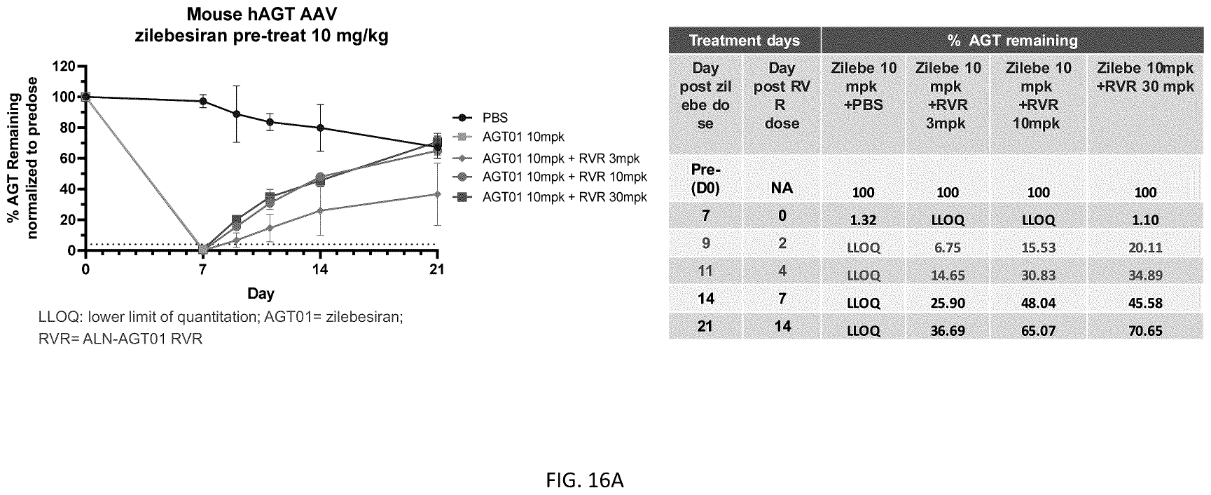

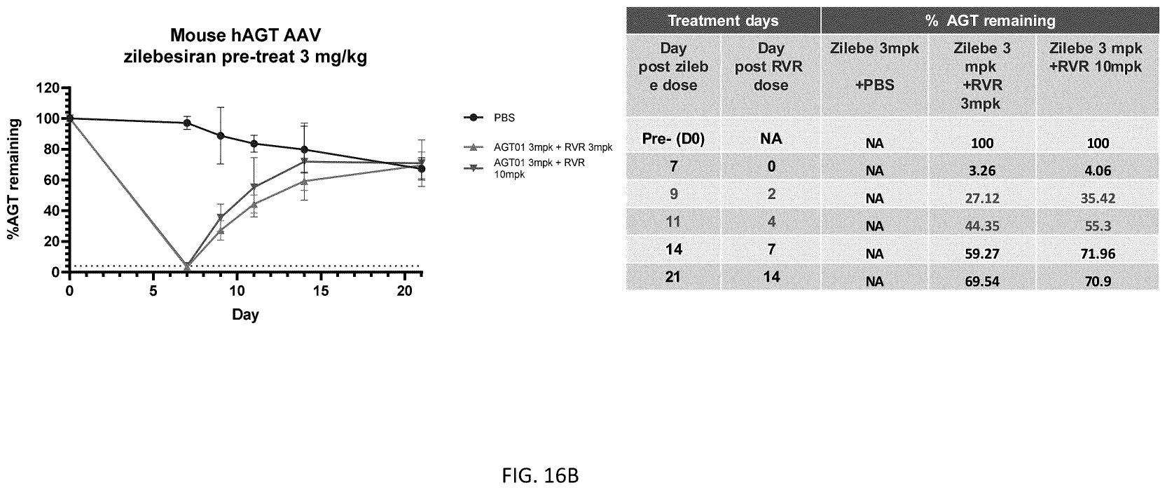

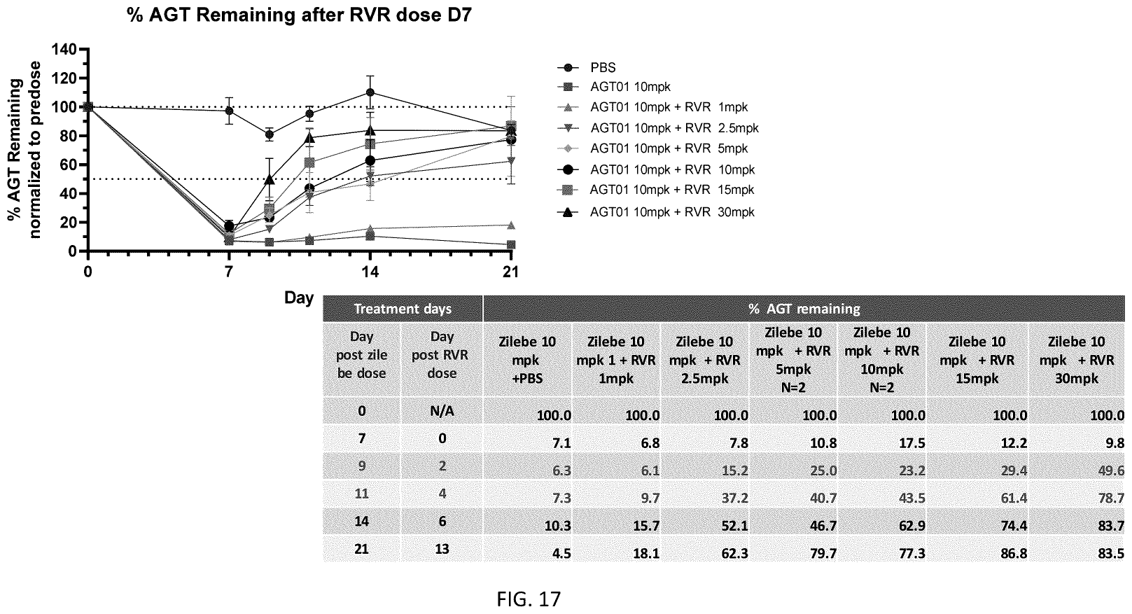

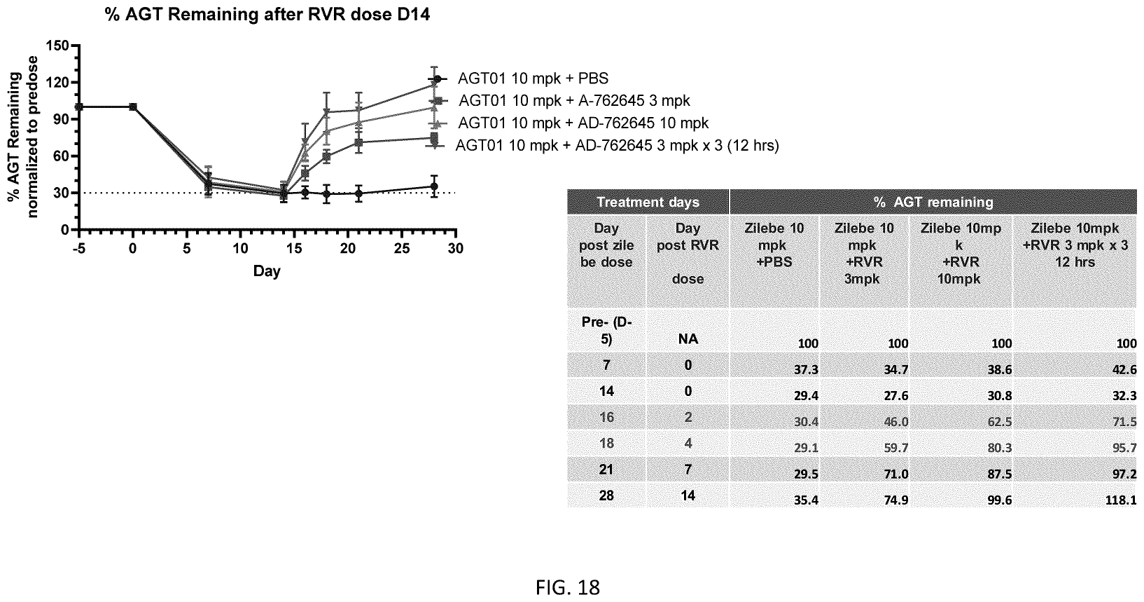

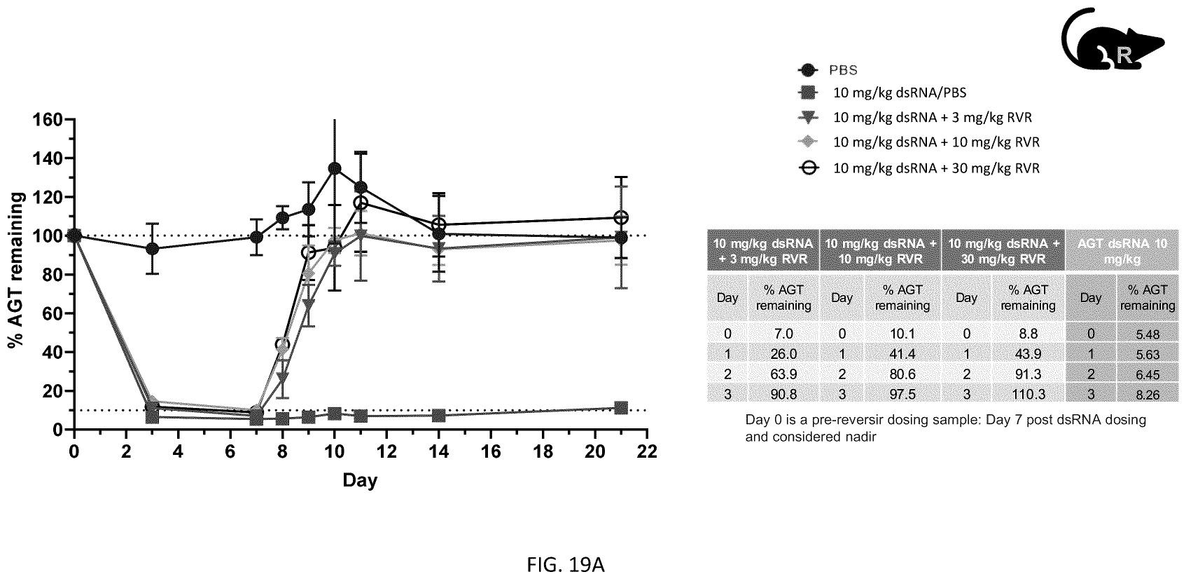

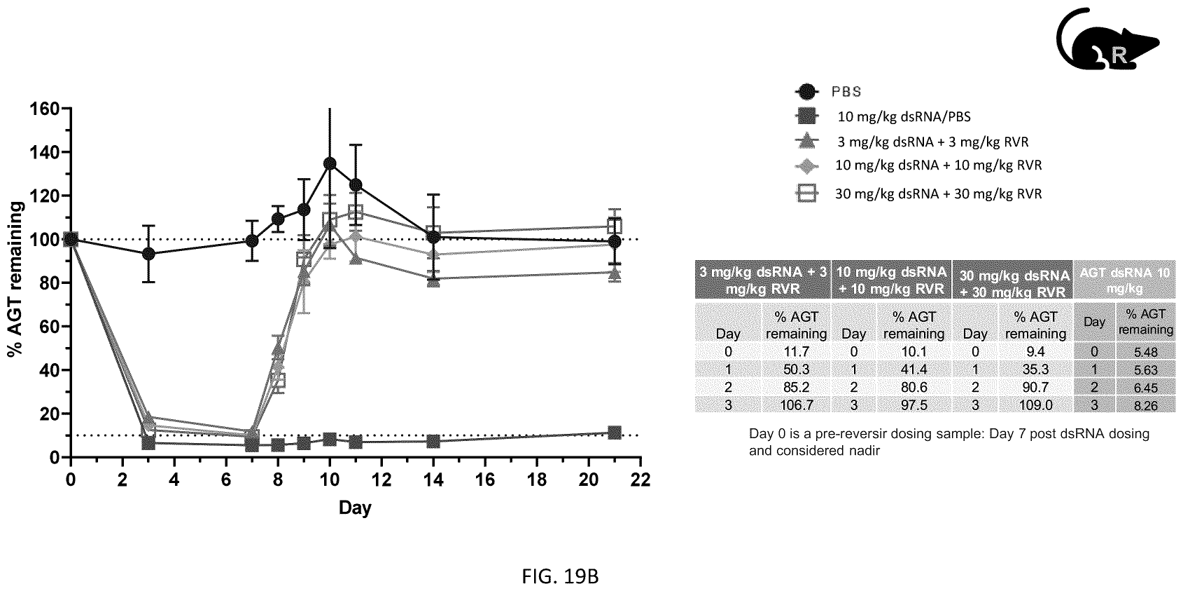

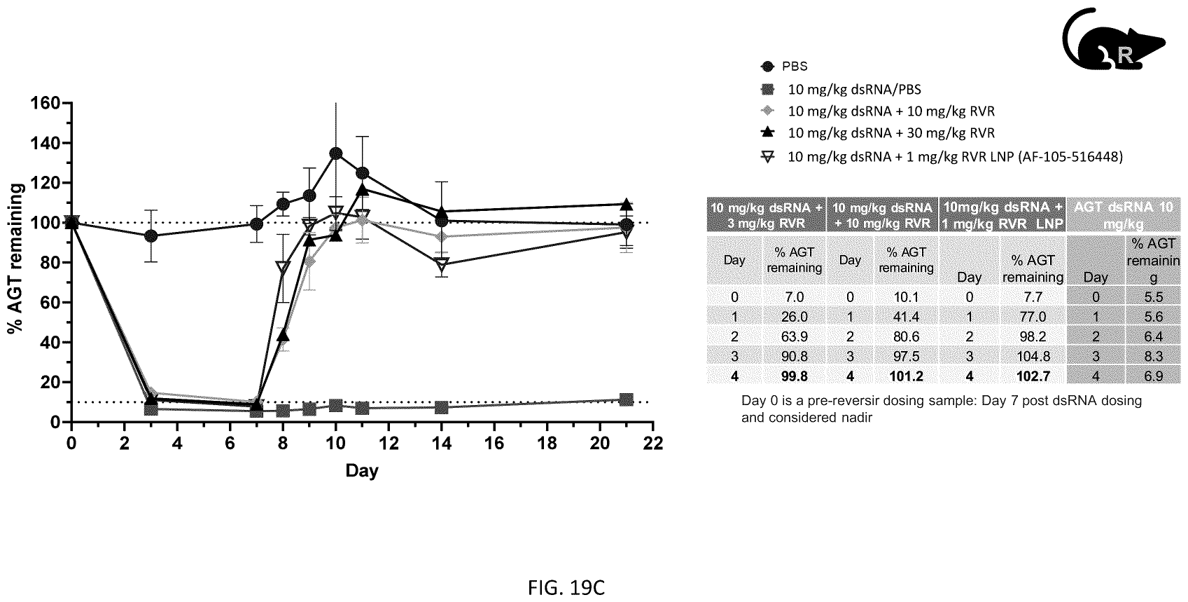

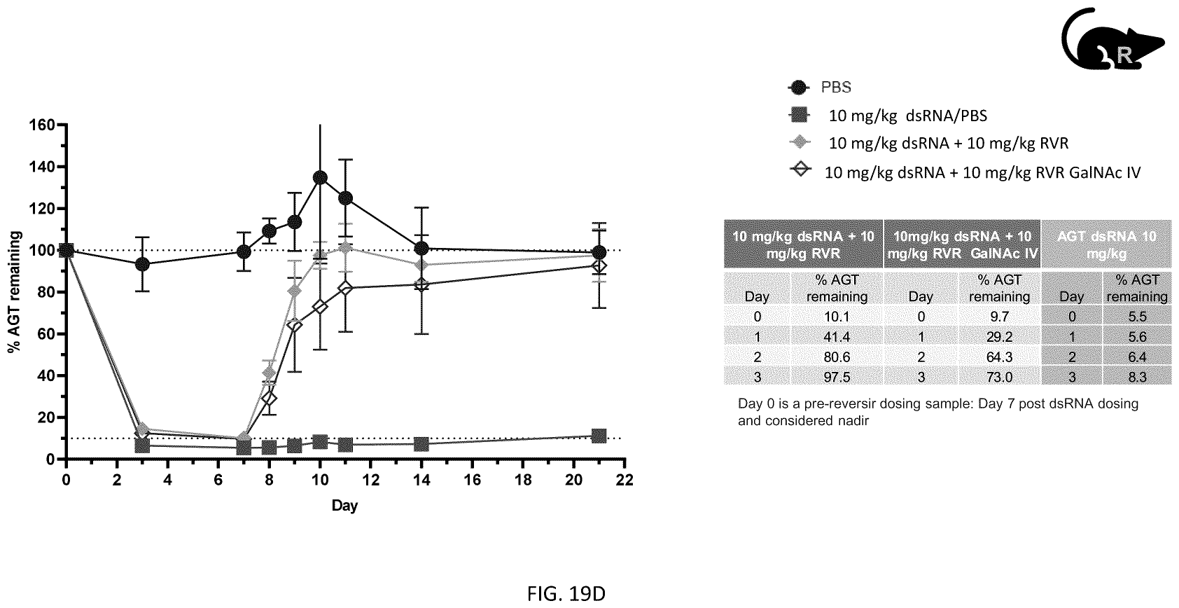

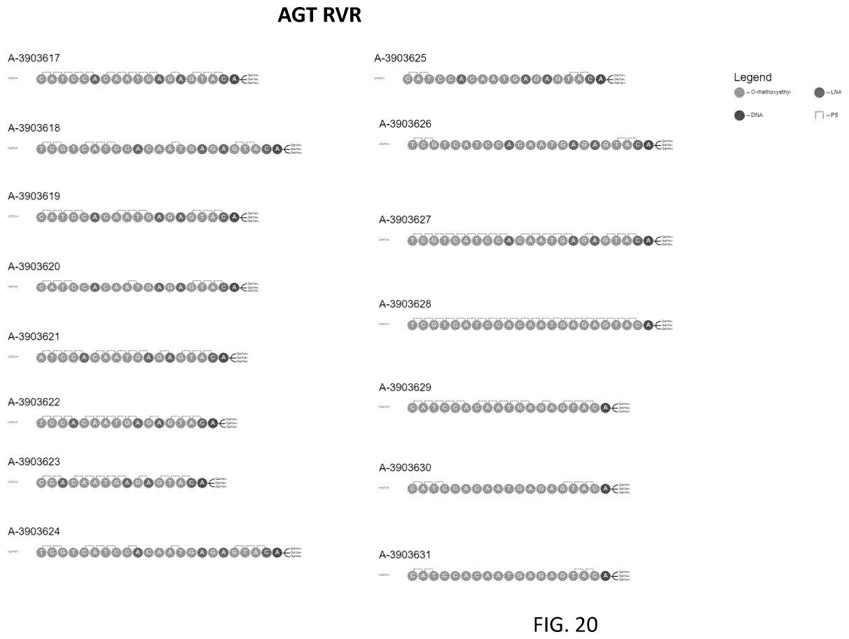

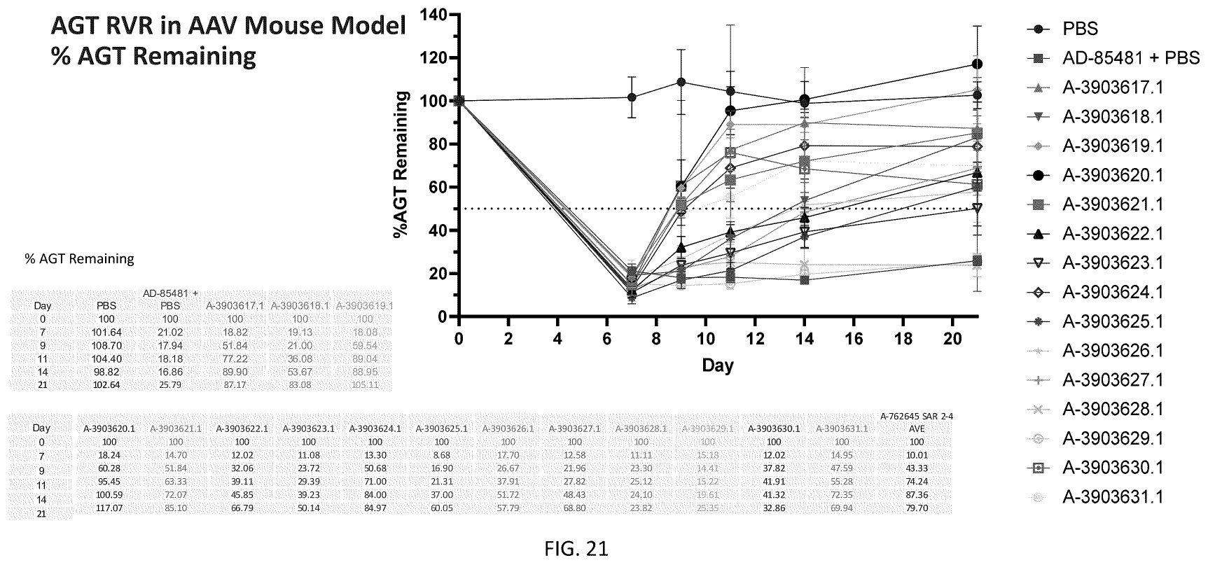

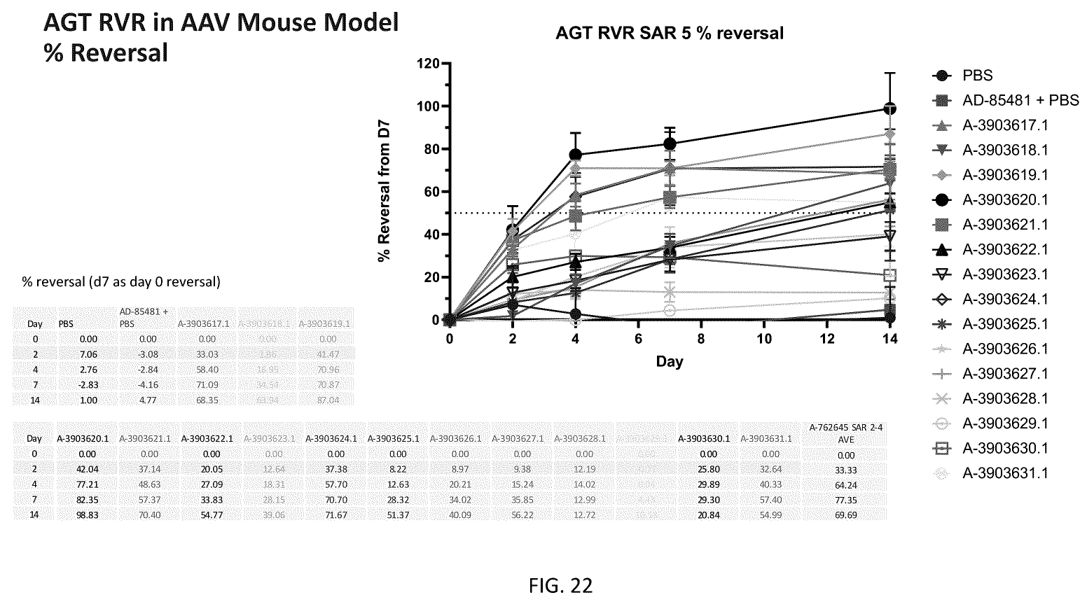

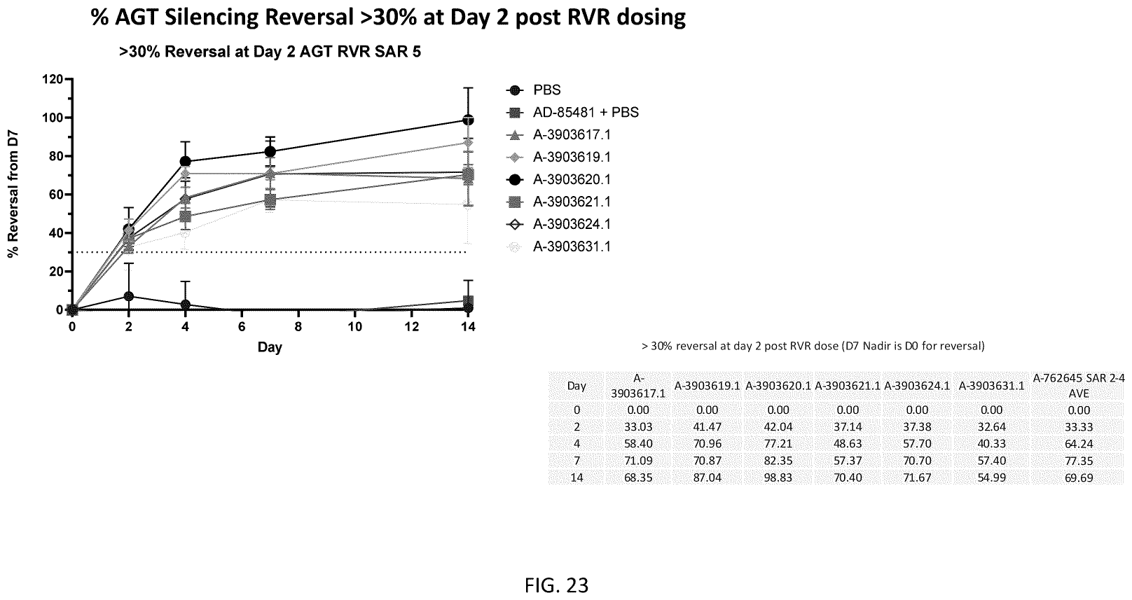

In a separate in vivo set of experiments, at pre-dose Day −21 wild-type mice (C57BL/6) were transduced with 2×10¹⁰ viral particles of an adeno-associated virus 8 (AAV8) vector encoding human AGT via intravenous injection. At day 0, mice were subcutaneously administered a single 3 mg/kg dose of AD-85481. At Day 7 groups of three mice were subcutaneously administered a single 1 mg/kg or 3 mg/kg dose of REVERSIRs A-762645, A-762689, or A-762722 (GalNAc-conjugated duplexes) (shown in FIG. 8A and Table 6), or PBS control.

Serum samples were collected at Days 0, 7, 9, 11, and 14. At Day 14, animals were sacrificed, liver samples were collected and snap-frozen in liquid nitrogen. Serum and liver mRNA was extracted and analyzed by the RT-QPCR method.

Human AGT mRNA levels were compared to a housekeeping gene, GAPDH and, as depicted in FIG. 8B, 15-mer, 16-mer, and 18-mer REVERSIRs potently inhibited the RNAi inhibitory activity of AD-85481, with the 15-mer and 18-mer REVERSIRs, A-762722 and A-762645, having the most potent activity.

In view of the foregoing results, A-762722 and A-762645 were assessed for activity in non-human primates. The study design is depicted in FIG. 9. Briefly, non-human primates were subcutaneously administered a single 3 mg/kg dose of AD-85481 at Day 0. At Day 22, groups of 3 animals were either subcutaneously administered a single 1 mg/kg or 3 mg/kg dose of A-762722 or A-762645 (GalNAc-conjugated duplexes), or intravenously administered a single 0.3 mg/kg dose of LNP formulated A-762722 or A-762645, or PBS control. At the intervals indicated, serum samples were collected and the level of AGT mRNA was analyzed by the RT-QPCR method.

As depicted in FIGS. 10A and 10B, the 18-mer, A-762645, effectively reversed the RNAi inhibitory activity of AD-85481. As was observed in mice, both intravenously administered LNP formulated REVERSIRs and subcutaneously administered GalNAc-conjugated REVERSIRs had a rapid onset of action, however, the LNP formulated REVERSIRs had less durability than the subcutaneously administered GalNAc-conjugated REVERSIRs. In addition, as was also observed in mice, A-762645 was effective with a 1:1 dose of REVERSIR: AD-85481.

Additional 18-mer REVERSIRs having fewer phosphorothioate linkages than the most effective and durable REVERSIR identified, A-762645, in order to minimize the risk of poor safety were designed and synthesized. The modified nucleotide sequences of a subset of these REVERSIRs are provided in FIG. 11A and shown in Table 6.

These REVERSIRs were assayed in vivo as described above.

At pre-dose Day −21 wild-type mice (C57BL/6) were transduced with 2×10¹⁰ viral particles of an adeno-associated virus 8 (AAV8) vector encoding human AGT via intravenous injection. At day 0, mice were subcutaneously administered a single 3 mg/kg dose of AD-85481. At Day 7 groups of three mice were subcutaneously administered a single 1 mg/kg or 3 mg/kg dose of REVERSIRs A-762645, A-809917, A809918, A-809919, A-809920, A-809921, A-809922, A-809923, A-809924, A809924, or A-809925 (GalNAc-conjugated duplexes), or PBS control.

At Days 0, 7, 9, 11, and 14 serum samples were collected. At Day 21 animals were sacrificed, liver samples were collected and snap-frozen in liquid nitrogen. Serum and liver mRNA was extracted and human AGT mRNA levels were analyzed by the RT-QPCR method.

Human AGT mRNA levels were compared to a housekeeping gene, GAPDH and it was demonstrated that all of the REVERSIRs were similarly efficacious at inhibiting the RNAi inhibitory activity of AD-85481, within and across all cohorts of mice tested (FIG. 11B).

In separate sets of experiments, mice were transduced with 2×10¹¹ viral particles of an adeno-associated virus 8 (AAV8) vector encoding human AGT via intravenous injection. At day 0, mice were subcutaneously administered a single 3 mg/kg dose of AD-85481. At Day 7 groups of three mice were subcutaneously administered a single 1 mg/kg or 3 mg/kg dose of REVERSIRs A-762645, A809918, A-809925, A2423818, or A2423819 (GalNAc-conjugated duplexes) (FIG. 12A), or PBS control.

At Days 0, 7, 9, 11, 14, and 21 serum samples were collected. At Day 21 animals were sacrificed, liver samples were collected and snap-frozen in liquid nitrogen. Serum and liver mRNA was extracted and analyzed by the RT-QPCR method.

The results of one set of experiments is presented in FIG. 12B and the results of another set of experiments is presented in FIG. 12C. The data demonstrate that all of the REVERSIRs assessed achieved at least some inhibition of the RNAi inhibitory activity of AD-85481 with near complete reversal of AD-85481 inhibitory activity for the 18-mer REVERSIRs comprising 14 or 10 phosphorothioate linkages, A-762645 and A-809918, and the 22-mer REVERSIR comprising 8 phosphorothioate linkages, A-2423818. The reversal effect is, however, lost with a reduction in phosphorothioate linkages across the 18-mer REVERSIRs (i.e., A-809925) but can be compensated for with an increase in length, e.g., to a 22-mer REVERSIR, as exemplified by the 22-mer REVERSIR, A-2423818 comprising 8 phosphorothioate linkages. The data also demonstrate a dose response for each of the assayed REVERSIRs.

In summary, REVERSIRs capable of complete reversal of AD-85481 RNAi inhibitory activity in mice were identified and demonstrated to be effective in non-human primates. It was also demonstrated that the number of phosphorothiate linkages of these REVERSIRs could be reduced without affecting efficacy and the most efficacious reversal was observed with REVERSIRs comprising 14, 10, and 6 phosphorothiate linkages.

### Example 4. Effect of REVERSIRs in Non-Human Primates and Safety and Toxicity Analysis of REVERSIRs

The REVERSIRs demonstrated to inhibit the RNAi inhibitory activity of AD-85481 described in Example 3, A-762645, A-809918, A-809925, and A-2423818, were assessed for efficacy in non-human primates. At Day 0, female non-human primates were subcutaneously administered a single 3 mg/kg dose of AD-85481. At Day 21, the primates (n=3) were subcutaneously administered a single 3 mg/kg dose of A-762645, A-809918, A-809925, or A-2423818 (GalNAc-conjugated duplexes). Serum samples were collected at Days −7, −14, 0, 3, 7, 14, 21, 22, 23, 24, 25, and 28. Serum mRNA was extracted and analyzed by the RT-QPCR method.

As depicted in FIGS. 13A-13C all of the REVERSIRs reversed the inhibition of AD-85481 with A-762645 achieving 10% reversal of serum AGT levels 2 days after administration and at a 1:1 AD-85481:REVERSIR ratio, a desirable clinical feature.

A-762645, A-809918, A-809925, and A-2423818 were further assessed for safety and toxicity. In particular, in a diluted human whole blood transfection assay (24-hour incubation), there were no pro-inflammatory responses (FIG. 14A) for any of the REVERSIRs and there were no antisense oligonucleotide-like proinflammatory responses observed in a 6-hour human whole blood assay (FIG. 14B) for any of the REVERSIRs. Furthermore, in the non-human primates platelet counts and whole blood cell counts were within the normal range throughout the study above for all of the REVERSIRs (FIG. 14C).

In addition, in a rat toxicity study, administration of a single 30 mg/kg or 100 mg/kg dose of A-762645, A-809918, or A-809925 did not results in any clinical signs, organ or body weight changes, gross findings or organ weight changes or clinical pathology changes. Histopathology of the liver, kidney, administration site, and adrenal glands were all within normal ranges.

### Example 5. Effect of REVERSIRs in Non-Human Primates and Pharmacology Analysis of REVERSIRs

The REVERSIR A-762645 was assessed for efficacy in non-human primates. At Day 0, female non-human primates (n=3) were subcutaneously administered a single 3 mg/kg dose of AD-85481. At Day 22, a subcutaneous dose of A-762645 was administered at 1 mg/kg or 3 mg/kg or an intravenous dose of AFO11-762645 (A-762645 in an LNP formulation) was administered at 0.3 mg/kg. Serum samples were collected at Days −7, 0, 3, 7, 14, 21, 28, 35, 42, 49, 56, 63, 70, 77, 84, 91 and 98. Serum mRNA was extracted and analyzed by the RT-QPCR method.

As depicted in FIG. 15A, use of an increased REVERSIR (RVR)/dsRNA ratio or use of an LNP formulation was demonstrated to improve the reversal kinetics. The LNP formulated REVERSIR achieved faster kinetics but the reversal of AGT knockdown was not as durable as observed for the GalNAc form of the REVERSIR. Further, as depicted in FIG. 13A, A-762645 demonstrated the most favorable pharmacodynamic profile to reverse the AD-85481 (Zilebesiran) mediated knockdown of AGT.

In a further study, at Day 0, female non-human primates were subcutaneously administered a single 10 mg/kg dose of AD-85481 (equivalent to approximately 600 mg dose in a human subject). At Day 22, the primates were subcutaneously administered a single dose of 10 mg/kg (n=2); a single dose of 30 mg/kg; or a split dose of 30 mg/kg (split as three doses of 10 mg/kg each at 24 hour intervals), of the A-762645-GalNAc conjugated REVERSIR; or PBS as control. Serum samples were collected at Days 0, 7, 14, 21, 28, 35, 42, 49, 56, 63, 70, 77, 84, 91 and 98. Serum mRNA was extracted and analyzed by the RT-QPCR method.

As depicted in FIG. 15B, A-762645 resulted in potent and durable pharmacology following AD-85481 (Zilebesiran) dosing in non-human primates. Further, split dosing (3 doses of 10 mg/kg each at 24 hour intervals) at the highest dose level improved REVERSIR pharmacodynamics compared to a single dose, while a split dosing of lower doses did not improve REVERSIR pharmacodynamics compared to a single dose (data not shown), suggesting that a saturation state is reached at high doses, likely due to receptor availability at a given time.

In a further study, at Day 22, the primates were administered a single 10 mg/kg, 20 mg/kg, or 30 mg/kg dose of the A-762645-GalNAc conjugated REVERSIR subcutaneously or A-762645-LNP formulated intravenously. Serum samples were collected at Days 22, 23, 24, 25, 26, 29, and 36. Serum mRNA was extracted and analyzed by the RT-QPCR method. As depicted in FIG. 15C, the subcutaneous delivery of the REVERSIR demonstrated a better pharmacodynamic profile relative to intravenous delivery of the REVERSIR at all dose levels. In particular, subcutaneous delivery of REVERSIR in non-human primates resulted in faster reversal of AGT knockdown compared with intravenous infusion administration of REVERSIR.

### Example 6. Effect of REVERSIR A-762645 in Humanized Mouse Models

A-762645 was selected and assessed for in vivo activity in different humanized mouse models. In a first study, the efficacy of A-762645 was assessed in an hAGT-AAV mouse model pretreated with AD-85481 (Zilebesiran). The human AGT gene was expressed in the mouse hepatocytes by transduction with liver-specific AAV8 virus. At day 0, mice were subcutaneously administered a single 3 mg/kg (FIG. 16A) or 10 mg/kg (FIG. 16B) dose of AD-85481. At Day 7, groups of three mice were subcutaneously administered a single 3 mg/kg, 10 mg/kg, or 30 mg/kg dose of the REVERSIR A-762645-GalNAc conjugated; or PBS control.

At Days 0, 7, 9, 11, 14, and 21 serum samples were collected. At Day 21 the animals were sacrificed, liver samples were collected and snap-frozen in liquid nitrogen. Serum and liver mRNA was extracted and analyzed by the RT-QPCR method. Human AGT mRNA levels were compared to a housekeeping gene, GAPDH and, as depicted in FIGS. 16A and 16B, the REVERSIR potently inhibited the RNAi inhibitory activity of AD-85481.

In a second study, the efficacy of A-762645 was assessed in an hAGT transgenic mouse model pretreated with AD-85481 (Zilebesiran). Briefly, the human AGT gene was randomly inserted in multiple areas in the mouse genome (generated during embryonic stem cell stage). At day 0, mice were subcutaneously administered a single 10 mg/kg dose of AD-85481. At Day 7, groups of three mice were subcutaneously administered a single 1 mg/kg, 2.5 mg/kg, 5 mg/kg, 10 mg/kg, 15 mg/kg or 30 mg/kg dose of REVERSIR A-762645; or PBS control.

At Days 0, 7, 9, 11, 14, and 21 serum samples were collected. At Day 21 the animals were sacrificed, liver samples were collected and snap-frozen in liquid nitrogen. Serum and liver mRNA was extracted and analyzed by the RT-QPCR method. Human AGT mRNA levels were compared to a housekeeping gene, GAPDH and, as depicted in FIG. 17, the REVERSIR potently inhibited the RNAi inhibitory activity of AD-85481.

In a third study, the efficacy of A-762645 was assessed in a PXB mouse model pretreated with AD-85481 (Zilebesiran). Briefly, in PXB mice, the liver was repopulated with human hepatocytes while other liver cells (Kupffer cells, endothelial cells, stellate cell etc.) remained of mouse origin. At day 0, mice were subcutaneously administered a single 10 mg/kg dose of AD-85481. At Day 14, groups of three mice were subcutaneously administered a single dose of 3 mg/kg, or 10 mg/kg; or a split dose of 3 mg/kg×3 (12 hours) of REVERSIR A-762645, or PBS control.

At Days −5, 0, 7, 14, 16, 18, 21 and 28 serum samples were collected. At Day 28 the animals were sacrificed, liver samples were collected and snap-frozen in liquid nitrogen. Serum and liver mRNA was extracted and analyzed by the RT-QPCR method. Human AGT mRNA levels were compared to a housekeeping gene, GAPDH and, as depicted in FIG. 18, the REVERSIR potently inhibited the RNAi inhibitory activity of AD-85481.

### Example 7. Effect of REVERSIR A-762645 in a Rat Model

The efficacy of A-762645 was assessed in a rat model pretreated with AD-85481 (Zilebesiran). Briefly, at day 0, rats (n=3) were subcutaneously administered a single 3 mg/kg, 10 mg/kg or 30 mg/kg dose of AD-85481. At Day 7, groups of three rats were subcutaneously administered a single 3 mg/kg, 10 mg/kg, or 30 mg/kg dose of REVERSIR A-762645-GalNAc conjugated, intravenously administered a single 10 mg/kg dose of A-762645 comprising a GalNAc ligand, intravenously administered a single 1 mg/kg dose of AF-105-516448 (REVERSIR in an LNP formulation); or PBS control. Further, the intravenous dose was administered as a bolus in the rat's tail vein.

At Days 0, 3, 7, 8, 9, 10, 11, 14, and 21 serum samples were collected. At Day 21 the animals were sacrificed, liver samples were collected and snap-frozen in liquid nitrogen. Serum and liver mRNA was extracted and analyzed by the RT-QPCR method. AGT mRNA levels were compared to a housekeeping gene, GAPDH.

As depicted in FIG. 19A, a higher ratio of REVERSIR to dsRNA increased the kinetics of reversal of AGT knockdown, similar to the observation in non-human primates. Further, as depicted in FIG. 19B, the AD-85481 dsRNA load did not affect the reversal of AGT knockdown. Further, as depicted in FIG. 19C, the reversal of AGT knockdown with the REVERSIR in an LNP formulation was faster relative to reversal with the same REVERSIR comprising a GalNAc ligand. Further, as depicted in FIG. 19D, bolus intravenous dosing did not result in faster kinetics over subcutaneous administration of the REVERSIR.

### Example 8. Effect of REVERSIRs in a Humanized Mouse Model

The efficacy of REVERSIRs A-3903617, A-3903618, A-3903619, A-3903620, A-3903621, A-3903622, A-3903623, A-3903624, A-3903625, A-3903626, A-3903627. A-3903628. A-3903629. A-3903630, and A-3903631 (FIG. 20) was assessed in a mouse AAV model pretreated with AD-85481 (Zilebesiran). Briefly, mice were transduced with AAV serotype 8 expressing human AGT (hAGT). 14 days after transduction on Day 0 of study, mice were administered phosphate-buffered saline (PBS) or AD-85481 at 3 mg/kg via subcutaneous injection. On Day 7 mice were administered phosphate-buffered saline (PBS) or the REVERSIR at 3 mg/kg via subcutaneous injection. Serum samples were collected on Day 0, 7, 9, 11, 14 and 21. Serum AGT protein was measured by ELISA. As depicted in FIGS. 21-23, the REVERSIRs potently inhibited the RNAi inhibitory activity of AD-85481.

TABLE 6
Exemplary REVERSIRs of the Invention

| | | SEQ | Unmodified | SEQ |
|---|---|---|---|---|
| | | ID | Oligonucleotide | ID |
| Oligo ID | Modified Oligonucleotide Sequence 5′ to 3′ | NO: | Sequence 5′ to 3′ | NO: |
| A-516448 | gscsusasuscscsusgsgsgscsasas(Aln)(Aln)(Aln)(Tln)cs(Aln)dAL96 | 95 | GCUAUCCUGGGCAAAAATCAA | 118 |
| A-762635 | csasuscscsas(m5Cln)asasusgs(Aln)gs(Aln)gs(Tln)as(m5Cln)dAL96 | 96 | CAUCCACAAUGAGAGTACA | 119 |
| A-762636 | csasuscscs(Aln)csasasusgs(Aln)gs(Aln)gs(Tln)as(m5Cln)dAL96 | 97 | CAUCCACAAUGAGAGTACA | 120 |
| A-762637 | csasuscs(m5Cln)ascsasasusgs(Aln)gs(Aln)gs(Tln)as(m5Cln)dAL96 | 98 | CAUCCACAAUGAGAGTACA | 121 |
| A-762638 | csasus(m5Cln)csascsasasusgs(Aln)gs(Aln)gs(Tln)as(m5Cln)dAL96 | 99 | CAUCCACAAUGAGAGTACA | 122 |
| A-762645 | csasuscscs(Aln)csasasusgs(Aln)gs(Aln)gsusas(m5Cln)dAL96 | 100 | CAUCCACAAUGAGAGUACA | 123 |
| A-762655 | csasuscscs(Aln)csasas(Tln)gs(Aln)gsasgsusas(m5Cln)dAL96 | 101 | CAUCCACAATGAGAGUACA | 124 |
| A-762680 | uscscs(Aln)csasasusgs(Aln)gs(Aln)gs(Tln)as(m5Cln)dAL96 | 102 | UCCACAAUGAGAGTACA | 125 |
| A-762689 | uscscs(Aln)csasasusgs(Aln)gs(Aln)gsusas(m5Cln)dAL96 | 103 | UCCACAAUGAGAGUACA | 126 |
| A-762701 | uscscs(Aln)cs(Aln)asusgs(Aln)gsasgsusas(m5Cln)dAL96 | 104 | UCCACAAUGAGAGUACA | 127 |
| A-762722 | cscs(Aln)csasasusgs(Aln)gs(Aln)gsusas(m5Cln)dAL96 | 105 | CCACAAUGAGAGUACA | 128 |
| A-809917 | csasuscc(Aln)csasasusg(Aln)g(Aln)gsusas(m5Cln)dAL96 | 106 | CAUCCACAAUGAGAGUACA | 129 |
| A-809918 | csasuscsc(Aln)casasusg(Aln)g(Aln)gsusas(m5Cln)dAL96 | 107 | CAUCCACAAUGAGAGUACA | 130 |
| A-809919 | csasuscc(Aln)csasasusg(Aln)gs(Aln)gsusa(m5Cln)dAL96 | 108 | CAUCCACAAUGAGAGUACA | 131 |
| A-809920 | csasuscc(Aln)csaasusg(Aln)g(Aln)gsusa(m5Cln)dAL96 | 109 | CAUCCACAAUGAGAGUACA | 132 |
| A-809921 | csasuscsc(Aln)caasusg(Aln)g(Aln)gsusa(m5Cln)dAL96 | 110 | CAUCCACAAUGAGAGUACA | 133 |
| A-809922 | csasusccs(Aln)casausg(Aln)g(Aln)gsusa(m5Cln)dAL96 | 111 | CAUCCACAAUGAGAGUACA | 134 |
| A-809923 | csasuscc(Aln)csaasugs(Aln)g(Aln)gsuas(m5Cln)dAL96 | 112 | CAUCCACAAUGAGAGUACA | 135 |
| A-809924 | csasuscc(Aln)casaugs(Aln)g(Aln)gsua(m5Cln)dAL96 | 113 | CAUCCACAAUGAGAGUACA | 136 |
| A-809925 | csasuscc(Aln)caasug(Aln)g(Aln)gsusa(m5Cln)dAL96 | 114 | CAUCCACAAUGAGAGUACA | 137 |
| A-809925 | csasuscc(Aln)caasug(Aln)g(Aln)gsusa(m5Cln)dAL96 | 115 | CAUCCACAAUGAGAGUACA | 138 |
| A-2423818 | uscsgucsasuscc(Aln)caasug(Aln)g(Aln)gsusa(m5Cln)dAL96 | 116 | UCGUCAUCCACAAUGAGAGUACA | 139 |
| A-2423819 | uscscs(Aln)caasug(Aln)g(Aln)gsusa(m5Cln)dAL96 | 117 | UCCACAAUGAGAGUACA | 140 |

1. A single stranded oligonucleotide for inhibiting RNAi activity of a double stranded ribonucleic acid (dsRNA) agent comprising a thermally destabilizing nucleotide in the antisense strand,
wherein the single stranded oligonucleotide comprises a nucleotide sequence substantially complementary to the antisense strand of the dsRNA agent,
wherein the single stranded oligonucleotide is 16-30 nucleotides in length,
wherein substantially all of the nucleotides of the single stranded oligonucleotide comprise a nucleotide modification, and
wherein at least three of the nucleotide modifications are a high affinity nucleotide modification.

2. The single stranded oligonucleotide of claim 1,
(i) wherein the thermally destabilizing nucleotide modification is selected from the group consisting of an abasic modification; a mismatch with the opposing nucleotide in the duplex; a destabilizing sugar modification, a 2′-deoxy modification, an acyclic nucleotide, an unlocked nucleic acid (UNA), and a glycerol nucleic acid (GNA);
(ii) wherein substantially all of the nucleotides comprise a nucleotide modification selected from the group consisting of a 2′-O-alkyl modification, a 2′ substituted alkoxy modification, a 2′-substituted alkyl modification, a 2′-halo modification, a deoxynucleotide modification, a locked nucleic acid (LNA) modification, a D-Methyleneoxy (4′-CH2-O-2′) locked nucleic acid (LNA) modification, a 2-O-(2-Methoxyethyl) (MOE) modification, bridged nucleic acid (2′,4′-BNA), 2-O-Ethyl (cEt), and a 2′-O-methyl modification;
(iii) wherein all of the nucleotides comprise a nucleotide modification selected from the group consisting of a 2′-O-alkyl modification, a 2′-substituted alkoxy modification, a 2′-substituted alkyl modification, a 2′-halo modification, a deoxynucleotide modification, a D-Methyleneoxy (4′-CH2-O-2′) locked nucleic acid (LNA) modification, bridged nucleic acid (2′,4′-BNA), 2′-O-Ethyl (cEt), and a 2′-O-methyl modification;
(iv) wherein at least four of the nucleotide modifications are a high affinity nucleotide modification;
(v) wherein at least five of the nucleotide modifications are a high affinity nucleotide modification; and/or
(vi) further comprising at least five phosphorothioate internucleotide modifications.

3-5. (canceled)

6. The single stranded oligonucleotide of claim 1,
(i) wherein at least two of the high affinity nucleotide modifications are at positions 2 and 6; positions 2 and 5; positions 2 and 7; positions 2 and 8; positions 2 and 9; positions 2 and 14; positions 2 and 15; and/or positions 2 and 16, counting from the 3′-end of the oligonucleotide;
(ii) wherein the high affinity nucleotide modifications are at positions 2, 6, 8, and 14; 2, 4, 5, 6, and 7; 2, 4, 6, 8, and 13; 2, 4, 6, 8, and 14; 2, 4, 6, 8, and 15; 2, 4, 6, 8, and 16; 2, 8, 10, and 14; 2, 4, 6, 8, and 14; or 2, 8, 12, and 14, counting from the 3′-end of the oligonucleotide;
(iii) wherein at least one of the nucleotides comprising a high affinity nucleotide modification is base paired with the nucleotide comprising the thermally destabilizing nucleotide in the antisense strand of the dsRNA agent; and/or
(iv) wherein the high affinity modification is selected from the group consisting of a locked nucleic acid (LNA) modification, a constrained Ethyl nucleic acid (cEtNA) modification, and a bridged nucleic acid (BNA) modification.

7-10. (canceled)

11. The single stranded oligonucleotide of claim 1, wherein the single stranded oligonucleotide is conjugated to at least one ligand.

12. The single stranded oligonucleotide of claim 11,
(i) wherein the ligand is an N-acetylgalactosamine (GalNAc) derivative;
(ii) wherein the ligand is one or more GalNAc derivatives attached through a monovalent, bivalent, or trivalent branched linker;
(iii) wherein the ligand is 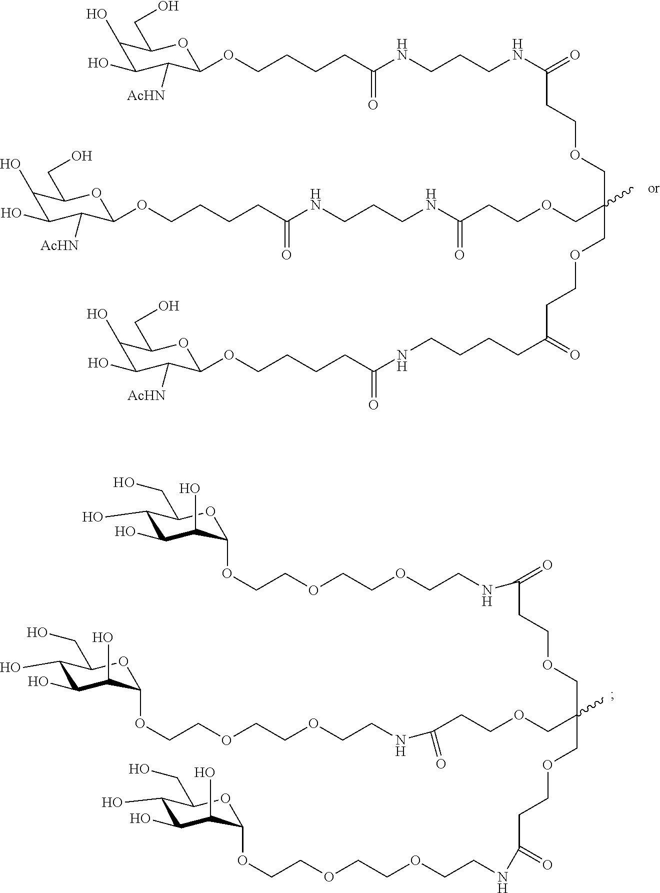
(iv) wherein the ligand is conjugated to a nucleoside comprising a deoxy sugar in the single stranded oligonucleotide, optionally wherein the deoxy sugar is a 2′-deoxy ribose; and/or
(v) wherein the ligand is conjugated to 3′-terminus of the single stranded oligonucleotide.

13-17. (canceled)

18. A single stranded oligonucleotide for inhibiting RNAi activity of a double stranded ribonucleic acid (dsRNA) agent comprising a thermally destabilizing nucleotide in the antisense strand,
wherein the single stranded oligonucleotide comprises a nucleotide sequence substantially complementary to the antisense strand of the dsRNA agent,
wherein the single stranded oligonucleotide is 18-24 nucleotides in length,
wherein the single stranded oligonucleotide comprises at least five phosphorothioate internucleotide modifications and is represented by formula (I): 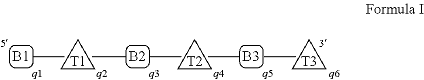
wherein:
B1, B2 and B3 each independently represent a nucleotide comprising a nucleotide modification independently selected from the group consisting of a 2′-deoxy, 2′-ribo, 2′-O-alkyl modification, a 2′-substituted alkoxy modification, a 2′-substituted alkoxy alkyl modification, a 2′-substituted alkyl modification, and a 2′-halo modification;
T1, T2, and T3 each independently represent a nucleotide comprising a nucleotide modification selected from the group consisting of a deoxynucleotide modification, a D-Methyleneoxy (4′-CH2-O-2′) locked nucleic acid (LNA) modification, a 2′O-(2-Methoxyethyl) (MOE) modification, a bridged nucleic acid (2′,4′-BNA) modification, 2′-O-Ethyl (cEt) modification, a 2′-deoxy-2′-Fluoro, and a 2′-O-methyl modification;
q¹, q³ and q⁵ are each independently 3-12 nucleotides in length;
q², q⁴ and q⁶ are independently 1-6 nucleotide(s) in length; and
wherein the single stranded oligonucleotide is conjugated to at least one ligand.

19. The single stranded oligonucleotide of claim 1,
(i) wherein the single stranded oligonucleotide comprises 5-15 phosphorothioate internucleotide modifications; 5-14 phosphorothioate internucleotide modifications; 5-13 phosphorothioate internucleotide modifications; 5-12 phosphorothioate internucleotide modifications; 5-11 phosphorothioate internucleotide modifications; 5-10 phosphorothioate internucleotide modifications; 5-9 phosphorothioate internucleotide modifications; 5-8 phosphorothioate internucleotide modifications; 5-7 phosphorothioate internucleotide modifications; or 5-6 phosphorothioate internucleotide modifications: or
(ii) wherein the single stranded oligonucleotide comprises 6-14 phosphorothioate internucleotide modifications.

20. (canceled)

21. (canceled)

22. The single stranded oligonucleotide of claim 1,
(i) wherein the single stranded oligonucleotide is at least about 90% complementary to the entire length of the antisense strand of the dsRNA agent;
(ii) wherein the single stranded oligonucleotide is 90% complementary to nucleotides 2-16 of the antisense stand of the dsRNA agent;
(iii) wherein the single stranded oligonucleotide is fully complementary to the antisense strand of the dsRNA agent;
(iv) wherein the nucleotide sequence of the antisense strand of the dsRNA agent comprises the nucleotide sequence 5′-UGUACUCUCAUUGUGGAUGACGA-3′ of SEQ ID NO: 9;
(v) wherein the nucleotide sequence of the antisense strand of the dsRNA agent comprises the nucleotide sequence 5′-usGfsuac(Tgn)cucauugUfgGfaugacsgsa-3′ of SEQ ID NO: 11, wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-O-fluoroadenosine-3′-phosphate, 2′-O-fluorocytidine-3′-phosphate, 2′-O-fluoroguanosine-3′-phosphate, and 2′-O-fluorouridine-3′-phosphate, respectively; dT is a deoxy-thymine; s is a phosphorothioate linkage; and (Tgn) is thymidine-glycol nucleic acid (GNA)S-isomer;
(vi) wherein the nucleotide sequence of the single stranded oligonucleotide is at least 90% identical to the entire nucleotide sequence of any one of the unmodified nucleotide sequences in Table 6; and/or
(vii) wherein the nucleotide sequence of the single stranded oligonucleotide comprises a modified nucleotide sequence differing by no more than 4 modified nucleotides from any one of the modified nucleotide sequences in Table 6.

23-28. (canceled)

29. The single stranded oligonucleotide of claim 18,
(i) wherein the ligand is an N-acetylgalactosamine (GalNAc) derivative;
(ii) wherein the ligand is one or more GalNAc derivatives attached through a monovalent, bivalent, or trivalent branched linker;
(iii) wherein the ligand is 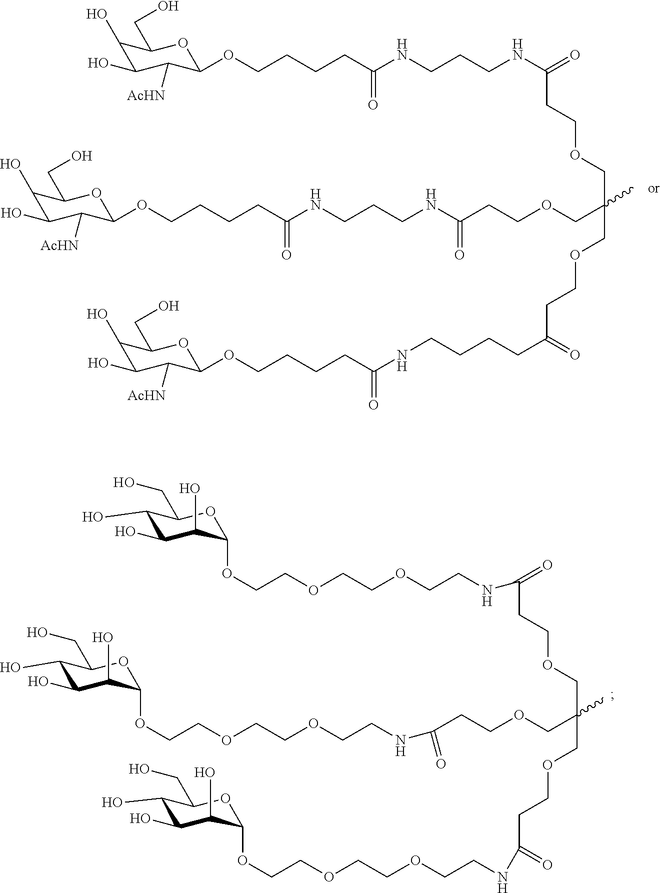
(iv) wherein the ligand is conjugated to a nucleoside comprising a deoxy sugar in the single stranded oligonucleotide, optionally wherein the deoxy sugar is a 2′-deoxy ribose; and/or
(v) wherein the ligand is conjugated to 3′-terminus of the single stranded oligonucleotide.

30-35. (canceled)

36. A pharmaceutical composition comprising the single stranded oligonucleotide of claim 1.

37-40. (canceled)

41. An isolated cell comprising the single stranded oligonucleotide of claim 1.

42. A method of inhibiting the RNAi inhibitory activity of a dsRNA agent comprising a thermally destabilizing nucleotide modification in the antisense strand, the method comprising
contacting the dsRNA agent with the single stranded oligonucleotide of claim 1, thereby inhibiting the RNAi inhibitory activity of a dsRNA agent comprising a thermally destabilizing nucleotide modification in the antisense strand.

43. (canceled)

44. (canceled)

45. A method of treating a subject in need thereof, the method comprising
administering to the subject a therapeutically effective amount of the single stranded oligonucleotide of claim 1, thereby treating the subject.

46. The method of claim 45, wherein the subject in need thereof was previously administered a double stranded RNAi agent that inhibits the expression of a target gene and comprises a thermally destabilizing nucleotide modification in the antisense strand.

47. The method of claim 46, wherein the target gene is angiotensinogen (AGT), and/or wherein the subject in need thereof is suffering from hypotension, hyperkalemia, or renal dysfunction.

48-50. (canceled)

51. The method of claim 45, further comprising administering to the subject an additional therapy or therapeutic agent selected from the group consisting of increased dietary fluid/salt, fludrocortisone/midodrine treatment, intravenous fluids, vasopressor medications, down-titration or interruption of concomitant antihypertensive medications, a low potassium diet, thiazide/loop diuretic medications, oral potassium binders, calcium, glucose, insulin, and hemodialysis, or combinations thereof.

52. The method of claim 45, wherein the single stranded oligonucleotide is administered to the subject subcutaneously or intravenously.

53. (canceled)

54. The method of claim 45, wherein the dose of the single stranded oligonucleotide is at a ratio of about 1:1, 2:1 or 3:1 to the dose of dsRNA agent previously administered to the subject.

55. A method of ameliorating in a subject a side effect of a dsRNA agent which inhibits the expression of a target gene and comprises a thermally destabilizing nucleotide modification in the antisense strand, the method comprising
administering to the subject an effective amount of the single stranded oligonucleotide of claim 1, thereby ameliorating the side effect of the dsRNA agent in the subject.

56-66. (canceled)

67. A kit comprising the single stranded oligonucleotide of claim 1, and instructions for use, and optionally, means for administration of the single stranded oligonucleotide.

68. (canceled)


SEQUENCE LISTING (140 sequences)

SEQ ID NO: 1 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 2 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O,4'-C-methylene adenosine-3'-phosphate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 3 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O,4'-C-methylene 5-methyluridine-3'-phosphate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 4 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O,4'-C-methylene guanosine-3'-phosphate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 5 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O,4'-C-methylene adenosine-3'-phosphate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 6 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O,4'-C-methylene guanosine-3'-phosphate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 7 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O,4'-C-methylene adenosine-3'-phosphate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 8 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O,4'-C-methylene guanosine-3'-phosphate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 9 - RNA, 23 nt, synthetic construct
  source 1..23: mol_type = other RNA
tgtactctcattgtggatgacga

SEQ ID NO: 10 - RNA, 21 nt, synthetic construct
  source 1..21: mol_type = other RNA
gtcatccacaatgagagtaca

SEQ ID NO: 11 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluoroguanosine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = Thymidine-glycol nucleic acid (GNA), S-Isomer
  modified_base 7: mod_base = OTHER; note = 2'-O-methylcytidine-3'-phosphate
  modified_base 8: mod_base = OTHER; note = 2'-O-methyluridine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyladenosine-3'-phosphate
  modified_base 21: mod_base = OTHER; note = 2'-O-methylcytidine-3'-phosphorothioate
  modified_base 22: mod_base = OTHER; note = 2'-O-methylguanosine-3'-phosphorothioate
  modified_base 23: mod_base = OTHER; note = 2'-O-methyladenosine
tgtactctcattgtggatgacga

SEQ ID NO: 12 - RNA, 21 nt, synthetic construct
  source 1..21: mol_type = other RNA
  modified_base 1: mod_base = OTHER; note = 2'-O-methylguanosine-3'-phosphorothioate
  modified_base 2: mod_base = OTHER; note = 2'-O-methyluridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methylguanosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methylcytidine-3'-phosphate
  modified_base 21: mod_base = OTHER; note = 2'-O-methyladenosine
gtcatccacaatgagagtaca

SEQ ID NO: 13 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O,4'-C-methylene 5-methyluridine-3'-phosphate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 14 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O,4'-C-methylene adenosine-3'-phosphate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 15 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O,4'-C-methylene adenosine-3'-phosphate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 16 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O,4'-C-methylene 5-methylcytidine-3'-phosphate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 17 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O,4'-C-methylene adenosine-3'-phosphate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 18 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O,4'-C-methylene 5-methylcytidine-3'-phosphate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 19 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O,4'-C-methylene 5-methylcytidine-3'-phosphate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 20 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O,4'-C-methylene 5-methyluridine-3'-phosphate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 21 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O,4'-C-methylene adenosine-3'-phosphate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 22 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O,4'-C-methylene 5-methylcytidine-3'-phosphate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 23 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O,4'-C-methylene 5-methyluridine-3'-phosphate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 24 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O,4'-C-methylene guanosine-3'-phosphate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 25 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O,4'-C-methylene 5-methylcytidine-3'-phosphate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 26 - DNA, 23 bp, synthetic construct
  source 1..23: mol_type = other DNA
  modified_base 1: mod_base = OTHER; note = 2'-O,4'-C-methylene 5-methyluridine-3'-phosphate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 27 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 28 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O,4'-C-methylene adenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 29 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O,4'-C-methylene 5-methyluridine-3'-phosphate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 30 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O,4'-C-methylene guanosine-3'-phosphate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 31 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 32 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O,4'-C-methylene guanosine-3'-phosphate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 33 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 34 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O,4'-C-methylene guanosine-3'-phosphate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 35 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O,4'-C-methylene 5-methyluridine-3'-phosphate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 36 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O,4'-C-methylene adenosine-3'-phosphate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 37 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O,4'-C-methylene adenosine-3'-phosphate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 38 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O,4'-C-methylene 5-methylcytidine-3'-phosphate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 39 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O,4'-C-methylene adenosine-3'-phosphate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 40 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O,4'-C-methylene 5-methylcytidine-3'-phosphate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 41 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O,4'-C-methylene 5-methylcytidine-3'-phosphate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 42 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O,4'-C-methylene 5-methyluridine-3'-phosphate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 43 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O,4'-C-methylene adenosine-3'-phosphate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 44 - DNA, 19 bp, synthetic construct
  source 1..19: mol_type = other DNA
  modified_base 1: mod_base = OTHER; note = 2'-O,4'-C-methylene 5-methylcytidine-3'-phosphate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 45 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 46 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O,4'-C-methylene adenosine-3'-phosphate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 47 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O,4'-C-methylene 5-methyluridine-3'-phosphate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 48 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O,4'-C-methylene guanosine-3'-phosphate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 49 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 50 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O,4'-C-methylene guanosine-3'-phosphate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 51 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O,4'-C-methylene adenosine-3'-phosphate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 52 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O,4'-C-methylene guanosine-3'-phosphate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 53 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O,4'-C-methylene 5-methyluridine-3'-phosphate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 54 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O,4'-C-methylene adenosine-3'-phosphate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 55 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O,4'-C-methylene adenosine-3'-phosphate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 56 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O,4'-C-methylene 5-methylcytidine-3'-phosphate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 57 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O,4'-C-methylene adenosine-3'-phosphate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 58 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O,4'-C-methylene 5-methylcytidine-3'-phosphate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 59 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O,4'-C-methylene 5-methylcytidine-3'-phosphate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 60 - DNA, 17 bp, synthetic construct
  source 1..17: mol_type = other DNA
  modified_base 1: mod_base = OTHER; note = 2'-O,4'-C-methylene 5-methyluridine-3'-phosphate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 61 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O,4'-C-methylene adenosine-3'-phosphate
  modified_base 17: mod_base = OTHER; note = 2'-O,4'-C-methylene guanosine-3'-phosphate
  modified_base 18: mod_base = OTHER; note = 2'-O,4'-C-methylene adenosine-3'-phosphate
  modified_base 19: mod_base = OTHER; note = 2'-O-methylguanosine-3'-phosphorothioate
  modified_base 20: mod_base = OTHER; note = 2'-O,4'-C-methylene 5-methyluridine-3'-phosphate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 62 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O,4'-C-methylene adenosine-3'-phosphate
  modified_base 17: mod_base = OTHER; note = 2'-O,4'-C-methylene guanosine-3'-phosphate
  modified_base 18: mod_base = OTHER; note = 2'-O-methyladenosine-3'-phosphorothioate
  modified_base 19: mod_base = OTHER; note = 2'-O,4'-C-methylene guanosine-3'-phosphate
  modified_base 20: mod_base = OTHER; note = 2'-O,4'-C-methylene 5-methyluridine-3'-phosphate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 63 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O,4'-C-methylene adenosine-3'-phosphate
  modified_base 17: mod_base = OTHER; note = 2'-O,4'-C-methylene guanosine-3'-phosphate
  modified_base 18: mod_base = OTHER; note = 2'-O,4'-C-methylene adenosine-3'-phosphate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O,4'-C-methylene adenosine-3'-phosphate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 64 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O,4'-C-methylene adenosine-3'-phosphate
  modified_base 17: mod_base = OTHER; note = 2'-O,4'-C-methylene guanosine-3'-phosphate
  modified_base 18: mod_base = OTHER; note = 2'-O-methyladenosine-3'-phosphorothioate
  modified_base 19: mod_base = OTHER; note = 2'-O,4'-C-methylene guanosine-3'-phosphate
  modified_base 20: mod_base = OTHER; note = 2'-O-methyluridine-3'-phosphorothioate
  modified_base 21: mod_base = OTHER; note = 2'-O,4'-C-methylene adenosine-3'-phosphate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 65 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O-methylcytidine-3'-phosphorothioate
  modified_base 10: mod_base = OTHER; note = 2'-O-methyladenosine-3'-phosphorothioate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O,4'-C-methylene guanosine-3'-phosphate
  modified_base 18: mod_base = OTHER; note = 2'-O,4'-C-methylene adenosine-3'-phosphate
  modified_base 19: mod_base = OTHER; note = 2'-O,4'-C-methylene guanosine-3'-phosphate
  modified_base 20: mod_base = OTHER; note = 2'-O-methyluridine-3'-phosphorothioate
  modified_base 21: mod_base = OTHER; note = 2'-O,4'-C-methylene adenosine-3'-phosphate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 66 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O,4'-C-methylene 5-methylcytidine-3'-phosphate
  modified_base 10: mod_base = OTHER; note = 2'-O,4'-C-methylene adenosine-3'-phosphate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O,4'-C-methylene guanosine-3'-phosphate
  modified_base 20: mod_base = OTHER; note = 2'-O,4'-C-methylene 5-methyluridine-3'-phosphate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 67 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O,4'-C-methylene 5-methylcytidine-3'-phosphate
  modified_base 10: mod_base = OTHER; note = 2'-O,4'-C-methylene adenosine-3'-phosphate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O,4'-C-methylene adenosine-3'-phosphate
  modified_base 19: mod_base = OTHER; note = 2'-O-methylguanosine-3'-phosphorothioate
  modified_base 20: mod_base = OTHER; note = 2'-O,4'-C-methylene 5-methyluridine-3'-phosphate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 68 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O,4'-C-methylene 5-methylcytidine-3'-phosphate
  modified_base 10: mod_base = OTHER; note = 2'-O,4'-C-methylene adenosine-3'-phosphate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O,4'-C-methylene guanosine-3'-phosphate
  modified_base 18: mod_base = OTHER; note = 2'-O-methyladenosine-3'-phosphorothioate
  modified_base 19: mod_base = OTHER; note = 2'-O,4'-C-methylene guanosine-3'-phosphate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 69 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O,4'-C-methylene 5-methylcytidine-3'-phosphate
  modified_base 9: mod_base = OTHER; note = 2'-O-methylcytidine-3'-phosphorothioate
  modified_base 10: mod_base = OTHER; note = 2'-O,4'-C-methylene adenosine-3'-phosphate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O,4'-C-methylene guanosine-3'-phosphate
  modified_base 20: mod_base = OTHER; note = 2'-O,4'-C-methylene 5-methyluridine-3'-phosphate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 70 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O,4'-C-methylene 5-methylcytidine-3'-phosphate
  modified_base 9: mod_base = OTHER; note = 2'-O-methylcytidine-3'-phosphorothioate
  modified_base 10: mod_base = OTHER; note = 2'-O,4'-C-methylene adenosine-3'-phosphate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O,4'-C-methylene adenosine-3'-phosphate
  modified_base 19: mod_base = OTHER; note = 2'-O-methylguanosine-3'-phosphorothioate
  modified_base 20: mod_base = OTHER; note = 2'-O,4'-C-methylene 5-methyluridine-3'-phosphate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 71 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O,4'-C-methylene 5-methylcytidine-3'-phosphate
  modified_base 9: mod_base = OTHER; note = 2'-O-methylcytidine-3'-phosphorothioate
  modified_base 10: mod_base = OTHER; note = 2'-O,4'-C-methylene adenosine-3'-phosphate
  modified_base 11: mod_base = OTHER; note = 2'-O-methylcytidine-3'-phosphorothioate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O,4'-C-methylene guanosine-3'-phosphate
  modified_base 18: mod_base = OTHER; note = 2'-O-methyladenosine-3'-phosphorothioate
  modified_base 19: mod_base = OTHER; note = 2'-O,4'-C-methylene guanosine-3'-phosphate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 72 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O,4'-C-methylene 5-methylcytidine-3'-phosphate
  modified_base 10: mod_base = OTHER; note = 2'-O-methyladenosine-3'-phosphorothioate
  modified_base 11: mod_base = OTHER; note = 2'-O,4'-C-methylene 5-methylcytidine-3'-phosphate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O-methyladenosine-3'-phosphorothioate
  modified_base 19: mod_base = OTHER; note = 2'-O,4'-C-methylene guanosine-3'-phosphate
  modified_base 20: mod_base = OTHER; note = 2'-O,4'-C-methylene 5-methyluridine-3'-phosphate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 73 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O,4'-C-methylene 5-methylcytidine-3'-phosphate
  modified_base 10: mod_base = OTHER; note = 2'-O-methyladenosine-3'-phosphorothioate
  modified_base 11: mod_base = OTHER; note = 2'-O,4'-C-methylene 5-methylcytidine-3'-phosphate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O-methylguanosine-3'-phosphorothioate
  modified_base 18: mod_base = OTHER; note = 2'-O,4'-C-methylene adenosine-3'-phosphate
  modified_base 19: mod_base = OTHER; note = 2'-O-methylguanosine-3'-phosphorothioate
  modified_base 20: mod_base = OTHER; note = 2'-O,4'-C-methylene 5-methyluridine-3'-phosphate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 74 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphorothioate
  modified_base 4: mod_base = OTHER; note = 2'-O-methyluridine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphorothioate
  modified_base 9: mod_base = OTHER; note = 2'-O,4'-C-methylene 5-methylcytidine-3'-phosphate
  modified_base 10: mod_base = OTHER; note = 2'-O-methyladenosine-3'-phosphorothioate
  modified_base 11: mod_base = OTHER; note = 2'-O,4'-C-methylene 5-methylcytidine-3'-phosphate
  modified_base 12: mod_base = OTHER; note = 2'-O-methyladenosine-3'-phosphorothioate
  modified_base 13: mod_base = OTHER; note = 2'-O-methyladenosine-3'-phosphorothioate
  modified_base 14: mod_base = OTHER; note = 2'-O-methyluridine-3'-phosphorothioate
  modified_base 15: mod_base = OTHER; note = 2'-O-methylguanosine-3'-phosphorothioate
  modified_base 16: mod_base = OTHER; note = 2'-O-methyladenosine-3'-phosphorothioate
  modified_base 17: mod_base = OTHER; note = 2'-O,4'-C-methylene guanosine-3'-phosphate
  modified_base 18: mod_base = OTHER; note = 2'-O-methyladenosine-3'-phosphorothioate
  modified_base 19: mod_base = OTHER; note = 2'-O,4'-C-methylene guanosine-3'-phosphate
  modified_base 20: mod_base = OTHER; note = 2'-O-methyluridine-3'-phosphorothioate
  modified_base 21: mod_base = OTHER; note = 2'-O-methyladenosine-3'-phosphorothioate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 75 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O,4'-C-methylene guanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O,4'-C-methylene 5-methyluridine-3'-phosphate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 76 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O,4'-C-methylene guanosine-3'-phosphate
  modified_base 14: mod_base = OTHER; note = 2'-O-methyladenosine-3'-phosphorothioate
  modified_base 15: mod_base = OTHER; note = 2'-O,4'-C-methylene guanosine-3'-phosphate
  modified_base 16: mod_base = OTHER; note = 2'-O,4'-C-methylene 5-methyluridine-3'-phosphate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 77 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O,4'-C-methylene guanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O,4'-C-methylene adenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 78 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O,4'-C-methylene guanosine-3'-phosphate
  modified_base 14: mod_base = OTHER; note = 2'-O-methyladenosine-3'-phosphorothioate
  modified_base 15: mod_base = OTHER; note = 2'-O,4'-C-methylene guanosine-3'-phosphate
  modified_base 16: mod_base = OTHER; note = 2'-O-methyluridine-3'-phosphorothioate
  modified_base 17: mod_base = OTHER; note = 2'-O,4'-C-methylene adenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 79 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O,4'-C-methylene guanosine-3'-phosphate
  modified_base 14: mod_base = OTHER; note = 2'-O,4'-C-methylene adenosine-3'-phosphate
  modified_base 15: mod_base = OTHER; note = 2'-O,4'-C-methylene guanosine-3'-phosphate
  modified_base 16: mod_base = OTHER; note = 2'-O-methyluridine-3'-phosphorothioate
  modified_base 17: mod_base = OTHER; note = 2'-O,4'-C-methylene adenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 80 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O,4'-C-methylene adenosine-3'-phosphate
  modified_base 11: mod_base = OTHER; note = 2'-O,4'-C-methylene guanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O,4'-C-methylene 5-methyluridine-3'-phosphate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 81 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O,4'-C-methylene adenosine-3'-phosphate
  modified_base 11: mod_base = OTHER; note = 2'-O,4'-C-methylene guanosine-3'-phosphate
  modified_base 12: mod_base = OTHER; note = 2'-O-methyladenosine-3'-phosphorothioate
  modified_base 13: mod_base = OTHER; note = 2'-O,4'-C-methylene guanosine-3'-phosphate
  modified_base 14: mod_base = OTHER; note = 2'-O,4'-C-methylene 5-methyluridine-3'-phosphate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 82 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O,4'-C-methylene adenosine-3'-phosphate
  modified_base 11: mod_base = OTHER; note = 2'-O,4'-C-methylene guanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O,4'-C-methylene adenosine-3'-phosphate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 83 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O,4'-C-methylene adenosine-3'-phosphate
  modified_base 11: mod_base = OTHER; note = 2'-O,4'-C-methylene guanosine-3'-phosphate
  modified_base 12: mod_base = OTHER; note = 2'-O-methyladenosine-3'-phosphorothioate
  modified_base 13: mod_base = OTHER; note = 2'-O,4'-C-methylene guanosine-3'-phosphate
  modified_base 14: mod_base = OTHER; note = 2'-O-methyluridine-3'-phosphorothioate
  modified_base 15: mod_base = OTHER; note = 2'-O,4'-C-methylene adenosine-3'-phosphate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 84 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O-methyladenosine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methyladenosine-3'-phosphorothioate
  modified_base 11: mod_base = OTHER; note = 2'-O,4'-C-methylene guanosine-3'-phosphate
  modified_base 12: mod_base = OTHER; note = 2'-O,4'-C-methylene adenosine-3'-phosphate
  modified_base 13: mod_base = OTHER; note = 2'-O,4'-C-methylene guanosine-3'-phosphate
  modified_base 14: mod_base = OTHER; note = 2'-O-methyluridine-3'-phosphorothioate
  modified_base 15: mod_base = OTHER; note = 2'-O,4'-C-methylene adenosine-3'-phosphate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 85 - DNA, 13 bp, synthetic construct
  source 1..13: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methyluridine-3'-phosphorothioate
  modified_base 5: mod_base = OTHER; note = 2'-O-methylguanosine-3'-phosphorothioate
  modified_base 6: mod_base = OTHER; note = 2'-O,4'-C-methylene adenosine-3'-phosphate
  modified_base 7: mod_base = OTHER; note = 2'-O,4'-C-methylene guanosine-3'-phosphate
  modified_base 8: mod_base = OTHER; note = 2'-O,4'-C-methylene adenosine-3'-phosphate
  modified_base 9: mod_base = OTHER; note = 2'-O-methylguanosine-3'-phosphorothioate
  modified_base 10: mod_base = OTHER; note = 2'-O,4'-C-methylene 5-methyluridine-3'-phosphate
  modified_base 11: mod_base = OTHER; note = 2'-O-methyladenosine-3'-phosphorothioate
  modified_base 12: mod_base = OTHER; note = 2'-O,4'-C-methylene 5-methylcytidine-3'-phosphate
  modified_base 13: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..12: note = RNA
  misc_feature 13: note = DNA
caatgagagtaca

SEQ ID NO: 86 - DNA, 13 bp, synthetic construct
  source 1..13: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methyluridine-3'-phosphorothioate
  modified_base 5: mod_base = OTHER; note = 2'-O-methylguanosine-3'-phosphorothioate
  modified_base 6: mod_base = OTHER; note = 2'-O,4'-C-methylene adenosine-3'-phosphate
  modified_base 7: mod_base = OTHER; note = 2'-O,4'-C-methylene guanosine-3'-phosphate
  modified_base 8: mod_base = OTHER; note = 2'-O-methyladenosine-3'-phosphorothioate
  modified_base 9: mod_base = OTHER; note = 2'-O,4'-C-methylene guanosine-3'-phosphate
  modified_base 10: mod_base = OTHER; note = 2'-O,4'-C-methylene 5-methyluridine-3'-phosphate
  modified_base 11: mod_base = OTHER; note = 2'-O-methyladenosine-3'-phosphorothioate
  modified_base 12: mod_base = OTHER; note = 2'-O,4'-C-methylene 5-methylcytidine-3'-phosphate
  modified_base 13: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..12: note = RNA
  misc_feature 13: note = DNA
caatgagagtaca

SEQ ID NO: 87 - DNA, 13 bp, synthetic construct
  source 1..13: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methyluridine-3'-phosphorothioate
  modified_base 5: mod_base = OTHER; note = 2'-O-methylguanosine-3'-phosphorothioate
  modified_base 6: mod_base = OTHER; note = 2'-O,4'-C-methylene adenosine-3'-phosphate
  modified_base 7: mod_base = OTHER; note = 2'-O,4'-C-methylene guanosine-3'-phosphate
  modified_base 8: mod_base = OTHER; note = 2'-O,4'-C-methylene adenosine-3'-phosphate
  modified_base 9: mod_base = OTHER; note = 2'-O-methylguanosine-3'-phosphorothioate
  modified_base 10: mod_base = OTHER; note = 2'-O-methyluridine-3'-phosphorothioate
  modified_base 11: mod_base = OTHER; note = 2'-O,4'-C-methylene adenosine-3'-phosphate
  modified_base 12: mod_base = OTHER; note = 2'-O,4'-C-methylene 5-methylcytidine-3'-phosphate
  modified_base 13: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..12: note = RNA
  misc_feature 13: note = DNA
caatgagagtaca

SEQ ID NO: 88 - DNA, 13 bp, synthetic construct
  source 1..13: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methyluridine-3'-phosphorothioate
  modified_base 5: mod_base = OTHER; note = 2'-O-methylguanosine-3'-phosphorothioate
  modified_base 6: mod_base = OTHER; note = 2'-O,4'-C-methylene adenosine-3'-phosphate
  modified_base 7: mod_base = OTHER; note = 2'-O,4'-C-methylene guanosine-3'-phosphate
  modified_base 8: mod_base = OTHER; note = 2'-O-methyladenosine-3'-phosphorothioate
  modified_base 9: mod_base = OTHER; note = 2'-O,4'-C-methylene guanosine-3'-phosphate
  modified_base 10: mod_base = OTHER; note = 2'-O-methyluridine-3'-phosphorothioate
  modified_base 11: mod_base = OTHER; note = 2'-O,4'-C-methylene adenosine-3'-phosphate
  modified_base 12: mod_base = OTHER; note = 2'-O,4'-C-methylene 5-methylcytidine-3'-phosphate
  modified_base 13: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..12: note = RNA
  misc_feature 13: note = DNA
caatgagagtaca

SEQ ID NO: 89 - DNA, 13 bp, synthetic construct
  source 1..13: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphorothioate
  modified_base 4: mod_base = OTHER; note = 2'-O-methyluridine-3'-phosphorothioate
  modified_base 5: mod_base = OTHER; note = 2'-O-methylguanosine-3'-phosphorothioate
  modified_base 6: mod_base = OTHER; note = 2'-O-methyladenosine-3'-phosphorothioate
  modified_base 7: mod_base = OTHER; note = 2'-O,4'-C-methylene guanosine-3'-phosphate
  modified_base 8: mod_base = OTHER; note = 2'-O,4'-C-methylene adenosine-3'-phosphate
  modified_base 9: mod_base = OTHER; note = 2'-O,4'-C-methylene guanosine-3'-phosphate
  modified_base 10: mod_base = OTHER; note = 2'-O-methyluridine-3'-phosphorothioate
  modified_base 11: mod_base = OTHER; note = 2'-O,4'-C-methylene adenosine-3'-phosphate
  modified_base 12: mod_base = OTHER; note = 2'-O,4'-C-methylene 5-methylcytidine-3'-phosphate
  modified_base 13: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..12: note = RNA
  misc_feature 13: note = DNA
caatgagagtaca

SEQ ID NO: 90 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O,4'-C-methylene adenosine-3'-phosphate
  modified_base 4: mod_base = OTHER; note = 2'-O,4'-C-methylene guanosine-3'-phosphate
  modified_base 5: mod_base = OTHER; note = 2'-O,4'-C-methylene adenosine-3'-phosphate
  modified_base 6: mod_base = OTHER; note = 2'-O-methylguanosine-3'-phosphorothioate
  modified_base 7: mod_base = OTHER; note = 2'-O,4'-C-methylene 5-methyluridine-3'-phosphate
  modified_base 8: mod_base = OTHER; note = 2'-O-methyladenosine-3'-phosphorothioate
  modified_base 9: mod_base = OTHER; note = 2'-O,4'-C-methylene 5-methylcytidine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
tgagagtaca

SEQ ID NO: 91 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O,4'-C-methylene adenosine-3'-phosphate
  modified_base 4: mod_base = OTHER; note = 2'-O,4'-C-methylene guanosine-3'-phosphate
  modified_base 5: mod_base = OTHER; note = 2'-O-methyladenosine-3'-phosphorothioate
  modified_base 6: mod_base = OTHER; note = 2'-O,4'-C-methylene guanosine-3'-phosphate
  modified_base 7: mod_base = OTHER; note = 2'-O,4'-C-methylene 5-methyluridine-3'-phosphate
  modified_base 8: mod_base = OTHER; note = 2'-O-methyladenosine-3'-phosphorothioate
  modified_base 9: mod_base = OTHER; note = 2'-O,4'-C-methylene 5-methylcytidine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
tgagagtaca

SEQ ID NO: 92 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O,4'-C-methylene adenosine-3'-phosphate
  modified_base 4: mod_base = OTHER; note = 2'-O,4'-C-methylene guanosine-3'-phosphate
  modified_base 5: mod_base = OTHER; note = 2'-O,4'-C-methylene adenosine-3'-phosphate
  modified_base 6: mod_base = OTHER; note = 2'-O-methylguanosine-3'-phosphorothioate
  modified_base 7: mod_base = OTHER; note = 2'-O-methyluridine-3'-phosphorothioate
  modified_base 8: mod_base = OTHER; note = 2'-O,4'-C-methylene adenosine-3'-phosphate
  modified_base 9: mod_base = OTHER; note = 2'-O,4'-C-methylene 5-methylcytidine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
tgagagtaca

SEQ ID NO: 93 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O,4'-C-methylene adenosine-3'-phosphate
  modified_base 4: mod_base = OTHER; note = 2'-O,4'-C-methylene guanosine-3'-phosphate
  modified_base 5: mod_base = OTHER; note = 2'-O-methyladenosine-3'-phosphorothioate
  modified_base 6: mod_base = OTHER; note = 2'-O,4'-C-methylene guanosine-3'-phosphate
  modified_base 7: mod_base = OTHER; note = 2'-O-methyluridine-3'-phosphorothioate
  modified_base 8: mod_base = OTHER; note = 2'-O,4'-C-methylene adenosine-3'-phosphate
  modified_base 9: mod_base = OTHER; note = 2'-O,4'-C-methylene 5-methylcytidine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
tgagagtaca

SEQ ID NO: 94 - DNA, 10 bp, synthetic construct
  source 1..10: mol_type = other DNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O-methyladenosine-3'-phosphorothioate
  modified_base 4: mod_base = OTHER; note = 2'-O,4'-C-methylene guanosine-3'-phosphate
  modified_base 5: mod_base = OTHER; note = 2'-O,4'-C-methylene adenosine-3'-phosphate
  modified_base 6: mod_base = OTHER; note = 2'-O,4'-C-methylene guanosine-3'-phosphate
  modified_base 7: mod_base = OTHER; note = 2'-O-methyluridine-3'-phosphorothioate
  modified_base 8: mod_base = OTHER; note = 2'-O,4'-C-methylene adenosine-3'-phosphate
  modified_base 9: mod_base = OTHER; note = 2'-O,4'-C-methylene 5-methylcytidine-3'-phosphate
  modified_base 10: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..9: note = RNA
  misc_feature 10: note = DNA
tgagagtaca

SEQ ID NO: 95 - DNA, 21 bp, synthetic construct
  source 1..21: mol_type = other DNA
  modified_base 1: mod_base = OTHER; note = 2'-O-methylguanosine-3'-phosphorothioate
  modified_base 2: mod_base = OTHER; note = 2'-O-methylcytidine-3'-phosphorothioate
  modified_base 3: mod_base = OTHER; note = 2'-O-methyluridine-3'-phosphorothioate
  modified_base 4: mod_base = OTHER; note = 2'-O-methyladenosine-3'-phosphorothioate
  modified_base 5: mod_base = OTHER; note = 2'-O-methyluridine-3'-phosphorothioate
  modified_base 6: mod_base = OTHER; note = 2'-O-methylcytidine-3'-phosphorothioate
  modified_base 7: mod_base = OTHER; note = 2'-O-methylcytidine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O-methylguanosine-3'-phosphorothioate
  modified_base 11: mod_base = OTHER; note = 2'-O-methylguanosine-3'-phosphorothioate
  modified_base 12: mod_base = OTHER; note = 2'-O-methylcytidine-3'-phosphorothioate
  modified_base 13: mod_base = OTHER; note = 2'-O-methyladenosine-3'-phosphorothioate
  modified_base 14: mod_base = OTHER; note = 2'-O-methyladenosine-3'-phosphorothioate
  modified_base 15: mod_base = OTHER; note = 2'-O,4'-C-methylene adenosine-3'-phosphate
  modified_base 16: mod_base = OTHER; note = 2'-O,4'-C-methylene adenosine-3'-phosphate
  modified_base 17: mod_base = OTHER; note = 2'-O,4'-C-methylene adenosine-3'-phosphate
  modified_base 18: mod_base = OTHER; note = 2'-O,4'-C-methylene 5-methyluridine-3'-phosphate
  modified_base 19: mod_base = OTHER; note = 2'-O-methylcytidine-3'-phosphorothioate
  modified_base 20: mod_base = OTHER; note = 2'-O,4'-C-methylene adenosine-3'-phosphate
  modified_base 21: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..20: note = RNA
  misc_feature 21: note = DNA
gctatcctgggcaaaaatcaa

SEQ ID NO: 96 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O,4'-C-methylene 5-methylcytidine-3'-phosphate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O,4'-C-methylene 5-methyluridine-3'-phosphate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 97 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O,4'-C-methylene adenosine-3'-phosphate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O,4'-C-methylene 5-methyluridine-3'-phosphate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 98 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O,4'-C-methylene 5-methylcytidine-3'-phosphate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O,4'-C-methylene 5-methyluridine-3'-phosphate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 99 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O,4'-C-methylene 5-methylcytidine-3'-phosphate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O,4'-C-methylene 5-methyluridine-3'-phosphate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 100 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O,4'-C-methylene adenosine-3'-phosphate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 101 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphorothioate
  modified_base 6: mod_base = OTHER; note = 2'-O,4'-C-methylene adenosine-3'-phosphate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O,4'-C-methylene 5-methyluridine-3'-phosphate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O-methyladenosine-3'-phosphorothioate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 102 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O,4'-C-methylene adenosine-3'-phosphate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O,4'-C-methylene adenosine-3'-phosphate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O,4'-C-methylene 5-methyluridine-3'-phosphate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 103 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O,4'-C-methylene adenosine-3'-phosphate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O,4'-C-methylene adenosine-3'-phosphate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 104 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O,4'-C-methylene adenosine-3'-phosphate
  modified_base 5: mod_base = OTHER; note = 2'-O-methylcytidine-3'-phosphorothioate
  modified_base 6: mod_base = OTHER; note = 2'-O,4'-C-methylene adenosine-3'-phosphate
  modified_base 7: mod_base = OTHER; note = 2'-O-methyladenosine-3'-phosphorothioate
  modified_base 8: mod_base = OTHER; note = 2'-O-methyluridine-3'-phosphorothioate
  modified_base 9: mod_base = OTHER; note = 2'-O-methylguanosine-3'-phosphorothioate
  modified_base 10: mod_base = OTHER; note = 2'-O,4'-C-methylene adenosine-3'-phosphate
  modified_base 11: mod_base = OTHER; note = 2'-O-methylguanosine-3'-phosphorothioate
  modified_base 12: mod_base = OTHER; note = 2'-O-methyladenosine-3'-phosphorothioate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphorothioate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 105 - DNA, 16 bp, synthetic construct
  source 1..16: mol_type = other DNA
  modified_base 1: mod_base = OTHER; note = 2'-O-methylcytidine-3'-phosphorothioate
  modified_base 2: mod_base = OTHER; note = 2'-O-methylcytidine-3'-phosphorothioate
  modified_base 3: mod_base = OTHER; note = 2'-O,4'-C-methylene adenosine-3'-phosphate
  modified_base 4: mod_base = OTHER; note = 2'-O-methylcytidine-3'-phosphorothioate
  modified_base 5: mod_base = OTHER; note = 2'-O-methyladenosine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylguanosine-3'-phosphorothioate
  modified_base 9: mod_base = OTHER; note = 2'-O,4'-C-methylene adenosine-3'-phosphate
  modified_base 10: mod_base = OTHER; note = 2'-O-methylguanosine-3'-phosphorothioate
  modified_base 11: mod_base = OTHER; note = 2'-O,4'-C-methylene adenosine-3'-phosphate
  modified_base 12: mod_base = OTHER; note = 2'-O-methylguanosine-3'-phosphorothioate
  modified_base 13: mod_base = OTHER; note = 2'-O-methyluridine-3'-phosphorothioate
  modified_base 14: mod_base = OTHER; note = 2'-O-methyladenosine-3'-phosphorothioate
  modified_base 15: mod_base = OTHER; note = 2'-O,4'-C-methylene 5-methylcytidine-3'-phosphate
  modified_base 16: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..15: note = RNA
  misc_feature 16: note = DNA
ccacaatgagagtaca

SEQ ID NO: 106 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphate
  modified_base 5: mod_base = OTHER; note = 2'-O-methylcytidine-3'-phosphate
  modified_base 6: mod_base = OTHER; note = 2'-O,4'-C-methylene adenosine-3'-phosphate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 107 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphate
  modified_base 6: mod_base = OTHER; note = 2'-O,4'-C-methylene adenosine-3'-phosphate
  modified_base 7: mod_base = OTHER; note = 2'-O-methylcytidine-3'-phosphate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 108 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphate
  modified_base 5: mod_base = OTHER; note = 2'-O-methylcytidine-3'-phosphate
  modified_base 6: mod_base = OTHER; note = 2'-O,4'-C-methylene adenosine-3'-phosphate
  modified_base 7: mod_base = OTHER; note = 2'-O-methylcytidine-3'-phosphorothioate
  modified_base 8: mod_base = OTHER; note = 2'-O-methyladenosine-3'-phosphorothioate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 109 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphate
  modified_base 5: mod_base = OTHER; note = 2'-O-methylcytidine-3'-phosphate
  modified_base 6: mod_base = OTHER; note = 2'-O,4'-C-methylene adenosine-3'-phosphate
  modified_base 7: mod_base = OTHER; note = 2'-O-methylcytidine-3'-phosphorothioate
  modified_base 8: mod_base = OTHER; note = 2'-O-methyladenosine-3'-phosphate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 110 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphorothioate
  modified_base 5: mod_base = OTHER; note = 2'-O-methylcytidine-3'-phosphate
  modified_base 6: mod_base = OTHER; note = 2'-O,4'-C-methylene adenosine-3'-phosphate
  modified_base 7: mod_base = OTHER; note = 2'-O-methylcytidine-3'-phosphate
  modified_base 8: mod_base = OTHER; note = 2'-O-methyladenosine-3'-phosphate
  modified_base 9: mod_base = OTHER; note = 2'-O-methyladenosine-3'-phosphorothioate
  modified_base 10: mod_base = OTHER; note = 2'-O-methyluridine-3'-phosphorothioate
  modified_base 11: mod_base = OTHER; note = 2'-O-methylguanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 111 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphate
  modified_base 5: mod_base = OTHER; note = 2'-O-methylcytidine-3'-phosphorothioate
  modified_base 6: mod_base = OTHER; note = 2'-O,4'-C-methylene adenosine-3'-phosphate
  modified_base 7: mod_base = OTHER; note = 2'-O-methylcytidine-3'-phosphate
  modified_base 8: mod_base = OTHER; note = 2'-O-methyladenosine-3'-phosphorothioate
  modified_base 9: mod_base = OTHER; note = 2'-O-methyladenosine-3'-phosphate
  modified_base 10: mod_base = OTHER; note = 2'-O-methyluridine-3'-phosphorothioate
  modified_base 11: mod_base = OTHER; note = 2'-O-methylguanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 112 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphate
  modified_base 5: mod_base = OTHER; note = 2'-O-methylcytidine-3'-phosphate
  modified_base 6: mod_base = OTHER; note = 2'-O,4'-C-methylene adenosine-3'-phosphate
  modified_base 7: mod_base = OTHER; note = 2'-O-methylcytidine-3'-phosphorothioate
  modified_base 8: mod_base = OTHER; note = 2'-O-methyladenosine-3'-phosphate
  modified_base 9: mod_base = OTHER; note = 2'-O-methyladenosine-3'-phosphorothioate
  modified_base 10: mod_base = OTHER; note = 2'-O-methyluridine-3'-phosphate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphate
  modified_base 17: mod_base = OTHER; note = 2'-O-methyladenosine-3'-phosphorothioate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 113 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphate
  modified_base 5: mod_base = OTHER; note = 2'-O-methylcytidine-3'-phosphate
  modified_base 6: mod_base = OTHER; note = 2'-O,4'-C-methylene adenosine-3'-phosphate
  modified_base 7: mod_base = OTHER; note = 2'-O-methylcytidine-3'-phosphate
  modified_base 8: mod_base = OTHER; note = 2'-O-methyladenosine-3'-phosphorothioate
  modified_base 9: mod_base = OTHER; note = 2'-O-methyladenosine-3'-phosphate
  modified_base 10: mod_base = OTHER; note = 2'-O-methyluridine-3'-phosphate
  modified_base 11: mod_base = OTHER; note = 2'-O-methylguanosine-3'-phosphorothioate
  modified_base 12: mod_base = OTHER; note = 2'-O,4'-C-methylene adenosine-3'-phosphate
  modified_base 13: mod_base = OTHER; note = 2'-O-methylguanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphate
  modified_base 17: mod_base = OTHER; note = 2'-O-methyladenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 114 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphate
  modified_base 5: mod_base = OTHER; note = 2'-O-methylcytidine-3'-phosphate
  modified_base 6: mod_base = OTHER; note = 2'-O,4'-C-methylene adenosine-3'-phosphate
  modified_base 7: mod_base = OTHER; note = 2'-O-methylcytidine-3'-phosphate
  modified_base 8: mod_base = OTHER; note = 2'-O-methyladenosine-3'-phosphate
  modified_base 9: mod_base = OTHER; note = 2'-O-methyladenosine-3'-phosphorothioate
  modified_base 10: mod_base = OTHER; note = 2'-O-methyluridine-3'-phosphate
  modified_base 11: mod_base = OTHER; note = 2'-O-methylguanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 115 - DNA, 19 bp, synthetic construct
  source 1..19: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphorothioate
  modified_base 4: mod_base = OTHER; note = 2'-O-methylcytidine-3'-phosphate
  modified_base 5: mod_base = OTHER; note = 2'-O-methylcytidine-3'-phosphate
  modified_base 6: mod_base = OTHER; note = 2'-O,4'-C-methylene adenosine-3'-phosphate
  modified_base 7: mod_base = OTHER; note = 2'-O-methylcytidine-3'-phosphate
  modified_base 8: mod_base = OTHER; note = 2'-O-methyladenosine-3'-phosphate
  modified_base 9: mod_base = OTHER; note = 2'-O-methyladenosine-3'-phosphorothioate
  modified_base 10: mod_base = OTHER; note = 2'-O-methyluridine-3'-phosphate
  modified_base 11: mod_base = OTHER; note = 2'-O-methylguanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphate
  modified_base 14: mod_base = OTHER; note = 2'-O,4'-C-methylene adenosine-3'-phosphate
  modified_base 15: mod_base = OTHER; note = 2'-O-methylguanosine-3'-phosphorothioate
  modified_base 16: mod_base = OTHER; note = 2'-O-methyluridine-3'-phosphorothioate
  modified_base 17: mod_base = OTHER; note = 2'-O-methyladenosine-3'-phosphate
  modified_base 18: mod_base = OTHER; note = 2'-O,4'-C-methylene 5-methylcytidine-3'-phosphate
  modified_base 19: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..18: note = RNA
  misc_feature 19: note = DNA
catccacaatgagagtaca

SEQ ID NO: 116 - DNA, 23 bp, synthetic construct
  source 1..23: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphate
  modified_base 4: mod_base = OTHER; note = 2'-O-methyluridine-3'-phosphate
  modified_base 5: mod_base = OTHER; note = 2'-O-methylcytidine-3'-phosphorothioate
  modified_base 6: mod_base = OTHER; note = 2'-O-methyladenosine-3'-phosphorothioate
  modified_base 7: mod_base = OTHER; note = 2'-O-methyluridine-3'-phosphorothioate
  modified_base 8: mod_base = OTHER; note = 2'-O-methylcytidine-3'-phosphate
  modified_base 9: mod_base = OTHER; note = 2'-O-methylcytidine-3'-phosphate
  modified_base 10: mod_base = OTHER; note = 2'-O,4'-C-methylene adenosine-3'-phosphate
  modified_base 11: mod_base = OTHER; note = 2'-O-methylcytidine-3'-phosphate
  modified_base 12: mod_base = OTHER; note = 2'-O-methyladenosine-3'-phosphate
  modified_base 13: mod_base = OTHER; note = 2'-O-methyladenosine-3'-phosphorothioate
  modified_base 14: mod_base = OTHER; note = 2'-O-methyluridine-3'-phosphate
  modified_base 15: mod_base = OTHER; note = 2'-O-methylguanosine-3'-phosphate
  modified_base 16: mod_base = OTHER; note = 2'-O,4'-C-methylene adenosine-3'-phosphate
  modified_base 17: mod_base = OTHER; note = 2'-O-methylguanosine-3'-phosphate
  modified_base 18: mod_base = OTHER; note = 2'-O,4'-C-methylene adenosine-3'-phosphate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine-3'-phosphate
  modified_base 22: mod_base = OTHER; note = 2'-O,4'-C-methylene 5-methylcytidine-3'-phosphate
  modified_base 23: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..22: note = RNA
  misc_feature 23: note = DNA
tcgtcatccacaatgagagtaca

SEQ ID NO: 117 - DNA, 17 bp, synthetic construct
  source 1..17: mol_type = other DNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methylcytidine-3'-phosphorothioate
  modified_base 4: mod_base = OTHER; note = 2'-O,4'-C-methylene adenosine-3'-phosphate
  modified_base 5: mod_base = OTHER; note = 2'-O-methylcytidine-3'-phosphate
  modified_base 6: mod_base = OTHER; note = 2'-O-methyladenosine-3'-phosphate
  modified_base 7: mod_base = OTHER; note = 2'-O-methyladenosine-3'-phosphorothioate
  modified_base 8: mod_base = OTHER; note = 2'-O-methyluridine-3'-phosphate
  modified_base 9: mod_base = OTHER; note = 2'-O-methylguanosine-3'-phosphate
  modified_base 10: mod_base = OTHER; note = 2'-O,4'-C-methylene adenosine-3'-phosphate
  modified_base 11: mod_base = OTHER; note = 2'-O-methylguanosine-3'-phosphate
  modified_base 12: mod_base = OTHER; note = 2'-O,4'-C-methylene adenosine-3'-phosphate
  modified_base 13: mod_base = OTHER; note = 2'-O-methylguanosine-3'-phosphorothioate
  modified_base 14: mod_base = OTHER; note = 2'-O-methyluridine-3'-phosphorothioate
  modified_base 15: mod_base = OTHER; note = 2'-O-methyladenosine-3'-phosphate
  modified_base 16: mod_base = OTHER; note = 2'-O,4'-C-methylene 5-methylcytidine-3'-phosphate
  modified_base 17: mod_base = OTHER; note = 2'-deoxyadenosine-3'-phosphate ((2S,4R)-1-[29-[[2-( acetylamino)-2-deoxy-beta-D-galactopyranosyl]oxy]-14,14-bi s[[3-[[3-[[5-[[2-(acetylamino)-2-deoxy-beta-D-galactopyran osyl]oxy]-1-oxopentyl]amino]propyl]amino]-3-oxopropoxy]met hyl]-1,12,19,25-tetraoxo-16-oxa-13,20,24-triazanonacos-1-y l]-4-hydroxy-2-pyrrolidinyl)methyl ester
  misc_feature 1..16: note = RNA
  misc_feature 17: note = DNA
tccacaatgagagtaca

SEQ ID NO: 118 - RNA, 21 nt, synthetic construct
  source 1..21: mol_type = other RNA
  modified_base 18: mod_base = OTHER; note = 5'-methyluridine-3'-phosphate
gctatcctgggcaaaaatcaa

SEQ ID NO: 119 - RNA, 19 nt, synthetic construct
  source 1..19: mol_type = other RNA
  modified_base 16: mod_base = OTHER; note = 5'-methyluridine-3'-phosphate
catccacaatgagagtaca

SEQ ID NO: 120 - RNA, 19 nt, synthetic construct
  source 1..19: mol_type = other RNA
  modified_base 16: mod_base = OTHER; note = 5'-methyluridine-3'-phosphate
catccacaatgagagtaca

SEQ ID NO: 121 - RNA, 19 nt, synthetic construct
  source 1..19: mol_type = other RNA
  modified_base 16: mod_base = OTHER; note = 5'-methyluridine-3'-phosphate
catccacaatgagagtaca

SEQ ID NO: 122 - RNA, 19 nt, synthetic construct
  source 1..19: mol_type = other RNA
  modified_base 16: mod_base = OTHER; note = 5'-methyluridine-3'-phosphate
catccacaatgagagtaca

SEQ ID NO: 123 - RNA, 19 nt, synthetic construct
  source 1..19: mol_type = other RNA
catccacaatgagagtaca

SEQ ID NO: 124 - RNA, 19 nt, synthetic construct
  source 1..19: mol_type = other RNA
  modified_base 10: mod_base = OTHER; note = 5'-methyluridine-3'-phosphate
catccacaatgagagtaca

SEQ ID NO: 125 - RNA, 17 nt, synthetic construct
  source 1..17: mol_type = other RNA
  modified_base 14: mod_base = OTHER; note = 5'-methyluridine-3'-phosphate
tccacaatgagagtaca

SEQ ID NO: 126 - RNA, 17 nt, synthetic construct
  source 1..17: mol_type = other RNA
tccacaatgagagtaca

SEQ ID NO: 127 - RNA, 17 nt, synthetic construct
  source 1..17: mol_type = other RNA
tccacaatgagagtaca

SEQ ID NO: 128 - RNA, 16 nt, synthetic construct
  source 1..16: mol_type = other RNA
ccacaatgagagtaca

SEQ ID NO: 129 - RNA, 19 nt, synthetic construct
  source 1..19: mol_type = other RNA
catccacaatgagagtaca

SEQ ID NO: 130 - RNA, 19 nt, synthetic construct
  source 1..19: mol_type = other RNA
catccacaatgagagtaca

SEQ ID NO: 131 - RNA, 19 nt, synthetic construct
  source 1..19: mol_type = other RNA
catccacaatgagagtaca

SEQ ID NO: 132 - RNA, 19 nt, synthetic construct
  source 1..19: mol_type = other RNA
catccacaatgagagtaca

SEQ ID NO: 133 - RNA, 19 nt, synthetic construct
  source 1..19: mol_type = other RNA
catccacaatgagagtaca

SEQ ID NO: 134 - RNA, 19 nt, synthetic construct
  source 1..19: mol_type = other RNA
catccacaatgagagtaca

SEQ ID NO: 135 - RNA, 19 nt, synthetic construct
  source 1..19: mol_type = other RNA
catccacaatgagagtaca

SEQ ID NO: 136 - RNA, 19 nt, synthetic construct
  source 1..19: mol_type = other RNA
catccacaatgagagtaca

SEQ ID NO: 137 - RNA, 19 nt, synthetic construct
  source 1..19: mol_type = other RNA
catccacaatgagagtaca

SEQ ID NO: 138 - RNA, 19 nt, synthetic construct
  source 1..19: mol_type = other RNA
catccacaatgagagtaca

SEQ ID NO: 139 - RNA, 23 nt, synthetic construct
  source 1..23: mol_type = other RNA
tcgtcatccacaatgagagtaca

SEQ ID NO: 140 - RNA, 17 nt, synthetic construct
  source 1..17: mol_type = other RNA
tccacaatgagagtaca